(19) United States
(12) Patent Application Publication
(10) Pub. No.: US 2026/0002180 A1
(43) Pub. Date: Jan. 1, 2026

(54) PLATFORM FOR BIO-BASED PRODUCTION OF HIGH LEVELS OF O-PHOSPHOSERINE, CYSTEATE, OR TAURINE

(71) Applicant: Natáur Inc., Baltimore, MD (US)

(72) Inventor: Frank Turano, Baltimore, MD (US)

(73) Assignee: Natáur Inc., Baltimore, MD (US)

(21) Appl. No.: 19/317,818

(22) Filed: Sep. 3, 2025

Related U.S. Application Data
(63) Continuation of application No. PCT/US2025/023839, filed on Apr. 9, 2025.
(60) Provisional application No. 63/632,872, filed on Apr. 11, 2024.

(51) Int. Cl. C12P 11/00; C12N 9/10; C12N 9/88; C12N 15/52

(52) U.S. Cl. CPC C12P 11/00; C12N 9/1085; C12N 9/88; C12N 15/52; C12Y 205/01076; C12Y 401/01011; C12Y 401/01015

(57) ABSTRACT

Methods for the fermentative production of O-phosphoserine, cysteate, or taurine in microbes or unicellular organisms that contain a serB mutation that either decreases serB expression, reduces the amount of the serB gene product or results in a serB gene product with low enzymatic activity. Genetic modifications of the O-phosphoserine, cysteate, or taurine and/or substrate biosynthetic pathways in unicellular organisms that include bacteria, algae, microalgae, diatoms, yeast, or fungi are disclosed. Also disclosed are fermentation and processing methods for the production of various O-phosphoserine-, cysteate-, or taurine-containing products and the use of the cells, fermentation broth or extracts that contain O-phosphoserine, cysteate, or taurine to produce products for use in food, feed, beverages, dietary and health supplements, cosmetics, personal care, pharmaceuticals, agricultural production, or surfactants.

### CROSS-REFERENCE TO RELATED APPLICATIONS

This application is a continuation of International Application No. PCT/US2025/023839, filed Apr. 9, 2025, which claims priority to, and the benefit of, U.S. Provisional Application No. 63/632,872, filed on Apr. 11, 2024, the entire contents of which are incorporated by reference herein in their entirety for all purposes.

### SEQUENCE LISTING

The present application is being filed along with a Sequence Listing in electronic format. The Sequence Listing is entitled “5130_0105_seq.xml,” created on Aug. 29, 2025, and is 455,439 bytes in size. The information in the electronic format of the Sequence Listing is incorporated herein by reference in its entirety.

### FIELD

The present invention is in the field of the production of O-phosphoserine, cysteate, or taurine by microbes or unicellular organisms.

### BACKGROUND

### O-Phosphoserine

In all organisms, O-phosphoserine is the precursor for the synthesis of the amino acid, serine, and, in eukaryotic proteins, it is the most prevalent post-translational modification.[1] In animals and humans, O-phosphoserine is a regulator of neural stem cell proliferation and differentiation.[2] O-phosphoserine can be used in composites and scaffolds to promote bone tissue regeneration [3-5] or in hard tissue cements and adhesives [6, 7] and hydrogels. [8] O-phosphoserine has been shown to play a neuroprotective role in primary open-angle glaucoma.[9] Additionally, O-phosphoserine can be directed into peptide synthesis by modifying the elongation factor-Tu. [1]

### Cysteate

Cysteate is a precursor for the synthesis of the sulfonic acid, taurine. Cysteate can be used in diets as a precursor of taurine for cats and is used in beauty creams and lotions.

### Taurine

Taurine, a sulfonic acid, is an essential nutrient for humans and animals. [11-16] It is needed for cardiovascular, skeletal muscle, vision, and nervous system function [17] and has been linked with overall human wellness and longevity. [11] Taurine is used as an ingredient, required in some cases by the FDA, in numerous products including infant formula, pet food, animal feed, energy drinks, nutraceuticals, pharmaceuticals, personal care/cosmetics, and plant growth enhancers. Taurine is naturally occurring in meat and other animal products [18] but little to no taurine is produced in plants. Taurine can be added as an ingredient in (or taken as a supplement to) plant-based food or feed diets to meet taurine nutritional requirements.[15, 19, 20]

### O-Phosphoserine Pathway

Carbon from glucose enters the serine biosynthetic pathway by conversion of glycerate 1,3-bisphosphate into glycerate 3-phosphate by the pgk gene product phosphoglycerate kinase.[21] Glycerate 3-phosphate is converted into 3-phosphohydroxypyruvate by the product of serA, 3-phosphoglycerate dehydrogenase. The serA gene product is sensitive to feedback inhibition by serine, however, the inhibition can be removed by the deletion of the last 197 amino acids (serA_{Δ197})[22]. 3-phosphohydroxypyruvate is converted into O-phosphoserine by the product of serC, phosphoserine aminotransferase, and O-phosphoserine is converted into serine by the product of serB, phosphoserine phosphatase. The increased availability of O-phosphoserine can be achieved by down regulating or silencing (knock out) serB or by decreasing the activity of the serB gene product, phosphoserine phosphatase, by mutagenesis or site-specific modification.

### Cysteate Production Via the O-Phosphoserine Pathway

O-phosphoserine and sulfite are converted into cysteate by cysteate synthase (CS) or by some threonine synthase (TS) gene products.[23]

### Taurine Production Via the O-Phosphoserine Pathway

Cysteate (cysteic acid) is converted into taurine by removal of a carboxyl-group by cysteate-specific, cysteic acid decarboxylase (CAD), or promiscuous decarboxylase, glutamate decarboxylase (GAD), sulfinoalanine decarboxylase (SAD), or the decarboxylase portion of the cysteine synthetase/PLP decarboxylase (partCS/PLP-DC), activity.

### PLP Requiring Enzymes

The enzymes, CS, TS, CAD, SAD, GAD and partCS/PLP-DC require the cofactor pyridoxal 5′-phosphate (PLP) for enzymatic activity. In biological systems there two different PLP biosynthesis pathways, the deoxy xylulose 5-phosphate (DXP)-dependent pathway and DXP-independent pathway, and a salvage PLP pathway [24]. To maximize O-phosphoserine, cysteate, or taurine production, PLP, pyridoxine, or a pyridoxine salt can be added to the media to increase the activity of the O-phosphoserine, cysteate, or taurine producing enzymes. Alternatively, endogenous PLP can be increased with increased expression of components of the PLP biosynthetic pathway [25-27] or by increasing the expression of components of the alterative PLP pathway [28, 29]. Genes in the PLP pathways include gapA/B, pdxB, pdxF, pdxA, dxs, pdxJ, pdxH, pdxS and pdxT.

### Sulfur Metabolism

Sulfur-based precursors for cysteate or taurine biosynthesis come from the sulfur uptake and reduction pathways. The sulfate-thiosulfate uptake pathway is controlled by the products of sbp, cysP, cysU, cysW, and cysA. Sulfate and thiosulfate are bound by the products of sbp and cysP, respectively, and transported into the cell by the products of cysU, cysW, and cysA.[30] Sulfate is converted into 3′-phosphoadenosine-5′-phosphosulfate (PAPS) by the products of cysDNC, ATP sulfurylase and APS kinase. PAPS is converted into adenosine-3′,5′-diphosphate (PAP) and sulfite by the product of cysH, PAPS reductase. The product of cysQ, PAP nucleotidase, is involved in PAPS regeneration. Sulfite is converted into sulfide by the products of cysIJ. O-acetyl-L-serine and sulfide are converted into cysteine by CysK and CysM. CysM also synthesizes S-sulfocysteine from O-acetyl-L-serine and thiosulfate.[31] The S-sulfocysteine is converted into cysteine by glutaredoxin (NrdH) or Grx.

### O-Phosphoserine, Cysteate and Taurine Transporters

The regulation of the import and export of small molecules is an effective way to increase the production of targeted molecules during microbial fermentation. The deletion of uptake genes has been shown to increase production of several amino acids and small molecules. Conversely, increased export of several amino acids and small molecules have been shown to increase their production during fermentation. The silencing or deletion of genes and their corresponding peptides that may be involved in the import of O-phosphoserine, cysteate, or taurine include: sdaC, cycA, sstT, tdcC, or cyuP (yhaO). Increased expression of the genes and their corresponding gene products that may be involved in the export of O-phosphoserine, cysteate, or taurine include: emaA (ydeD), alaE (ygaW), yfik, cefA, cefB, rhtA, rhtB, rhtC, gabP, tauP, gadC or yhiM.

### Sulfonic Acid Degradation

In the absence of sulfur, bacteria utilize the sulfonic acid uptake and degradation pathway or the taurine uptake and degradation pathway to mobilize carbon, nitrogen or sulfur.[33-36] Genes and their corresponding peptides involved in the uptake and degradation of taurine are usually on the same operon, tauABCD[37] and ssuEADCB,[38] and induced in the absence of nitrogen[39, 40] or sulfur[33] or in the presence of taurine.[36, 41] In other bacteria, such as C. glutamicum, the genes and their corresponding peptides involved in sulfonic acid, taurine, uptake and degradation are in the ssuDICBA and sueABCD2 operons.[42] The above mentioned pathways also import and degrade cysetate.

The genes for the taurine degradation enzymes, tauX and tauY, encode taurine dehydrogenase (TDH).[40] tauD encodes taurine dioxygenase (TDO),[33] tpa encodes taurine-pyruvate aminotransferase (TPAT),[43] and ssuD and ssuE encode the two-component alkanesulfonate monooxygenase, 2CASM.[34] The above mentioned pathways also import and degrade cysteate.

### Transcriptional Regulators

Several global regulators of sulfur metabolism exist in bacteria. The cysB gene product is a LysR-type transcriptional activator of genes involved in sulfur uptake and reduction and cysteine metabolism. CysB is highly conserved in gram-negative bacteria.[44] In Corynebacterium glutamicum, a transcriptional regulator, methionine/cysteine biosynthetic repressor (McbR),[45] represses the expression of genes involved in sulfur assimilation and cysteine biosynthesis. The translational regulators, Cbl and TauR, control the expression and induction of the taurine degradation pathways in bacteria.[33, 43] Cbl is a LysR-type transcriptional regulator of the sulfonic acid uptake and degradation pathway or the taurine uptake and degradation pathway in several bacteria.[38, 46] The cbl gene is found in Proteobacteria including members of the Alphaproteobacteria, Betaproteobacteria, and Gammaproteobacteria. Bacteria that lack Cbl transcriptional regulators have a member of the McbR subfamily of activators, which include TauR, that control the taurine uptake and degradation system. TauR is found in Rhizobiales and Rhodobacterales of the Alphaproteobacteria, in Burkholderiaceae and Comamonadaceae of the Betaproteobacteria, in Enterobacteriales, Oceanospirillales and Psychromonadales from the Gammaproteobacteria, and in Rhizobiales and Rhodobacter of the Alphaproteobacteria. The above-mentioned pathways also regulate the importation and degradation of cysteate.

### Fermentation Conditions and Nutrient Media

In the described invention O-phosphoserine, cysteate, or taurine is produced by fermentation. Methods to produce chemical compounds by batch fermentation, fed-batch fermentation, continuous fermentation or in tanks or ponds are well known to one with ordinary skill in the art.[47-57] The culture medium to be used in the present invention is dependent upon the requirements of the microorganism used in production. Descriptions of defined media for various microorganisms are found in the literature.[58-60] Carbon sources can be used individually or combined and can include sugar and carbohydrates such as glucose, sucrose, lactose, fructose, maltose, molasses, starch and cellulose, oils and fats, fatty acids, alcohols, and organic acids. Nitrogen sources can be used individually or as a mixture and can include organic nitrogen-containing compounds such as peptones, tryptone, casein amino acids, yeast extract, meat extract, malt extract, corn steep liquor, soybean meal and urea or inorganic compounds such as ammonium sulfate, ammonium chloride, ammonium phosphate, ammonium carbonate and ammonium nitrate. Potassium and phosphate sources can include potassium chloride, monopotassium phosphate, dipotassium phosphate, monosodium phosphate, and disodium phosphate. Magnesium sulfate or iron sulfate, micronutrients, amino acids and vitamins are also necessary for growth.

To control the pH of the culture, compounds such as sodium hydroxide, potassium hydroxide, ammonia, ammonium hydroxide or acids such as phosphoric acid or sulfuric acid are used. To control the foaming, anti-foaming agents are used. Aerobic conditions are maintained by mixing or introducing air or oxygen into the culture. The dissolved oxygen is 15% to 40%, depending on the growth phase and microorganism. The temperature of the culture is 25° C. to 40° C., preferably at 30° C. to 37° C., depending on the microorganism. Growth of the cell culture is maintained until maximum O-phosphoserine, cysteate, or taurine production is reached, typically within 10 hours to 100 hours, preferably 15 hours to 30 hours.

In the described invention, the fermentation broth contains O-phosphoserine, cysteate, or taurine, the cell mass of the microorganism, organic by-products of the fermentative process, and any remaining components of the medium.

The concentration of O-phosphoserine, cysteate, or taurine synthesized can be determined at various times throughout fermentation using thin layer chromatography (TLC), amino acid analyzers, high-performance liquid chromatography (HPLC), mass spectrometry (MS), electrospray ionization mass spectrometry (ESI-MS), and liquid chromatography tandem mass spectrometry (LC-MS/MS).

### Downstream Processing: Separation and Purification

In the described invention O-phosphoserine, cysteate, or taurine is processed or purified to make a product. The specific downstream processing to be used is dependent upon several factors including whether O-phosphoserine, cysteate, or taurine exists in the cells (or biomass) or in the liquid, the form of the desired final O-phosphoserine, cysteate, or taurine product such as liquid or powder, and the desired purity and/or moisture level. In some product applications, the processing may include drying the cells and media to the appropriate concentration and dryness. In some product applications, the processing may include purifying or partially purifying the O-phosphoserine, cysteate, or taurine. To decrease cost and increase efficiency, the volume can be decreased at various times throughout downstream processing by concentrating or removing water by evaporation, using e.g. a falling film evaporator, reverse osmosis or nanofiltration.

If the O-phosphoserine, cysteate, or taurine is in the liquid of the fermentation broth, the liquid can be separated from the biomass by centrifugation, filtration, decantation, or a combination thereof. Additional processing of the O-phosphoserine-, cysteate-, or taurine-containing liquid may include concentration or drying or a purification step for the manufacturing of a O-phosphoserine, cysteate, or taurine product according to the invention. The purification step may be selected from the group consisting of chromatographic techniques[51] or membrane-based processes[61] including ion exchange chromatography[61], ultra-filtration, precipitation, pH adjustment and nanofiltration,[62] treatment with activated carbon[63] or crystallization. The purification step or any combination thereof may be repeated until the O-phosphoserine, cysteate, or taurine is purified to the desired specification such as for purity and moisture.

If the O-phosphoserine, cysteate, or taurine is in the cells of the fermentation broth, the cells can be separated from the liquid by centrifugation, filtration, decantation, or a combination thereof. The O-phosphoserine-, cysteate-, or taurine-containing cells can be concentrated and used as a product or the cells can be disrupted by chemical agents, pressure, mechanical force, or ultrasonification to release their contents. The disrupted cells with their contents can be concentrated or dried and used as a product or the contents can be further processed to produce single cell proteins that can be concentrated or dried for use as a product. Alternatively, O-phosphoserine, cysteate, or taurine in the disrupted cells can be separated from the cellular debris by centrifugation, filtration or decantation or a combination thereof, followed by further purification as described above.

If the O-phosphoserine, cysteate, or taurine is in both the liquid and the cells in the fermentation broth, the liquid and cells can be separated, and treated separately, as described above or concentrated together. The O-phosphoserine-, cysteate-, or taurine-containing concentrate can be used for the manufacturing of a product according to the invention or further processed by purification as described above.

The O-phosphoserine-, cysteate-, or taurine-containing product can be in different forms such as liquid, powder, paste, capsule or tablet.

### SUMMARY

The present invention provides methods for a cost-effective fermentative production of O-phosphoserine, cysteate, or taurine by microbes or unicellular organisms. Methods are presented for the optimization of O-phosphoserine, cysteate, or taurine production through genetic improvements of microbes or unicellular organisms, refinement of growth and fermentation conditions and nutrient media, and enhancement of downstream processing or purification of O-phosphoserine, cysteate, or taurine.

The invention provides methods for the fermentative production of O-phosphoserine, cysteate, or taurine in microbes or unicellular organisms that contain a serB mutation that either decreases serB expression, reduces the amount of the serB gene product or results in a serB gene product with low enzymatic activity. More particularly, the invention encompasses the use of serB mutants with polynucleotides for O-phosphoserine, cysteate, or taurine biosynthetic enzymes in combination with polynucleotides sulfur (sulfate, or thiosulfate) uptake, reduction and assimilation and/or the use of polynucleotides for peptides that degrade or transport O-phosphoserine, cysteate, or taurine to increase O-phosphoserine, cysteate, or taurine in cells or export O-phosphoserine, cysteate, or taurine into the media. The invention also relates to fermentation and processing methods for the production of various products produced from the cells, fermentation broth or extracts that contain O-phosphoserine, cysteate, or taurine.

For purposes of promoting an understanding of the principles of the invention, reference will now be made to particular embodiments of the invention and specific language will be used to describe the same. The materials, methods and examples are illustrative only and not limiting.

In some embodiments, the unicellular organisms contain one or more exogenous polynucleotides that is operably linked to a promoter. In other embodiments, the expression of the endogenous polynucleotides of the microbe or unicellular organisms is modified with an exogenous promoter.

In one embodiment, the invention consists of microbes or unicellular organisms that have a mutant serB.

In one embodiment, the invention consists of microbes or unicellular organisms that have a mutant serB and a cysteate biosynthetic gene containing the exogenous polynucleotides, TS or CS, and a modified sulfur-based pathway to have increased expression of cysPUWA, cysDNC, cysQ, and knock-outs of tauD, ssuD, and ssuE to inhibit cysteate degradation or knock-outs of cuyA, tauABCD, ssuEADCB, ssuDICBA or sueABCD2 to inhibit degradation and reuptake of cysteate into the cell.

In one embodiment, the invention consists of microbes or unicellular organisms that have a mutant serB and a taurine biosynthetic pathway containing the exogenous polynucleotides, TS or CS and SAD, CAD, GAD or partCS/PLP-DC and a modified sulfur-based pathway to have increased expression of cysPUWA, cysDNC, cysQ, and knock-outs of tauD, ssuD, and ssuE to inhibit taurine degradation or knock-outs of tauABCD, ssuEADCB, ssuDICBA or sueABCD2 to inhibit degradation and reuptake of taurine into the cell.

In one embodiment, the invention consists of microbes or unicellular organisms that have a mutant serB and a taurine biosynthetic pathway containing the exogenous polynucleotides, TS or CS and CS and SAD, CAD, GAD or partCS/PLP-DC, and a modified O-phosphoserine pathway to have increased expression of pgk, serA_{Δ197}, or serC and a modified sulfur-based pathway to have increased expression of cysPUWA, cysDNC, cysQ or cysH, and knock-outs of tauD, ssuD, and ssuE to inhibit taurine degradation or knock-outs of tauABCD, ssuEADCB, ssuDICBA or sueABCD2 to inhibit taurine degradation and reuptake of taurine into the cell.

In another embodiment, the invention consists of unicellular organisms that have a O-phosphoserine, cysteate, or taurine biosynthetic pathway with silenced or deletion of genes and their corresponding peptides for the import of O-phosphoserine, cysteate, or taurine, these may include one or more of the following: sdaC, cycA, sstT, tdcC, or cyuP (yhaO).

In another embodiment, the invention consists of unicellular organisms that have a O-phosphoserine, cysteate, or taurine biosynthetic pathway with increased expression of the genes for export of O-phosphoserine, cysteate, or taurine, the genes may include one or more of the following: emaA (ydeD), alaE (ygaW), yfik, cefA, cefB, rhtA, rhtB, rhtC, gabP, tauP, gadC, yhiM or AAperm.

In another embodiment, the invention consists of unicellular organisms that have peptides of the O-phosphoserine, cysteate, or taurine biosynthetic pathway in close proximity to each other or to peptides involved in the production of precursors or to exporters of O-phosphoserine, cysteate, or taurine by forming molecular scaffolds, channels, or cages.

In another embodiment, the invention consists of unicellular organisms that produce O-phosphoserine, cysteate, or taurine production is increased with addition of exogenous PLP, pyridoxine or a pyridoxine salt or the organism has increased expression of one of more genes in the PLP-biosynthetic pathway to increase production of O-phosphoserine, cysteate, or taurine.

The invention includes modified or mutant unicellular organisms including bacteria, yeast, fungi, or unicellular algae that produce O-phosphoserine, cysteate, or taurine for use in food, feed, beverages, dietary and health supplements, cosmetics, personal care, pharmaceuticals, or agricultural production.

The invention also describes methods to grow the cells by fermentation and describes media formulations in which to grow the cells for the production of O-phosphoserine, cysteate, or taurine or a O-phosphoserine, cysteate, or taurine product that may be a liquid, powder, paste, capsule or tablet.

The invention also describes methods to process the cells or the media in which the cells were grown to make a range of products that include pure O-phosphoserine, cysteate, or taurine or a O-phosphoserine-, cysteate-, or taurine-containing product.

### BRIEF DESCRIPTION OF THE DRAWINGS

FIG. 1. Sequence alignment of E. coli serB gene products from wild-type (WT; SEQ ID NO: 40) and mutant genes: a single mutation (D128N or M147T) and a double mutation (D128N & M147T) (SEQ ID NO:258; SEQ ID NO:260; SEQ ID NO:262). Mutated amino acid residues are indicated in bold.

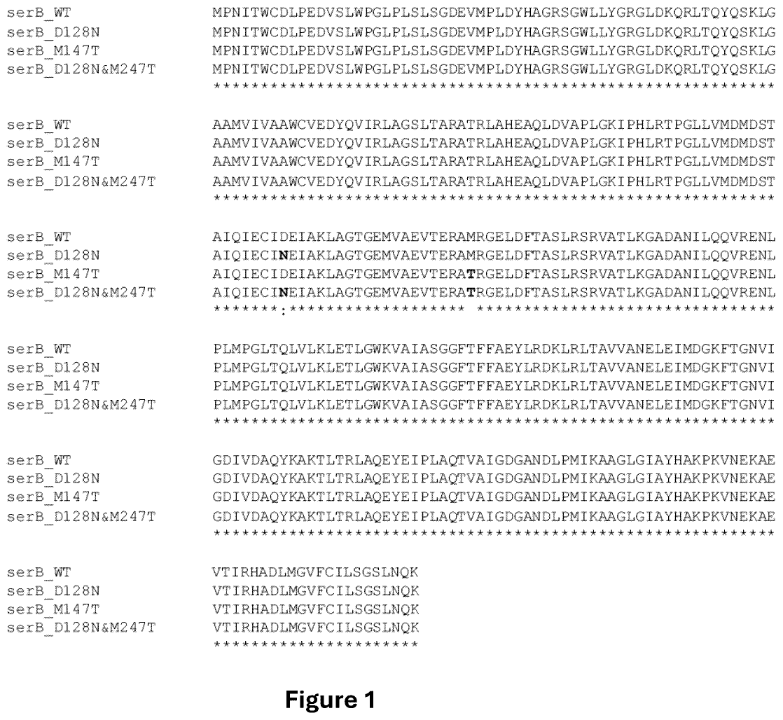

FIG. 2. Results from HPLC analysis of taurine-producing microbes that contain a WT serB (Red) or mutant M147T serB (Blue). Samples were analyzed by HPLC. O-phosphoserine, NH3, and taurine are indicated. The taurine level in the microbe containing the serB_M147T mutation is 7× higher than that of the microbe containing the WT serB.

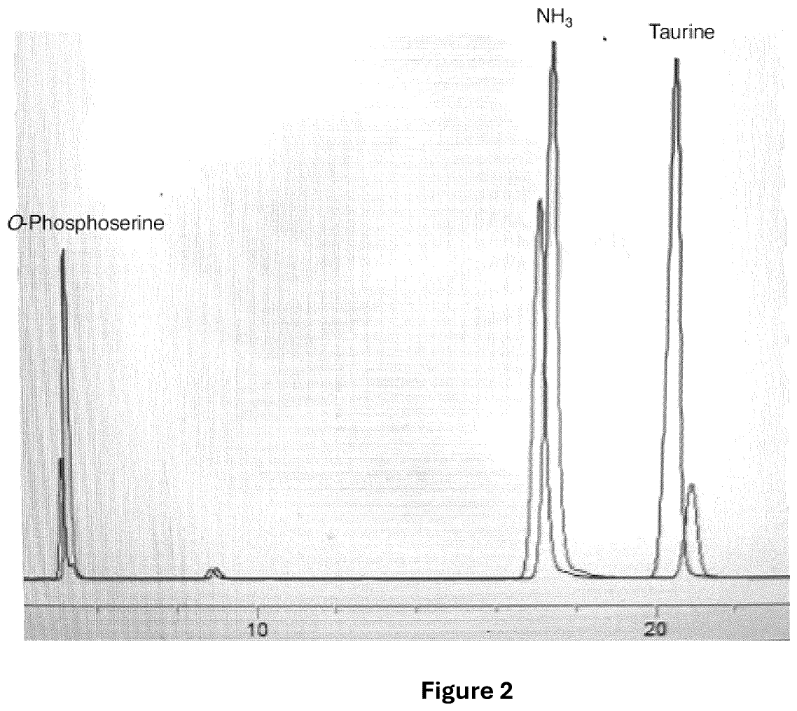

### DETAILED DESCRIPTION

The present invention provides methods for the production of O-phosphoserine, cysteate, or taurine in microbes or unicellular organisms that contain a serB mutation that either decreases serB expression, reduces the amount of the serB gene product or results in a serB gene product with low enzymatic activity. In preferred embodiments, the invention provides methods for the genetic modification of unicellular organisms with the serB mutation and genes that encode proteins in the biosynthetic pathway, the O-phosphoserine pathway, and for the increased transport, reduction and assimilation of sulfur together with silenced or knocked-out genes for the degradation of O-phosphoserine, cysteate, or taurine or precursors or knocked-out operons for O-phosphoserine, cysteate, or taurine uptake and degradation. The invention also provides methods of using unicellular organisms including bacteria, microalgae, fungi, yeast, and algae with increased levels of endogenous O-phosphoserine, cysteate, or taurine for use in food, feed, beverages, dietary and health supplements, cosmetics, personal care, pharmaceuticals, bio-matrices or agricultural production.

This invention presents methods for the modification of microbes or unicellular organisms with at least one serB mutation to increase O-phosphoserine or combined with one or more exogenous polynucleotides for peptides from one or more cysteate, or taurine biosynthetic pathway consisting of the groups: Group 1: CS or TS or Group 2: CS or TS and SAD, GAD, CAD or partCS/PLP-DC.

This invention presents methods for the modification of unicellular organisms with at least one serB mutation with modifications to increase expression of one or more polynucleotides for peptides in O-phosphoserine-based or sulfate-based pathways comprising of: pgk, serA_{Δ197}, serC, nrdH, sbp, cysUWA, cysPUWA, cysDNC, cysQ, and cysH.

This invention presents methods for modification of unicellular organisms with at least one serB mutation with modifications to block O-phosphoserine, cysteate, or taurine uptake and degradation by silencing, mutating or knocking out one or more of the following operons: tauABCD, ssuEADCB, ssuDICBA or sueABCD2.

This invention presents methods for the modification of unicellular organisms with at least one serB mutation with modifications to block O-phosphoserine, cysteate, or taurine degradation or catabolism by methods of silencing, mutating or knocking out one or more of the following genes: tauX, tauY, tauD, tpa, ssuD, ssuE, or ssul.

This invention presents methods for the modification of unicellular organisms with at least one serB mutation with modifications to block cysteate degradation by methods of silencing, mutating or knocking out for the cysteate degradation enzyme: cuyA.

This invention presents methods for the modification of unicellular organisms with at least one serB mutation with modifications to control the expression of one or more translational regulator genes, cbl, cysB, tauR, or mcbR, in the sulfate-based, O-phosphoserine, cysteate, or taurine pathways.

Below is a non-limiting list of example polynucleotides that are suitable for of the invention. Other suitable polynucleotides for use in accordance with the invention may be obtained by the identification of polynucleotides by selective hybridize to the polynucleotides to the named polypeptide by hybridization under low stringency conditions, moderate stringency conditions, or high stringency conditions. Still other suitable polynucleotides for use in accordance with the invention may be obtained by the identification of similar polynucleotides that have substantial identity of the nucleic acid of or encode polypeptides that have substantial identity to amino acid sequence of when it used as a reference for sequence comparison.

Suitable polynucleotides for CDO are provided in SEQ ID NO:1; SEQ ID NO:3; SEQ ID NO: 5; SEQ ID NO:7 and encode the peptides with amino acid sequences of SEQ ID NO:2; SEQ ID NO: 4; SEQ ID NO:6; SEQ ID NO:8, respectively.

Suitable polynucleotides for SAD (CSAD) are provided in SEQ ID NO:9; SEQ ID NO:11; SEQ ID NO:13 and encode the peptides with amino acid sequences of SEQ ID NO:10; SEQ ID NO: 12; SEQ ID NO:14, respectively.

A suitable polynucleotide for GAD is provided in SEQ ID NO:15 and encodes the peptide with amino acid sequence of SEQ ID NO:16.

Suitable polynucleotides for CS/PLP-DC are provided in SEQ ID NO:17; SEQ ID NO: 78; SEQ ID NO:198; SEQ ID NO:200; SEQ ID NO:202 and encode the peptides with amino acid sequences of SEQ ID NO:18; SEQ ID NO:79; SEQ ID NO: 199; SEQ ID NO:201; SEQ ID NO: 203, respectively.

A suitable polynucleotide for ADO is provided in SEQ ID NO: 19 and encodes the peptide with amino acid sequence of SEQ ID NO:20.

Suitable polynucleotides for CL are provided in SEQ ID NO:21; SEQ ID NO:23 and encode the peptides with amino acid sequences of SEQ ID NO:22; SEQ ID NO:24, respectively.

Suitable polynucleotides for CS or TS are provided in SEQ ID NO:25; SEQ ID NO:27 and encode the peptides with amino acid sequences of SEQ ID NO:26; SEQ ID NO:28, respectively.

Suitable polynucleotides for ilvA are provided in SEQ ID NO:136; SEQ ID NO:140 and encode the peptides with amino acid sequences of SEQ ID NO:137; SEQ ID NO:141, respectively.

A suitable polynucleotide for ilvA_{L447F} is provided in SEQ ID NO:29 and encodes the peptide with amino acid sequence of SEQ ID NO:30.

Suitable polynucleotides for PAPS-AS are provided in SEQ ID NO:31; SEQ ID NO:33 and encode the peptides with amino acid sequences of SEQ ID NO:32; SEQ ID NO:34, respectively.

A suitable polynucleotide for pgk is provided in SEQ ID NO:35 and encodes the peptide with amino acid sequence of SEQ ID NO:36.

A suitable polynucleotide for serA_{Δ197} is provided in SEQ ID NO:37 and encodes the peptide with amino acid sequence of SEQ ID NO:38.

A suitable polynucleotide for serB is provided in SEQ ID NO:39 and encodes the peptide with amino acid sequence of SEQ ID NO:40.

A suitable polynucleotide for serC is provided in SEQ ID NO:41 and encodes the peptide with amino acid sequence of SEQ ID NO:42.

A suitable polynucleotide for cysE_{M20IR} is provided in SEQ ID NO:43 and encodes the peptide with amino acid sequence of SEQ ID NO:44.

Suitable polynucleotides for cysK are provided in SEQ ID NO:45; SEQ ID NO:147 and encode the peptides with amino acid sequences of SEQ ID NO:46; SEQ ID NO:148, respectively.

A suitable polynucleotide for cysDNC is provided in SEQ ID NO:47 and encodes the peptides with amino acid sequences of SEQ ID NO:48; SEQ ID NO:49; SEQ ID NO:50.

A suitable polynucleotide for cysQ is provided in SEQ ID NO:51 and encodes the peptide with amino acid sequence of SEQ ID NO:52.

A suitable polynucleotide for cysH is provided in SEQ ID NO:53 and encodes the peptide with amino acid sequence of SEQ ID NO:54.

A suitable polynucleotide for cysIJ is provided in SEQ ID NO:55 and encodes the peptides with amino acid sequences of SEQ ID NO:57; SEQ ID NO:56.

A suitable polynucleotide for cysB is provided in SEQ ID NO:58 and encodes the peptide with amino acid sequence of SEQ ID NO:59.

A suitable polynucleotide for tauX is provided in SEQ ID NO:60 and encodes the peptide with amino acid sequence of SEQ ID NO:61.

A suitable polynucleotide for tauY is provided in SEQ ID NO:62 and encodes the peptide with amino acid sequence of SEQ ID NO:63.

A suitable polynucleotide for tauD is provided in SEQ ID NO:64 and encodes the peptide with amino acid sequence of SEQ ID NO:65.

A suitable polynucleotide for tpa is provided in SEQ ID NO:66 and encodes the peptide with amino acid sequence of SEQ ID NO:67.

A suitable polynucleotide for tauABCD is provided in SEQ ID NO:68.

A suitable polynucleotide for ssuEADCB is provided in SEQ ID NO:69.

Suitable polynucleotides for ssuD are provided in SEQ ID NO:70; SEQ ID NO:72 and encode the peptides with amino acid sequences of SEQ ID NO:71; SEQ ID NO:73, respectively.

Suitable polynucleotides for ssuE are provided in SEQ ID NO:74; SEQ ID NO:76 and encode the peptides with amino acid sequences of SEQ ID NO:75; SEQ ID NO:77, respectively.

Suitable polynucleotides for ridA are provided in SEQ ID NO:80; SEQ ID NO:149; SEQ ID NO: 151 and encode the peptides with amino acid sequences of SEQ ID NO:81; SEQ ID NO: 150; SEQ ID NO:152, respectively.

A suitable polynucleotide for tdcF is provided in SEQ ID NO:82 and encodes the peptide with amino acid sequence of SEQ ID NO:83.

A suitable polynucleotide for rutC is provided in SEQ ID NO:84 and encodes the peptide with amino acid sequence of SEQ ID NO:85.

A suitable polynucleotide for cuyA is provided in SEQ ID NO:86 and encodes the peptide with amino acid sequence of SEQ ID NO:87.

Suitable polynucleotides for cbl are provided in SEQ ID NO: 88; SEQ ID NO:90 and encode the peptides with amino acid sequences of SEQ ID NO:89; SEQ ID NO:91, respectively.

Suitable polynucleotides for tauR are provided in SEQ ID NO:92; SEQ ID NO:94 and encode the peptides with amino acid sequences of SEQ ID NO:93; SEQ ID NO:95, respectively.

A suitable polynucleotide for mcbR is provided in SEQ ID NO:96 and encodes the peptide with amino acid sequence of SEQ ID NO:97.

A suitable polynucleotide for cysM is provided in SEQ ID NO:98 and encodes the peptide with amino acid sequence of SEQ ID NO:99.

Suitable polynucleotides for sdaA are provided in SEQ ID NO:100; SEQ ID NO: 102 and encode the peptides with amino acid sequences of SEQ ID NO:101; SEQ ID NO:103, respectively.

Suitable polynucleotides for glyA are provided in SEQ ID NO:104; SEQ ID NO:106 and encode the peptides with amino acid sequences of SEQ ID NO:105; SEQ ID NO:107, respectively.

A suitable polynucleotide for tnaA is provided in SEQ ID NO: 108 and encodes the peptide with amino acid sequence of SEQ ID NO:109.

A suitable polynucleotide for cysPUWA is provided in SEQ ID NO:110 and encodes the peptides with amino acid sequences of SEQ ID NO:111; SEQ ID NO:112; SEQ ID NO:113; SEQ ID NO:114.

A suitable polynucleotide for nrdh is provided in SEQ ID NO: 143 and encodes the peptide with amino acid sequence of SEQ ID NO:144.

A suitable polynucleotide for sbp is provided in SEQ ID NO:160 and encodes the peptide with amino acid sequence of SEQ ID NO:161.

A suitable polynucleotide for ssuC is provided in SEQ ID NO:162 and encodes the peptide with amino acid sequence of SEQ ID NO:163.

A suitable polynucleotide for ssuB is provided in SEQ ID NO:164 and encodes the peptide with amino acid sequence of SEQ ID NO:165.

A suitable polynucleotide for ssuA is provided in SEQ ID NO:166 and encodes the peptide with amino acid sequence of SEQ ID NO:167.

A suitable polynucleotide for ssuDICBA is provided in SEQ ID NO:168.

A suitable polynucleotide for ssul is provided in SEQ ID NO:169 and encodes the peptide with amino acid sequence of SEQ ID NO:170.

A suitable polynucleotide for sueA is provided in SEQ ID NO: 172 and encodes the peptide with amino acid sequence of SEQ ID NO:173.

A suitable polynucleotide for sueB is provided in SEQ ID NO:174 and encodes the peptide with amino acid sequence of SEQ ID NO:175.

A suitable polynucleotide for sueC is provided in SEQ ID NO: 176 and encodes the peptide with amino acid sequence of SEQ ID NO:177.

A suitable polynucleotide for sueD2 is provided in SEQ ID NO:178 and encodes the peptide with amino acid sequence of SEQ ID NO:179.

A suitable polynucleotide for sueABCD2 is provided in SEQ ID NO:180.

Suitable polynucleotides for gadC are provided in SEQ ID NO:184; SEQ ID NO:186; SEQ ID NO:188 and encode the peptides with amino acid sequences of SEQ ID NO:185; SEQ ID NO: 187; SEQ ID NO:189, respectively.

A suitable polynucleotide for yhiM is provided in SEQ ID NO:190 and encodes the peptide with amino acid sequence of SEQ ID NO:191.

Suitable polynucleotides for AAperm are provided in SEQ ID NO:192; SEQ ID NO:194; SEQ ID NO:196 and encode the peptides with amino acid sequences of SEQ ID NO:193; SEQ ID NO: 1195; SEQ ID NO:197, respectively.

Suitable polynucleotides for cysteate, or taurine transporters or exporters are provided in SEQ ID NO:204; SEQ ID NO:206; SEQ ID NO:208; SEQ ID NO:210; SEQ ID NO:212; SEQ ID NO: 214; SEQ ID NO:216; SEQ ID NO:218; SEQ ID NO:220; SEQ ID NO:222 and encode the peptides with amino acid sequences of SEQ ID NO:205; SEQ ID NO:207; SEQ ID NO:209; SEQ ID NO:211; SEQ ID NO:213; SEQ ID NO:215; SEQ ID NO:217; SEQ ID NO:219; SEQ ID NO: 221; SEQ ID NO:223, respectively.

Suitable polynucleotides for O-phosphoserine, transporters or exporters are provided in SEQ ID NO:204; SEQ ID NO:206; SEQ ID NO:208; SEQ ID NO:210; SEQ ID NO:212; SEQ ID NO: 214; SEQ ID NO:216; SEQ ID NO:218; SEQ ID NO:220; SEQ ID NO:222 and encode the peptides with amino acid sequences of SEQ ID NO:205; SEQ ID NO:207; SEQ ID NO:209; SEQ ID NO:211; SEQ ID NO:213; SEQ ID NO:215; SEQ ID NO:217; SEQ ID NO:219; SEQ ID NO: 221; SEQ ID NO:223, respectively.

Suitable polynucleotides for cysteate, or taurine transporters or importers are provided in SEQ ID NO:224; SEQ ID NO:226; SEQ ID NO:228; SEQ ID NO:230; SEQ ID NO:232 and encode the peptides with amino acid sequences of SEQ ID NO:225; SEQ ID NO:227; SEQ ID NO: 229; SEQ ID NO:231; SEQ ID NO:233, respectively.

Suitable polynucleotides for O-phosphoserine, cysteate, or taurine transporters or importers are provided in SEQ ID NO:224; SEQ ID NO:226; SEQ ID NO:228; SEQ ID NO:230; SEQ ID NO:232 and encode the peptides with amino acid sequences of SEQ ID NO:225; SEQ ID NO: 227; SEQ ID NO:229; SEQ ID NO:231; SEQ ID NO:233, respectively.

Suitable polynucleotides for pdxS are provided in SEQ ID NO:242; SEQ ID NO:244 and encode the peptides with amino acid sequences of SEQ ID NO:243; SEQ ID NO:245, respectively.

Suitable polynucleotides for pdxT are provided in SEQ ID NO:246; SEQ ID NO:248 and encode the peptides with amino acid sequences of SEQ ID NO:247; SEQ ID NO:249, respectively.

Suitable polynucleotide for entC is provided in SEQ ID NO:250; and encode the peptide with amino acid sequences of SEQ ID NO:251.

Suitable polynucleotide for mutant serB is provided in SEQ ID NO:257; SEQ ID NO: 259; SEQ ID NO:261; and encode the peptide with amino acid sequences of SEQ ID NO: 258; SEQ ID NO:260; SEQ ID NO:262.

The invention is not limited to the use of these amino acid sequences. Amino acid sequences comprising of the variation of the enzymes and transcription factors listed are included within the scope of the present invention and are considered substantially or sufficiently similar to a reference amino acid sequence. Although it is not intended that the present invention be limited by any theory by which it achieves its advantageous result, it is believed that the identity between amino acid sequences that is necessary to maintain proper functionality is related to maintenance of the tertiary structure of the polypeptide such that specific interactive sequences will be properly located and will have the desired activity, and it is contemplated that a polypeptide including these interactive sequences in proper spatial context will have activity.

Another manner in which similarity may exist between two amino acid sequences is where there is conserved substitution between a given amino acid of one group. The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

One of ordinary skill in the art will recognize that changes in the amino acid sequences, such as individual substitutions, deletions or additions to a nucleic acid, peptide, polypeptide, or protein sequence which alters, adds or deletes a single amino acid or a small percentage of amino acids in the encoded sequence is “sufficiently similar” when the alteration results in the substitution of an amino acid with a chemically similar amino acid.

It is therefore understood that the invention encompasses more than the specific polynucleotides encoding the proteins described herein. For example, modifications to a sequence, such as deletions, insertions, or substitutions in the sequence, which produce “silent” changes that do not substantially affect the functional properties of the resulting polypeptide are expressly contemplated by the present invention. It is known by those of ordinary skill in the art, “universal” code is not completely universal. Some mitochondrial and bacterial genomes diverge from the universal code, e.g., some termination codons in the universal code specify amino acids in the mitochondria or bacterial codes. Thus, each silent variation of a nucleic acid, which encodes a polypeptide of the present invention, is implicit in each described polypeptide sequence and incorporated in the descriptions of the invention.

It is understood that alterations in a nucleotide sequence, which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

When the nucleic acid is prepared or altered synthetically, one of ordinary skill in the art can take into account the known codon preferences for the intended host where the nucleic acid is to be expressed. For example, although nucleic acid sequences of the present invention may be expressed in different species, sequences can be modified to account for the specific codon preferences and GC-content preferences of the organism, as these preferences have been shown to differ.[64-69]

### Cloning Techniques

Unless mentioned otherwise, the techniques employed or contemplated herein are standard methodologies well known to one of ordinary skill in the art. Specific terms, while employed below and defined at the end of this section, are used in a descriptive sense only and not for purposes of limitation. The practice of the present invention will employ, unless otherwise indicated, conventional techniques of botany, microbiology, mycology, phycology, tissue culture, molecular biology, chemistry, biochemistry, biotechnology, and recombinant DNA technology, which are within the skill of the art.[70-77]

A suitable polynucleotide for use in accordance with the invention may be obtained by cloning techniques using cDNA or genomic libraries, DNA, or cDNA from bacteria, algae, microalgae, diatoms, yeast or fungi which are available commercially or which may be constructed using standard methods known to persons of ordinary skill in the art. Suitable nucleotide sequences may be isolated from DNA libraries obtained from a wide variety of species by means of nucleic acid hybridization or amplification methods, such as polymerase chain reaction (PCR) procedures, using as probes or primers nucleotide sequences selected in accordance with the invention.

Furthermore, nucleic acid sequences may be constructed or amplified using chemical synthesis. The product of amplification is termed an amplicon. Moreover, if the particular nucleic acid sequence is of a length that makes chemical synthesis of the entire length impractical, the sequence may be broken up into smaller segments that may be synthesized and ligated together to form the entire desired sequence by methods known in the art. Alternatively, individual components or DNA fragments may be amplified by PCR and adjacent fragments can be amplified together using fusion-PCR,[78] overlap-PCR[79] or chemical (de novo) synthesis[80-84] using a vendor (e.g. DNA2.0, GE life technologies, GENEART, Gen9, GenScript) by methods known in the art.

The recombinant expression cassette or DNA construct includes a promoter that directs transcription in a unicellular organism, operably linked to the polynucleotide of the invention described herein. A variety of different types of promoters are described and used. As used herein, a polynucleotide is “operably linked” to a promoter or other nucleotide sequence when it is placed into a functional relationship with the promoter or other nucleotide sequence. The functional relationship between a promoter and a desired polynucleotide insert typically involves the polynucleotide and the promoter sequences being contiguous such that transcription of the polynucleotide sequence will be facilitated. Two nucleic acid sequences are further said to be operably linked if the nature of the linkage between the two sequences does not (1) result in the introduction of a frame-shift mutation; (2) interfere with the ability of the promoter region sequence to direct the transcription of the desired nucleotide sequence, or (3) interfere with the ability of the desired nucleotide sequence to be transcribed by the promoter sequence region. Typically, the promoter element is generally upstream (i.e., at the 5′ end) of the nucleic acid insert coding sequence.

While a promoter sequence can be ligated to a coding sequence prior to insertion into a vector, in other embodiments, a vector is selected that includes a promoter operable in the host cell into which the vector is to be inserted. In addition, certain preferred vectors have a region that codes a ribosome binding site positioned between the promoter and the site at which the DNA sequence is inserted so as to be operatively associated with the DNA sequence of the invention to produce the desired polypeptide, i.e., the DNA sequence of the invention in-frame.

Gene expression cassettes may contain one or more polynucleotides (genes), each operably linked with a promoter and terminator to form a series of monocistronic mRNAs or the genes can be arranged with one promoter and terminator to form a single polycistronic mRNA. A wide variety of operable cassettes are known to those of ordinary skill in the art.

### Suitable Promoters

A wide variety of promoters are known to those of ordinary skill in the art, as are other regulatory elements that can be used alone or in combination with promoters.

### Terminators

In addition to the selection of a suitable promoter, the DNA constructs require an appropriate transcriptional terminator to be attached downstream (3′), after the stop codon (TGA, TAG or TAA) of the desired gene of the invention for proper expression in unicellular organisms. Several such terminators are available and known to persons of ordinary skill in the art

### Selectable Markers

Selectable markers usually confer resistance to an antibiotic, herbicide or chemical or provide color change, which aid the identification of transformed organisms. Selectable markers are available and known to persons of ordinary skill in the art.

### Plastid Transit Peptides

The invention can be targeted for transformation into the chloroplast. Chloroplast targeted transformation systems for algae are known by those of ordinary skill in the art.

### Suitable Vectors

A wide variety of vectors may be employed to transform a unicellular organism with a construct made or selected in accordance with the invention, including high- or low-copy number plasmids, phage vectors and cosmids. Vector systems, expression cassettes, culture methods, and transformation methods are known by those of ordinary skill in the art. Although the preferred embodiment of the invention is expressed in unicellular organisms, other embodiments may include expression in prokaryotic or unicellular eukaryotic organisms including, but not limited to, yeast, fungi, algae, microalgae, or microbes.

### Expression in Prokaryotes

Protocols for transformation as well as commonly used vectors with control sequences are known to those of ordinary skill in the art. Those of ordinary skill in the art know the molecular techniques and DNA vectors that are used in bacterial systems. In bacteria one messenger RNA can encode for one peptide (referred to as monocistronic) or several independent peptides (referred to as polycistronic). It is known to those of ordinary skill in the art that a portion of a polycistronic messenger RNA can be knocked-out or that heterologous or exogenous genes can be expressed on a monocistronic or polycistronic messenger RNA. Genes can be expressed by modification of bacterial DNA (genomic) through the use of knock-in, gene insertion, or by allelic exchange.[85-90] Specific gene targeting has been used in bacteria using PCR-based methods, and CRISPR/Cas.[92-94]

### Expression in Algae, Microalgae, and Non-Plant Eukaryotes

Protocols for transformation as well as commonly used vectors with control sequences include promoters for transcription initiation, optionally with an operator, together with ribosome binding site sequences for use in algae, microalgae, and non-plant eukaryotes (including yeast and fungi) are known to those of ordinary skill in the art. Specific gene targeting systems have been used in algae including ZFNs[95] and transcription activator-like effector nucleases (TALENs).[96]

One of ordinary skill in the art recognizes that modifications could be made to a protein of the present invention without diminishing its biological activity. Some modifications may be made to facilitate the cloning, expression, targeting or to direct the location of the polypeptide in the host, or for the purification. Such modifications are known to those of ordinary skill in the art and include, for example, a methionine added at the amino terminus to provide an initiation site, additional nucleic acids to insert a restriction site or a termination.

### Gene Silencing by Mutagenesis or Recombinant Technologies

Genetic modification to silence or inactivate genes or their corresponding gene products of unicellular organisms can be conducted by radiation-, chemical- or UV-based mutagenesis followed by specific screening for biochemical traits or pathways.[97-102] Radiation-based mutations can silence or inactive a gene or the corresponding gene product by DNA breakage and repair. Chemical- or UV-based mutations usually result in single DNA basepair changes. Mutations can silence or inactive a gene or the corresponding gene product by one of the following: (1) introduction of a frame-shift mutation; (2) introduction of premature stop codon; (3) interference with the ability of the promoter region sequence to direct the transcription of the desired nucleotide sequence, (4) interference with the ability of the desired nucleotide sequence to be transcribed by the promoter sequence region or (5) introduction of an amino acid substitution in the gene product to reduce or inhibit activity (enzymatic activity or binding) or interfere with the function of the gene product.

Targeted gene silencing or knockouts can be made in unicellular organisms using phage or viruses,[104-109] transposons,[110-114] PCR-assisted targeting,[115-118] recombinases or by allelic exchange,[85-90] targeted and random bacterial gene disruptions using a group II intron (Targetron),[119, 120] ZNFs,[121] TALENs, CRISPER-Cas9 or clustered regularly interspaced short palindromic repeats interference (CRISPi).[92-94, 123-126] In addition, RNA-mediated methods,[127-132] or regulatory RNAs[133-135] have been used to silence or suppress gene expression in unicellular organisms and these techniques and protocols are well known to one with ordinary skill in the art.

### Suitable Unicellular Organisms

A wide variety of unicellular host cells may be used in the invention, including prokaryotic and unicellular eukaryotic host cells. These cells or organisms may include yeast, fungi, algae, microalgae, microbes, or unicellular photosynthetic organisms. Preferred host cells for this invention are bacteria including, archaebacteria and cubacteria. Proteobacteria such as members of Alphaproteobacteria, Betaproteobacteria, Gammaproteobacteria, Deltaproteobacteria, and Epsilonproteobacteria can host the invention. Other bacteria including Methanotrophs and Methylobacterium can be used with the invention. Other bacterial genera that can host the invention include, but are not limited to Escherichia, Bacillus, Salmonella, Lactococcus, Lactobacillus, Streptococcus, Brevibacterium and Coryneform bacteria. Some specific bacterial species that can be used for the invention include, but are not limited to, Bacillus subtilis, Brevibacterium ammoniagene, Corynebacterium crenatum, Corynebacterium pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans, and Saccharomyces cerevisiae.

Unicellular algae, unicellular photosynthetic organisms, and microscopic algae (microphytes or microalgae) cells may be used in the invention. These include, but are not limited to diatoms, green algae (Chlorophyta), and members of the Euglenophyta, Dinoflagellata, Chrysophyta, Phacophyta, red algae (Rhodophyta), Heterokontophyta, and Cyanobacteria. The invention can also be used to increase the O-phosphoserine, cysteate, or taurine by binding with a binding protein or knocking out genes for O-phosphoserine, cysteate, or taurine degradation in algae that have been shown to synthesize O-phosphoserine, cysteate, or taurine or may have the capability to synthesize O-phosphoserine, cysteate, or taurine. These include but are not limited to Coccomyxa species, Chlorella species, Trebouxia impressa, Tetraselmis species, Chlamydomonas reinhardtii, Micromonas pusilla, Ostreococcus tauri, Navicula radiosa, Phaeodactylum tricornutum, Pseudo-nitzschia multiseries, Fragilariopsis cylindrus, Thalassiosira weissflogii, Nannochloropsis oceanica, Aureococcus anophagefferens, Saccharina japonica, Sargassum species and Bigelowiella natans.

Protozoa that may be used in the invention include, but are not limited, to ciliates, amoebae and flagellates. Yeast and unicellular fungi that can be used include, but are not limited to Ashbya gossypii, Blakeslea trispora, Candida flareri, Eremothecium ashbyii, Mortierella isabellina, Pichia pastoris, Saccharomyces cerevisiae, and Saccharomyces pombe.

### Pharmaceutical Compositions

The invention provides pharmaceutical compositions that comprise extracts of one or more modified unicellular organisms described above. Extracts containing O-phosphoserine, cysteate, or taurine can be used to synthesize or manufacture derivatives,[136, 137] conjugates[138] or polymers[139] that may have a wide range of commercial and medicinal applications.[140] Some derivatives can function as organogelators[141] or dyes[142] and can be used in nanosensor synthesis.[143] Some derivatives have anticonvulsant[136] or anti-cancer[144] properties. Other derivatives are used in the treatment of alcoholism.[145, 146] Taurine-conjugated carboxyethylester-polyrotaxanes increase anticoagulant activity.[147] Taurine-containing polymers may increase wound healing.[148, 149] Taurine linked polymers such as poly gamma-glutamic acid-sulfonates are biodegradable and may have applications in the development of drug delivery systems, environmental materials, tissue engineering, and medical materials.[150] Extracts from O-phosphoserine-, cysteate-, or taurine-containing cells or cultures may be used in pharmaceutical or medicinal compositions in the treatment of congestive heart failure, high blood pressure, hepatitis, autoimmune disorders high cholesterol, fibrosis, epilepsy, autism, attention deficit-hyperactivity disorder, retinal degeneration, multiple sclerosis, diabetes, and alcoholism. They are also used to improve mental performance, cognitive disorders, multiple sclerosis disease or as an antioxidant.

Pharmaceutically acceptable vehicles of O-phosphoserine, cysteate, or taurine, or their derivatives, conjugates, or polymers include tablets, capsules, gel, ointment, film, patch, powder or dissolved in liquid form.

### Nutritional Supplements and Feeds

Recombinant cells containing O-phosphoserine, cysteate, or taurine may be consumed or used to make extracts for nutritional supplements. Recombinant cells or cell cultures that contain O-phosphoserine, cysteate, or taurine may be used for human consumption. Extracts from the recombinant cells may be used as nutritional supplements, as an antioxidant or to improve physical or mental performance. The extracts may be used in the form of a liquid, powder, capsule or tablet.

Recombinant cells containing O-phosphoserine, cysteate, or taurine may be used as fish or animal feed or used to make extracts for the supplementation of animal feed and may be in the form of a liquid, powder, capsule or tablet.

### Enhancer of Plant Growth or Yield

Recombinant cells that contain O-phosphoserine, cysteate, or taurine may be used as an enhancer for plant growth or yield. Extracts from transgenic cells or use of cell cultures containing O-phosphoserine, cysteate, or taurine may be used as plant enhancers in the form of a liquid, powder, capsule or tablet.

### Definitions

The term “polynucleotide” refers to a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The terms “amplified” and “amplification” refer to the construction of multiple copies of a nucleic acid sequence or multiple copies complementary to the nucleic acid sequence using at least one of the nucleic acid sequences as a template. Amplification can be achieved by chemical synthesis using any of the following methods, such as solid-phase phosphoramidate technology or the polymerase chain reaction (PCR). Other amplification systems include the ligase chain reaction system, nucleic acid sequence-based amplification, Q-Beta Replicase systems, transcription-based amplification system, and strand displacement amplification. The product of amplification is termed an amplicon.

As used herein “promoter” includes reference to a region of DNA upstream from the start of transcription and involved in recognition and binding of RNA polymerase, either I, II or III, and other proteins to initiate transcription. Promoters include necessary nucleic acid sequences near the start site of transcription, such as, in the case of a polymerase II type promoter, a TATA element. A promoter also optionally includes distal enhancer or repressor elements, which can be located as far as several thousand base pairs from the start site of transcription. In bacteria, the promoter includes a Shine-Dalgarno or ribosomal binding site that can include the sequence AGGAGG (−35 box) and a Pribnow box or RNA polymerase binding site that can include the sequence TATAAT (−10 box).

The term “algal promoter” refers to a promoter capable of initiating transcription in algal cells.

The term “foreign promoter” refers to a promoter, other than the native, or natural, promoter, which promotes transcription of a length of DNA of viral, bacterial or eukaryotic origin, including those from microbes, plants, plant viruses, invertebrates or vertebrates.

The term “microbe” refers to any microorganism (including both eukaryotic and prokaryotic microorganisms), such as bacteria, fungi, yeast, bacteria, algae and protozoa, as well as other unicellular organisms.

The term “constitutive” refers to a promoter that is active under most environmental and developmental conditions, such as, for example, but not limited to, the CaMV 35S promoter.

The term “inducible promoter” refers to a promoter that is under chemical (including biomolecules such as sugars, organic acids or amino acids) or environmental control.

The terms “encoding” and “coding”” refer to the process by which a polynucleotide, through the mechanisms of transcription and translation, provides the information to a cell from which a series of amino acids can be assembled into a specific amino acid sequence to produce a functional polypeptide, such as, for example, an active enzyme or ligand binding protein.

The terms “polypeptide,” “peptide,” “protein” and “gene product” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. Amino acids may be referred to by their commonly known three-letter or one-letter symbols. Amino acid sequences are written left to right in amino to carboxy orientation, respectively. Numeric ranges are inclusive of the numbers defining the range.

The terms “residue,” “amino acid residue,” and “amino acid” are used interchangeably herein to refer to an amino acid that is incorporated into a protein, polypeptide, or peptide. The amino acid may be a naturally occurring amino acid and may encompass known analogs of natural amino acids that can function in a similar manner as the naturally occurring amino acids.

The term “degradation” in reference to the “taurine degradation pathway”, “taurine degradation enzymes”, “taurine degradation system”, and “taurine degradation proteins” refers to the process of breakdown, catabolism or dissimilation of taurine.

Decreased catalytic activity means decreasing affinity for a given substrate and/or a given cofactor, and/or increasing the binding of a given inhibitor, these measures can be obtained determination by enzymatic analysis to obtain Michaelis-Menten constants these methods are known to those of ordinary skill in the art. Decreased enzyme activity can also be achieved by increasing protein turnover or by destabilizing the protein.

The terms “sulfinoalanine decarboxylase” and “SAD” refer to the protein that catalyzes the following reactions:

$$3 - sulfinoalanine = hypotaurine + {CO}_{2}$$

$$3 - sulfinoalanine = taurine + {CO}_{2}$$

NOTE: SAD is another name for cysteine-sulfinate decarboxylase, L-cysteine sulfinic acid decarboxylase, cysteine-sulfinate decarboxylase, CADCase/CSADCase, CSAD, cysteic decarboxylase, cysteine sulfinic acid decarboxylase, cysteine sulfinate decarboxylase, sulfoalanine decarboxylase, sulphinoalanine decarboxylase, cysteate decarboxylase (CAD), cysteic acid decarboxylase, and 3-sulfino-L-alanine carboxy-lyase.
NOTE: the SAD reaction is also catalyzed by some cysteine sulfonic acid decarboxylases or cysteate decarboxylases (CAD), glutamic acid decarboxylases (GAD) or partCS/PLP-DC. Although called GAD some GAD enzymes have been shown to catalyze the SAD reaction.[151, 152]

Other names for taurine are 2-aminoethane sulfonic acid, aminoethanesulfonate, L-taurine, taurine ethyl ester, and taurine ketoisocaproic acid 2-aminoethane sulfinate.

The terms “threonine synthase” and “TS” refer to the protein that catalyzes the following reaction:
NOTE: some O-phosphoserine and sulfite=cysteate
TS is another name for cysteate synthase (CS).

The terms “taurine-pyruvate aminotransferase” and “TPAT” refer to the protein that catalyzes the following reaction:

$$taurine + pyruvate = L - alanine + 2 - sulfoacetaldehyde$$

TPAT is another name for taurine transaminase or taurine transaminase aminotransferase. The term “Tpa” refers to the gene that encodes TPAT.

The terms “taurine dehydrogenase” and “TDH” refer to the protein that catalyzes the following reaction:

$$taurine + water = ammonia + 2 - sulfoacetaldehyde$$

TDH is another name for taurine: oxidoreductase, taurine: ferricytochrome-c oxidoreductase,

The term “tauX” or “tauY” refers to the genes that encode for the small and large subunits of TDH, respectively.

The terms “taurine dioxygenase” and “TDO” refer to the protein that catalyzes the following reaction:

$$taurine + 2 - oxoglutarate = {O}_{2} = sulfite + aminoacetaldehyde + succinate + {CO}_{2}$$

TDO is another name for 2-aminoethanesulfonate dioxygenase, alpha-ketoglutarate-dependent taurine dioxygenase, taurine, or 2-oxoglutarate: O₂ oxidoreductase.

The term “tauD” refers to the gene that encodes TDO.

The term “two-component alkanesulfonate monooxygenase” or “2CASM” catalyzes the following reaction:

$$taurine + {O}_{2} + {FMNH}_{2} = Aminoacetaldehyde + {SO}_{3}^{2} + {H}_{2} O + FMN$$

$$or$$

$$taurine + {O}_{2} + {Thioredoxin}_{red} = Aminoacetaldehyde + {SO}_{3}^{2} + {H}_{2} O + {Thioredoxin}_{ox}$$

The term “ssuDE”, “ssuD” or “ssuE” refers to the genes that encode the two-component alkanesulfonate monooxygenase (2CASM).

The terms “cysteine synthetase/PLP decarboxylase” and “CS/PLP-DC” refer to the protein that catalyzes the following reactions:

$$2 - aminocrylate + PAPS = taurine$$

$$O - phosphoserine + PAPS = taurine$$

$$O - acetyl - L - serine + hydrogren sulfide = taurine$$

$$O - phosphoserine + sulfite = taurine$$

The terms “portion of the cysteine synthetase/PLP decarboxylase” and “partCS/PLP-DC” refers to the protein that catalyzes a decarboxylase reaction which cleaves carbon-carbon bonds and includes, but is not limited to, the following substrate and end-products:

$$Homocysteate = homotaurine + {CO}_{2}$$

$$Cysteine + Sulfite = Cysteate$$

$$Cysteic acid ( cysteate ) = 2 - aminoethane sulfonate ( taurine ) + {CO}_{2}$$

$$3 - sulfinoalanine = hypotaurine + {CO}_{2}$$

$$Glutamate = 4 - aminobutanoate + {CO}_{2}$$

NOTE: the partCS/PLP-DC has sulfinoalanine decarboxylase (SAD), cysteine sulfonic acid decarboxylase, cysteate decarboxylase (CAD) or glutamic acid decarboxylases (GAD) activity.
NOTE: the truncated CS/PLP-DC has cysteine lyase (CL) or homocysteine lyase.

Another name for 4-aminobutanoate is gamma-aminobutyric acid (GABA).

PLP synthase is an enzyme complex derived from the gene products of the of the pdxS and pdxT genes. pdxS is also called Pdx1, SnzP, or YaaD and pdxT genes is also called to Pdx2, SnoP, or YaaE.

The term “recombinant” includes reference to a cell or vector that has been modified by the introduction of a heterologous nucleic acid. Recombinant cells express genes that are not normally found in that cell or express native genes that are otherwise abnormally expressed, under-expressed, or not expressed at all as a result of deliberate human intervention, or expression of the native gene may have reduced or eliminated as a result of deliberate human intervention.

The term “recombinant expression cassette” refers to a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “transgenic” includes reference to a unicellular, which comprises within its genome a heterologous polynucleotide. Generally, the heterologous polynucleotide is integrated within the genome such that the polynucleotide is passed on to successive generations. The heterologous polynucleotide may be integrated into the genome alone or as part of a recombinant expression cassette. “Transgenic” is also used to include any cell the genotype of which has been altered by the presence of heterologous nucleic acid including those cells altered or created by budding or conjugation propagation from the initial transgenic cell.

The term “vector” includes reference to a nucleic acid used in transfection or transformation of a host cell and into which can be inserted a polynucleotide.

The following terms are used to describe the sequence relationships between two or more nucleic acids or polynucleotides or polypeptides: “reference sequence,” “comparison window,” “sequence identity,” “percentage of sequence identity,” and “substantial identity.”

The term “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “comparison window” includes reference to a contiguous and specified segment of a polynucleotide sequence, where the polynucleotide sequence may be compared to a reference sequence and the portion of the polynucleotide sequence in the comparison window may comprise additions or deletions (i.e., gaps) when it is compared to the reference sequence for optimal alignment. The comparison window is usually at least 20 contiguous nucleotides in length, and optionally can be 30, 40, 50, 100 or longer. Those of ordinary skill in the art understand that the inclusion of gaps in a polynucleotide sequence alignment introduces a gap penalty, and it is subtracted from the number of matches.

Methods of alignment of nucleotide and amino acid sequences for comparison are well known to those of ordinary skill in the art. The local homology algorithm, BESTFIT,[153] can perform an optimal alignment of sequences for comparison using a homology alignment algorithm called GAP,[154] search for similarity using Tfasta and Fasta,[155] by computerized implementations of these algorithms widely available on-line or from various vendors (Intelligenetics, Genetics Computer Group). CLUSTAL allows for the alignment of multiple sequences[156-158] and program PileUp can be used for optimal global alignment of multiple sequences.[159] The BLAST family of programs can be used for nucleotide or protein database similarity searches. BLASTN searches a nucleotide database using a nucleotide query. BLASTP searches a protein database using a protein query. BLASTX searches a protein database using a translated nucleotide query that is derived from a six-frame translation of the nucleotide query sequence (both strands). TBLASTN searches a translated nucleotide database using a protein query that is derived by reverse-translation. TBLASTX search a translated nucleotide database using a translated nucleotide query.

GAP[154] maximizes the number of matches and minimizes the number of gaps in an alignment of two complete sequences. GAP considers all possible alignments and gap positions and creates the alignment with the largest number of matched bases and the fewest gaps. It also calculates a gap penalty and a gap extension penalty in units of matched bases. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62.[160]

Unless otherwise stated, sequence identity or similarity values refer to the value obtained using the BLAST 2.0 suite of programs using default parameters.[161] As those of ordinary skill in the art understand that BLAST searches assume that proteins can be modeled as random sequences and that proteins comprise regions of nonrandom sequences, short repeats, or enriched for one or more amino acid residues, called low-complexity regions. These low-complexity regions may be aligned between unrelated proteins even though other regions of the protein are entirely dissimilar. Those of ordinary skill in the art can use low-complexity filter programs to reduce number of low-complexity regions that are aligned in a search. These filter programs include, but are not limited to, the SEG[162, 163] and XNU.[164]

The terms “sequence identity” and “identity” are used in the context of two nucleic acid or polypeptide sequences and include reference to the residues in the two sequences, which are the same when aligned for maximum correspondence over a specified comparison window. When the percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conserved substitutions, the percent sequence identity may be adjusted upwards to correct for the conserved nature of the substitution. Sequences, which differ by such conservative substitutions, are said to have “sequence similarity” or “similarity.” Scoring for a conservative substitution allows for a partial rather than a full mismatch,[165] thereby increasing the percentage sequence similarity.

The term “percentage of sequence identity” means the value determined by comparing two optimally aligned sequences over a comparison window, wherein the portion of the polynucleotide sequence in the comparison window may comprise gaps (additions or deletions) when compared to the reference sequence for optimal alignment. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90%, and most preferably at least 95%, compared to a reference sequence using one of the alignment programs described using standard parameters. One of ordinary skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 50-100%. Another indication that nucleotide sequences are substantially identical is if two molecules hybridize to each low stringency conditions, moderate stringency conditions or high stringency conditions. Yet another indication that two nucleic acid sequences are substantially identical is if the two polypeptides immunologically cross-react with the same antibody in a western blot, immunoblot or ELISA assay.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm[154]. Thus, a peptide is substantially identical to a second peptide, for example, where the two peptides differ only by a conserved substitution. Another indication that amino acid sequences are substantially identical is if two polypeptides immunologically cross-react with the same antibody in a western blot, immunoblot or ELISA assay. In addition, a peptide can be substantially identical to a second peptide when they differ by a non-conservative change if the epitope that the antibody recognizes is substantially identical.

The invention provides isolated cells comprising DNA which does not express a functional enzymes, some isolated cells of the invention comprise (i) exogenous DNA which disrupts the expression of the gene or renders the corresponding peptide for the degradation enzyme non-functional (ii) a basepair mutation that disrupts the expression of the gene or renders the corresponding peptide for the degradation enzyme non-functional, or (iii) a deletion of the entire polynucleotide or a portion of the polynucleotide which disrupts the expression of the gene or renders the corresponding peptide for the degradation enzyme non-functional. The non-functional DNA could be due to changes in the promoter, a portion of the coding region or terminator to a polynucleotide which encodes the enzyme in a manner where the gene products are not functional. The invention also provides isolated cells comprising non-functional genes or gene products of enzymes from the suppression or decreased accumulation of the corresponding RNA due to antisense RNA or RNA interference.

### NUMBERED EMBODIMENTS

### Additional Embodiments

Embodiment 1. A method for taurine production comprising: growing a unicellular organism in a shaker flask to produce at least 0.25 g/liter of taurine from the unicellular organism.

Embodiment 2. A method for taurine production comprising: growing a unicellular organism in a fermentor or bioreactor to produce at least 15 g/liter of taurine from the unicellular organism.

Embodiment 3. The method of embodiment 1 or 2, wherein the unicellular organism expresses one or more exogenous taurine biosynthetic pathways and contains one or more of the following: a deletion of at least one operon for taurine or sulfonate uptake and degradation; deletion of a gene in the enterobactin or enterochelin biosynthetic pathway: increased expression of genes in sulfate or thiosulfate transport, or sulfur reduction or sulfur assimilation; increased expression of a gene in the PLP pathway; increased expression of a gene in the serine biosynthetic pathway; increased expression of a gene in the cysteine biosynthetic pathway; increased expression of a gene in the 2-aminoacrylate biosynthetic pathway; or a deletion of at least one gene in the degradation of taurine, serine, cysteate or 2-aminoacrylate.

Embodiment 4. The method of any one of the previous embodiments, further comprising: isolating the taurine to produce taurine having a purity level of greater than 10% purity, greater than 25% purity, greater than 50% purity, greater than 75% purity, or greater than 98% purity.

Embodiment 5. The method of any one of the previous embodiments, wherein the unicellular organism is E. coli and the growing step is conducted in a medium that contains at least 5 g/L ammonium sulfate, at least 6 g/L dibasic potassium phosphate, at least 3 g/L monobasic sodium phosphate, at least 0.5 g/L magnesium sulfate, at least 6 g/L glucose, at least 0.1 g/L typtone, and at least 0.05 g/L yeast extract.

Embodiment 6. The method of any one of the previous embodiments, wherein the unicellular organism is C. glutamicum and the growing step is conducted in a medium that contains at least 2 g/L yeast extract, 40 g/L glucose, 10 g/L calcium carbonate, 15 g/L ammonium sulfate, 1 g/L dibasic potassium phosphate, and 1 g/L monobasic potassium phosphate.

Embodiment 7. A method for cysteate production comprising: growing a unicellular organism in a shaker flask to produce at least 0.1 g/liter of cysteate from the unicellular organism.

Embodiment 8. A method for cysteate production comprising: growing a unicellular organism in a fermentor or bioreactor to produce at least 1 g/liter of cysteate from the unicellular organism.

Embodiment 9. The method of embodiment 7 or 8, wherein the unicellular organism expresses one or more exogenous cysteate biosynthetic pathways and contains one or more of the following: a deletion of at least one operon for taurine or sulfonate uptake and degradation; deletion of a gene in the enterobactin or enterochelin biosynthetic pathway: increased expression of genes in sulfate or thiosulfate transport, or sulfur reduction or sulfur assimilation; increased expression of a gene in the PLP pathway increased expression of a gene in the aspartate pathway; increased expression of a gene in the threonine pathway; or a deletion of at least one gene in the degradation of cysteate.

Embodiment 10. The method of any one of embodiments 7 to 9, further comprising: isolating the cysteate to produce cysteate having a purity level of greater than 10% purity, greater than 25% purity, greater than 50% purity, greater than 75% purity, or greater than 98% purity.

Embodiment 11. The method of any one of the previous embodiments, wherein the unicellular organism is a microbe.

Embodiment 12. The method of embodiment 11, wherein the microbe is selected from the group consisting of Proteobacteria, Alphaproteobacteria, Betaproteobacteria, Deltaproteobacteria, Epsilonproteobacteria, Methanotrophs, Methylobacterium, Escherichia, Bacillus, Salmonella, Lactococcus, Lactobacillus, Streptococcus, Brevibacterium, and coryneform bacteria.

Embodiment 13. The method of embodiment 11, wherein the microbe is selected from the group consisting of Bacillus subtilis, Brevibacterium ammoniagene, Corynebacterium crenatum, Corynebacterim pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans, and Saccharomyces cerevisiae.

Embodiment 14. The method of any one of the previous embodiments, wherein the cells of the unicellular organism are chemically, physically, or mechanically disrupted, dried, and used in food, feed, beverages, dietary and health supplements, cosmetics, personal care, pharmaceuticals, agricultural production, or surfactants.

Embodiment 15. A recombinant microbe or unicellular organism, comprising: a polynucleotide comprising a sequence encoding a mutated serB gene, and one or more exogenous polynucleotides comprising a sequence encoding a threonine synthase (TS) polypeptide or cysteate synthase (CS) polypeptide, wherein the mutated serB gene has reduced expression, is configured to have a reduced amount of a serB gene product, or encodes a serB gene product with reduced enzymatic activity, and wherein the recombinant microbe or unicellular organism has a modified biosynthetic pathway.

Embodiment 16. The recombinant microbe or unicellular organism of Embodiment 15, further comprising one or more exogenous polynucleotides comprising a sequence encoding a sulfinoalanine decarboxylase (SAD) polypeptide, a cysteic acid decarboxylase (CAD) polypeptide, a glutamate decarboxylase (GAD) polypeptide, or a polypeptide corresponding to a decarboxylase portion of the cysteine synthetase/PLP decarboxylase (partCS/PLP-DC).

Embodiment 17. The recombinant microbe or unicellular organism of Embodiments 15 or 16, wherein the modified biosynthetic pathway comprises a modified sulfur-based pathway that inhibits cysteate degradation or inhibits taurine degradation.

Embodiment 18. The recombinant microbe or unicellular organism of Embodiment 17, wherein modified sulfur-based pathway comprises increased expression of cysPUWA, cysDNC, and cysQ, and a gene knock-out of tauD, ssuD, and ssuE.

Embodiment 19. The recombinant microbe or unicellular organism of Embodiments 17 or 18, wherein recombinant microbe or unicellular organism has a gene knock-out of cuyA, tauABCD, ssuEADCB, ssuDICBA, sueABCD2, or a combination thereof.

Embodiment 20. The recombinant microbe or unicellular organism of Embodiments 17 or 18, wherein recombinant microbe or unicellular organism has a gene knock-out of tauABCD, ssuEADCB, ssuDICBA or sueABCD2, or a combination thereof.

Embodiment 21. The recombinant microbe or unicellular organism of Embodiment 16, wherein the modified biosynthetic pathway comprises a modified O-phosphoserine pathway comprising increased expression of pgk, serA_{Δ197}, serC, or a combination thereof, and wherein the recombinant microbe or unicellular organism further comprises a modified sulfur-based pathway.

Embodiment 22. The recombinant microbe or unicellular organism of Embodiment 21, wherein the modified sulfur-based pathway comprises increased expression of cysPUWA, cysDNC, cysQ or cysH, and a gene knock-out of tauD, ssuD, and ssuE, or wherein the modified sulfur-based pathway comprises a gene knock-out of tauABCD, ssuEADCB, ssuDICBA, sueABCD2, or a combination thereof.

Embodiment 23. A recombinant microbe or unicellular organism, wherein the recombinant microbe or unicellular organism has increased production of O-phosphoserine, cysteate, taurine, or a combination thereof.

Embodiment 24. The recombinant microbe or unicellular organism of Embodiment 23, wherein the recombinant microbe or unicellular organism has reduced import of O-phosphoserine, cysteate, taurine, or a combination thereof, or wherein the recombinant microbe or unicellular organism has increased export of O-phosphoserine, cysteate, taurine, or a combination thereof.

Embodiment 25. The recombinant microbe or unicellular organism of Embodiment 24, wherein the recombinant microbe or unicellular organism has a deletion or silencing mutation in sdaC, cycA, sstT, tdcC, or cyuP (yhaO), or a combination thereof.

Embodiment 26. The recombinant microbe or unicellular organism of Embodiment 24, wherein the recombinant microbe or unicellular organism has increased expression of emaA (ydeD), alaE (ygaW), yfik, cefA, cefB, rhtA, rhtB, rhtC, gabP, tauP, gadC, yhiM, AAperm, or a combination thereof.

Embodiment 27. The recombinant microbe or unicellular organism of Embodiment 23, wherein the recombinant microbe or unicellular organism comprises one or more polypeptides of the O-phosphoserine, cysteate, or taurine biosynthetic pathway configured to be in close proximity to each other by forming molecular scaffolds, channels, or cages, wherein the recombinant microbe or unicellular organism comprises one or more polypeptides of the O-phosphoserine, cysteate, or taurine biosynthetic pathway configured to be in close proximity to polypeptides involved in the production of precursors by forming molecular scaffolds, channels, or cages, or wherein the recombinant microbe or unicellular organism comprises one or more polypeptides of the O-phosphoserine, cysteate, or taurine biosynthetic pathway configured to be in close proximity to exporters of O-phosphoserine, cysteate, or taurine by forming molecular scaffolds, channels, or cages.

Embodiment 28. The recombinant microbe or unicellular organism of Embodiment 23, wherein the recombinant microbe or unicellular organism is contacted with exogenous PLP, pyridoxine or a pyridoxine salt.

Embodiment 29. The recombinant microbe or unicellular organism of Embodiment 23, wherein the recombinant microbe or unicellular organism has increased expression of one of more genes in the PLP-biosynthetic pathway to increase production of O-phosphoserine, cysteate, or taurine.

Embodiment 30. The recombinant microbe or unicellular organism of any one of Embodiments 15-29, wherein the recombinant microbe or unicellular organism comprises bacteria, yeast, fungi, or unicellular algae.

Embodiment 31. A method for increased taurine production in a recombinant microbe or unicellular organism, comprising growing a unicellular organism under conditions to produce taurine, wherein the recombinant microbe or unicellular organism has a modified biosynthetic pathway.

Embodiment 32. The method of Embodiment 31, wherein the modified biosynthetic pathway comprises:
i. a deletion of at least one operon for taurine or sulfonate uptake and degradation;
ii. a deletion of a gene in the enterobactin or enterochelin biosynthetic pathway:
iii. an increased expression of genes in sulfate or thiosulfate transport, or sulfur reduction or sulfur assimilation;
iv. an increased expression of a gene in the PLP pathway;
v. an increased expression of a gene in the serine biosynthetic pathway;
vi. an increased expression of a gene in the cysteine biosynthetic pathway;
vii. an increased expression of a gene in the 2-aminoacrylate biosynthetic pathway;
viii. a deletion of at least one gene in the degradation of taurine, serine, cysteate or 2-aminoacrylate; or
ix. any combination of (i) to (viii).

Embodiment 33. The method of Embodiments 31 or 32, wherein the recombinant microbe or unicellular organism comprises a polynucleotide comprising a sequence encoding a mutated serB gene.

Embodiment 34. The method of Embodiment 33, wherein the mutated serB gene in the recombinant microbe or unicellular organism has reduced expression, is configured to have a reduced amount of a serB gene product, or encodes a serB gene product with reduced enzymatic activity.

Embodiment 35. The method of any one of Embodiments 33-34, wherein the recombinant microbe or unicellular organism comprises: (i) one or more exogenous polynucleotides comprising a sequence encoding a threonine synthase (TS) polypeptide or cysteate synthase (CS) polypeptide; and (ii) one or more exogenous polynucleotides comprising a sequence encoding a sulfinoalanine decarboxylase (SAD) polypeptide, a cysteic acid decarboxylase (CAD) polypeptide, a glutamate decarboxylase (GAD) polypeptide, or a polypeptide corresponding to a decarboxylase portion of the cysteine synthetase/PLP decarboxylase (partCS/PLP-DC), or a combination thereof.

Embodiment 36. The method of Embodiment 35, wherein the recombinant microbe or unicellular organism comprises a modified sulfur-based pathway comprising increased expression of cysPUWA, cysDNC, and cysQ, and a gene knock-out of tauD, ssuD, and ssuE to inhibit taurine degradation.

Embodiment 37. The method of Embodiment 35, wherein the recombinant microbe or unicellular organism comprises a modified sulfur-based pathway comprising a gene knock-out of tauABCD, ssuEADCB, ssuDICBA, sueABCD2, or a combination thereof to inhibit degradation and reuptake of taurine into the cell.

Embodiment 38. The method of any one of Embodiments 33-34, wherein the recombinant microbe or unicellular organism comprises (i) a modified O-phosphoserine pathway comprising increased expression of pgk, serA_{Δ197}, serC, or a combination thereof; and (ii) and a modified sulfur-based pathway comprising (a) increased expression of cysPUWA, cysDNC, cysQ or cysH, and a gene knock-out of tauD, ssuD, and ssuE to inhibit taurine degradation, or (b) a gene knock-out of tauABCD, ssuEADCB, ssuDICBA, sueABCD2, or a combination thereof to inhibit taurine degradation and reuptake of taurine into the cell.

Embodiment 39. The method of any one of Embodiments 31-38, wherein the growing step comprises incubating the recombinant microbe or unicellular organism in a shaker flask to produce at least 0.25 g/liter of taurine from the recombinant microbe or unicellular organism.

Embodiment 40. The method of any one of Embodiments 31-38, wherein the growing step comprises incubating the recombinant microbe or unicellular organism in a fermenter or bioreactor to produce at least 15 g/liter of taurine from the recombinant microbe or unicellular organism.

Embodiment 41. The method of any one of Embodiments 31-40, further comprising: isolating the taurine, wherein the isolated taurine has a purity level of greater than 10% purity, greater than 25% purity, greater than 50% purity, greater than 75% purity, or greater than 98% purity.

Embodiment 42. The method of any one of Embodiments 31-41, wherein the recombinant microbe or unicellular organism is E. coli.

Embodiment 43. The method of Embodiment 42, wherein the growing step is conducted in a medium comprising at least 5 g/L ammonium sulfate, at least 6 g/L dibasic potassium phosphate, at least 3 g/L monobasic sodium phosphate, at least 0.5 g/L magnesium sulfate, at least 6 g/L glucose, at least 0.1 g/L typtone, and at least 0.05 g/L yeast extract.

Embodiment 44. The method of any one of Embodiments 31-41, wherein the recombinant microbe or unicellular organism is C. glutamicum.

Embodiment 45. The method of Embodiment 44, wherein the growing step is conducted in a medium comprising at least 2 g/L yeast extract, 40 g/L glucose, 10 g/L calcium carbonate, 15 g/L ammonium sulfate, 1 g/L dibasic potassium phosphate, and 1 g/L monobasic potassium phosphate.

Embodiment 46. A method for increased cysteate production in a recombinant microbe or unicellular organism, comprising growing a unicellular organism under conditions to produce cysteate, wherein the recombinant microbe or unicellular organism has a modified biosynthetic pathway.

Embodiment 47. The method of Embodiment 46, wherein the modified biosynthetic pathway comprises:
i. a deletion of at least one operon for taurine or sulfonate uptake and degradation;
ii. a deletion of a gene in the enterobactin or enterochelin biosynthetic pathway:
iii. an increased expression of genes in sulfate or thiosulfate transport, sulfur reduction, or sulfur assimilation;
iv. an increased expression of a gene in the PLP pathway
v. an increased expression of a gene in the aspartate pathway;
vi. an increased expression of a gene in the threonine pathway;
vii. a deletion of at least one gene in the degradation of cysteate; or
viii. any combination of (i) to (vii).

Embodiment 48. The method of Embodiments 46 or 47, wherein the recombinant microbe or unicellular organism comprises a polynucleotide comprising a sequence encoding a mutated serB gene.

Embodiment 49. The method of Embodiment 48, wherein the mutated serB gene in the recombinant microbe or unicellular organism has reduced expression, is configured to have a reduced amount of a serB gene product, or encodes a serB gene product with reduced enzymatic activity.

Embodiment 50. The method of any one of Embodiment 48 or 49, wherein the recombinant microbe or unicellular organism comprises one or more exogenous polynucleotides comprising a sequence encoding a threonine synthase (TS) polypeptide or cysteate synthase (CS) polypeptide.

Embodiment 51. The method of Embodiment 50, wherein the recombinant microbe or unicellular organism comprises a modified sulfur-based pathway comprising increased expression of cysPUWA, cysDNC, and cysQ, and a gene knock-out of tauD, ssuD, and ssuE to inhibit cysteate degradation.

Embodiment 52. The method of Embodiment 50, wherein the recombinant microbe or unicellular organism comprises a modified sulfur-based pathway comprising a gene knock-out of cuyA, tauABCD, ssuEADCB, ssuDICBA, sueABCD2, or a combination thereof to inhibit degradation and reuptake of cysteate into the cell.

Embodiment 53. The method of anyone of Embodiments 46-52, wherein the growing step comprises incubating the recombinant microbe or unicellular organism in a shaker flask to produce at least 0.1 g/liter of cysteate from the recombinant microbe or unicellular organism.

Embodiment 54. The method of any one of Embodiments 46-52, wherein the growing step comprises incubating the unicellular organism in a fermentor or bioreactor to produce at least 1 g/liter of cysteate from the recombinant microbe or unicellular organism.

Embodiment 55. The method of any one of Embodiments 46-54, further comprising: isolating the cysteate, wherein the isolated cysteate has a purity level of greater than 10% purity, greater than 25% purity, greater than 50% purity, greater than 75% purity, or greater than 98% purity.

Embodiment 56. The method of any one of Embodiments 46-55, wherein the recombinant microbe or unicellular organism is selected from the group consisting of Bacillus subtilis, Brevibacterium ammoniagene, Corynebacterium crenatum, Corynebacterim pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans, and Saccharomyces cerevisiae.

Embodiment 57. A method for increased production of O-phosphoserine, cysteate, taurine, or a combination thereof, comprising growing a unicellular organism under conditions to produce O-phosphoserine, cysteate, taurine, or a combination thereof, wherein the recombinant microbe or unicellular organism has a modified biosynthetic pathway.

Embodiment 58. The method of Embodiment 57, wherein the recombinant microbe or unicellular organism has reduced import of O-phosphoserine, cysteate, taurine, or a combination thereof, or wherein the recombinant microbe or unicellular organism has increased export of O-phosphoserine, cysteate, taurine, or a combination thereof.

Embodiment 59. The method of Embodiment 58, wherein the recombinant microbe or unicellular organism has a deletion or silencing mutation in sdaC, cycA, sstT, tdcC, or cyuP (yhaO), or a combination thereof.

Embodiment 60. The method of Embodiment 58, wherein the recombinant microbe or unicellular organism has increased expression of emaA (ydeD), alaE (ygaW), yfik, cefA, cefB, rhtA, rhtB, rhtC, gabP, tauP, gadC, yhiM, AAperm, or a combination thereof.

Embodiment 61. The method of Embodiment 57, wherein the recombinant microbe or unicellular organism comprises one or more polypeptides of the O-phosphoserine, cysteate, or taurine biosynthetic pathway configured to be in close proximity to each other by forming molecular scaffolds, channels, or cages, wherein the recombinant microbe or unicellular organism comprises one or more polypeptides of the O-phosphoserine, cysteate, or taurine biosynthetic pathway configured to be in close proximity to polypeptides involved in the production of precursors by forming molecular scaffolds, channels, or cages, or wherein the recombinant microbe or unicellular organism comprises one or more polypeptides of the O-phosphoserine, cysteate, or taurine biosynthetic pathway configured to be in close proximity to exporters of O-phosphoserine, cysteate, or taurine by forming molecular scaffolds, channels, or cages.

Embodiment 62. The method of Embodiment 57, wherein the recombinant microbe or unicellular organism is contacted with exogenous PLP, pyridoxine or a pyridoxine salt.

Embodiment 63. The method of Embodiment 57, wherein the recombinant microbe or unicellular organism has increased expression of one of more genes in the PLP-biosynthetic pathway to increase production of O-phosphoserine, cysteate, or taurine.

Embodiment 64. The method of any one of Embodiments 31-63, further comprising, after the growing step, disrupting the recombinant microbe or unicellular organism, and drying the disrupted recombinant microbe or unicellular organism to prepare a dried product.

Embodiment 65. The method of Embodiment 64, wherein the disrupting step uses chemical, physical, or mechanical means.

Embodiment 66. The method of any one of Embodiments 57-65, wherein the recombinant microbe or unicellular organism comprises bacteria, yeast, fungi, or unicellular algae.

Embodiment 67. A food product, feed product, beverage product, dietary or health supplement product, cosmetic product, personal care product, pharmaceutical product, agricultural production product, or surfactant comprising the dried product prepared by the method of any one of Embodiments 64-66.

Embodiment 68. The recombinant microbe or unicellular organism of any one of Embodiments 15-30, wherein the recombinant microbe or unicellular organism is a dried product.

Embodiments 69. A food product, feed product, beverage product, dietary or health supplement product, cosmetic product, personal care product, pharmaceutical product, agricultural production product, or surfactant comprising the dried product of Embodiment 68.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

The publications and other materials used herein to illuminate the background of the invention or provide additional details respecting the practice, are incorporated by reference, and for convenience are respectively grouped in the References.

### REFERENCES

[1] H.-S. Park, M. J. Hohn, T. Umehara, L.-T. Guo, E. M. Osborne, J. Benner, C. J. Noren, J. Rinehart, D. Söll, Expanding the Genetic Code of Escherichia coli with Phosphoserine, Science 333 (6046) (2011) 1151-1154.
[2] J. P. Saxe, H. Wu, T. K. Kelly, M. E. Phelps, Y. E. Sun, H. I. Kornblum, J. Huang, A phenotypic small-molecule screen identifies an orphan ligand-receptor pair that regulates neural stem cell differentiation, Chem Biol 14 (9) (2007) 1019-30.
[3] C. L. Salgado, B. I. B. Teixeira, F. J. M. Monteiro, Biomimetic Composite Scaffold With Phosphoserine Signaling for Bone Tissue Engineering Application, Frontiers in Bioengineering and Biotechnology 7 (2019).
[4] M. G. Raucci, M. A. Alvarez-Perez, S. Meikle, L. Ambrosio, M. Santin, Poly(Epsilon-Lysine) Dendrons Tethered with Phosphoserine Increase Mesenchymal Stem Cell Differentiation Potential of Calcium Phosphate Gels, Tissue Engineering Part A 20 (3-4) (2013) 474-485.
[5] A. Reinstorf, M. Ruhnow, M. Gelinsky, W. Pompe, U. Hempel, K. W. Wenzel, P. Simon, Phosphoserine—a convenient compound for modification of calcium phosphate bone cement collagen composites, J Mater Sci Mater Med 15 (4) (2004) 451-5.
[6] T. Renner, P. Otto, A. C. Kübler, S. Hölscher-Doht, U. Gbureck, Novel adhesive mineral-organic bone cements based on phosphoserine and magnesium phosphates or oxides, J Mater Sci Mater Med 34 (4) (2023) 14.
[7] J. L. Bystrom, M. Pujari-Palmer, Phosphoserine Functionalized Cements Preserve Metastable Phases, and Reprecipitate Octacalcium Phosphate, Hydroxyapatite, Dicalcium Phosphate, and Amorphous Calcium Phosphate, during Degradation, In Vitro, J Funct Biomater 10 (4) (2019).
[8] Y. Liu, G. Wang, H. Luo, B. Zhao, M. Liao, Q. Dai, M. Li, Q. Li, X. Cao, Phosphoserine enhanced Cu-doped bioactive glass dynamic dual-network hydrogel for craniofacial bone defect repair, Regenerative Biomaterials 10 (2023) rbad054.
[9] S. Z. Scalinci, M. Lugaresi, L. Scorolli, M. Ralli, A. Greco, V. Pantaleone, S. Taurone, F. Franzone, Neuroprotective role of phosphoserine in primary open-angle glaucoma patients, Eur Rev Med Pharmacol Sci 24 (19) (2020) 9780-9786.
   [10] S. E. Edgar, M. A. Hickman, M. M. Marsden, J. G. Morris, Q. R. Rogers, Dietary cysteic acid serves as a precursor of taurine for cats, J Nutr 124 (1) (1994) 103-9.
[11] B. N. Ames, Prolonging healthy aging: Longevity vitamins and proteins, Proc Natl Acad Sci USA 115 (43) (2018) 10836.
[12] R. Lourenco, M. E. Camilo, Taurine: a conditionally essential amino acid in humans? An overview in health and disease, Nutr Hosp 17 (6) (2002) 262-70.
[13] P. J. Markwell, K. E. Earle, Taurine: An essential nutrient for the cat. A brief review of the biochemistry of its requirement and the clinical consequences of deficiency, Nutrition Research 15 (1995) 53-58.
[14] H. Ripps, W. Shen, Review: taurine: a “very essential” amino acid, Molecular Vision 18 (2012) 2673-86.
[15] G. P. Salze, D. A. Davis, Taurine: a critical nutrient for future fish feeds, Aquaculture 437 (2015) 215-229.
[16] P. P. Stapleton, R. P. Charles, H. P. Redmond, D. J. Bouchier-Hayes, Taurine and human nutrition, Clinical Nutrition 16 (1997) 103-8.
[17] Y. Yamori, T. Taguchi, A. Hamada, K. Kunimasa, H. Mori, M. Mori, Taurine in health and diseases: consistent evidence from experimental and epidemiological studies, J Biomed Sci 17 Suppl 1 (2010) S6.
[18] R. J. Huxtable, Physiological actions of taurine, Physiological Reviews 72 (1992) 101-63.
[19] G. Wu, Important roles of dietary taurine, creatine, carnosine, anserine and 4-hydroxyproline in human nutrition and health, Amino Acids 52 (3) (2020) 329-360.
[20] R. V. A. Zafalon, L. W. Risolia, T. H. A. Vendramini, R. B. Ayres Rodrigues, V. Pedrinelli, F. A. Teixeira, M. F. Rentas, M. P. Perini, I. C. Alvarenga, M. A. Brunetto, Nutritional inadequacies in commercial vegan foods for dogs and cats, PLOS One 15 (2020) e0227046.
[21] M. Rojas-Pirela, D. Andrade-Alviárez, V. Rojas, U. Kemmerling, A. J. Cáceres, P. A. Michels, J. L. Concepción, W. Quiñones, Phosphoglycerate kinase: structural aspects and functions, with special emphasis on the enzyme from Kinetoplastea, Open Biology (2020).
[22] P. Peters-Wendisch, M. Stolz, H. Etterich, N. Kennerknecht, H. Sahm, L. Eggeling, Metabolic engineering of Corynebacterium glutamicum for L-serine production, Applied and Environmental Microbiology 71 (2005) 7139-7144.
[23] D. E. Graham, S. M. Taylor, R. Z. Wolf, S. C. Namboori, Convergent evolution of coenzyme M biosynthesis in the Methanosarcinales: cysteate synthase evolved from an ancestral threonine synthase, The Biochemical Journal 424 (2009) 467-478.
[24] T. Mukherjee, J. Hanes, I. Tews, S. E. Ealick, T. P. Begley, Pyridoxal phosphate: Biosynthesis and catabolismbiochimica et Biophysica Acta (BBA)—Proteins and Proteomics 1814 (11) (2011) 1585-1596.
[25] W. Ma, W. Cao, B. Zhang, K. Chen, Q. Liu, Y. Li, P. Ouyang, Engineering a pyridoxal 5′-phosphate supply for cadaverine production by using Escherichia coli whole-cell biocatalysis, Sci Rep 5 (1) (2015) 15630.
[26] H. Han, B. Xu, W. Zeng, J. Zhou, Regulating the biosynthesis of pyridoxal 5′-phosphate with riboswitch to enhance L-DOPA production by Escherichia coli whole-cell biotransformation, Journal of Biotechnology 321 (2020) 68-77.
[27] W. Zheng, K. Chen, S. Fang, X. Cheng, G. Xu, L. Yang, J. Wu, Construction and Application of PLP Self-sufficient Biocatalysis System for Threonine Aldolase, Enzyme and Microbial Technology 141 (2020) 109667.
[28] J. Kim, J. P. Kershner, Y. Novikov, R. K. Shoemaker, S. D. Copley, Three serendipitous pathways in E. coli can bypass a block in pyridoxal-5′-phosphate synthesis, Molecular Systems Biology 6 (1) (2010) 436.
[29] J. Kim, J. J. Flood, M. R. Kristofich, C. Gidfar, A. B. Morgenthaler, T. Fuhrer, U. Sauer, D. Snyder, V. S. Cooper, C. C. Ebmeier, W. M. Old, S. D. Copley, Hidden resources in the Escherichia coli genome restore PLP synthesis and robust growth after deletion of the essential gene pdxB, Proc Natl Acad Sci USA 116 (48) (2019) 24164-24173.
[30] M. Hryniewicz, A. Sirko, A. Pałucha, A. Böck, D. Hulanicka, Sulfate and thiosulfate transport in Escherichia coli K-12: identification of a gene encoding a novel protein involved in thiosulfate binding, Journal of bacteriology 172 (6) (1990) 3358-3366.
[31] C. Zhao, Y. Kumada, H. Imanaka, K. Imamura, K. Nakanishi, Cloning, overexpression, purification, and characterization of O-acetylserine sulfhydrylase-B from Escherichia coli. Protein expression and purification 47 (2006) 607-613.
[32] C. Wang, J. Wu, B. Shi, J. Shi, Z. Zhao, Improving L-serine formation by Escherichia coli by reduced uptake of produced L-serine, Microb Cell Fact 19 (1) (2020) 66.
[33] J. R. van der Ploeg, M. A. Weiss, E. Saller, H. Nashimoto, N. Saito, M. A. Kertesz, T. Leisinger, Identification of sulfate starvation-regulated genes in Escherichia coli: a gene cluster involved in the utilization of taurine as a sulfur source, Journal of Bacteriology 178 (18) (1996) 5438-5446.
[34] J. R. van der Ploeg, N. J. Cummings, T. Leisinger, I. F. Connerton, Bacillus subtilis genes for the utilization of sulfur from aliphatic sulfonates, Microbiology 144 (9) (1998) 2555-2561.
[35] C. Brüggemann, K. Denger, A. M. Cook, J. Ruff, Enzymes and genes of taurine and isethionate dissimilation in Paracoccus denitrificans, Microbiology 150 (4) (2004) 805-816.
[36] K. Denger, J. Ruff, D. Schleheck, A. M. Cook, Rhodococcus opacus expresses the xsc gene to utilize taurine as a carbon source or as a nitrogen source but not as a sulfur source, Microbiology 150 (6) (2004) 1859-1867.
[37] J. R. van der Ploeg, M. A. Weiss, E. Saller, H. Nashimoto, N. Saito, M. A. Kertesz, T. Leisinger, Identification of sulfate starvation-regulated genes in Escherichia coli: A gene cluster involved in the utilization of taurine as a sulfur source, Journal of Bacteriology 178 (1996) 5438-5446.
[38] J. R. van der Ploeg, R. Iwanicka-Nowicka, T. Bykowski, M. M. Hryniewicz, T. Leisinger, The Escherichia coli ssuEADCB Gene Cluster Is Required for the Utilization of Sulfur from Aliphatic Sulfonates and Is Regulated by the Transcriptional Activator Cbl, J Biol Chem 274 (41) (1999) 29358-29365.
[39] K. Denger, T. H. M. Smits, A. M. Cook, Genome-enabled analysis of the utilization of taurine as sole source of carbon or of nitrogen by Rhodobacter sphaeroides 2.4.1, Microbiology 152 (11) (2006) 3197-3206.
[40] Z. Krejcik, D. Schleheck, K. Hollemeyer, A. M. Cook, A five-gene cluster involved in utilization of taurine-nitrogen and excretion of sulfoacetaldehyde by Acinetobacter radioresistens SH164, Archives of microbiology 194 (10) (2012) 857-63.
[41] A. K. Gorzynska, K. Denger, A. M. Cook, T. H. M. Smits, Inducible transcription of genes involved in taurine uptake and dissimilation by Silicibacter pomeroyi DSS-3T, Archives of microbiology 185 (5) (2006) 402-406.
[42] D. J. Koch, C. Rückert, D. A. Rey, A. Mix, A. Pühler, J. Kalinowski, Role of the ssu and seu genes of Corynebacterium glutamicum ATCC 13032 in utilization of sulfonates and sulfonate esters as sulfur sources, Applied and environmental microbiology 71 (10) (2005) 6104-6114.
[43] R. T. Novak, R. F. Gritzer, E. R. Leadbetter, W. Godchaux, Phototrophic utilization of taurine by the purple nonsulfur bacteria Rhodopseudomonas palustris and Rhodobacter sphaeroides, Microbiology 150 (6) (2004) 1881-1891.
[44] Y. Song, C. Yang, G. Chen, Y. Zhang, Z. Seng, Z. Cai, C. Zhang, L. Yang, J. Gan, H. Liang, Molecular insights into the master regulator CysB-mediated bacterial virulence in Pseudomonas aeruginosa, Molecular Microbiology 111 (5) (2019) 1195-1210.
[45] D. A. Rey, A. Pühler, J. Kalinowski, The putative transcriptional repressor McbR, member of the TetR-family, is involved in the regulation of the metabolic network directing the synthesis of sulfur containing amino acids in Corynebacterium glutamicum, Journal of Biotechnology 103 (2003) 61-65.
[46] J. R. van der Ploeg, R. Iwanicka-Nowicka, M. A. Kertesz, T. Leisinger, M. M. Hryniewicz, Involvement of CysB and Cbl regulatory proteins in expression of the tauABCD operon and other sulfate starvation-inducible genes in Escherichia coli, J Bacteriol 179 (24) (1997) 7671-8.
[47] A. L. Demain, The business of biotechnology, Industrial Biotechnology 3 (2007) 269-283.
[48] J. A. Roubos, G. van Straten, A. J. B. van Boxtel, An evolutionary strategy for fed-batch bioreactor optimization; concepts and performance, Journal of Biotechnology 67 (2-3) (1999) 173-187.
[49] T. Oka, Amino acids, production processes, in: M. C. Flickinger, S. W. Drew (Eds.), Encyclopedia of Bioprocess Technology: Fermentation, Biocatalysis, and Bioseparation, Wiley, London, 1999.
[50] M. A. Borowitzka, Commercial production of microalgae: ponds, tanks, tubes and fermenters, Journal of Biotechnology 70 (1-3) (1999) 313-321.
[51] T. Hermann, Industrial production of amino acids by Coryneform bacteria, Journal of Biotechnology 104 (2003) 155-172.
[52] M. Ikeda, Amino acid production processes, Advances in biochemical engineering/biotechnology 79 (2003) 1-35.
[53] A. Richmond, Q. Hu, Handbook of Microalgal Culture: Biotechnology and Applied Phycology, Wiley-Blackwell, Hoboken, New Jersey, 2013.
[54] K. H. Cardozo, T. Guaratini, M. P. Barros, V. R. Falcao, A. P. Tonon, N. P. Lopes, S. Campos, M. A. Torres, A. O. Souza, P. Colepicolo, E. Pinto, Metabolites from algae with economical impact, Comparative biochemistry and physiology. Toxicology & pharmacology: CBP 146 (1-2) (2007) 60-78.
[55] J. J. Milledge, Commercial application of microalgae other than as biofuels: a brief review, Reviews in Environmental Science and Biotechnology 10 (2011) 31-41.
[56] Q. Xu, S. Li, H. Huang, J. Wen, Key technologies for the industrial production of fumaric acid by fermentation, Biotechnol Adv 30 (6) (2012) 1685-1696.
[57] L. Dufossé, M. Fouillaud, Y. Caro, S. A. S. Mapari, N. Sutthiwong, Filamentous fungi are large-scale producers of pigments and colorants for the food industry, Current Opinion in Biotechnology 26 (2014) 56-61.
[58] D. M. Jones, Manual of Methods for General Bacteriology, J Clin Pathol 34 (9) (1981) 1069-1069.
[59] M. A. Oberhardt, R. Zarecki, S. Gronow, E. Lang, H.-P. Klenk, U. Gophna, E. Ruppin, Harnessing the landscape of microbial culture media to predict new organism-media pairings, Nature Communications 6 (1) (2015) 8493.
[60] M. A. Richards, V. Cassen, B. D. Heavner, N. E. Ajami, A. Herrmann, E. Simeonidis, N. D. Price, MediaDB: A database of microbial growth conditions in defined media, PLOS ONE 9 (2014) e103548.
[61] R. Kumar, D. Vikramachakravarthi, P. Pal, Production and purification of glutamic acid: A critical review towards process intensification, Chemical Engineering and Processing 81 (2014) 59-71.
[62] J. Ecker, T. Raab, M. Harasek, Nanofiltration as key technology for the separation of LA and AA, Fuel and Energy Abstracts 389 (2011).
[63] H. Li, T. Qiu, Y. Chen, Y. Cao, Separation of gamma-aminobutyric acid from fermented broth, Journal of Indian Microbiology Biotechnology 38 (2011) 1955-1959.
[64] J. L. Bennetzen, B. D. Hall, Codon selection in yeast, J Biol Chem 257 (6) (1982) 3026-31.
[65] M. Gouy, C. Gautier, Codon usage in bacteria: correlation with gene expressivity, Nucleic Acids Research 10 (22) (1982) 7055-7074.
[66] W. H. Campbell, G. Gowri, Codon Usage in Higher Plants, Green Algae, and Cyanobacteria, Plant Physiol 92 (1) (1990) 1-11.
[67] E. S. Douglas, L. S. Penny, The Plastid Genome of the Cryptophyte Alga, Guillardia theta: Complete Sequence and Conserved Synteny Groups Confirm Its Common Ancestry with Red Algae, Journal of Molecular Evolution 48 (2) 236-244.
[68] H. S. Yoon, K. M. Müller, R. G. Sheath, F. D. Ott, D. Bhattacharya, Defining the major lineages of red algae (rhodophyta), Journal of Phycology 42 (2) (2006) 482-492.
[69] S. P. Fletcher, M. Muto, S. P. Mayfield, Optimization of Recombinant Protein Expression in the Chloroplasts of Green Algae, in: R. León, A. Galván, E. Fernández (Eds.), Transgenic Microalgae as Green Cell Factories, Springer New York, New York, NY, 2007, pp. 90-98.
[70] J. H. Langenheim, K. V. Thimann, Botany: Plant Biology and its Relation to Human Affairs, John Wiley & Sons Inc., New York, 1982.
[71] I. K. Vasil, Cell Culture and Somatic Cell Genetics of Plants: Laboraory Procedures and Their Applications, Academic Press, Orlando, 1984.
[72] R. Stanier, J. Ingrahm, M. Wheelis, P. Painter, The Microbial World, 5 ed., Prentice-Hall, New Jersey, 1986.
[73] O. D. Dhringra, J. B. Sinclair, Basic plant pathology methods, CRC Press, Boca Raton, FL, 1985.
[74] T. Maniatis, E. F. Fritsch, J. Sambrook, Molecular Cloning: A Laboratory Manual: DNA Cloning, Cold Spring Harbor, New York, 1985.
[75] Gait, Oligonucleotide Synthesis-A Practical Approach, IRL Press, Washington, D.C., 1984.
[76] D. D. Hames, S. J. Higgins, Nucleic Acid Hybridization: A Practical Approach, IRL Press, Washington D.C., 1984.
[77] J. D. Watson, M. Gilman, J. Witowski, M. Zoller, Recombinant DNA, Scientific American Books, New York, 1992.
[78] E. Szewczyk, T. Nayak, C. E. Oakley, H. Edgerton, Y. Xiong, N. Taheri-Talesh, S. A. Osmani, B. R. Oakley, Fusion PCR and gene targeting in Aspergillus nidulans, Nature Protocols 1 (2006) 3111-3121.
[79] S. N. Ho, H. D. Hunt, R. M. Horton, J. K. Pullen, L. R. Pease, Site-directed mutagenesis by overlap extension using the polymerase chain reaction, Gene 77 (1989) 51-59.
[80] M. Fuhrmann, W. Oertel, P. Hegemann, A synthetic gene coding for the green fluorescent protein (GFP) is a versatile reporter in Chlamydomonas reinhardtii, Plant Journal 19 (1999) 353-361.
[81] W. Mandecki, T. J. Bolling, FokI method of gene synthesis, Gene 68 (1988) 101-107.
[82] W. P. Stemmer, Crameri, A., Ha, K. D., Brennan, T. M. and Heyneker, H. L., Single-step assembly of a gene and entire plasmid from large numbers of oligodeoxyribonucleotides, Gene 164 (1995) 49-53.
[83] X. Gao, P. Yo, A. Keith, T. J. Ragan, T. K. Harris, Thermodynamically balanced inside-out (TBIO) PCR-based gene synthesis: a novel method of primer design for high-fidelity assembly of longer gene sequences, Nucleic Acids Research 31 (2003) e143.
[84] L. Young, Q. Dong, Two-step total gene synthesis method, Nucleic Acids Research 32 (2004) e59.
[85] J. M. Reyrat, V. Pelicic, B. Gicquel, R. Rappuoli, Counterselectable markers: untapped tools for bacterial genetics and pathogenesis, Infection and immunity 66 (9) (1998) 4011-7.
[86] N. Nakashima, K. Miyazaki, Bacterial cellular engineering by genome editing and gene silencing, International journal of molecular sciences 15 (2) (2014) 2773-93.
[87] J. L. Ried, A. Collmer, An nptI-sacB-sacR cartridge for constructing directed, unmarked mutations in gram-negative bacteria by marker exchange-eviction mutagenesis, Gene 57 (2-3) (1987) 239-46.
[88] K. C. Murphy, K. G. Campellone, A. R. Poteete, PCR-mediated gene replacement in Escherichia coli, Gene 246 (1-2) (2000) 321-30.
[89] W. Sun, S. Wang, R. Curtiss, Highly efficient method for introducing successive multiple scarless gene deletions and markerless gene insertions into the Yersinia pestis chromosome, Appl Environ Microbiol 74 (2008) 4241-4245.
[90] N. Costantino, D. L. Court, Enhanced levels of A Red-mediated recombinants in mismatch repair mutants, Proc Natl Acad Sci USA 100 (26) (2003) 15748-15753.
[91] K. A. Datsenko, B. L. Wanner, One-step inactivation of chromosomal genes in Escherichia coli K-12 using PCR products, Proc Natl Acad Sci USA 97 (12) (2000) 6640-6645.
[92] L. Lv, Y.-L. Ren, J.-C. Chen, Q. Wu, G.-Q. Chen, Application of CRISPRi for prokaryotic metabolic engineering involving multiple genes, a case study: Controllable P (3HB-co-4HB) biosynthesis, Metabolic Engineering 29 (2015) 160-168.
[93] J. M. Peters, M. R. Silvis, D. Zhao, J. S. Hawkins, C. A. Gross, L. S. Qi, Bacterial CRISPR: accomplishments and prospects, Current opinion in microbiology 27 (2015) 121-6.
[94] K. Selle, R. Barrangou, Harnessing CRISPR-Cas systems for bacterial genome editing, Trends in Microbiology 23 (4) (2015) 225-232.
[95] I. Sizova, A. Greiner, M. Awasthi, S. Kateriya, P. Hegemann, Nuclear gene targeting in Chlamydomonas using engineered zinc-finger nucleases, The Plant Journal 73 (5) (2013) 873-882.
[96] F. Daboussi, S. Leduc, A. Maréchal, G. Dubois, V. Guyot, C. Perez-Michaut, A. Amato, A. Falciatore, A. Juillerat, M. Beurdeley, D. F. Voytas, L. Cavarec, P. Duchateau, Genome engineering empowers the diatom Phaeodactylum tricornutum for biotechnology, Nat Commun 5 (2014).
[97] J. H. Miller, A short course in bacterial genetics: a laboratory manual and handbook for Escherichia coli and related bacteria, Cold Spring Harbor Laboratory Press, Plainview, N.Y., 1992.
[98] S. Parekh, V. A. Vinci, R. J. Strobel, Improvement of microbial strains and fermentation processes, Appl Microbiol Biotechnol 54 (3) (2000) 287-301.
[99] S. L. Forsburg, The art and design of genetic screens: yeast, Nature reviews. Genetics 2 (9) (2001) 659-668.
[100] T. Flynn, M. L. Ghirardi, M. Seibert, Accumulation of O2-tolerant phenotypes in H2-producing strains of Chlamydomonas reinhardtii by sequential applications of chemical mutagenesis and selection, International Journal of Hydrogen Energy 27 (11-12) (2002) 1421-1430.
[101] T. T. Y. Doan, J. P. Obbard, Enhanced intracellular lipid in Nannochloropsis sp. via random mutagenesis and flow cytometric cell sorting, Algal Research 1 (1) (2012) 17-21.
[102] M. Hlavova, Z. Turoczy, K. Bisova, Improving microalgae for biotechnology—From genetics to synthetic biology, Biotechnology advances 33 (6 Pt 2) (2015) 1194-1203.
[103] C. Merlin, S. McAteer, M. Masters, Tools for characterization of Escherichia coli genes of unknown function, Journal of Bacteriology 184 (2002) 4573-81.
[104] A. G. Bernheim, V. K. Libis, A. B. Lindner, E. H. Wintermute, Phage-mediated Delivery of Targeted sRNA Constructs to Knock Down Gene Expression in E. coli, (109) (2016) e53618.
[105] T. Fujiwara, M. Ohnuma, M. Yoshida, T. Kuroiwa, T. Hirano, Gene Targeting in the Red Alga Cyanidioschyzon merolae: Single- and Multi-Copy Insertion Using Authentic and Chimeric Selection Markers, PLOS ONE 8 (9) (2013) e73608.
[106] R. Zhang, W. Patena, U. Armbruster, S. S. Gang, S. R. Blum, M. C. Jonikas, High-Throughput Genotyping of Green Algal Mutants Reveals Random Distribution of Mutagenic Insertion Sites and Endonucleolytic Cleavage of Transforming DNA, The Plant cell 26 (4) (2014) 1398-1409.
[107] R. M. Dent, C. M. Haglund, B. L. Chin, M. C. Kobayashi, K. K. Niyogi, Functional Genomics of Eukaryotic Photosynthesis Using Insertional Mutagenesis of Chlamydomonas reinhardtii, Plant Physiol 137 (2) (2005) 545-556.
[108] S. L. Colombo, S. V. Pollock, K. A. Eger, A. C. Godfrey, J. E. Adams, C. B. Mason, J. V. Moroney, Use of the bleomycin resistance gene to generate tagged insertional mutants of Chlamydomonas reinhardtii that require elevated CO2 for optimal growth, Functional Plant Biology 29 (3) (2002) 231-241.
[109] D. Gonzalez-Ballester, W. Pootakham, F. Mus, W. Yang, C. Catalanotti, L. Magneschi, A. de Montaigu, J. J. Higuera, M. Prior, A. Galván, E. Fernandez, A. R. Grossman, Reverse genetics in Chlamydomonas: a platform for isolating insertional mutants, Plant Methods 7 (1) (2011) 1-13.
[110] N. Kleckner, J. Bender, S. Gottesman, Uses of transposons with emphasis on Tn10, Methods Enzymol 204 (1991) 139-80.
[111] H. Kojima, Y. Kawata, A mini-transposon/transposase complex as a new tool for the genetic transformation of microalgae, in: H. Kojima, Y. K. Lee (Eds.), Photosynthetic Microorganisms in Environment Biotechnology, Springer-Verlag, Berlin, Germany, 2001, pp. 41-61.
[112] D. Wu-Scharf, B.-r. Jeong, C. Zhang, H. Cerutti, Transgene and Transposon Silencing in Chlamydomonas reinhardtii by a DEAH-Box RNA Helicase, Science 290 (5494) (2000) 1159-1162.
[113] J. A. Casas-Mollano, J. Rohr, E. J. Kim, E. Balassa, K. van Dijk, H. Cerutti, Diversification of the core RNA interference machinery in Chlamydomonas reinhardtii and the role of DCL1 in transposon silencing, Genetics 179 (1) (2008) 69-81.
[114] I. Y. Goryshin, J. Jendrisak, L. M. Hoffman, R. Meis, W. S. Reznikoff, Insertional transposon mutagenesis by electroporation of released Tn5 transposition complexes, Nat Biotech 18 (1) (2000) 97-100.
[115] K. A. Datsenko, B. L. Wanner, One-step inactivation of chromosomal genes in Escherichia coli K-12 using PCR products, Proc Natl Acad Sci USA 97 (2000).
[116] A. Baudin, O. Ozier-Kalogeropoulos, A. Denouel, F. Lacroute, C. Cullin, A simple and efficient method for direct gene deletion in Saccharomyces cerevisiae, Nucleic Acids Research 21 (14) (1993) 3329-3330.
[117] M. S. Longtine, A. Mckenzie Iii, D. J. Demarini, N. G. Shah, A. Wach, A. Brachat, P. Philippsen, J. R. Pringle, Additional modules for versatile and economical PCR-based gene deletion and modification in Saccharomyces cerevisiae, Yeast (Chichester, England) 14 (10) (1998) 953-961.
[118] M. D. Krawchuk, W. P. Wahls, High-efficiency gene targeting in Schizosaccharomyces pombe using a modular, PCR-based approach with long tracts of flanking homology, Yeast (Chichester, England) 15 (13) (1999) 1419-1427.
[119] J. Zhong, M. Karberg, A. M. Lambowitz, Targeted and random bacterial gene disruption using a group II intron (targetron) vector containing a retrotransposition-activated selectable marker, Nucleic Acids Research 31 (6) (2003) 1656-1664.
[120] A. Minoda, R. Sakagami, F. Yagisawa, T. Kuroiwa, K. Tanaka, Improvement of culture conditions and evidence for nuclear transformation by homologous recombination in a red alga, Cyanidioschyzon merolae 10D, Plant & cell physiology 45 (6) (2004) 667-71.
[121] J.-C. Epinat, S. Arnould, P. Chames, P. Rochaix, D. Desfontaines, C. Puzin, A. Patin, A. Zanghellini, F. Pâques, E. Lacroix, A novel engineered meganuclease induces homologous recombination in yeast and mammalian cells, Nucleic Acids Research 31 (11) (2003) 2952-2962.
[122] T. Li, S. Huang, X. Zhao, D. A. Wright, S. Carpenter, M. H. Spalding, D. P. Weeks, B. Yang, Modularly assembled designer TAL effector nucleases for targeted gene knockout and gene replacement in eukaryotes, Nucleic Acids Research (2011).
[123] Lei S. Qi, Matthew H. Larson, Luke A. Gilbert, Jennifer A. Doudna, Jonathan S. Weissman, Adam P. Arkin, Wendell A. Lim, Repurposing CRISPR as an RNA-Guided Platform for Sequence-Specific Control of Gene Expression, Cell 152 (5) (2013) 1173-1183.
[124] W. Jiang, D. Bikard, D. Cox, F. Zhang, L. A. Marraffini, RNA-guided editing of bacterial genomes using CRISPR-Cas systems, Nature biotechnology 31 (3) (2013) 233-9.
[125] J. E. DiCarlo, J. E. Norville, P. Mali, X. Rios, J. Aach, G. M. Church, Genome engineering in Saccharomyces cerevisiae using CRISPR-Cas systems, Nucleic Acids Research 41 (7) (2013) 4336-43.
[126] J. Z. Jacobs, K. M. Ciccaglione, V. Tournier, M. Zaratiegui, Implementation of the CRISPR-Cas9 system in fission yeast, Nat Commun 5 (2014).
[127] T. Zhao, W. Wang, X. Bai, Y. Qi, Gene silencing by artificial microRNAs in Chlamydomonas, The Plant Journal 58 (1) (2009) 157-164.
[128] T. Si, M. HamediRad, H. Zhao, Regulatory RNA-assisted genome engineering in microorganisms, Current opinion in biotechnology 36 (2015) 85-90.
[129] J. Meng, G. Kanzaki, D. Meas, C. K. Lam, H. Crummer, J. Tain, H. H. Xu, A genome-wide inducible phenotypic screen identifies antisense RNA constructs silencing Escherichia coli essential genes, FEMS microbiology letters 329 (1) (2012) 45-53.
[130] H. Xiao, H. Zhao, Genome-wide RNAi screen reveals the E3 SUMO-protein ligase gene SIZ1 as a novel determinant of furfural tolerance in Saccharomyces cerevisiae, Biotechnology for Biofuels 7 (1) (2014) 1-11.
[131] Z. Bao, H. Xiao, J. Liang, L. Zhang, X. Xiong, N. Sun, T. Si, H. Zhao, Homology-Integrated CRISPR-Cas (HI-CRISPR) System for One-Step Multigene Disruption in Saccharomyces cerevisiae, ACS Synthetic Biology 4 (5) (2015) 585-594.
[132] M. D. De Backer, B. Nelissen, M. Logghe, J. Viaene, I. Loonen, S. Vandoninck, R. de Hoogt, S. Dewaele, F. A. Simons, P. Verhasselt, G. Vanhoof, R. Contreras, W. H. M. L. Luyten, An antisense-based functional genomics approach for identification of genes critical for growth of Candida albicans, Nat Biotech 19 (3) (2001) 235-241.
[134] D. Na, S. M. Yoo, H. Chung, H. Park, J. H. Park, S. Y. Lee, Metabolic engineering of Escherichia coli using synthetic small regulatory RNAs, Nat Biotech 31 (2) (2013) 170-174.
[135] M. Ohnuma, O. Misumi, T. Fujiwara, S. Watanabe, K. Tanaka, T. Kuroiwa, Transient gene suppression in a red alga, Cyanidioschyzon merolae 10D, Protoplasma 236 (1-4) (2009) 107-12.
[136] A. Molnar, A. Bassett, E. Thuenemann, F. Schwach, S. Karkare, S. Ossowski, D. Weigel, D. Baulcombe, Highly specific gene silencing by artificial microRNAs in the unicellular alga Chlamydomonas reinhardtii, Plant J 58 (2009) 165-174.
[137] L. Andersen, L.-O. Sundman, I.-B. Inge-Britt Linden, P. Kontro, S. O. Simo, Synthesis and anticonvulsant properties of some 2-Aminoethanesulfonic acid (Taurine) derivatives, Journal of Pharmaceutical Sciences 73 (1984) 106-108.
[138] C. Herdeis, C. E. Weis, β-Aminoethanesulphonylazide their use for the preparation of 2-aminoethane-sulphonamide (taurylamide), taurolidine or taurultam and their acid addition salts, United States, 1999.
[139] K.-Y. Tserng, D. L. Hachey, P. D. Klein, An improved procedure for the synthesis of glycine and taurine conjugates of bile acids, Journal of Lipid Research 18 (1977) 404-407.
[140] D. W. Fong, J. E. Hoots, Synthesis of tagged polymers by post-polymerization (trans) amidation reaction, Nalco Chemical Company, United States, 1992.
[141] S. Seeberger, R. J. Griffin, I. R. Hardcastle, B. T. Golding, A new strategy for the synthesis of taurine derivatives using the ‘safety-catch’ principle for the protection of sulfonic acids, Org Biomol Chem 5 (2007) 132-138.
[142] M. Suzuki, Y. Nakajima, T. Sato, H. Shirai, K. Hanabusa, Fabrication of TiO2 using L-lysine-based organogelators as organic templates: control of the nanostructures, Chem Commun (4) (2006) 377-379.
[143] S. A. Mikhalenko, L. I. Soloveva, E. A. Lukyanets, Phthalocyanines and related compounds: XXXVIII. Synthesis of symmetric taurine- and choline-substituted phthalocyanines, Russ J Gen Chem 74 (2004) 1775-1800.
[144] R. Capone, S. Blake, M. R. Restrepo, J. Yang, M. Mayer, Designing Nanosensors Based on Charged Derivatives of Gramicidin A, Journal of the American Chemical Society 129 (2007) 9737-9745.
[145] R. C. Gupta, T. Win, S. Bittner, Taurine analogues; A new class of therapeutics: Retrospect and prospects Current Medicinal Chemistry 12 (2005) 2021-2039.
[146] B. A. Johnson, Update on neuropharmacological treatments for alcoholism: Scientific basis and clinical findings, Biochemical Pharmacology 75 (2008) 34-56.
[147] S. Tambour, E. Quertemont, Preclinical and clinical pharmacology of alcohol dependence, Fundamental and Clinical Pharmacology 21 (2007) 9-28.
[148] Y. K. Joung, Y. Sengoku, T. Ooya, K. D. Park, N. Yui, Anticoagulant supramolecular-structured polymers: Synthesis and anticoagulant activity of taurine-conjugated carboxyethylester-polyrotaxanes, Science and Technology of Advanced Materials 6 (2005) 484-490.
[149] N. Özmeriç, G. Özcan, C. M. Haytaç, E. E. Alaaddinoglu, M. F. Sargon, S. Senel, Chitosan film enriched with an antioxidant agent, taurine, in fenestration defects, Journal of Biomedical Materials Research Part A 51 (2000) 500-503.
[150] Z. Degim, N. Öelebi, H. Sayan, A. Babül, D. Erdoğan, G. Take, An investigation on skin wound healing in mice with a taurinechitosan gel formulation, Amino Acids 22 (2002) 187-198.
[151] M. Matsusaki, T. Serizawa, A. Kishida, T. Endo, M. Akashi, Novel functional biodegradable polymer: Synthesis and anticoagulant activity of poly(γ-Glutamic Acid) sulfonate (γ-PGA-sulfonate), Bioconjugate Chemistry 13 (2002) 23-28.
[151] P. Liu, X. Ge, H. Ding, H. Jiang, B. M. Christensen, J. Li, Role of Glutamate Decarboxylase-like Protein 1 (GADL1) in Taurine Biosynthesis, J Biol Chem 287 (49) (2012) 40898-40906.
[152] I. Winge, K. Teigen, A. Fossbakk, E. Mahootchi, R. Kleppe, F. Sköldberg, O. Kämpe, J. Haavik, Mammalian CSAD and GADL1 have distinct biochemical properties and patterns of brain expression, Neurochemistry International 90 (2015) 173-184.
[153] T. F. Smith, M. S. Waterman, Comparison of biosequence, Advances in Applied Mathematics 2 (1981) 482-489.
[154] S. B. Needleman, C. D. Wunsch, A general method applicable to the search for similarities in the amino acid sequence of two proteins, Journal of Molecular Biology 48 (1970) 443-453.
[155] W. R. Pearson, D. J. Lipman, Improved tools for biological sequence comparison, Proceedings of the National Academy of Sciences of the United States of America 85 (1988) 2444-2448.
[156] D. G. Higgins, A. J. Bleasby, R. Fuchs, CLUSTAL V: improved software for multiple sequence alignment, Computer Applications in the Biosciences 8 (2) (1992) 189-191.
[157] D. G. Higgins, P. M. Sharp, CLUSTAL: a package for performing multiple sequence alignment on a microcomputer, Gene 73 (1) (1988) 237-244.
[158] D. G. Higgins, P. M. Sharp, Fast and sensitive multiple sequence alignments on a microcomputer, Computer Applications in the Biosciences 5 (2) (1989) 151-153.
[159] D. F. Feng, R. F. Doolittle, Progressive sequence alignment as a prerequisite to correct phylogenetic trees, Journal of Molecular Evolution 25 (4) (1987) 351-360.
[160] S. Henikoff, J. Henikoff, Amino acid substitution matrices from protein blocks Proceedings of the National Academy of Sciences of the United States of America 89 (1989) 10915-10919.
[161] S. F. Altschul, T. L. Madden, A. A. Schaffer, J. Zhang, Z. Zhang, W. Miller, D. J. Lipman, Gapped BLAST and PSI-BLAST: a new generation of protein database search programs, Nucleic Acids Research 25 (1997) 3389-3402.
[162] J. C. Wootton, S. Federhen, Statistics of local complexity in amino acid sequences and sequence databases, Comput Chem 17 (1993) 149-163.
[163] J. C. Wootton, S. Federhen, Analysis of compositionally biased regions in sequence databases, Methods Enzymol 266 (1996) 554-571.
[164] J.-M. Claverie, D. J. States, Information enhancement methods for large scale sequence analysis, Comput Chem 17 (1993) 191-201.
[165] E. W. Myers, W. Miller, Optimal alignments in linear-space, Computer Applications in the Biological Sciences 4 (1988) 11-17.
J. Buchholz, A. Schwentner, B. Brunnenkan, C. Gabris, S. Grimm, R. Gerstmeir, R. Takors, B. J. Eikmanns, B. Blombach, Platform engineering of Corynebacterium glutamicum with reduced pyruvate dehydrogenase complex activity for improved production of L-lysine, L-valine, and 2-ketoisovalerate, Appl Environ Microbiol 79 (18) (2013) 5566-75.

### Example 1

### Development of a Taurine-Producing Microbe that Contains ΔtauABCD, ΔssuEADCB and ΔentC and the Taurine Producing Pathway CDO-SAD

Step 1: Use chemical synthesis to make a ΔtauABCD polynucleotide (SEQ ID NO:116). Clone the polynucleotide into the vector pTOF25 and transform into an E. coli K12 strain to knockout tauABCD (SEQ ID NO:68) using the recombination methods of Merlin et al.[103]

Step 2: Use chemical synthesis to make a ΔssuEADCB polynucleotide (SEQ ID NO:115). Clone the polynucleotide into the vector pTOF25 and transform into the ΔtauABCD strain (from Step 1 EXAMPLE 1) to knockout ssuEADCB (SEQ ID NO:69) using the recombination methods of Merlin et al.[103]

Step 3: Use chemical synthesis to make a ΔentC polynucleotide (SEQ ID NO:252). Clone the polynucleotide into the vector pTOF25 and transform into the ΔtauABCD/ΔssuEADCB strain (from Step 2 EXAMPLE 1) to knock out entC (SEQ ID NO:250) using the recombination methods of Merlin et al.

Step 4: Use chemical synthesis to make an operable polynucleotide with CDO fused in frame with SAD. Clone the CDO-SAD into a bacterial expression vector so it is functional as follows:
a. The CDO gene is as follows: Derived from SEQ ID NO:1; SEQ ID NO:3; SEQ ID NO: 5 or SEQ ID NO:7 without the stop codon, optimized for expression in the host cell line and encoding a CDO peptide (SEQ ID NO:2; SEQ ID NO:4; SEQ ID NO:6 or SEQ ID NO: 8) and
b. The SAD gene is as follows: Derived from SEQ ID NO:9; SEQ ID NO: 11 or SEQ ID NO: 13 optimized for expression in the host cell line and encoding a SAD peptide from SEQ ID NO:10; SEQ ID NO:12; or SEQ ID NO:14.

Step 5: Transform the vector with the functional CDO-SAD construct (from Step 4, EXAMPLE 1) into the E. coli tauABCD, ΔssuEADCB and ΔentC strain (from Step 3, EXAMPLE 1) and confirm the presence of the DNA construct.

### Example 2

### Development of a Taurine-Producing Microbe that Contains ΔtauABCD, ΔssuEADCB, and ΔentC Strain and the Taurine Producing Pathway TS and partCS/PLP-DC

Step 1: Use chemical synthesis to make an operable polynucleotide with TS and a partCS/PLP-DC peptide by cloning the assembly into a bacterial expression vector so it is functional as follows:
a. The TS gene is as follows: Derived from SEQ ID NO:25; SEQ ID NO:27; SEQ ID NO: 253; or SEQ ID NO:255, optimized for expression in the host cell line and encodes a TS peptide from SEQ ID NO:26; SEQ ID NO:28; SEQ ID NO:254; or SEQ ID NO:256, respectively; and
b. The partCS/PLP-DC gene is derived from one of the following: (i) SEQ ID NO: 17 by removing nucleotides 4 through 1413 (corresponding to the native transit and cysteine synthetase peptides), optimized for expression in the host cell line and encoding a partCS/PLP-DC peptide from Micromonas pusilla (SEQ ID NO:18 minus amino acids 2 through 471), (ii) SEQ ID NO:78 by removing nucleotides 4 through 1413 (corresponding to the native transit and cysteine synthetase peptides), optimized for expression in the host cell line and encoding a partCS/PLP-DC peptide from Ostreococcus tauri (SEQ ID NO:79 minus amino acids 2 through 471), (iii) SEQ ID NO: 198 by removing nucleotides 4 through 1725 (corresponding to the native transit and cysteine synthetase peptides), optimized for expression in the host cell line and encoding a partCS/PLP-DC peptide from Bathycoccus prasinos (SEQ ID NO:199 minus amino acids 2 through 575), (iv) SEQ ID NO:200 by removing nucleotides 4 through 1440 (corresponding to the native transit and cysteine synthetase peptides), optimized for expression in the host cell line and encoding a partCS/PLP-DC peptide from Micromonas commode (SEQ ID NO:201 minus amino acids 2 through 480), or (v) SEQ ID NO:202 minus amino acids 2 through 471), SEQ ID NO: 198 by removing nucleotides 4 through 1464 (corresponding to the native transit and cysteine synthetase peptides), optimized for expression in the host cell line and encoding a partCS/PLP-DC peptide from Pycnococcus provasolii (SEQ ID NO:78 minus amino acids 2 through 488).

Step 2: Transform the vector with the functional TS/partCS/PLP-DC construct (from Step 1, EXAMPLE 2) into the E. coli ΔtauABCD, ΔssuEADCB and ΔentC strain (from Step 3, EXAMPLE 1) and confirm the presence of the DNA construct.

### Example 3

### Development of a Taurine-Producing Microbe in a Strain that Contains ΔtauABCD, ΔssuEADCB and ΔentC and the Taurine Producing Pathway CS/PLP-DC

Step 1: Make an operable polynucleotide with CS/PLP-DC peptide by cloning the assembly into a bacterial expression vector so it is functional as follows:
a. The CS/PLP-DC gene is derived from one of the following: (i) SEQ ID NO:11, by removing nucleotides 4 through 234, (corresponding to the native transit peptide) optimized for expression in yeast, and encoding a CS/PLP-DC peptide from Micromonas pusilla (SEQ ID NO:12 minus amino acids 2 through 78), (ii) SEQ ID NO:78 optimized for expression in the host cell line and encoding a CS/PLP-DC peptide from Ostreococcus tauri (SEQ ID NO:79), (iii) SEQ ID NO:198 by removing nucleotides 4 through 525 (corresponding to the native transit peptide), optimized for expression in the host cell line and encoding a CS/PLP-DC peptide from Bathycoccus prasinos (SEQ ID NO: 199 minus amino acids 2 through 175), (iv) SEQ ID NO:200 by removing nucleotides 4 through 240 (corresponding to the native transit peptide), optimized for expression in the host cell line and encoding a CS/PLP-DC peptide from Micromonas commode (SEQ ID NO:201 minus amino acids 2 through 80), or (v) SEQ ID NO:202 by removing nucleotides 4 through 297 (corresponding to the native transit peptide), optimized for expression in the host cell line and encoding a CS/PLP-DC peptide from Pycnococcus provasolii (SEQ ID NO:78 minus amino acids 2 through 99).

Step 2: Transform the vector with the functional CS/PLP-DC construct (from Step 1, EXAMPLE 3) into the E. coli ΔentC, ΔtauABCD and ΔssuEADCB strain (from Step 3, EXAMPLE 1) and confirm the presence of the DNA construct.

### Example 4

### Development of a Taurine-Producing Microbe that Contains ΔtauABCD, ΔssuEADCB, and ΔentC Strain and the Taurine Producing Pathway with a CL and SAD

Step 1: Use chemical synthesis to make an operable polynucleotide with CL and a SAD peptide by cloning the assembly into a bacterial expression vector so it is functional as follows:
a. The CL gene is as follows: Derived from SEQ ID NO:21 or SEQ ID NO:23, optimized for expression in the host cell line and encodes a CL peptide from SEQ ID NO:22 or SEQ ID NO: 24, respectively; and
b. The SAD gene is as follows: Derived from SEQ ID NO:9; SEQ ID NO:11 or SEQ ID NO: 13 optimized for expression in the host cell line and encoding a SAD peptide from SEQ ID NO: 10; SEQ ID NO:12; or SEQ ID NO:14.

Step 2: Transform the vector with the functional CL/partCS/PLP-DC construct (from Step 1, EXAMPLE 4) into the E. coli ΔtauABCD, ΔssuEADCB and ΔentC strain (from Step 3, EXAMPLE 1) and confirm the presence of the DNA construct.

### Example 5

### Development of a Taurine-Producing Microbe that Contains ΔtauABCD, ΔssuEADCB, and ΔentC Strain and the Taurine Producing Pathway with a Truncated CS/PLP-DC and SAD

Step 1: Use chemical synthesis to make an operable polynucleotide with truncated CS/PLP-DC and a SAD peptide by cloning the assembly into a bacterial expression vector so it is functional as follows:
a. The truncated CS/PLP-DC gene is derived from one of the following: (i) SEQ ID NO: 11, by removing nucleotides 4 through 234, (corresponding to the native transit peptide) and truncated after nucleotide 1407 with the addition of a stop codon, optimized for expression in the host cell line and encoding a truncated CS/PLP-DC peptide from Micromonas pusilla (SEQ ID NO:12 minus amino acids 2 through 78 and truncated after amino acid 469), (ii) SEQ ID NO:78, truncated after nucleotide 1410 with the addition of a stop codon optimized for expression in the host cell line and encoding a truncated CS/PLP-DC peptide from Ostreococcus tauri (SEQ ID NO:79 truncated after amino acid 470), (iii) SEQ ID NO:198 by removing nucleotides 4 through 525 (corresponding to the native transit peptide) and truncated after nucleotide 1731 with the addition of a stop codon, optimized for expression in the host cell line and encoding a truncated CS/PLP-DC peptide from Bathycoccus prasinos (SEQ ID NO:199 minus amino acids 2 through 175 and truncated at amino acid 577), (iv) SEQ ID NO:200 by removing nucleotides 4 through 240 (corresponding to the native transit peptide) and truncated after nucleotide 1425 with the addition of a stop codon, optimized for expression in the host cell line and encoding a truncated CS/PLP-DC peptide from Micromonas commode (SEQ ID NO:201 minus amino acids 2 through 80 and truncated after amino acid 475), or (v) SEQ ID NO: 202 minus amino acids 2 through 297) and truncated after nucleotide 1464 with the addition of a stop codon, optimized for expression in the host cell line and encoding a truncated CS/PLP-DC peptide from Pycnococcus provasolii (SEQ ID NO:78 minus amino acids 2 through 99 and truncated after amino acid 488); and;
b. The SAD gene is as follows: Derived from SEQ ID NO:9; SEQ ID NO:11 or SEQ ID NO: 13 optimized for expression in the host cell line and encoding a SAD peptide from SEQ ID NO:10; SEQ ID NO:12; or SEQ ID NO:14.

Step 2: Transform the vector with the functional truncated CS/PLP-DC and SAD construct (from Step 1, EXAMPLE 5) into the E. coli ΔtauABCD, ΔssuEADCB and ΔentC strain (from Step 3, EXAMPLE 1) and confirm the presence of the DNA construct.

### Example 6

### Development of a Taurine-Producing Microbe in a Strain that Contains ΔtauABCD, ΔssuEADCB and ΔentC Strain and Includes Expression of Genes for PLP Production

Step 1: Make an operable polynucleotide with a functional PLP synthase, pdxS and pdxT peptides, with a different selectable maker from those used in the vectors in EXAMPLES 1-5, as follows:
a. The pdxS derived from SEQ ID NO:242 or SEQ ID NO:244, optimized for expression in the host cell line and encodes PLP synthase, pdxS peptide, of SEQ ID NO:243; SEQ ID NO: 245, respectively; and
b. The pdxT derived from SEQ ID NO:246 or SEQ ID NO:248, optimized for expression in the host cell line and encodes a PLP synthase, pdxT peptide, of SEQ ID NO:247 or SEQ ID NO:249, respectively.

Step 2: Co-transform the pdxS/pdxT containing vector (from Step 1, EXAMPLE 6) into an E. coli ΔtauABCD, ΔssuEADCB and ΔentC strain with one of the following taurine biosynthetic pathways, either CDO-SAD (Step 5, EXAMPLE 1), TS and partCS/PLP-DC (Step 2, EXAMPLE 2), CS/PLP-DC construct (from Step 2, EXAMPLE 3), CL and SAD (Step 2, EXAMPLE 4), or truncated CS/PLP-DC and SAD (Step 2, EXAMPLE 5), confirm the presence of the DNA constructs.

### Example 7

### Development of a Taurine-Producing Microbe in a Strain that Contains ΔtauABCD, ΔssuEADCB and ΔentC and Includes a Constitutively Expressed PUWA and Expression of Genes for PLP Production Operon

Step 1: Use chemical synthesis to make a trcPUWA polynucleotide (SEQ ID NO:118). Clone the polynucleotide into the vector pTOF25 and transform into the ΔtauABCD, ΔssuEADCB and ΔentC E. coli K12 strain (from Step 2 EXAMPLE 1) to knock in a constitutive promoter to replace the native promoter for cysPUWA (SEQ ID NO:110) using the recombination methods of Merlin et al.[103]

Step 2: Co-transform the pdxS/pdxT containing vector (from Step 1, EXAMPLE 6) into an E. coli ΔtauABCD, ΔssuEADCB, ΔentC, trcPUWA strain vector (from Step 1, EXAMPLE 7) with one of the following taurine biosynthetic pathways: CDO-SAD (Step 4, EXAMPLE 1), TS and partCS/PLP-DC (Step 1, EXAMPLE 2), CS/PLP-DC construct (from Step 1, EXAMPLE 3), CL and SAD (Step 1, EXAMPLE 4), or truncated CS/PLP-DC and SAD (Step 1, EXAMPLE 5). Confirm the presence of the DNA construct.

### Example 8

### Development of a Taurine-Producing Microbe in a Strain that Contains ΔtauABCD, ΔssuEADCB, ΔentC and trcPUWA and Includes a Constitutively Expressed DNC and Genes for PLP Production

Step 1: Use chemical synthesis to make a trcDNC polynucleotide (SEQ ID NO:117). Clone the polynucleotide into the vector pTOF25 and transform into the ΔtauABCD, ΔssuEADCB and ΔentC E. coli K12 strain (from Step 2 EXAMPLE 1) to knock in a constitutive promoter to replace the native promoter for cysDNC (SEQ ID NO:47) using the recombination methods of Merlin et al.[103]

Step 2: Co-transform the pdxS/pdxT containing vector (from Step 1, EXAMPLE 6) into an E. coli ΔtauABCD, ΔssuEADCB, ΔentC, trcPUWA, cysDNC strain vector (from Step 1, EXAMPLE 8) with one of the following taurine biosynthetic pathways: CDO-SAD (Step 4, EXAMPLE 1), TS and partCS/PLP-DC (Step 1, EXAMPLE 2), CS/PLP-DC construct (from Step 1, EXAMPLE 3), CL and SAD (Step 1, EXAMPLE 4), or truncated CS/PLP-DC and SAD (Step 1, EXAMPLE 5). Confirm the presence of the DNA construct.

### Example 9

### Development of Another High Taurine-Producing Microbe

Step 1: Generate a DNA fragment using genomic DNA from C. glutamicum and the primer pairs, SEQ ID NO: 122 and SEQ ID NO:123. Generate a second DNA fragment using genomic DNA from C. glutamicum and the primer pairs, SEQ ID NO:124 and SEQ ID NO:125. Purify each DNA fragment and use them in overlap PCR with primers SEQ ID NO: 122 and SEQ ID NO: 125 to make a knockout fragment for ssuE (SEQ ID NO:76). Clone the resulting fragment into the pK19mobsacB vector and transform into C. glutamicum to replace ssuE with the ssuE knockout fragment by homologous recombination as described by Buchholz et al.[166]

Step 2: Make a ΔmcbR in the ΔssuE strain (from Step 1, EXAMPLE 9) using the synthetic polynucleotide (SEQ ID NO:142) and recombination methods as described by Buchholz et al.[166]

Step 3: Make a ΔilvA in the ΔssuE/ΔmcbR strain (from Step 2, EXAMPLE 9) using the synthetic polynucleotide (SEQ ID NO:139) and recombination methods as described by Buchholz et al.[166]

Step 4: Make a ΔglyA in the ΔssuE/ΔmcbR/ΔilvA strain (from Step 3, EXAMPLE 9) using the synthetic polynucleotide (SEQ ID NO:138) and recombination methods as described by Buchholz et al.[166]

Step 5: Use chemical synthesis to make an operable polycistronic CDO/SAD polynucleotide optimized for expression in the host cell line as follows:
a. The CDO gene is derived from SEQ ID NO:1 and encodes a CDO peptide from Danio rerio (SEQ ID NO:2); and
b. The SAD gene is derived from SEQ ID NO:9 and encodes a SAD peptide from Danio rerio (SEQ ID NO:10).

Step 6: Use chemical synthesis to make an operable polycistronic pgk/serA_{Δ197}/serC/serB polynucleotide.
a. The pgk gene is derived from SEQ ID NO:35 and encodes a pgk peptide from C. glutamicum (SEQ ID NO: 36); and
b. The serA_{Δ197} gene is derived from SEQ ID NO:37 and encodes the serA_{Δ197} peptide from C. glutamicum (SEQ ID NO:38); and
c. The serC gene is derived from SEQ ID NO:41 and encodes the serC peptide from C. glutamicum (SEQ ID NO:42); and
d. The serB gene is derived from SEQ ID NO:39 and encodes the serB peptide from C. glutamicum (SEQ ID NO:40); and

Step 8: Co-transform the vectors with the functional CDO/SAD (from Step 5, EXAMPLE 9) and pgk/serA_{Δ197}/serC/serB (from Step 6, EXAMPLE 9 into the ΔssuE/ΔmcbR/ΔilvA/glyA strain (from Step 4, EXAMPLE 9) and confirm the presence of the DNA construct.

### Example 10

### Microbial Fermentation Process to Produce at Least 0.25 g/L Taurine in a Shaker Flask

Step 1: Grow a seed culture of taurine-producing bacteria (from EXAMPLES 1, 2, 3, 4, 5, 6, 7 or 8) in LB broth with the appropriate antibiotic(s) for 12-20 hours on a rotary shaker at 37° C. and 250 rpm.

Step 2: Inoculate production media with 1/50 volume of seed culture. The production media contains ammonium sulfate (5 g/L), dibasic potassium phosphate (6 g/L), monobasic sodium phosphate 3 g/L, magnesium sulfate 0.5 g/L, glucose 6 g/L, typtone 0.1 g/L and yeast extract 0.05 g/L, with or without antibiotic(s), and pH 7.0. Grow taurine-producing bacteria in production media in beveled flasks for 20-48 hours in a rotary shaker at 300 rpm and 33° C.

Step 3: Separate cells from broth by centrifugation.

Step 4: Determine the taurine concentration in the cells and cleared broth by HPLC.

### Example 11

### Microbial Fermentation Process to Produce at Least 15 g/L Taurine in a Fermentor

Step 1: Grow the seed culture of taurine-producing bacteria (from EXAMPLES 1, 2, 3, 4, 5, 6, 7, or 8) in LB broth with the appropriate antibiotic(s) for 12-20 hours on a rotary shaker at 250 rpm and 37° C.

Step 2: Conduct batch fermentation in a 2 L bioreactor using production media from Step 2 and EXAMPLE 13 plus an antifoaming agent. Maintain pH at 7.0 with ammonium hydroxide, temperature at 33° C., and dissolved oxygen above 20% by adjusting the agitation speed and airflow.

Step 3: Separate cells from broth by centrifugation.

Step 4: Determine the taurine concentration in the cells and cleared broth by HPLC.

### Example 12

### Another Microbial Fermentation Process to Produce at Least 0.25 g/L Taurine in a Shaker Flask

Step 1. Grow the seed culture of taurine-producing bacteria (from EXAMPLE 9) in LB broth with 0.5% glucose with the appropriate antibiotic(s) for 24 hours on a rotary shaker at 200 rpm and 30° C. for 48 hours.

Step 2: Inoculate production media with 1/10 volume of seed culture. The production media contains yeast extract (2 g/L), glucose (40 g/L), calcium carbonate (10 g/L), ammonium sulfate (15 g/L), dibasic potassium phosphate (1 g/L), monobasic potassium phosphate (1 g/L), sodium chloride (2 g/L), calcium chloride (80 mg/L), ferric chloride (3 mg/L), zinc sulfate heptahydrate (0.9 mg/L), cupric sulfate (0.2 mg/L), manganese sulfate (0.4 mg/L), sodium molybdate (0.1 mg/L), sodium borate (0.3 mg/L), magnesium sulfate (1 g/L), thiamine hydrochloride (0.2 mg/L), biotin (0.2 mg/L), with or without antibiotic(s), pH 7.0. Grow taurine-producing bacteria in production media in beveled flasks for 24 hours in a rotary shaker at 250 rpm and 30° C.

Step 3: Separate cells from broth by centrifugation,

Step 4: Determine the taurine concentration in the cells and cleared broth by HPLC.

### Example 13

### Another Microbial Fermentation Process to Produce at Least 15 g/L Taurine in a Fermentor

Step 1: Grow the seed culture of taurine-producing bacteria (from EXAMPLE 9) in LB broth with the appropriate antibiotic(s) for 24 hours on a rotary shaker at 200 rpm and 30° C.

Step 2: Conduct batch fermentation in a 2 L bioreactor with production media from Step 2, EXAMPLE 15 plus an antifoaming agent. Maintain pH at 7.0 with potassium hydroxide and phosphoric acid, temperature at 30° C., and dissolved oxygen above 20% by adjusting the agitation speed and airflow.

Step 3: Separate cells from broth by centrifugation.

Step 4: Determine the taurine concentration in the cells and cleared broth by HPLC.

### Example 14

### Purification of Taurine to at Least 90% from Cleared Broth

Step 1: Purify taurine from the cleared broth (Step 3, EXAMPLES 10-13) by cation exchange as follows:
a. Concentrate solution with ultrafiltration membrane; and
b. Adjust pH of the cleared broth solution to pH>8.0 with NaOH; and
c. Wash basic exchange column with 0.1N acid and water,
d. Add taurine solution to the activated basic exchange column; and
e. Elute taurine with weak acid.

Step 2: Dry down solution to crystal or powder form.

Step 3: Determine taurine concentration by HPLC.

### Example 18

### Purification of Taurine of at Least 90% from Fermented Cells

Step 1: Suspend cells (from Step 3, EXAMPLES 15 OR 16) in 0.1N HCl.

Step 2: Disrupt cells by chemical agents, pressure, mechanical force, or ultrasonification to release their contents.

Step 3: Separate cellular debris from supernatant by centrifugation.

Step 4: Purify taurine from the supernatant (Step 3, EXAMPLES 18) by basic exchange as described in Steps 1a through 1e, EXAMPLE 17.

Step 5: Dry down solution to crystal or powder form.

Step 6: Determine taurine concentration by HPLC.

### OTHER ASPECTS

While the subject matter of this disclosure has been described and shown in considerable detail with reference to certain illustrative aspects, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

Other aspects, advantages, and modifications are within the scope of the following claims.

What is claimed is:

1. A recombinant microbe or unicellular organism, comprising:
a polynucleotide comprising a sequence encoding a mutated serB gene, and one or more exogenous polynucleotides comprising a sequence encoding a threonine synthase (TS) polypeptide or cysteate synthase (CS) polypeptide,
wherein the mutated serB gene has reduced expression, is configured to have a reduced amount of a serB gene product, or encodes a serB gene product with reduced enzymatic activity, and
wherein the recombinant microbe or unicellular organism has a modified biosynthetic pathway.

2. The recombinant microbe or unicellular organism of claim 1, further comprising one or more exogenous polynucleotides comprising a sequence encoding a sulfinoalanine decarboxylase (SAD) polypeptide, a cysteic acid decarboxylase (CAD) polypeptide, a glutamate decarboxylase (GAD) polypeptide, or a polypeptide corresponding to a decarboxylase portion of the cysteine synthetase/PLP decarboxylase (partCS/PLP-DC).

3. The recombinant microbe or unicellular organism of claim 1, wherein the modified biosynthetic pathway comprises a modified sulfur-based pathway that inhibits cysteate degradation or inhibits taurine degradation.

4. The recombinant microbe or unicellular organism of claim 3, wherein:
a) modified sulfur-based pathway comprises increased expression of cysPUWA, cysDNC, and cysQ, and a gene knock-out of tauD, ssuD, and ssuE;
b) recombinant microbe or unicellular organism has a gene knock-out of cuyA, tauABCD, ssuEADCB, ssuDICBA, sueABCD2, or a combination thereof; or
c) recombinant microbe or unicellular organism has a gene knock-out of tauABCD, ssuEADCB, ssuDICBA or sueABCD2, or a combination thereof.

5. The recombinant microbe or unicellular organism of claim 2, wherein the modified biosynthetic pathway comprises a modified O-phosphoserine pathway comprising increased expression of pgk, serA_{Δ197}, serC, or a combination thereof, and wherein the recombinant microbe or unicellular organism further comprises a modified sulfur-based pathway.

6. The recombinant microbe or unicellular organism of claim 5, wherein the modified sulfur-based pathway comprises increased expression of cysPUWA, cysDNC, cysQ or cysH, and a gene knock-out of tauD, ssuD, and ssuE, or wherein the modified sulfur-based pathway comprises a gene knock-out of tauABCD, ssuEADCB, ssuDICBA, sueABCD2, or a combination thereof.

7. A recombinant microbe or unicellular organism, wherein the recombinant microbe or unicellular organism has increased production of O-phosphoserine, cysteate, taurine, or a combination thereof.

8. The recombinant microbe or unicellular organism of claim 7, wherein:
a) the recombinant microbe or unicellular organism has reduced import of O-phosphoserine, cysteate, taurine, or a combination thereof, or wherein the recombinant microbe or unicellular organism has increased export of O-phosphoserine, cysteate, taurine, or a combination thereof;
b) the recombinant microbe or unicellular organism comprises one or more polypeptides of the O-phosphoserine, cysteate, or taurine biosynthetic pathway configured to be in close proximity to each other by forming molecular scaffolds, channels, or cages;
c) the recombinant microbe or unicellular organism comprises one or more polypeptides of the O-phosphoserine, cysteate, or taurine biosynthetic pathway configured to be in close proximity to polypeptides involved in the production of precursors by forming molecular scaffolds, channels, or cages;
d) wherein the recombinant microbe or unicellular organism comprises one or more polypeptides of the O-phosphoserine, cysteate, or taurine biosynthetic pathway configured to be in close proximity to exporters of O-phosphoserine, cysteate, or taurine by forming molecular scaffolds, channels, or cages;
e) the recombinant microbe or unicellular organism is contacted with exogenous PLP, pyridoxine or a pyridoxine salt; or
f) the recombinant microbe or unicellular organism has increased expression of one of more genes in the PLP-biosynthetic pathway to increase production of O-phosphoserine, cysteate, or taurine.

9. The recombinant microbe or unicellular organism of claim 8, wherein:
a) the recombinant microbe or unicellular organism has a deletion or silencing mutation in sdaC, cycA, sstT, tdcC, or cyuP (yhaO), or a combination thereof; or
b) the recombinant microbe or unicellular organism has increased expression of emaA (ydeD), alaE (ygaW), yfik, cefA, cefB, rhtA, rhtB, rhtC, gabP, tauP, gadC, yhiM AAperm, or a combination thereof.

10. The recombinant microbe or unicellular organism of claim 1, wherein the recombinant microbe or unicellular organism comprises bacteria, yeast, fungi, or unicellular algae.

11. A method for increased taurine production in a recombinant microbe or unicellular organism, comprising growing a unicellular organism under conditions to produce taurine, wherein the recombinant microbe or unicellular organism has a modified biosynthetic pathway.

12. The method of claim 11, wherein the modified biosynthetic pathway comprises:
(i) a deletion of at least one operon for taurine or sulfonate uptake and degradation;
(ii) a deletion of a gene in the enterobactin or enterochelin biosynthetic pathway:
(iii) an increased expression of genes in sulfate or thiosulfate transport, or sulfur reduction or sulfur assimilation;
(iv) an increased expression of a gene in the PLP pathway;
(v) an increased expression of a gene in the serine biosynthetic pathway;
(vi) an increased expression of a gene in the cysteine biosynthetic pathway;
(vii) an increased expression of a gene in the 2-aminoacrylate biosynthetic pathway;
(viii) a deletion of at least one gene in the degradation of taurine, serine, cysteate or 2-aminoacrylate; or
(ix) any combination of (i) to (viii).

13. The method of claim 11, wherein the recombinant microbe or unicellular organism comprises a polynucleotide comprising a sequence encoding a mutated serB gene.

14. The method of claim 13, wherein:
a) the mutated serB gene in the recombinant microbe or unicellular organism has reduced expression, is configured to have a reduced amount of a serB gene product, or encodes a serB gene product with reduced enzymatic activity;
b) the recombinant microbe or unicellular organism comprises: (i) one or more exogenous polynucleotides comprising a sequence encoding a threonine synthase (TS) polypeptide or cysteate synthase (CS) polypeptide; and (ii) one or more exogenous polynucleotides comprising a sequence encoding a sulfinoalanine decarboxylase (SAD) polypeptide, a cysteic acid decarboxylase (CAD) polypeptide, a glutamate decarboxylase (GAD) polypeptide, or a polypeptide corresponding to a decarboxylase portion of the cysteine synthetase/PLP decarboxylase (partCS/PLP-DC), or a combination thereof; or
c) the recombinant microbe or unicellular organism comprises (i) a modified O-phosphoserine pathway comprising increased expression of pgk, serA_{Δ197}, serC, or a combination thereof; and (ii) and a modified sulfur-based pathway comprising (a) increased expression of cysPUWA, cysDNC, cysQ or cysH, and a gene knock-out of tauD, ssuD, and ssuE to inhibit taurine degradation, or (b) a gene knock-out of tauABCD, ssuEADCB, ssuDICBA, sueABCD2, or a combination thereof to inhibit taurine degradation and reuptake of taurine into the cell.

15. The method of claim 14, wherein
a) the recombinant microbe or unicellular organism comprises a modified sulfur-based pathway comprising increased expression of cysPUWA, cysDNC, and cysQ, and a gene knock-out of tauD, ssuD, and ssuE to inhibit taurine degradation; or
b) the recombinant microbe or unicellular organism comprises a modified sulfur-based pathway comprising a gene knock-out of tauABCD, ssuEADCB, ssuDICBA, sueABCD2, or a combination thereof to inhibit degradation and reuptake of taurine into the cell.

16. The method of claim 11, wherein the growing step comprises:
a) incubating the recombinant microbe or unicellular organism in a shaker flask to produce at least 0.25 g/liter of taurine from the recombinant microbe or unicellular organism; or
b) incubating the recombinant microbe or unicellular organism in a fermenter or bioreactor to produce at least 15 g/liter of taurine from the recombinant microbe or unicellular organism.

17. The method of claim 11, further comprising: isolating the taurine, wherein the isolated taurine has a purity level of greater than 10% purity, greater than 25% purity, greater than 50% purity, greater than 75% purity, or greater than 98% purity.

18. The method of claim 11, wherein the recombinant microbe or unicellular organism is E. coli or C. glutamicum.

19. The method of claim 18, wherein:
a) when the recombinant microbe or unicellular organism is E. coli, the growing step is conducted in a medium comprising at least 5 g/L ammonium sulfate, at least 6 g/L dibasic potassium phosphate, at least 3 g/L monobasic sodium phosphate, at least 0.5 g/L magnesium sulfate, at least 6 g/L glucose, at least 0.1 g/L typtone, and at least 0.05 g/L yeast extract; and
b) when the recombinant microbe or unicellular organism is C. glutamicum, the growing step is conducted in a medium comprising at least 2 g/L yeast extract, 40 g/L glucose, 10 g/L calcium carbonate, 15 g/L ammonium sulfate, 1 g/L dibasic potassium phosphate, and 1 g/L monobasic potassium phosphate.

20. A method for increased cysteate production in a recombinant microbe or unicellular organism, comprising growing a unicellular organism under conditions to produce cysteate, wherein the recombinant microbe or unicellular organism has a modified biosynthetic pathway.

21. The method of claim 20, wherein the modified biosynthetic pathway comprises:
(i) a deletion of at least one operon for taurine or sulfonate uptake and degradation;
(ii) a deletion of a gene in the enterobactin or enterochelin biosynthetic pathway:
(iii) an increased expression of genes in sulfate or thiosulfate transport, sulfur reduction, or sulfur assimilation;
(iv) an increased expression of a gene in the PLP pathway
(v) an increased expression of a gene in the aspartate pathway;
(vi) an increased expression of a gene in the threonine pathway;
(vii) a deletion of at least one gene in the degradation of cysteate; or
(viii) any combination of (i) to (vii).

22. The method of claim 20, wherein the recombinant microbe or unicellular organism comprises a polynucleotide comprising a sequence encoding a mutated serB gene.

23. The method of claim 22, wherein:
a) the mutated serB gene in the recombinant microbe or unicellular organism has reduced expression, is configured to have a reduced amount of a serB gene product, or encodes a serB gene product with reduced enzymatic activity; or
b) the recombinant microbe or unicellular organism comprises one or more exogenous polynucleotides comprising a sequence encoding a threonine synthase (TS) polypeptide or cysteate synthase (CS) polypeptide.

24. The method of claim 23, wherein:
a) the recombinant microbe or unicellular organism comprises a modified sulfur-based pathway comprising increased expression of cysPUWA, cysDNC, and cysQ, and a gene knock-out of tauD, ssuD, and ssuE to inhibit cysteate degradation; or
b) the recombinant microbe or unicellular organism comprises a modified sulfur-based pathway comprising a gene knock-out of cuyA, tauABCD, ssuEADCB, ssuDICBA, sueABCD2, or a combination thereof to inhibit degradation and reuptake of cysteate into the cell.

25. The method of claim 20, wherein:
a) the growing step comprises incubating the recombinant microbe or unicellular organism in a shaker flask to produce at least 0.1 g/liter of cysteate from the recombinant microbe or unicellular organism; or
b) the growing step comprises incubating the unicellular organism in a fermentor or bioreactor to produce at least 1 g/liter of cysteate from the recombinant microbe or unicellular organism.

26. The method of claim 20, further comprising: isolating the cysteate, wherein the isolated cysteate has a purity level of greater than 10% purity, greater than 25% purity, greater than 50% purity, greater than 75% purity, or greater than 98% purity.

27. The method of claim 20, wherein the recombinant microbe or unicellular organism is selected from the group consisting of Bacillus subtilis, Brevibacterium ammoniagene, Corynebacterium crenatum, Corynebacterim pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans, and Saccharomyces cerevisiae.

28. The method of claim 11, further comprising, after the growing step, disrupting the recombinant microbe or unicellular organism, and drying the disrupted recombinant microbe or unicellular organism to prepare a dried product.

29. A food product, feed product, beverage product, dietary or health supplement product, cosmetic product, personal care product, pharmaceutical product, agricultural production product, or surfactant comprising the dried product prepared by the method of claim 28.

30. The recombinant microbe or unicellular organism of claim 1, wherein the recombinant microbe or unicellular organism is a dried product.


SEQUENCE LISTING (262 sequences)

SEQ ID NO: 1 - DNA, 606 bp, Danio rerio
  source 1..606: mol_type = genomic DNA
atggagcagactgaagtcatgaagcccgagactctggaggatctgatcaaaactctgcatcagatcttccagagcgactccatcaatgtggaggaggtgcagaacctgatggagtcctaccagagcaacccgcaggactggatgaagttcgccaagttcgaccagtacaggtacaccaggaacctcgtggatgaaggaaacggaaagttcaacctgatgatcctgtgctggggtgaaggacacggcagcagcatccatgaccacacagactcgcactgcttcctgaagctgctgcagggtcagctgaaggagacgctgttcgactggcccgaccgcaagctgcagagcggcatgaagccccgcggccagagcgtgctgcaggagaaccagtgcgcgtacatcaacgactctctgggactccaccgtgtggagaatgtgagccacacagagccggccgtgagtctgcacctttacagtcctccgttccagagctgccgcacgtttgaccagcgcaccggacaccacaacaccgtcaagatgaccttctggagcaaatatggcgagaggacgccctatgagctgagcgtctcgcaggagaataactga

SEQ ID NO: 2 - protein, 201 aa, Danio rerio
MEQTEVMKPETLEDLIKTLHQIFQSDSINVEEVQNLMESYQSNPQDWMKFAKFDQYRYTRNLVDEGNGKFNLMILCWGEGHGSSIHDHTDSHCFLKLLQGQLKETLFDWPDRKLQSGMKPRGQSVLQENQCAYINDSLGLHRVENVSHTEPAVSLHLYSPPFQSCRTFDQRTGHHNTVKMTFWSKYGERTPYELSVSQENN

SEQ ID NO: 3 - DNA, 810 bp, Chlamydomonas reinhardtii
  source 1..810: mol_type = genomic DNA
atgtcttctatcatcgctatgcctatcaacgaggacggtgtcgttgtggtcgaccgcaagctgctgggcaacgaggtcgagagcaaggcccgctgcgcggacaccgcctgcaccgcggctgcgcccgccccgcccgccacggcggccgcgcccacctccatgccggagctgttgcaggcgttgcagcgcgccattgacgaggagaaggccactggccaggtcgccatcaacgctgtggaccagacgcccgagtccgctgcgcggctgagcgcccgcgtgcaggctctactctcggcctacaccagctccaactcgggcgactggcgacgctacgccatgttcaacgacatccactacgtgcgcaacctggtggatgccaatgaggactttgaactaattgttctttgttggaagcgcgggcaagtcagccgcgtgcacaaccacgccaacgcgcactgctggctggcggtgctggacggcgagatgcgcgagacgcagttccagcgcgcgtccgcgccgcccggctgccccgcgcccgcggcctcggagcacgatggcagcactgtgtacgtggagcccacacaggtgtccgacatgcgagtgggtgacgccggctacatcaacgactccatggcgctgcacaacgtggggtgttgcatgcccgccctggccgctggcgaggagggccccgagggcggggtgacgctgcactgctacgcccccccgattcgccgcgtcaagatctatgaggacagcaaggtcacggagcgcgtgcccggctactactccaagggcggagtgcgcgtttga

SEQ ID NO: 4 - protein, 269 aa, Chlamydomonas reinhardtii
MSSIIAMPINEDGVVVVDRKLLGNEVESKARCADTACTAAAPAPPATAAAPTSMPELLQALQRAIDEEKATGQVAINAVDQTPESAARLSARVQALLSAYTSSNSGDWRRYAMFNDIHYVRNLVDANEDFELIVLCWKRGQVSRVHNHANAHCWLAVLDGEMRETQFQRASAPPGCPAPAASEHDGSTVYVEPTQVSDMRVGDAGYINDSMALHNVGCCMPALAAGEEGPEGGVTLHCYAPPIRRVKIYEDSKVTERVPGYYSKGGVRV

SEQ ID NO: 5 - DNA, 606 bp, Pseudomonas aeruginosa
  source 1..606: mol_type = genomic DNA
atgtcatccatcctgcgcctcgaccgcctgcgccagttcatcggcgagctggcgacactgctcgacagccgtcccgacgaatccaccctgctcgcccaagcccaccccctgctggccgagctggtgcgccaggacgactggctgccggaagactgcgcccgccccgatccacagcgctaccaacagtacctgctgcatatcgactcacggcagcgcttctcggtggtcagcttcgtctgggggccgggccagatcacaccggtacacgatcatcgggtctggggcctgatcggcatgctccgcggggccgaatactcgcagccgtacgccttcgatgcgggggggcgtccgcatcccagcggagcccgtcgacgcctggagcccggcgaggtcgaagcgctgtcgccacgcattggcgacgtgcaccaggtgagcaacgccttcagcgaccgaacatccatcagtatccacgtctacggcgccaatatcggtgcggtacggcgtgccgtgttcagcgccgaaggtgaggaaaaacccttcatttccagctattccaacagccgcttgcccaatatctgggacctgtcgaaagagaaccccgcatga

SEQ ID NO: 6 - protein, 201 aa, Pseudomonas aeruginosa
MSSILRLDRLRQFIGELATLLDSRPDESTLLAQAHPLLAELVRQDDWLPEDCARPDPQRYQQYLLHIDSRQRFSVVSFVWGPGQITPVHDHRVWGLIGMLRGAEYSQPYAFDAGGRPHPSGARRRLEPGEVEALSPRIGDVHQVSNAFSDRTSISIHVYGANIGAVRRAVFSAEGEEKPFISSYSNSRLPNIWDLSKENPA

SEQ ID NO: 7 - DNA, 639 bp, unidentified
  source 1..639: mol_type = genomic DNA
atgacgcctacagccgcaggcagccggacctcaaccgagtttcaacaaggtcggaggactgaggtgagccaaaagctgcttgcatttgtggccgaggtgacacgctgtgtcgaaaacgttcaggacgaagtcatagtcgtttcgcgcgtcaggaaggcgatgagcgagcttgtgtcaacggatgactggttgcctgagacgatggcgctgtccgatcccaaattctatcagcaacatctgctatatggagaccctcttgatcgcttttcggtcgtgagcttcgtttggggaccgggccagcagacgccggtccatgaccattccgtgtggggtgtcatcggcatgttgcgaggatccgagatcaaccagaggtacggccggacggcctatgggatggacccgatcggtggttccgagcgactggagccaggcgacgtggaaatcgtgtcggaagcggttggcgacattcatcaagtgcgaaatgcgttcgatgaccgcgtgtccattagtatccactgctacggtgggaatatcgggagaatccgacgtcatgtcttcaaagatctcggtgagacccaagagtttgtctcgggttattccaatgctctcactcccaacctctgggcgccagcgcagtga

SEQ ID NO: 8 - protein, 191 aa, unidentified
MSQKLLAFVAEVTRCVENVQDEVIVVSRVRKAMSELVSTDDWLPETMALSDPKFYQQHLLYGDPLDRFSVVSFVWGPGQQTPVHDHSVWGVIGMLRGSEINQRYGRTAYGMDPIGGSERLEPGDVEIVSEAVGDIHQVRNAFDDRVSISIHCYGGNIGRIRRHVFKDLGETQEFVSGYSNALTPNLWAPAQ

SEQ ID NO: 9 - DNA, 1449 bp, Danio rerio
  source 1..1449: mol_type = genomic DNA
atggacgagtctgatgggaagctgttccttactgaggctttcaacataatcatggaagaaattcttaacaaaggaagggacttgaaggagaaggtttgtgagtggaaagatccagatcagctgagatctctcctggacctcgaacttcgggatcatggagaatgtcatgagaagctgctgcagagggttcgagatgtggccaaatacagcgtaaaaacttgtcatcctcggttcttcaatcagctgtttgctggcgtggactatcatgcactgacaggacggctcatcactgaaaccctcaataccagccaatacacctatgaagtggctccagtgtttgtcctgatggaggaggaagtgatcagtaagcttcgctctctggttggctggtcagaaggagatgggatcttttgtcctggaggatccatgtctaacatgtatgccattaacgtcgctcggtactgggcttttcctcaagtgaagacaaaaggcttgtgggccgcaccacggatggctatatttacatcacaacagagtcattactccgtgaaaaaaggagctgcgtttcttggtattggaacagaaaatgttttcattgtgcaagtggatgagagcggcagcatgataccagaagacctggaggcaaaaattgtgcaggcaaaatcccaagacgctgttccgtttttcgtaaacgccacagccggaaccacagtgcagggagcctttgaccctctgaagcgcatagctgacatatgtgaaagaaacggcatgtggatgcatgttgacgccgcatggggaggaagcgtgctgttttccaaaaagcacagacatctggttgcaggaatagaaagagcaaactcggtgacttggaatcctcacaaaatgcttctgacgggactgcagtgctctgtgattttgttcagagatactacgaatttgctcatgcactgtcacagtgccaaagccacatacttgttccagcaagacaagttctacgacacaagtctggacacgggcgacaaatccatccagtgtggccggaaggtggattgcctcaagctctggctcatgtggaaggcaatcggagctagtggtctttcacagcgtgtcgataaggcctttgccctcactaggtatttagttgaagaaatggagaaacgggagaatttccagctggtctgtaaggggccgtttgtgaacgtttgcttctggtttattccacccagtctgaaaggaaaggagaacagcccagattaccaggaaagactatccaaggtggcgccagtcattaaagagaggatgatgaagcgaggaacgatgatggtgggatatcagccaatggatgaacacgtcaacttcttccgcatggtggttgtttctccacagctcacaaccaaagacatggatttcttccttgatgagatggagaaactcgggaaggatctatga

SEQ ID NO: 10 - protein, 482 aa, Danio rerio
MDESDGKLFLTEAFNIIMEEILNKGRDLKEKVCEWKDPDQLRSLLDLELRDHGECHEKLLQRVRDVAKYSVKTCHPRFFNQLFAGVDYHALTGRLITETLNTSQYTYEVAPVFVLMEEEVISKLRSLVGWSEGDGIFCPGGSMSNMYAINVARYWAFPQVKTKGLWAAPRMAIFTSQQSHYSVKKGAAFLGIGTENVFIVQVDESGSMIPEDLEAKIVQAKSQDAVPFFVNATAGTTVQGAFDPLKRIADICERNGMWMHVDAAWGGSVLFSKKHRHLVAGIERANSVTWNPHKMLLTGLQCSVILFRDTTNLLMHCHSAKATYLFQQDKFYDTSLDTGDKSIQCGRKVDCLKLWLMWKAIGASGLSQRVDKAFALTRYLVEEMEKRENFQLVCKGPFVNVCFWFIPPSLKGKENSPDYQERLSKVAPVIKERMMKRGTMMVGYQPMDEHVNFFRMVVVSPQLTTKDMDFFLDEMEKLGKDL

SEQ ID NO: 11 - DNA, 1629 bp, unidentified
  source 1..1629: mol_type = genomic DNA
atggtgccccccgccttgcatgaagggttctgcagccctcgaggcaggacttgttgctctcaggtgggacacgtggagttgttggagagctgggaaacgcaggggaacaagctgagatgcgagcaagacctcctgctggccaaggttccctctcgcttccaccaccttgaggaagtggccgagctggatgatatcttcagggaggtgtatcctctgatccggcagtacgagacggagaacgcgctagcagacgagcacaaggtgctggagttcaggacgccagcggagctgaaggaggaggtggacgtggggctgcctgaggagggatctgtggagaaatttgtcgagggatgcagaagctctatgaagtacagcgtccgaacgagtcacccgcgcttcatgaaccagctctatgctggcagcgacccggcagggcaggtggcagagctgctcagtgctgtgctgaacaccaccatccacacgtacggggcagctcccttcttctccgtgctggagcggcaggtgatcgagaagctggggaggatgctggggtttcaggagcatgtcgacggcgtctttgcccccggaggctcgtacgcgaacatggtggcgctgatagttgcgaggaaccagcacttccctcatgtgcgggagcatggctggaggagcgacgacaaacctgttatcttcacttcttcccatgctcactactctgtcgccaaggctgccatgatcacggggatggggtcgaatcaagtggtcgctgtgcctacggacgagcagggaagaatgcagcctgcagcgctggaggaggagattatgcgagcaaaggagagcggacggaagcctttctacgtgagctgcacggcagggacgacagtgactggggcgtttgacccgattgacgagatctgtcagatatgtagaaggcatgagatgtggctgcacacggatggcgcgtggggaggagctgcaatattctcggaggagcacagaaatcttctacgaggagttgagggcgtcgatagcttctgcttgaatccgcacaagatgctgggggtcccgatgcagtgctccgtgctcatcctcaacaaccacgaggggcgctcgagaggagcaacagaggaagagagcttggatctcgggcagaagtcgctgcagtgcggaaggaaacctgattgcctaaagctctggctctgctggaagcgacatggaacccgcgggtttgcaaggagggtagatcgcgcgtataccttctcgcagaagttcgcagaaatggtcagaagggaccccaggttctacctgctgatggacccgatctcctgcaacgtctgcttcttctacctccctccctccctccggcagcagctggtggacagaaacctcaacgacttggaaaaggaggaggcgcagcggcagctcaaggagttccatgctcgactcggtcaggttactcagatcatctacaggaggatgcagaaagacggcaagatgctcatcaacttcagccctcttaaagacagagatctgcctcacttcttccgagccgtcatgatccagcagagagtaacggaagacgatcttgttttcatcctcgatcattttgaacatctgggaaaggacctctag

SEQ ID NO: 12 - protein, 542 aa, unidentified
MVPPALHEGFCSPRGRTCCSQVGHVELLESWETQGNKLRCEQDLLLAKVPSRFHHLEEVAELDDIFREVYPLIRQYETENALADEHKVLEFRTPAELKEEVDVGLPEEGSVEKFVEGCRSSMKYSVRTSHPRFMNQLYAGSDPAGQVAELLSAVLNTTIHTYGAAPFFSVLERQVIEKLGRMLGFQEHVDGVFAPGGSYANMVALIVARNQHFPHVREHGWRSDDKPVIFTSSHAHYSVAKAAMITGMGSNQVVAVPTDEQGRMQPAALEEEIMRAKESGRKPFYVSCTAGTTVTGAFDPIDEICQICRRHEMWLHTDGAWGGAAIFSEEHRNLLRGVEGVDSFCLNPHKMLGVPMQCSVLILNNHEGRSRGATEEESLDLGQKSLQCGRKPDCLKLWLCWKRHGTRGFARRVDRAYTFSQKFAEMVRRDPRFYLLMDPISCNVCFFYLPPSLRQQLVDRNLNDLEKEEAQRQLKEFHARLGQVTQIIYRRMQKDGKMLINFSPLKDRDLPHFFRAVMIQQRVTEDDLVFILDHFEHLGKDL

SEQ ID NO: 13 - DNA, 1425 bp, Spirula spirula
  source 1..1425: mol_type = genomic DNA
atgttcaacgccgccgactattccgacttgctgcaccaacttgaaaccttttttgcccaatcaaccccatcccaccctgttagccagcctcttgaccccgctcaactacgcgatcgcctccccctcaacctccctgctgagggcaaacccctagccgacctgaaaattgagaccgcggcgtacttacaccatgccgttaaaactgctcaccctagttatttcaatcagctttggggtggtgtcaatggcgcttgtctgatgggagaactgctcactagcgccaccaatacctcaatgtatacctatgaggttgctcccgtggcgaccctgattgagcaagccttgatcaccaagctgggtgaactgatcggcttcacccaccctgacggacaattcaccacagggggcagcaatggcaacctgatggcgatggcgatcgcccgtcatcgtgccatgccgacgcttaagcaagacggcatgctcaatgcccccccgttaattgcgtttgtttcggctgattctcactactcctttgccaaagccgcccatcttttaggtttaggaaccaaccaactctgggctgtgcccactgatgcccagggcaaactgaatgtagcagaattagaatcgctcatcaccaaagcccacagccttggggcgcagccgttttttgtggctggtacagccgggacaaccgtgcgcggagcctatgaccccttaaccgaaattgcagccgtggcccaacgggaggggctatggttccatgtggacggcgcttggggggcatcggtgctcttgagccctaaacaccgtgagctgatgcggggcgtggagctagccgactctgtcgtttgggatgcgcacaagatgatggggatgacgctgatgtgttcagtgctgctggtgaagcagcgggggcagctactcaataccttctcgacccaaggaacagactatattttccatgcggatgaatccgaacccgtggatttggggccgagcacgatgcattgcggacggcgggtagatgcagtgaagctctggttagcgtggcatcatctgggcgatcgcggttgggcagccctgattaaccattactttgagttggcagcctatgctgagtgtcttatccgtaaccagccgtatctggaattggtgactccccgcgaatcgctgaacctctgtttccgagtgttgcctcaaagcccagaccaggatgccaatcagctcaccttacgcgttcgacaagtcttgttgcagaaggggttggcgatggtgaactacgcccgtattggtaagcacatcattttccggctggtgatttgcaacaaccaaacccaatgcaccgacctcgatcgcctgtttgagcagatcggtgcgatagctcaccacttacaagcagaatttccacaggagaaaacttga

SEQ ID NO: 14 - protein, 474 aa, Spirula spirula
MFNAADYSDLLHQLETFFAQSTPSHPVSQPLDPAQLRDRLPLNLPAEGKPLADLKIETAAYLHHAVKTAHPSYFNQLWGGVNGACLMGELLTSATNTSMYTYEVAPVATLIEQALITKLGELIGFTHPDGQFTTGGSNGNLMAMAIARHRAMPTLKQDGMLNAPPLIAFVSADSHYSFAKAAHLLGLGTNQLWAVPTDAQGKLNVAELESLITKAHSLGAQPFFVAGTAGTTVRGAYDPLTEIAAVAQREGLWFHVDGAWGASVLLSPKHRELMRGVELADSVVWDAHKMMGMTLMCSVLLVKQRGQLLNTFSTQGTDYIFHADESEPVDLGPSTMHCGRRVDAVKLWLAWHHLGDRGWAALINHYFELAAYAECLIRNQPYLELVTPRESLNLCFRVLPQSPDQDANQLTLRVRQVLLQKGLAMVNYARIGKHIIFRLVICNNQTQCTDLDRLFEQIGAIAHHLQAEFPQEKT

SEQ ID NO: 15 - DNA, 1776 bp, Danio rerio
  source 1..1776: mol_type = genomic DNA
atggcggcgtctgcaccctcctcctcctcttctggcggcgttccggatcccaactcgacaaatttacagccaccttcctcaaactacgactggagtggagtcgctcatggatgtacaaggaagcttggaatgaagatatgtgggttcttgcagaagaacaataatgttgacgacaagggtcgaattgtcgggttgtttaacgaccagcagcccaggagtattttaacccgggacaacgagcgagattcccgcttcagacgcacagagacggacttctccaatctgtatgcaagagatctgcttcctgctaaaaatggcgaggagtacaccatgcagttcctgctggaggtggtggagatcctcactaactacgtgcgcaagaccttcgacagatccaccaaagtgctggacttccaccatccacaccagctgctggaaggcatggagggcttcaacctggagctgtgtgaccagcccgagagtctggagcagatcctggtggactgcagggacactctcaaatatggagtccggacaggtcacccaaggttttttaaccagctgtcttcaggactagacatcatcggtttagctggagaatggttgacctccactgccaacaccaacatgttcacgtatgagattgcgccagtgtttgtcctgatggagcagctcacactgaagaagatgcgagagattgtcggctggccgaacggagaaggagatggcattttctcaccaggaggagccatctccaacatgtacagcgtgatggtggctcgatataaacactatcctgagattaaaatcaaaggcatggcggcggctcccagactggtgctgttcacctcagaacacagtcactactctataaagaaggccagtgcagtgttgggtttcggcacagagaatctgatcctgctgagaacggatgaaagaggtcgagttattccagctgatttggaggccaaagtcattgacgccaagcagaagggctttgtgccgatgtttgtgaacgcaacggctggatctacagtgtacggagccttcgacccaatcaatgagatcgccgacatctgtgagaaatacaacatgtggcttcacgtagatggagcgtggggtggaggtttgctgatgtctagaaaacacaaacacaagctcagtggcattgagagagcaaactccgtcacctggaacccacacaagatgatgggtgttcctctacagtgctccgccattctggtccgggagaagggtcttctacagggctgtaattccatgtgcgctggatatctctttcagccggataagcagtatgacgtcacctatgacacgggggacaaggccatacagtgtggccgtcatgtagacatcttcaaattctggctcatgtggaagtcaaagggcactactggttttgagaagcacattgacaggtgtctggagctgtcggagtatctctaccacaagatcaagaacagagaaggatatgagatggtgtttcaaggggagccacagcacacaaatgtatgtttctggtacattcctccaagcctgcggcttctgccagatggagaggagaaacgacatcggcttcataaggtcgccccaaagatcaaggcactgatgatggagtgcgggacaacaatggtgggctaccagcctcagggtgagaaggttaacttcttcaggatggtggtctccaatccggcggttaccaggtctgacattgacttcctgatcgatgagatagaaagactgggacaggatttatag

SEQ ID NO: 16 - protein, 591 aa, Danio rerio
MAASAPSSSSSGGVPDPNSTNLQPPSSNYDWSGVAHGCTRKLGMKICGFLQKNNNVDDKGRIVGLFNDQQPRSILTRDNERDSRFRRTETDFSNLYARDLLPAKNGEEYTMQFLLEVVEILTNYVRKTFDRSTKVLDFHHPHQLLEGMEGFNLELCDQPESLEQILVDCRDTLKYGVRTGHPRFFNQLSSGLDIIGLAGEWLTSTANTNMFTYEIAPVFVLMEQLTLKKMREIVGWPNGEGDGIFSPGGAISNMYSVMVARYKHYPEIKIKGMAAAPRLVLFTSEHSHYSIKKASAVLGFGTENLILLRTDERGRVIPADLEAKVIDAKQKGFVPMFVNATAGSTVYGAFDPINEIADICEKYNMWLHVDGAWGGGLLMSRKHKHKLSGIERANSVTWNPHKMMGVPLQCSAILVREKGLLQGCNSMCAGYLFQPDKQYDVTYDTGDKAIQCGRHVDIFKFWLMWKSKGTTGFEKHIDRCLELSEYLYHKIKNREGYEMVFQGEPQHTNVCFWYIPPSLRLLPDGEEKRHRLHKVAPKIKALMMECGTTMVGYQPQGEKVNFFRMVVSNPAVTRSDIDFLIDEIERLGQDL

SEQ ID NO: 17 - DNA, 2958 bp, Micromonas pusilla
  source 1..2958: mol_type = genomic DNA
atgtccgcggcgacgggatcattatccctacccctactcgggcatctcgcgacctcgcgtaacgcacgcgcgcgtcggaaccgcgccgccgcggccatccccggcgtctccctcgggaaatcgacctcggttttcactccgcgaggtcctaagcgcatcgcgcgcgtcgtcacctcgaaggcgggcccgcattcgaaccctccgagggcgatatcgaccgtcgacgacgtcctcgcgttcaccgtgcccaccgacgagcccgcggccgagaccgcctcccccgccgacagcgactgcgaaggcgagttctgcgacatgaaggagagctcgtgcacgacgagggacctcatcggcagcacgccgctgctcgatctgagcgcgtactccctgaaccccaccgtgaagatcctcgcgaagtgcgagtacctcaacccgtccgggtccatcaaagaccgcatcgcgacgcacatcctggacaaggcgatcaagagcggcgatctcaagcccgggatgaccgtcgtcgcggcgacgtccgggaacaccggcgccgcgatcgcgatggcgtgcgcgttgcgcgggtacgactacatcgtcatcaccaacgagaagacgtccaaggagaaggtggacgcgatgagagcgtacggcggcgaggtgatcgtctccccgtccggggtgtccccggacgacccacagcactaccagaacatcgagaacaagctgtgcgaggagaaccccgggacgtactacggcgtggatcagtataacaacccgtacaacgcggacgcgtacgaggcgacgctcgggccggagatttggcgtcagagcgtgggcgcggtgacgcacttcatcgtcggcggcagcaccggcggcacggtcagcggcacggggaggtacttgaagcaagagaacccggacgtgaggatcgtcctcgcggacccgagagggagcgtgttctgggaccacgtcgtcaacggcgtcgccgccgacgacgtcaaggtgtccaagtcgtgggagacggagggcgtcggcaaggattccatccccgggtgcctcgacgtctcgatcgtggacgggatggtgcgcgcgacggacgagcaggcgttcggcgtgtgccgcgagctcgcgagcagcgacggcctcctcgtcggcggcagcagcggtctgaacctccacgcctcgcgcgtgttatccggcgacgtcgcggacgacagcgtcatcgtcacggtgttcccggacaacggcgtgaagtacctgtcgaagatttacaacgacgactggctcgactcgaagaagatgggcggcgcaaagaactcggacgggaacgcggagagagccgcggagtgcgaggtgtactggcgcccggacgcgctctcgttcgcggagcgaaaggcggcggcggacgccgccgccgccgccgccgtcgagggcgacaacctctggcccgaggacgagaccgagcgcgagctcaagttcctggaggaactcgcgccgaagctgacgcagtaccacagagactccatcaagggcgacgagcgcgtgcacagcaagctccagtccccggaggagctcgcggcgacgttcgccgccgcgggggcgcccatcgacctcgcggagggcgacgcccccgcgacggaggagcaactcgcgctcgcggtgcaggcggtcatggacaactcggtccgctcctcgcacccgatgttcttgaaccagctgtacgccggcgtcgacgtcgtcgcgctcgcgggggagtggaccgcgagcgcgttgaacgccaacgtgcacacgtttgaagtcgcgccggtgctcacggagattgagaaagccgtcctcgcgaaaaccgcgcggatgtggctgaacaagcccgggtctaagacgacgccgccgcacgacggtctgctcgtccccggcgggtccctggcgaacatgtactcgatgatcctcgcgcgcgatcgcgcggagccggaggcgaagaccaagggcgcgagcggcaacctcgtcgcgttttgctcggagcagtcgcactactcgtacaaaaagtccgcgatggtcatgggcctcgggatggacaacatgatcaaggtgaagtgcgaccagtccggcgcgatgatcccggcggagctcgagaaggcggttcaggaggccaagtcccggggcaaggtgccgttctacgtcggcaccaccgcggggtccaccgtgctcggcgcctttgacgactacgaaggctgcgcggacgtctgcgaaaagcacgacatgtggatgcacgtcgacggcgcgtggggcggcgccgcggcgctgtccccgacgagaaggcacaatctccagggcgcgaacagagcggactcgttctgctggaacccgcacaagatgctcgggttgccgctccagtgctccatcttcgtgacgaagcaacccggggcgctgtccaaggcgaacgccgcgcaggcggactacttgttccagccggacaagaacaacgccgccgcggacctcggcgaccgcacgattcagtgcggacgcaaggcggacgccctcaagatctggctcgcgtggaaggcgcgcggagacgaaggctgggcgaatctcgtggaccgctcctttggcctcgcggagtacgtcgaggcgtcggtgcgcgagcggtgcgaaaaagacggctcgttcgtcctcgccgcgcccgcgcagtgcgcgaacatcgggttctggtacgtgcccccgcgcctgaggccgttcgatgtcgagtccgcgaccgcggaccagctcacggagattgggttcgtcgccccgaagctgaaggaccggatgcaacggaccggggacgcgatgatcgggttccagccgatcgactcgatgaaccttccaaactttttccgactcgtgcttccaaactcgaggcacctgtcgaagaacgcgctcgacgctatgctcgatcgcatggacgacatgggcaaagacctgtga

SEQ ID NO: 18 - protein, 985 aa, Micromonas pusilla
MSAATGSLSLPLLGHLATSRNARARRNRAAAAIPGVSLGKSTSVFTPRGPKRIARVVTSKAGPHSNPPRAISTVDDVLAFTVPTDEPAAETASPADSDCEGEFCDMKESSCTTRDLIGSTPLLDLSAYSLNPTVKILAKCEYLNPSGSIKDRIATHILDKAIKSGDLKPGMTVVAATSGNTGAAIAMACALRGYDYIVITNEKTSKEKVDAMRAYGGEVIVSPSGVSPDDPQHYQNIENKLCEENPGTYYGVDQYNNPYNADAYEATLGPEIWRQSVGAVTHFIVGGSTGGTVSGTGRYLKQENPDVRIVLADPRGSVFWDHVVNGVAADDVKVSKSWETEGVGKDSIPGCLDVSIVDGMVRATDEQAFGVCRELASSDGLLVGGSSGLNLHASRVLSGDVADDSVIVTVFPDNGVKYLSKIYNDDWLDSKKMGGAKNSDGNAERAAECEVYWRPDALSFAERKAAADAAAAAAVEGDNLWPEDETERELKFLEELAPKLTQYHRDSIKGDERVHSKLQSPEELAATFAAAGAPIDLAEGDAPATEEQLALAVQAVMDNSVRSSHPMFLNQLYAGVDVVALAGEWTASALNANVHTFEVAPVLTEIEKAVLAKTARMWLNKPGSKTTPPHDGLLVPGGSLANMYSMILARDRAEPEAKTKGASGNLVAFCSEQSHYSYKKSAMVMGLGMDNMIKVKCDQSGAMIPAELEKAVQEAKSRGKVPFYVGTTAGSTVLGAFDDYEGCADVCEKHDMWMHVDGAWGGAAALSPTRRHNLQGANRADSFCWNPHKMLGLPLQCSIFVTKQPGALSKANAAQADYLFQPDKNNAAADLGDRTIQCGRKADALKIWLAWKARGDEGWANLVDRSFGLAEYVEASVRERCEKDGSFVLAAPAQCANIGFWYVPPRLRPFDVESATADQLTEIGFVAPKLKDRMQRTGDAMIGFQPIDSMNLPNFFRLVLPNSRHLSKNALDAMLDRMDDMGKDL

SEQ ID NO: 19 - DNA, 747 bp, Danio rerio
  source 1..747: mol_type = genomic DNA
atgccgaggaacaacaagacctcgctcatccagaaaatagcgagccaggcttatgcgactttcacaaactgctcttcctccgccattgaggataataataaggtgtttttagagcaccaggcagacttggtcgccctgctctccgacatcagggctgcggatctgaagattgcgccaccaacgaaagtctccacatcctccccttcggtccctccagtcacttacatgcacatctgcgagactgatgtcttcagcatgggggtgtttctcctgaaatctggcgcttccatccctctgcacgaccatcccgggatgcacggcatgctgaaggtcctgtacggtaaagtcagcatcaggtgttatgacaagttggataaagccgagagtgacactgagaggcactttgatcctccattgttggctttccagggagatgatgtgaggagagcagcgctgaggtcttcggggcagttttcggaactaagtggcccttgtgttttgagcccttttaaagacaaccttcatgagattgatgcagtggatggagcggctgcgtttcttgatatactggcaccgccgtatgaccctgacgatggacgggattgtcactactacagagttttacagactgctgggaaaaagtcagagcagagcggtgatgatgaagcttggcttttagagatcccccagccggatgatttctggtgtgggggtgagccgtaccctggtcctgaagtgactatttag

SEQ ID NO: 20 - protein, 249 aa, Danio rerio
MTSTVQKIAKQALATFRNPSVIGEHNKVFLENQSKLKSLLAEVRAADLKIAARTPESAPVPHQRIAPPVTYMHICETDSFSMGVFLLKTGASIPLHDHPGMYGMLKVIYGKVRISCFDRLDKPRDGASGVQFNPPLMPFQRGSLRPSVLKSVGEFTEDSSPCVLSPQQDNIHQIDAVDGPTAFLDILAPPYDPDEGRDCHYYKVLQAHSEAADKKSEVQDQGDVWLMEIPQPSEFWCGGEPYPGPKVTL

SEQ ID NO: 21 - DNA, 1200 bp, Gallus gallus
  source 1..1200: mol_type = genomic DNA
atggagggaggttacttgcggcccttcgcgcacttcggcacgcaggccatccacgccgggcaggagcccgagcagtggcgttcgggggctttggtgccgccggtctcgctctccaccacgttcaagcagcaggcgcccggcgagcacgcgggttatgattacagccggtgtggaaaccctacccgaacctgcctggagaaggctgtggctgtgctggatggagcgaaatactgtttggcctatgcttctggcttagctgctactgtgaatattactcacctgttaaaggcaggagatacaattatctgtatggatgatgtgtatggaggtacgaacagatacttccagcaaatagccaagaaaatgggtttaaaaataatttttgtggactgtacaaaattgaagtgcctggaagctgcaattacacctgagactaagcttgtttggattgaaacgcccacaaaccccacactgaaggtcattgatattcagggctgtgctgatgtgatccataagcacaaagatattctgctggcagtagacaatagttttatgtctgcgtatttccagcgtcctttgtccctgggggctgacatctgtatgtactctgcaaccaagtacatgaacgggcacagtgatgttgtaatgggactcgtttcagtaaatcgtgatgatgtgtatgaaaggctcaaattcttacaaggctctcttggagccattccatctccctttgactgctacctctgcaaccggggtttgaagactctgcagctgcggatgaatcaacacttccgcaatgcattggctgtgggtcgattccttgagtcaaatccccgcgtggagaaagtcatttaccctggattgccttcgcacccccagcacgagctggccaagcggcagtgcactggctgtcctgggatgctgtcattttacattaaaggaaatctcaaacatgctaccatctttctgaagagcataaaggtgttcacgctggctgagagcctgggaggatatgaaagcttagcagagcttccggctgtcatgactcatgcttcagtgcctaaagaagagagagaagctcttggaatcagcgatacattaattcgcctgtcggttggtttggaagatgaagaagatttactggaagacctagatcaagctctgaaagctgcaatcccagaccctaaggctttgaaatga

SEQ ID NO: 22 - protein, 399 aa, Gallus gallus
MEGGYLRPFAHFGTQAIHAGQEPEQWRSGALVPPVSLSTTFKQQAPGEHAGYDYSRCGNPTRTCLEKAVAVLDGAKYCLAYASGLAATVNITHLLKAGDTIICMDDVYGGTNRYFQQIAKKMGLKIIFVDCTKLKCLEAAITPETKLVWIETPTNPTLKVIDIQGCADVIHKHKDILLAVDNSFMSAYFQRPLSLGADICMYSATKYMNGHSDVVMGLVSVNRDDVYERLKFLQGSLGAIPSPFDCYLCNRGLKTLQLRMNQHFRNALAVGRFLESNPRVEKVIYPGLPSHPQHELAKRQCTGCPGMLSFYIKGNLKHATIFLKSIKVFTLAESLGGYESLAELPAVMTHASVPKEEREALGISDTLIRLSVGLEDEEDLLEDLDQALKAAIPDPKALK

SEQ ID NO: 23 - DNA, 1401 bp, Chlamydomonas reinhardtii
  source 1..1401: mol_type = genomic DNA
atgcgcagtgtttactctcctgccgcggagatgcccgctggcgtctctatgcgggcgcctgttcccgctgctcttaggtcagcggcccgcagcggcctgcacctgcgcaatgcgtctttccacacgcaccggccgagcgtggtggccccggcggctgccaaggacctggagcccacttctgcgagcagcaacggagcccccaagaacaacggcgtgcagttcactaagctgtcgaccactgccgtccacggcggcgagcgcggtggccgtccccgcgtggccgacgcgctgaccacgccgattgttcaaacctccacctatcacttccgcaacactgcggagctgattgagtacaatgagggtcggttcgattcgtatgagtatgggcgctatggcaacccgaccgcccaggcatgcgagaacaagatcaaggccctggagggcgctgaggactgcctggtttcggcgtcgggcatgaacgcggtgaccagcatgctgctgtcgctggtgccggcgggcggccacatcgtgaccacgtccgattgttactggcgcacccgccagttcatgcagaacttcctgcccaagatgaacatcggcgtcagcgtgattaagcccaacgatctggaggctctgcaggaggcgctggacaagcacagcgtgacgctgttcttctcggagtcgcccaccaacccctacctgcgctgcgtggacatcctcgccatctcgcgcctgtgccacgccaagggcgccttcgtgtgcgtcgactccaccttcgccacgcccatcaacacccgcgccctagagctgggcgccgacctggtgctgcactcggccaccaagtacctggccggccacaacgacgtgctggccggcgcgctgtgcggcaagaaggagctggtggagaaggtgcgcgagttccaccacatcatgggcggcgtggtggacccgcacgccgcctacctgctgctgcgcggcctcaagacgctggagctgcgcgtcacgcgccacaacgccagcgcaatggagatcgcgcggcggctggagagccaccccaaggtggtccgcgtctggtaccccggcctggagtcgcaccccgaccacgccatcgccaagcgccagatgagcggcttcggcggcgtggtgtcgttcgagacgcgcggcggtctgaacgagtgcatcagcttcatcgacaacgtgaagctgccgtacattgcgccgtcgctgggcggcgtggagtcgttgatagagatgccggcggtgcagagctactggggctttggaccggagcgccgcgcgcagattggcatcaaggagaacctgatccgcttcagcatcggcgtggaggacgtggaggacatctgggccgacctggtgcaggcgctggagtacgtgccgtaa

SEQ ID NO: 24 - protein, 466 aa, Chlamydomonas reinhardtii
MRSVYSPAAEMPAGVSMRAPVPAALRSAARSGLHLRNASFHTHRPSVVAPAAAKDLEPTSASSNGAPKNNGVQFTKLSTTAVHGGERGGRPRVADALTTPIVQTSTYHFRNTAELIEYNEGRFDSYEYGRYGNPTAQACENKIKALEGAEDCLVSASGMNAVTSMLLSLVPAGGHIVTTSDCYWRTRQFMQNFLPKMNIGVSVIKPNDLEALQEALDKHSVTLFFSESPTNPYLRCVDILAISRLCHAKGAFVCVDSTFATPINTRALELGADLVLHSATKYLAGHNDVLAGALCGKKELVEKVREFHHIMGGVVDPHAAYLLLRGLKTLELRVTRHNASAMEIARRLESHPKVVRVWYPGLESHPDHAIAKRQMSGFGGVVSFETRGGLNECISFIDNVKLPYIAPSLGGVESLIEMPAVQSYWGFGPERRAQIGIKENLIRFSIGVEDVEDIWADLVQALEYVP

SEQ ID NO: 25 - DNA, 1260 bp, Methanosarcina acetivorans
  source 1..1260: mol_type = genomic DNA
atgatagcaatgggaagattcatattaaaatgtctgaaatgcggcagagaatacagccaggaatacaggctgacctgcgagaatgacgactcctttttgcgggcggaataccttgaaaaaaaacttgagctgagaaagcagccagggataggaagatttcactcatggcttccggttcaggaagagcttactaccgaagccgggcccatcacgtacaaaagcgaagctcttgcgagggaacttgggctttcgaatctgtacatagggttcagcgggtactggcccgagaaaggagcttttatcaagacctgcagtttcaaagaactcgaagcccatcctacgatgcagcttctcaaggaatccgggggaaaagccatagtccttgcctctgcagggaatacggggagggcttttgcacatgtttcggcacttaccggaaccgatgtttatatcgtggttcccgactcaggcatccctaaactctggctgcctgaagaaccgaccgattccattcaccttatcagcatgactccggggaacgattacaccgatgctatcaaccttgcaggaagaattgcaaagcttcctggaatggtccctgaaggaggagccagaaacgttgccagaagagaaggaatgggtactgtaatgcttgatgcagccgtaaccataggaaagatgcctgatcactacttccaggctgtcggaagcgggacgggaggaatctcagcctgggaagcttctctgcgcctcagagaggacgggcgttttggttccaaacttccaaagctccagcttacccagaatctccccttcgttcccatgtataatgcatggcaagaaggcaggagggatataattcccgaaattgacatgaaagatgcaaagaagcggatcgaagagacctacgccactgtacttaccaaccgagcaccaccttactccgtgacaggcgggctctatgacgcacttgtcgatacggacgggataatgtatgcagtaagcaaagaagaagcccttgacgcaaaagcgctttttgagtcccttgaaggaatagatatccttcccccatctgccgttgctgctgcttccctcttaaaagccgtggaagccggaaatgtcggaaaggacgacactatcctcctgaacattgcaggcggaggtttcaaacggctgaaggaagacttcacactattccagattgaacctgaaattactgtctcgaacccggatgtgccgcttgaggaactgaagctctga

SEQ ID NO: 26 - protein, 419 aa, Methanosarcina acetivorans
MIAMGRFILKCLKCGREYSQEYRLTCENDDSFLRAEYLEKKLELRKQPGIGRFHSWLPVQEELTTEAGPITYKSEALARELGLSNLYIGFSGYWPEKGAFIKTCSFKELEAHPTMQLLKESGGKAIVLASAGNTGRAFAHVSALTGTDVYIVVPDSGIPKLWLPEEPTDSIHLISMTPGNDYTDAINLAGRIAKLPGMVPEGGARNVARREGMGTVMLDAAVTIGKMPDHYFQAVGSGTGGISAWEASLRLREDGRFGSKLPKLQLTQNLPFVPMYNAWQEGRRDIIPEIDMKDAKKRIEETYATVLTNRAPPYSVTGGLYDALVDTDGIMYAVSKEEALDAKALFESLEGIDILPPSAVAAASLLKAVEAGNVGKDDTILLNIAGGGFKRLKEDFTLFQIEPEITVSNPDVPLEELKL

SEQ ID NO: 27 - DNA, 1314 bp, unidentified
  source 1..1314: mol_type = genomic DNA
atgacatacggaaaaacaataggttatggatcgaatactgttcattacacgggggattcgaaatatgagcttgtatgcgcagaatgttcaaacatttttactgatgacagattgaaatgctgcaacgatggagcgctcctgcgtgcaaattacaggaataaacagctcatgttaaaggacgtccccgggatgggtaagttccacgactggctgccgatacatgagataatgaccacgaattcaggtcctgtcacctacaggagcactgaacttgcagcagaacttgggcttaacaacctgtacatctctttcaatggctactggcctgaaaaaggtgcgtacataaagacttgcagtttcaaggaacttgaggcttttcccacggtccagaaaaccagacgtgcaaaccgctcgctggtacttgcatcagcgggaaataccgcgcgcgctttttcccatgtatcagcgatgacaggacaggaagtgtatatagtggtgccttcaagcgggatgacacggatgtggctgcctgaagaacccacggattccatacacattatacaaatgggtaataactgtgattatacagattctatacatcttgcagacaggatcgccgcgctccccggtatgcaggcagaaggcggcgcaaggaacgtggcgcgaagggacggtatggggacggtgatgctggatgccgctgtgactatgaagcgcatgcctgaccattatttccaggctatagggagcgggacaggcggaatagcagcatgggaagcatcgctgcggctgctgggggatggaaggttcgcaggcggactgccgcagcttcatctttcacagaacctgccttttgccccaatgttcaatgcatggaaagcaggacggcgcgaaataatcagggacattgatatgcctgacccgaaaaaactcatacacgagatgtatgcagacatcctttcaaacagggagccgccatattcagtagctggcggcgtctatgacgcgctttgctctacacagggtacgatgtacgggatatccaatagcgaagcgaaaaaagcgcagcagttgttcgaacggtacgaagggatagatatatttgcacccgcaggagttgctgttgcgtcgctcatcgaagcggtaaatgaacataaagtcgatgcagaagatcatattgtgttaaatatcactggcggaggagtgaagcgccttgaaaaggaccataccctctcgaaaatcgagccagagtcgatcgttgaaaatgcgaatgttgcgcttgaagaggtaattaaaagaaaggtatttgcttga

SEQ ID NO: 28 - protein, 437 aa, unidentified
MTYLKTISSGTNTILHTWDPKYDLVCAGCSSVFTEDRLKCSNDGALLRANYRNKQLILKDVPGMGKFHDWLPIHEIMTTESGPVTYRSTELAAELGLNNLYISFNGYWPEKGAYIKTCSFKELEAFPTVQKTTGKNRSLVLASAGNTARAFSHVSAMTGQEVYIVVPSSGMLRMWLPEKPTDSIHIIQMGKNCDYTDSIHLADRIAALPGMQAEGGARNVARRDGMGTVMLDAAVTMKRMPDHYFQAIGSGTGGIAAWEAALRLREDGRFPGGLPQLHLSQNLPFAPMYNAWKAGRREIIKDIDMPDPKKLIHEMYADILSNREPPYSVAGGVYDALSSTKGTMYGISNNEAKKAQQLFERYEGIDIFAPAGVAVASLIEAVNEHKVDSEDHILLNITGGGVKRLEQDHTLSKIEPESIAENANVPLEEIIKRKVLA

SEQ ID NO: 29 - DNA, 1545 bp, Escherichia coli
  source 1..1545: mol_type = genomic DNA
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtggatgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgagtttccggaatcaccgggcgcgttcctgcgcttcctcaacacgctgggtacgtactggaacatttctttgttccactatcgcagccacggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 30 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDAVRGFGGEVLLHGANFDEAKAKAIELSQQQGFTWVPPFDHPMVIAGQGTLALELLQQDAHLDRVFVPVGGGGLAAGVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELGEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGAFLRFLNTLGTYWNISLFHYRSHGTDYGRVLAAFELGDHEPDFETRLNELGYDCHDETNNPAFRFFLAG

SEQ ID NO: 31 - DNA, 903 bp, unidentified
  source 1..903: mol_type = genomic DNA
atgactgaatctaagtttcctgtgaagacgagagagtttcacaaccaccattttgattcgacggtctggaatgattttgtattccgcgacgacgatattatcatttccacctatgcaaaatccggcacgacctggatgcagcaaattgtgtcccagttgctgttttccggagaggagggggtggaggttgctgagatgtcaccctggatggatttgcgagttcctccgaaagaagtcaagttgccggcggttgaggcgcagacgcaccgtcgatttttaaaaactcacttacctgtggatgcgctggtcttttcagaaaaagcaaagtacatctacatcggtcgggacgggcgtgatgttgtctggagtctttataatcatcatcttaatgccaacggagcctggtatgatgctctcaacaatacgccaggcagggttggcccggagatagggcctccctgcgcgtctgtctcgcagtatttcgacgattggctggaaaaagacggctatccattctggtcgctgtgggacaacgcccgcacctggtgggcgattcggcatttgccgaatgtgcacatggttcactttgcaaatctgaagtcagacatgcctggtgagattcgccgcattgcagaattcattggcacgcccgttgacgaatcgaaatgggagagcgtattaaagcattgtagcttcgactatatgaaggccaatgcgacaaagagtgttccacttggtggggcgttctgggatggcggcgcacaaacctttattcataaaggccagaatggccggtggcgtgatgtgctttcagccgaacagaataagcagtatgagcagcgtgcgcagtcagagcttggctccgactgtgcccattggtttgccactggtgagctcagataa

SEQ ID NO: 32 - protein, 300 aa, unidentified
MTESKFPVKTREFHNHHFDSTVWNDFVFRDDDIIISTYAKSGTTWMQQIVSQLLFSGEEGVEVAEMSPWMDLRVPPKEVKLPAVEAQTHRRFLKTHLPVDALVFSEKAKYIYIGRDGRDVVWSLYNHHLNANGAWYDALNNTPGRVGPEIGPPCASVSQYFDDWLEKDGYPFWSLWDNARTWWAIRHLPNVHMVHFANLKSDMPGEIRRIAEFIGTPVDESKWESVLKHCSFDYMKANATKSVPLGGAFWDGGAQTFIHKGQNGRWRDVLSAEQNKQYEQRAQSELGSDCAHWFATGELR

SEQ ID NO: 33 - DNA, 969 bp, unidentified
  source 1..969: mol_type = genomic DNA
atgccgcgcggatggacgaagacgcgcgcgtacgactcgcatcactttgacgccgacgcgtggtcggtggtgacgcctcgagcgggtgacgtcatcatcgccaccgcgtataaatctggcacgacgtggatgcaacagatcgtgtcgcaactcgtgttcgagggcgcggccccggcggcgttgggggagctctcgccgtgggtggatctgcgcgtgcccccgcgggaggtgaagcgagggatgatcgagggattgccctcgccccggatcttgaagacgcatcttccgacgacggggttggaatacgacgaaaacgcgaagtacatttacgtcgcgcgggacggccgcgacgcgttcatgtctttgatgaaccactataagaacggtaatgaagcgttttacggcgcgctgaacggccctgggttaaagggcgcacctttgcctacgtgggaagaggcgtgcgaaggcgagggcgacgagaaacttcgcgcgctttttgacaagtggctcaacacgccgtggggccagcacccgtgggaagaagacgggtggcctttctggtctctgttctataacatgaagacgtggtgggacgcgcgcgaatccaagaacatcatcttcgtgcatttttcggatttgaagaaggatttgaagggtcagatgcgacgcattgcgaagtttttgaacgccccgatcgatgaaagcaaattcgatgcgcaagtcacagcgtgcacgttcgagagcatgaagggtaacgccgcgagcgtcgcgcctctcggtggcgcgctgtggaagggcggtgcggagacgttcattaacaaaggtaccaacggccggtggaggaacgttctaaccaaggaacaagtcaagcagtacgagcaggtggctgagaaacggctgggtaaggactgcgcaaagtggctcgccaacggcggcgatatgaacggccgtgggtgcgtgatcatgtga

SEQ ID NO: 34 - protein, 322 aa, unidentified
MPRGWTKTRAYDSHHFDADAWSVVTPRAGDVIIATAYKSGTTWMQQIVSQLVFEGAAPAALGELSPWVDLRVPPREVKRGMIEGLPSPRILKTHLPTTGLEYDENAKYIYVARDGRDAFMSLMNHYKNGNEAFYGALNGPGLKGAPLPTWEEACEGEGDEKLRALFDKWLNTPWGQHPWEEDGWPFWSLFYNMKTWWDARESKNIIFVHFSDLKKDLKGQMRRIAKFLNAPIDESKFDAQVTACTFESMKGNAASVAPLGGALWKGGAETFINKGTNGRWRNVLTKEQVKQYEQVAEKRLGKDCAKWLANGGDMNGRGCVIM

SEQ ID NO: 35 - DNA, 1218 bp, Corynebacterium glutamicum
  source 1..1218: mol_type = genomic DNA
atggctgttaagaccctcaaggacttgctcgacgaaggcgtagacggacgccacgtcatcgttcgatctgacttcaatgttcccctcaacgatgaccgcgagatcaccgataagggccgaatcattgcctccctaccaacccttaaagcactgagcgaaggtggcgcaaaggtcatcgtcatggctcaccttggccgcccaaagggcgaggtcaacgagaagtactccctcgcacctgtcgctgaggcactctccgatgagcttggccagtacgttgcacttgccgcagacgttgttggcgaagacgcacacgagcgcgcaaacggcctgaccgagggcgacatcctgctcctggagaacgtgcgcttcgacccacgcgaaacctccaaggacgaggcagagcgcaccgctttcgctcaggagctcgcagctcttgcagcagacaacggcgcattcgtttctgacggcttcggtgttgtccaccgcgcacagacctccgtctacgacattgcaaagttgctgccacactacgctggcggactggtagagaccgagatttccgttctggaaaagatcgcagaatcaccagaggcaccatacgtagtggttctcggtggatccaaggtctctgacaagatcggtgttattgaggcgctggctgccaaggctgacaagatcatcgtcggtggcggcatgtgctacaccttcctcgcagctcagggacacaacgttcagcagtccctcctgcaggaagaaatgaaggctacctgcaccgacctgctcgcacgcttcggtgacaagatcgttctcccagttgacctggttgcagcatccgaatttaacaaggacgcagagaagcagatcgttgacctggactccatcccagaaggctggatgtctcttgacatcggaccagagtccgtcaagaacttcggtgaggttctcagcaccgctaagaccatcttctggaacggcccaatgggcgtgttcgagttcgcagcattctctgaaggcacccgcggcatcgcccaggccatcatcgatgcaactgcaggcaacgacgcattctccgttgttggcggtggcgactccgcagcatccgttcgcgtgctcggcctgaacgaagacggcttctcccacatctccaccggtggtggcgcatccctcgagtaccttgaaggcaaggaactcccaggcgttgcaattctcgctcagtaa

SEQ ID NO: 36 - protein, 405 aa, Corynebacterium glutamicum
MAVKTLKDLLDEGVDGRHVIVRSDFNVPLNDDREITDKGRIIASLPTLKALSEGGAKVIVMAHLGRPKGEVNEKYSLAPVAEALSDELGQYVALAADVVGEDAHERANGLTEGDILLLENVRFDPRETSKDEAERTAFAQELAALAADNGAFVSDGFGVVHRAQTSVYDIAKLLPHYAGGLVETEISVLEKIAESPEAPYVVVLGGSKVSDKIGVIEALAAKADKIIVGGGMCYTFLAAQGHNVQQSLLQEEMKATCTDLLARFGDKIVLPVDLVAASEFNKDAEKQIVDLDSIPEGWMSLDIGPESVKNFGEVLSTAKTIFWNGPMGVFEFAAFSEGTRGIAQAIIDATAGNDAFSVVGGGDSAASVRVLGLNEDGFSHISTGGGASLEYLEGKELPGVAILAQ

SEQ ID NO: 37 - DNA, 1002 bp, Corynebacterium glutamicum
  source 1..1002: mol_type = genomic DNA
atgagccagaatggccgtccggtagtcctcatcgccgataagcttgcgcagtccactgttgacgcgcttggagatgcagtagaagtccgttgggttgacggacctaaccgcccagaactgcttgatgcagttaaggaagcggacgcactgctcgtgcgttctgctaccactgtcgatgctgaagtcatcgccgctgcccctaacttgaagatcgtcggtcgtgccggcgtgggcttggacaacgttgacatccctgctgccactgaagctggcgtcatggttgctaacgcaccgacctctaatattcactccgcttgtgagcacgcaatttctttgctgctgtctactgctcgccagatccctgctgctgatgcgacgctgcgtgagggcgagtggaagcggtcttctttcaacggtgtggaaattttcggaaaaactgtcggtatcgtcggttttggccacattggtcagttgtttgctcagcgtcttgctgcgtttgagaccaccattgttgcttacgatccttacgctaaccctgctcgtgcggctcagctgaacgttgagttggttgagttggatgagctgatgagccgttctgactttgtcaccattcaccttcctaagaccaaggaaactgctggcatgtttgatgcgcagctccttgctaagtccaagaagggccagatcatcatcaacgctgctcgtggtggccttgttgatgagcaggctttggctgatgcgattgagtccggtcacattcgtggcgctggtttcgatgtgtactccaccgagccttgcactgattctcctttgttcaagttgcctcaggttgttgtgactcctcacttgggtgcttctactgaagaggctcaggatcgtgcgggtactgacgttgctgattctgtgctcaaggcgctggctggcgagttcgtggcggatgctgtgaacgtttccggtggtcgcgtgggcgaagaggttgctgtgtggatggatctggcttaa

SEQ ID NO: 38 - protein, 333 aa, Corynebacterium glutamicum
MSQNGRPVVLIADKLAQSTVDALGDAVEVRWVDGPNRPELLDAVKEADALLVRSATTVDAEVIAAAPNLKIVGRAGVGLDNVDIPAATEAGVMVANAPTSNIHSACEHAISLLLSTARQIPAADATLREGEWKRSSFNGVEIFGKTVGIVGFGHIGQLFAQRLAAFETTIVAYDPYANPARAAQLNVELVELDELMSRSDFVTIHLPKTKETAGMFDAQLLAKSKKGQIIINAARGGLVDEQALADAIESGHIRGAGFDVYSTEPCTDSPLFKLPQVVVTPHLGASTEEAQDRAGTDVADSVLKALAGEFVADAVNVSGGRVGEEVAVWMDLA

SEQ ID NO: 39 - DNA, 1341 bp, Corynebacterium glutamicum
  source 1..1341: mol_type = genomic DNA
atgtcgtgttccgcgctcagacatgagacaattgttgccgtgactgaactcatccagaatgaatcccaagaaatcgctgagctggaagccggccagcaggttgcattgcgtgaaggttatcttcctgcggtgatcacagtgagcggtaaagaccgcccaggtgtgactgccgcgttctttagggtcttgtccgctaatcaggttcaggtcttggacgttgagcagtcaatgttccgtggctttttgaacttggcggcgtttgtgggtatcgcacctgagcgtgtcgagaccgtcaccacaggcctgactgacaccctcaaggtgcatggacagtccgtggtggtggagctgcaggaaactgtgcagtcgtcccgtcctcgttcttcccatgttgttgtggtgttgggtgatccggttgatgcgttggatatttcccgcattggtcagaccctggcggattacgatgccaacattgacaccattcgtggtatttcggattaccctgtgaccggcctggagctgaaggtgactgtgccggatgtcagccctggtggtggtgaagcgatgcgtaaggcgcttgctgctcttacctctgagctgaatgtggatattgcgattgagcgttctggtttgctgcgtcgttctaagcgtctggtgtgcttcgattgtgattccacgttgatcactggtgaggtcattgagatgctggcggctcacgcgggcaaggaagctgaagttgcggcagttactgagcgtgcgatgcgcggtgagctcgatttcgaggagtctctgcgtgagcgtgtgaaggcgttggctggtttggatgcgtcggtgatcgatgaggtcgctgccgctattgagctgacccctggtgcgcgcaccacgatccgtacgctgaaccgcatgggttaccagaccgctgttgtttccggtggtttcatccaggtgttggaaggtttggctgaggagttggagttggattatgtccgcgccaacactttggaaatcgttgatggcaagctgaccggcaacgtcaccggaaagatcgttgaccgcgctgcgaaggctgagttcctccgtgagttcgctgcggattctggcctgaagatgtaccagactgtcgctgtcggtgatggcgctaatgacatcgatatgctctccgctgcgggtctgggtgttgctttcaacgcgaagcctgcgctgaaggagattgcggatacttccgtgaaccacccattcctcgacgaggttttgcacatcatgggcatttcccgcgacgagatcgatctggcggatcaggaagacggcactttccaccgcgttccattgaccaatgcctaa

SEQ ID NO: 40 - protein, 446 aa, Corynebacterium glutamicum
MSCSALRHETIVAVTELIQNESQEIAELEAGQQVALREGYLPAVITVSGKDRPGVTAAFFRVLSANQVQVLDVEQSMFRGFLNLAAFVGIAPERVETVTTGLTDTLKVHGQSVVVELQETVQSSRPRSSHVVVVLGDPVDALDISRIGQTLADYDANIDTIRGISDYPVTGLELKVTVPDVSPGGGEAMRKALAALTSELNVDIAIERSGLLRRSKRLVCFDCDSTLITGEVIEMLAAHAGKEAEVAAVTERAMRGELDFEESLRERVKALAGLDASVIDEVAAAIELTPGARTTIRTLNRMGYQTAVVSGGFIQVLEGLAEELELDYVRANTLEIVDGKLTGNVTGKIVDRAAKAEFLREFAADSGLKMYQTVAVGDGANDIDMLSAAGLGVAFNAKPALKEIADTSVNHPFLDEVLHIMGISRDEIDLADQEDGTFHRVPLTNA

SEQ ID NO: 41 - DNA, 1131 bp, Corynebacterium glutamicum
  source 1..1131: mol_type = genomic DNA
atgaccgacttccccaccctgccctctgagttcatccctggcgacggccgtttcggctgcggaccttccaaggttcgaccagaacagattcaggctattgtcgacggatccgcatccgtcatcggtacctcacaccgtcagccggcagtaaaaaacgtcgtgggttcaatccgcgagggactctccgacctcttctcccttccagaaggctacgagatcatcctttccctaggtggtgcgaccgcattctgggatgcagcaaccttcggactcattgaaaagaagtccggtcacctttctttcggtgagttctcctccaagttcgcaaaggcttctaagcttgctccttggctcgacgagccagagatcgtcaccgcagaaaccggtgactctccggccccacaggcattcgaaggcgccgatgttattgcatgggcacacaacgaaacctccactggcgccatggttccagttcttcgccccgaaggctctgaaggctccctggttgccattgacgcaacctccggcgctggtggactgccagtagacatcaagaactccgatgtttactacttctccccacagaagtgcttcgcatccgacggtggcctgtggcttgcagcgatgagcccagcagctctcgagcgcatcgagaagatcaacgcttccgatcgcttcatccctgagttcctcaacctgcagaccgcagtggataactccctgaagaaccagacctacaacaccccagctgttgctaccttgctgatgctggacaaccaggtcaagtggatgaactccaacggcggcctggatggaatggttgctcgcaccacagcaagctcctccgccctgtacaactgggctgaggctcgcgaggaggcatccccatacgtggcagatgcagctaagcgctccctcgttgtcggcaccatcgacttcgatgactccatcgacgcagcagtgatcgctaagatactgcgcgcaaacggcatcctggacaccgagccttaccgcaagctgggacgcaaccagctgcgcatcggtatgttcccagcgatcgattccaccgatgtggaaaagctcaccggagcaatcgacttcatcctcgatggcggttttgcaaggaagtaa

SEQ ID NO: 42 - protein, 376 aa, Corynebacterium glutamicum
MTDFPTLPSEFIPGDGRFGCGPSKVRPEQIQAIVDGSASVIGTSHRQPAVKNVVGSIREGLSDLFSLPEGYEIILSLGGATAFWDAATFGLIEKKSGHLSFGEFSSKFAKASKLAPWLDEPEIVTAETGDSPAPQAFEGADVIAWAHNETSTGAMVPVLRPEGSEGSLVAIDATSGAGGLPVDIKNSDVYYFSPQKCFASDGGLWLAAMSPAALERIEKINASDRFIPEFLNLQTAVDNSLKNQTYNTPAVATLLMLDNQVKWMNSNGGLDGMVARTTASSSALYNWAEAREEASPYVADAAKRSLVVGTIDFDDSIDAAVIAKILRANGILDTEPYRKLGRNQLRIGMFPAIDSTDVEKLTGAIDFILDGGFARK

SEQ ID NO: 43 - DNA, 822 bp, Escherichia coli
  source 1..822: mol_type = genomic DNA
atgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacggcggtgattgaaaacgacgtatcgattctgcaatctgtgacgcttggcggtacgggtaaatctggtggtgaccgtcacccgaaaattcgtgaaggtgtgaggattggcgcgggcgcgaaaatcctcggcaatattgaagttgggcgcggcgcgaagattggcgcaggttccgtggtgctgcaaccggtgccgccgcataccaccgccgctggcgttccggctcgtattgtcggtaaaccagacagcgataagccatcaatggatatggaccagcatttcaacggtattaaccatacatttgagtatggggatgggatctaa

SEQ ID NO: 44 - protein, 273 aa, Escherichia coli
MSCEELEIVWNNIKAEARTLADCEPMLASFYHATLLKHENLGSALSYMLANKLSSPIMPAIAIREVVEEAYAADPEMIASAACDIQAVRTRDPAVDKYSTPLLYLKGFHALQAYRIGHWLWNQGRRALAIFLQNQVSVTFQVDIHPAAKIGRGIMLDHATGIVVGETAVIENDVSILQSVTLGGTGKSGGDRHPKIREGVRIGAGAKILGNIEVGRGAKIGAGSVVLQPVPPHTTAAGVPARIVGKPDSDKPSMDMDQHFNGINHTFEYGDGI

SEQ ID NO: 45 - DNA, 972 bp, Escherichia coli
  source 1..972: mol_type = genomic DNA
atgagtaagatttttgaagataactcgctgactatcggtcacacgccgctggttcgcctgaatcgcatcggtaacggacgcattctggcgaaggtggaatctcgtaaccccagcttcagcgttaagtgccgtatcggtgccaacatgatttgggatgccgaaaagcgcggcgtgctgaaaccaggcgttgaactggttgaaccgaccagcggtaataccgggattgcactggcctatgtagctgccgctcgcggttacaaactcaccctgaccatgccagaaaccatgagtattgaacgccgcaagctgctgaaagcgttaggtgcaaacctggtgctgacggaaggtgctaaaggcatgaaaggcgcaatccaaaaagcagaagaaattgtcgccagcaatccagagaaatacctgctgctgcaacaattcagcaatccggcaaaccctgaaattcacgaaaagaccaccggtccggagatatgggaagataccgacggtcaggttgatgtatttattgctggcgttgggactggcggtacgctgactggcgtcagccgctacattaaaggcaccaaaggcaagaccgatcttatctctgtcgccgttgagccaaccgattctccagttatcgcccaggcgctggcaggtgaagagattaaacctggcccgcataaaattcagggtattggcgctggttttatcccggctaacctcgatctcaagctggtcgataaagtcattggcatcaccaatgaagaagcgatttctaccgcgcgtcgtctgatggaagaagaaggtattcttgcaggtatctcttctggagcagctgttgccgcggcgttgaaactacaagaagatgaaagctttaccaacaagaatattgtggttattctaccatcatcgggtgagcgttatttaagcaccgcattgtttgccgatctcttcactgagaaagaattgcaacagtaa

SEQ ID NO: 46 - protein, 323 aa, Escherichia coli
MSKIFEDNSLTIGHTPLVRLNRIGNGRILAKVESRNPSFSVKCRIGANMIWDAEKRGVLKPGVELVEPTSGNTGIALAYVAAARGYKLTLTMPETMSIERRKLLKALGANLVLTEGAKGMKGAIQKAEEIVASNPEKYLLLQQFSNPANPEIHEKTTGPEIWEDTDGQVDVFIAGVGTGGTLTGVSRYIKGTKGKTDLISVAVEPTDSPVIAQALAGEEIKPGPHKIQGIGAGFIPANLDLKLVDKVIGITNEEAISTARRLMEEEGILAGISSGAAVAAALKLQEDESFTNKNIVVILPSSGERYLSTALFADLFTEKELQQ

SEQ ID NO: 47 - DNA, 2943 bp, Escherichia coli
  source 1..2943: mol_type = genomic DNA
atggatcaaatacgacttactcacctgcggcaactggaggcggaaagcatccacattattcgcgaggtggcggcagaattctcaaatccggtgatgctctactctatcggtaaagattccagcgtcatgctgcatctggcgcgcaaggcgttttatccaggtacgctgcctttcccgttgctgcatgtcgataccggctggaaattccgcgagatgtatgagttccgcgatcgtactgctaaagcctacggctgcgaactgctggtgcataaaaacccggaaggcgtggcgatggggattaatccattcgtgcacggcagcgcgaaacataccgatattatgaaaactgaaggcctgaaacaggcgctgaacaaatacggttttgatgccgccttcggtggtgcgcgccgtgacgaagagaaatcccgcgctaaagagcgaatttactctttccgtgaccgcttccatcgctgggatccgaaaaatcagcgcccggagctgtggcacaactacaacgggcaaattaacaaaggcgaaagcatccgcgtcttcccgctctctaactggaccgagcaggatatctggcaatacatctggctggaaaatatcgacattgttccgctatatctcgctgcggaacgtccggttctggaacgcgacggtatgttgatgatgattgatgacaaccgtatcgacctgcaaccgggcgaagtgattaaaaaacggatggtgcgtttccgtacgctgggctgctggccgctgaccggtgcggtggagtcaaatgcacaaacactgccggaaatcatcgaagagatgctggtttccaccaccagtgaacgtcagggccgcgtgattgaccgcgaccaggcggggtctatggagctgaaaaaacgtcaggggtatttttaagatgaacaccgcacttgcacaacaaatcgccaatgaaggcggcgtcgaagcctggatgattgcgcaacaacataaaagcctgctgcgttttctgacctgtggtagcgtcgatgacggcaaaagtactctgattggtcgtctgctgcacgatacccgccaaatctacgaagatcagctctcatcgctgcataacgacagtaagcgtcacggcacccagggcgaaaagctggatctggctctgctggtggacggcctgcaagctgagcgcgaacagggcatcaccattgacgtggcctaccgctatttctctaccgagaagcgtaaatttattatcgccgacaccccagggcacgagcagtacacccgcaatatggcgactggcgcatcgacatgtgaactggcgatcttactgatcgatgcccgtaaaggcgtgctcgatcaaacccgtcgtcacagttttatctccacactgttggggatcaaacatctggtcgtggcgatcaacaaaatggatctggtggattacagtgaagagacgttcacccgtattcgtgaagattatttgacctttgccgggcagctgccgggtaatctggatatccgctttgtgccgctctctgcactggaaggcgacaacgtggcatcgcaaagtgaaagtatgccgtggtacagcggtccgacactgctcgaagtgctggaaaccgtggagatccagcgagtggtggatgctcagccaatgcgcttcccggtgcagtacgttaatcgcccgaatctcgattttcgtggttacgccggaacgctggcatccggtcgcgtggaagtcgggcaacgtgtcaaagtgctgccctctggtgtggaatcaaacgtcgcgcggatcgtgacttttgatggtgatcgcgaagaagcctttgccggagaagcgatcaccctggtgctgacggatgagatcgacatcagccgtggcgatctgctgctggcggcagacgaagcgttaccggcggtgcagagcgcgtcggtggatgtggtatggatggcggaacagccgctttctccagggcagagttacgacatcaaaattgccggtaagaagacgcgcgcgcgtgttgatggcattcgctatcaggttgatattaataaccttacccagcgtgaagttgaaaacctgccactgaatgggatcggcctcgtggatctcacttttgacgagccgctggtgttagatcgttatcaacaaaatccggtgacgggtgggctgatttttatcgatcgcctgagcaatgtgaccgtgggtgccggtatggtgcacgagccagttagccaggcaactgctgcgccatctgaattcagtgcattcgaactggaattgaatgctctggttcgtcgccactttccgcactggggcgcgcgcgatttgctgggggataaataatggcgctgcatgacgaaaacgtcgtctggcatagccatccggtcactgtgcaacaacgcgagctacaccacggtcatcgtggtgtagtgctgtggtttaccggcctctccgggtccggtaaatcaacggtcgccggggcgctggaggaggcgttacataaactcggcgtcagtacgtatctgctggatggcgacaatgttcgccacggattatgcagcgatctcggttttagcgatgccgatcgtaaagagaatatccgtcgcgtcggtgaagtggcgaatttgatggttgaagccggactggtggtgctgaccgcatttatctcgccacaccgcgccgaacgccagatggttcgcgaacgcgtaggagaagggcgctttatcgaagtgtttgtcgatacgccgctggcgatttgcgaagcccgcgatcccaaaggcttatataagaaagcgcgtgccggtgaactgcgcaactttacgggaatagattccgtttacgaagcgcctgaatcggcagaaattcatctcaatggtgaacaattagtaacaaatttggtacagcaattattagatctgttgagacagaacgatattatcagatcctga

SEQ ID NO: 48 - protein, 302 aa, Escherichia coli
MDQIRLTHLRQLEAESIHIIREVAAEFSNPVMLYSIGKDSSVMLHLARKAFYPGTLPFPLLHVDTGWKFREMYEFRDRTAKAYGCELLVHKNPEGVAMGINPFVHGSAKHTDIMKTEGLKQALNKYGFDAAFGGARRDEEKSRAKERIYSFRDRFHRWDPKNQRPELWHNYNGQINKGESIRVFPLSNWTEQDIWQYIWLENIDIVPLYLAAERPVLERDGMLMMIDDNRIDLQPGEVIKKRMVRFRTLGCWPLTGAVESNAQTLPEIIEEMLVSTTSERQGRVIDRDQAGSMELKKRQGYF

SEQ ID NO: 49 - protein, 475 aa, Escherichia coli
MNTALAQQIANEGGVEAWMIAQQHKSLLRFLTCGSVDDGKSTLIGRLLHDTRQIYEDQLSSLHNDSKRHGTQGEKLDLALLVDGLQAEREQGITIDVAYRYFSTEKRKFIIADTPGHEQYTRNMATGASTCELAILLIDARKGVLDQTRRHSFISTLLGIKHLVVAINKMDLVDYSEETFTRIREDYLTFAGQLPGNLDIRFVPLSALEGDNVASQSESMPWYSGPTLLEVLETVEIQRVVDAQPMRFPVQYVNRPNLDFRGYAGTLASGRVEVGQRVKVLPSGVESNVARIVTFDGDREEAFAGEAITLVLTDEIDISRGDLLLAADEALPAVQSASVDVVWMAEQPLSPGQSYDIKIAGKKTRARVDGIRYQVDINNLTQREVENLPLNGIGLVDLTFDEPLVLDRYQQNPVTGGLIFIDRLSNVTVGAGMVHEPVSQATAAPSEFSAFELELNALVRRHFPHWGARDLLGDK

SEQ ID NO: 50 - protein, 201 aa, Escherichia coli
MALHDENVVWHSHPVTVQQRELHHGHRGVVLWFTGLSGSGKSTVAGALEEALHKLGVSTYLLDGDNVRHGLCSDLGFSDADRKENIRRVGEVANLMVEAGLVVLTAFISPHRAERQMVRERVGEGRFIEVFVDTPLAICEARDPKGLYKKARAGELRNFTGIDSVYEAPESAEIHLNGEQLVTNLVQQLLDLLRQNDIIRS

SEQ ID NO: 51 - DNA, 741 bp, Escherichia coli
  source 1..741: mol_type = genomic DNA
atgttagatcaagtatgccagcttgcacggaatgcaggcgatgccattatgcaggtctacgacgggacgaaaccgatggacgtcgtcagcaaagcggacaattctccggtaacggcagcggatattgccgctcacaccgttatcatggacggtttacgtacgctgacaccggatgttccggtcctttctgaagaagatcctcccggttgggaagtccgtcagcactggcagcgttactggctggtagacccgctggatggtactaaagagtttattaaacgtaatggcgaattcaccgttaacattgcgctcattgaccatggcaaaccgattttaggcgtggtgtatgcgccggtaatgaacgtaatgtacagcgcggcagaaggcaaagcgtggaaagaagagtgcggtgtgcgcaagcagattcaggtccgcgatgcgcgcccgccgctggtggtgatcagccgttcccatgcggatgcggagctgaaagagtatctgcaacagcttggcgaacatcagaccacgtccatcggctcttcgctgaaattctgcctggtggcggaaggacaggcgcagctgtacccgcgcttcggaccaacgaatatttgggacaccgccgctggacatgctgtagctgcagctgccggagcgcacgttcacgactggcagggtaaaccgctggattacactccgcgtgagtcgttcctgaatccggggttcagagtgtctatttactaa

SEQ ID NO: 52 - protein, 246 aa, Escherichia coli
MLDQVCQLARNAGDAIMQVYDGTKPMDVVSKADNSPVTAADIAAHTVIMDGLRTLTPDVPVLSEEDPPGWEVRQHWQRYWLVDPLDGTKEFIKRNGEFTVNIALIDHGKPILGVVYAPVMNVMYSAAEGKAWKEECGVRKQIQVRDARPPLVVISRSHADAELKEYLQQLGEHQTTSIGSSLKFCLVAEGQAQLYPRFGPTNIWDTAAGHAVAAAAGAHVHDWQGKPLDYTPRESFLNPGFRVSIY

SEQ ID NO: 53 - DNA, 735 bp, Escherichia coli
  source 1..735: mol_type = genomic DNA
atgtccaaactcgatctaaacgccctgaacgaactgccgaaggtagatcgcattctggcgctggcggaaactaacgccgaactggaaaaactggacgctgaaggccgcgtagcctgggcgctggataatctgcccggtgaatatgtactttcttccagctttggcattcaggcggcggtgagcctgcatctggtgaatcaaattcgcccggatattccggtgatcctcaccgatacgggttacttgttcccggaaacctaccgctttattgacgagttaacggacaaactcaagctcaacctgaaagtgtaccgtgctaccgaaagcgcagcctggcaggaagcacgctacggaaaactgtgggagcagggcgttgaaggcattgaaaagtacaatgacatcaacaaagtcgaaccgatgaaccgggctctgaaagaactgaatgcgcaaacctggtttgctggcctgcgccgtgaacaatccggcagtcgtgccaatttaccggtgctggcaattcagcgtggcgtatttaaagtgctgccgattatcgactgggataaccgaactatttatcagtacctgcaaaaacatggcctgaaatatcacccattatgggatgaaggatatttatcggtgggcgatacccatacaacccgtaaatgggaacccggcatggcggaagaagaaacgcgtttctttggcttaaaaagggaatgtgggttacacgaagggtaa

SEQ ID NO: 54 - protein, 244 aa, Escherichia coli
MSKLDLNALNELPKVDRILALAETNAELEKLDAEGRVAWALDNLPGEYVLSSSFGIQAAVSLHLVNQIRPDIPVILTDTGYLFPETYRFIDELTDKLKLNLKVYRATESAAWQEARYGKLWEQGVEGIEKYNDINKVEPMNRALKELNAQTWFAGLRREQSGSRANLPVLAIQRGVFKVLPIIDWDNRTIYQYLQKHGLKYHPLWDEGYLSVGDTHTTRKWEPGMAEEETRFFGLKRECGLHEG

SEQ ID NO: 55 - DNA, 3512 bp, Escherichia coli
  source 1..3512: mol_type = genomic DNA
atgacgacacaggtcccaccttccgcgttgcttccgttgaacccggagcaactggcacgccttcaggcggccacgaccgatttaactcccacccagcttgcctgggtttctggctatttctggggcgtactcaatcagcagcctgctgcgcttgcagcgacgccagcgccagccgcagaaatgccgggtataactattatctccgcctcgcaaaccggcaatgcgcgccgggttgctgaagcattacgtgatgatttattagcagcaaaactgaacgttaagctggtgaacgcgggcgactataaattcaaacaaatcgccagcgaaaaactgctcatcgtagtgacgtcaacgcaaggggaaggggaaccgccggaagaagccgtcgcgctgcataagttcctgttctccaaaaaagcgccaaagctggaaaacaccgcgtttgccgtgtttagcctcggcgatagctcttatgaatttttctgccagtccgggaaagatttcgacagcaagctggcggaactgggtggtgaacgcctgctcgaccgtgtcgatgccgatgttgaataccaggctgctgccagcgagtggcgcgcccgcgtggttgatgcgcttaaatcgcgtgcgcctgtcgcggcaccttcgcaatccgtcgctactggcgcggtaaatgaaatccacaccagcccgtacagcaaagacgcgccgctggtggctagcctctctgttaaccagaaaattaccgggcgtaactctgaaaaagacgttcgccatatcgaaattgacttaggtgactcgggcatgcgttaccagccgggtgacgcgctgggcgtctggtatcagaacgatccggcactggtgaaagaacttgtcgaactgctgtggctgaaaggcgatgaacctgtcaccgtcgagggcaaaacgttgcctctgaacgaagcgctacagtggcacttcgaactgaccgtcaacaccgccaacattgttgagaattacgccacgcttacccgcagtgaaacactgctgccgctggtgggcgataaagcgaagttacagcattacgccgcgacgacgccgattgttgacatggtgcgtttctccccggcacagcttgatgccgaagcgctaattaatctgctgcgcccgctgacgccgcgtctgtattccatcgcctcctcgcaggcggaagtcgagaacgaagtacacgtcaccgttggtgtggtgcgttacgacgtggaaggccgcgcccgtgccggtggtgcctccagcttcctcgctgaccgcgtggaagaagagggcgaagtccgcgtatttatcgaacataacgataacttccgcctgccagccaatccagaaaccccggtgattatgattggcccaggcaccggtattgcgccgttccgcgcctttatgcagcaacgcgccgccgacgaagcgccaggtaaaaactggctgttctttggtaatccgcactttacggaagacttcctgtaccaggtggagtggcagcgctacgtcaaagatggcgtgctgacacgtatcgatcttgcctggtcgcgcgatcaaaaagaaaaagtttacgtacaagacaaactgcgcgaacagggcgcggagctgtggcgctggatcaatgatggtgcccacatttatgtctgcggcgacgctaatcgcatggcgaaagacgttgagcaggcacttctggaagtgattgccgaatttggtggcatggacaccgaagcggcggatgaatttttaagtgagctgcgcgtagagcgccgttatcagcgagatgtctactaatgagcgaaaaacatccagggcctttagtggtcgaaggaaaactgacagacgccgagcgcatgaagcatgaaagcaactacctgcgcggcaccattgcggaagatttaaacgacggtctgaccggcggctttaagggcgacaacttcctgctgattcgcttccacggcatgtatcagcaggatgaccgcgacatccgcgccgaacgtgctgaacagaagctggagccgcgccacgcgatgctgcttcgctgtcgtctgccgggtggggtgattaccactaaacagtggcaggcgatcgacaaatttgccggtgaaaacaccatctatggcagcattcgcctgaccaaccgccagacgtttcagttccacggcattctgaaaaagaacgtcaaaccggtgcaccagatgctgcactcggtcggtcttgatgcgctggcgacagctaacgacatgaaccgtaacgtactctgcacctcgaacccttacgagtcgcagctgcacgcggaagcgtacgagtgggcgaagaagatttctgagcatctgttgcctcgtacccgcgcgtatgcggagatctggctcgaccaggaaaaagtcgccactactgatgaagaaccgatcctcggccagacctacctgccgcgtaaattcaaaaccacggtagtgatcccgccacagaacgatatcgatctgcacgccaacgacatgaacttcgtggcgatcgccgaaaacggcaagctggtgggctttaacctgttggtgggcggtgggctttccatcgaacacggcaacaagaaaacctacgcccgcaccgcgagtgagtttggctatctgccgctggagcatacgctggcggtggccgaagccgtcgtgacaactcagcgtgactggggtaaccgaaccgatcgtaaaaatgccaaaaccaaatacacgctggagcgcgtgggggttgagacgtttaaagcggaagtggagcgtcgcgcggggatcaaatttgaaccgatccgtccatatgagttcaccggacgaggcgatcgtattggctgggttaagggcattgatgataactggcacctgacgctgtttatcgaaaatggtcgcatccttgattatccggcgcgtccgctgaaaaccggcctgctggagatcgcgaagatccacaaaggcgatttccgcattacggcgaaccagaatctgatcatcgccggtgtaccggaaagcgagaaagcgaagatcgagaagatcgccaaagagagcgggttaatgaatgccgtcacgccgcagcgtgaaaactcgatggcttgcgtgtcattcccgacttgcccgctggcgatggcggaagcagagcgtttcctgccgtcttttatcgacaacatcgataatttaatggcgaaacatggtgtcagcgatgagcatatcgtgatgcgtgtaacaggctgcccgaacggttgtggtcgcgcgatgctggcggaagtgggcctggtgggtaaagcgccgggtcgctacaacctgcatcttggcggcaaccgcattgggacacgtatcccacggatgtataaagaaaacatcaccgagccggaaatcctggcgtcgcttgatgaactgatagggcgctgggcgaaagagcgcgaagcgggtgaaggcttcggcgactttacggtgcgtgcgggcatcattcgcccggtgctcgatccggcgcgtgatttgtgggattaa

SEQ ID NO: 56 - protein, 599 aa, Escherichia coli
MTTQVPPSALLPLNPEQLARLQAATTDLTPTQLAWVSGYFWGVLNQQPAALAATPAPAAEMPGITIISASQTGNARRVAEALRDDLLAAKLNVKLVNAGDYKFKQIASEKLLIVVTSTQGEGEPPEEAVALHKFLFSKKAPKLENTAFAVFSLGDSSYEFFCQSGKDFDSKLAELGGERLLDRVDADVEYQAAASEWRARVVDALKSRAPVAAPSQSVATGAVNEIHTSPYSKDAPLVASLSVNQKITGRNSEKDVRHIEIDLGDSGMRYQPGDALGVWYQNDPALVKELVELLWLKGDEPVTVEGKTLPLNEALQWHFELTVNTANIVENYATLTRSETLLPLVGDKAKLQHYAATTPIVDMVRFSPAQLDAEALINLLRPLTPRLYSIASSQAEVENEVHVTVGVVRYDVEGRARAGGASSFLADRVEEEGEVRVFIEHNDNFRLPANPETPVIMIGPGTGIAPFRAFMQQRAADEAPGKNWLFFGNPHFTEDFLYQVEWQRYVKDGVLTRIDLAWSRDQKEKVYVQDKLREQGAELWRWINDGAHIYVCGDANRMAKDVEQALLEVIAEFGGMDTEAADEFLSELRVERRYQRDVY

SEQ ID NO: 57 - protein, 570 aa, Escherichia coli
MSEKHPGPLVVEGKLTDAERMKHESNYLRGTIAEDLNDGLTGGFKGDNFLLIRFHGMYQQDDRDIRAERAEQKLEPRHAMLLRCRLPGGVITTKQWQAIDKFAGENTIYGSIRLTNRQTFQFHGILKKNVKPVHQMLHSVGLDALATANDMNRNVLCTSNPYESQLHAEAYEWAKKISEHLLPRTRAYAEIWLDQEKVATTDEEPILGQTYLPRKFKTTVVIPPQNDIDLHANDMNFVAIAENGKLVGFNLLVGGGLSIEHGNKKTYARTASEFGYLPLEHTLAVAEAVVTTQRDWGNRTDRKNAKTKYTLERVGVETFKAEVERRAGIKFEPIRPYEFTGRGDRIGWVKGIDDNWHLTLFIENGRILDYPARPLKTGLLEIAKIHKGDFRITANQNLIIAGVPESEKAKIEKIAKESGLMNAVTPQRENSMACVSFPTCPLAMAEAERFLPSFIDNIDNLMAKHGVSDEHIVMRVTGCPNGCGRAMLAEVGLVGKAPGRYNLHLGGNRIGTRIPRMYKENITEPEILASLDELIGRWAKEREAGEGFGDFTVRAGIIRPVLDPARDLWD

SEQ ID NO: 58 - DNA, 975 bp, Escherichia coli
  source 1..975: mol_type = genomic DNA
atgaaattacaacaacttcgctatattgttgaggtggtcaatcataacctgaatgtctcatcaacagcggaaggactttacacatcacaacccgggatcagtaaacaagtcagaatgctggaagacgagctaggcattcaaattttttcccgaagcggcaagcacctgacgcaggtaacgccagcagggcaagaaataattcgtatcgctcgcgaagtcctgtcgaaagtcgatgccataaaatcggttgccggagagcacacctggccggataaaggttcactgtatatcgccaccacgcatacccaggcacgctacgcattaccaaacgtcatcaaaggctttattgagcgttatcctcgcgtttctttgcatatgcaccagggctcgccgacacaaattgctgatgccgtctctaaaggcaatgctgatttcgctatcgccacagaagcgctgcatctgtatgaagatttagtgatgttaccgtgctaccactggaatcgggctattgtagtcactccggatcacccgctggcaggcaaaaaagccattaccattgaagaactggcgcaatatccgttggtgacatataccttcggctttaccggacgttcagaactggatactgcctttaatcgcgcagggttaacgccgcgtatcgttttcacggcaacggatgctgacgtcattaaaacttacgtccggttagggctgggggtaggggtcattgccagcatggcggtggatccggtcgccgatcccgaccttgtgcgtgttgatgctcacgatatcttcagccacagtacaaccaaaattggttttcgccgtagtactttcttgcgcagttatatgtatgatttcattcagcgttttgcaccgcatttaacgcgtgatgtcgttgatgcggctgtcgcattgcgctctaatgaagaaattgaggtcatgtttaaagatataaaactgccggaaaaataa

SEQ ID NO: 59 - protein, 324 aa, Escherichia coli
MKLQQLRYIVEVVNHNLNVSSTAEGLYTSQPGISKQVRMLEDELGIQIFSRSGKHLTQVTPAGQEIIRIAREVLSKVDAIKSVAGEHTWPDKGSLYIATTHTQARYALPNVIKGFIERYPRVSLHMHQGSPTQIADAVSKGNADFAIATEALHLYEDLVMLPCYHWNRAIVVTPDHPLAGKKAITIEELAQYPLVTYTFGFTGRSELDTAFNRAGLTPRIVFTATDADVIKTYVRLGLGVGVIASMAVDPVADPDLVRVDAHDIFSHSTTKIGFRRSTFLRSYMYDFIQRFAPHLTRDVVDAAVALRSNEEIEVMFKDIKLPEK

SEQ ID NO: 60 - DNA, 387 bp, Roseobacter denitrificans
  source 1..387: mol_type = genomic DNA
atgaccaaaacactgacagctcaggacttgtccgacacctttgacgccttcaatcgccatgacgttgatggcgtcatgacacatttcgccgatgattgcgtgttctacaccgtgggcggggatgaagcctatggcgccaaagtcgaaggcgcagaagcgattgccaaagcattctctgccgtctgggcgggcatgaaggacgcccattgggatcatcacagccactttgtgcatggggatcgcgccgtatccgaatggacgttctccggaactggcgcggacggcatgcgcatcgaagcacagggcgctgacctctttaccctgcgcgacggcaagatcatcgtgaaacaggccctgcgcaaatcccgcccgcccttcaaggcttaa

SEQ ID NO: 61 - protein, 128 aa, Roseobacter denitrificans
MTKTLTAQDLSDTFDAFNRHDVDGVMTHFADDCVFYTVGGDEAYGAKVEGAEAIAKAFSAVWAGMKDAHWDHHSHFVHGDRAVSEWTFSGTGADGMRIEAQGADLFTLRDGKIIVKQALRKSRPPFKA

SEQ ID NO: 62 - DNA, 1395 bp, Roseobacter denitrificans
  source 1..1395: mol_type = genomic DNA
atgccacatagaccaaagcactggcccaaggccagctacgatcccaaatacgatcctatcgtcgacgcgggtcccggtcacaaccgggaccacgcaccgacctattggattggtacggcggggacgccacctgaagatgacgggccggtgtcgggtgacatcgatgcggatgtcgtcgttgtcggctctggctatacaggtctgtctaccgcaatccacctggcgaaggaccacggcatcaaggcgcatgtccttgaagccaacacagtcgcctggggctgttccacccgcaatggcgggcaggcacagatttcttccggtcgtctcaagcggtcggagtggatcaagcggtggggcgtggatgtcgccaaaggcatgcacgccgaggtctgtgaagccttcgaactgttcaatgatctgatcgggtcagatgacattgattgcgacccgcaaaccgggggccatttctatattgcccaccgcgaaaaggtcatggcgaagctggaaaaggaatgtgccgtcctgaacgacacgtttggctatggctctcgcattctgtcgcgcgacgaactacacgaaaaatacgtgcgggatcaggaagcacacggtgccctttgggaaccggacgggacctcgatccacgcggcaaaactggccttcagctacgtgcgtcttgcgcgcaaactcggcgccaagatccacacggccagcccggtcatggggtggaagaccgtgaacggtgtgcatcacctcaccacgcccggtggcacggtgcgcgcacgtgccgtggccttggcgacagcgggctacacaccgccggggctgaacgaaaagaccaagcaccggctcatgccgatcctgtcaaactccatcgtgacgcgtccgctgagcgatgaggaaaaggcgggatgcggttttcaggtgaaatctccgctgactgacacgcgcaccttgcggcactactaccgctatctgcccgacggacgggtccagatcggcagccgcagtgcgattacaggtcgagacgcagagaaccccagacatctggagcttctgcagaaaggtctctatcgcaagttccccgtgctcgaaggcattgaactggattactcctggtggggatgggtggatgtcagccatgacatgatgccacgcattttccagccaaacccgaagcaaacaatcttttatgcgatgggctacggcggcaacggggtgatgtattccgcacaggccggcaagcgcatggcgcaaatggttgcgggcgaaggcaaggacctcaaacttccgatcttcacctcgcaactgccaagccacggtgttctgacacccttccgcaggttgggccagcgcatggcctacccctactactaccttcgcgatgaaattctctga

SEQ ID NO: 63 - protein, 464 aa, Roseobacter denitrificans
MPHRPKHWPKASYDPKYDPIVDAGPGHNRDHAPTYWIGTAGTPPEDDGPVSGDIDADVVVVGSGYTGLSTAIHLAKDHGIKAHVLEANTVAWGCSTRNGGQAQISSGRLKRSEWIKRWGVDVAKGMHAEVCEAFELFNDLIGSDDIDCDPQTGGHFYIAHREKVMAKLEKECAVLNDTFGYGSRILSRDELHEKYVRDQEAHGALWEPDGTSIHAAKLAFSYVRLARKLGAKIHTASPVMGWKTVNGVHHLTTPGGTVRARAVALATAGYTPPGLNEKTKHRLMPILSNSIVTRPLSDEEKAGCGFQVKSPLTDTRTLRHYYRYLPDGRVQIGSRSAITGRDAENPRHLELLQKGLYRKFPVLEGIELDYSWWGWVDVSHDMMPRIFQPNPKQTIFYAMGYGGNGVMYSAQAGKRMAQMVAGEGKDLKLPIFTSQLPSHGVLTPFRRLGQRMAYPYYYLRDEIL

SEQ ID NO: 64 - DNA, 852 bp, Escherichia coli
  source 1..852: mol_type = genomic DNA
atgagtgaacgtctgagcattaccccgctggggccgtatatcggcgcacaaatttcgggtgccgacctgacgcgcccgttaagcgataatcagtttgaacagctttaccatgcggtgctgcgccatcaggtggtgtttctacgcgatcaagctattacgccgcagcagcaacgcgcgctggcccagcgttttggcgaattgcatattcaccctgtttacccgcatgccgaaggggttgacgagatcatcgtgctggatacccataacgataatccgccagataacgacaactggcataccgatgtgacatttattgaaacgccacccgcaggggcgattctggcagctaaagagttaccttcgaccggcggtgatacgctctggaccagcggtattgcggcctatgaggcgctctctgttcccttccgccagctgctgagtgggctgcgtgcggagcatgatttccgtaaatcgttcccggaatacaaataccgcaaaaccgaggaggaacatcaacgctggcgcgaggcggtcgcgaaaaacccgccgttgctacatccggtggtgcgaacgcatccggtgagcggtaaacaggcgctgtttgtgaatgaaggctttactacgcgaattgttgatgtgagcgagaaagagagcgaagccttgttaagttttttgtttgcccatatcaccaaaccggagtttcaggtgcgctggcgctggcaaccaaatgatattgcgatttgggataaccgcgtgacccagcactatgccaatgccgattacctgccacagcgacggataatgcatcgggcgacgatccttggggataaaccgttttatcgggcggggtaa

SEQ ID NO: 65 - protein, 283 aa, Escherichia coli
MSERLSITPLGPYIGAQISGADLTRPLSDNQFEQLYHAVLRHQVVFLRDQAITPQQQRALAQRFGELHIHPVYPHAEGVDEIIVLDTHNDNPPDNDNWHTDVTFIETPPAGAILAAKELPSTGGDTLWTSGIAAYEALSVPFRQLLSGLRAEHDFRKSFPEYKYRKTEEEHQRWREAVAKNPPLLHPVVRTHPVSGKQALFVNEGFTTRIVDVSEKESEALLSFLFAHITKPEFQVRWRWQPNDIAIWDNRVTQHYANADYLPQRRIMHRATILGDKPFYRAG

SEQ ID NO: 66 - DNA, 1392 bp, Roseobacter denitrificans
  source 1..1392: mol_type = genomic DNA
atggacggcaatttcaatgaaaatgatatctcccgcgtcgtcgaagcagaccgcgcgcatatctggcaccatctgagccagcacaaaccttacgagacaacagacccgcgcatcattgtcgaaggcaagggcatgaaggtttgggaccagaagggcaaagagcatcttgatgccgtctccggtggggtctggaccgtcaatgtcggctatggccgcgaacgcatcgccaacgccgtgcgggaccagttggtcaagttgaactatttcgccggctccgcaggctccatccccggtgccatgttcgccgagcgtctgatcgagaagatgccggggctgagccgcgtttattactgcaattccggctccgaggcgaatgaaaaagccttcaagatggtccgccagatcgcgcacaaacgctatggcggcaaaaagcacaaggtgctttatcgcgagcgtgactatcacggcaccaccatttccgccctttccgcaggcgggcaggacgaacggaacgcacaatatggccccttcacgcccggtttcgtgcgcgtgccccattgccttgaataccgcgcctttgaacaggaaggggcgccacaggaaaactacggtgtctgggcggcggatcagatcgaaaaggtaatcctcgccgaagggcccgataccgtgggcggcctgtgccttgaaccggtcactgcaggtggcggggtgatcacgccccccgatggctactgggagcgtgtgcaggaaatctgccacaaatacgacatcctgctgcatatcgacgaggtcgtatgcggcgtcggtcggaccggcacatggttcggctatcagcactacggcatccagccggatatggtcacgatggccaagggtgtcgcgtccggttacgcggcgatcgcctgccttgtgaccaatgaaaaagtcttcgacatgttcaaggatgacgcctcggatccgctgaactacttccgcgacatctcgacctttgggggctgcacggcgggtccggcagctgcgctggaaaacctgtcgatcatcgaagaagaaggcctgctggacaacaccacggaacagggggcctatatgctcgactgtctgggcggcttgatggacaagcacaagatcatcggccaggtgcgcggcaaggggctgttcctcggtgccgaactggtcgaggatcgcgacacgcgcaaaccggttgacgaaaggctcgcgcaagcggtggtcgcggactgcatgcaacagggtgtgatcatcggcgtgaccaaccgctctctgccgggcaagaacaacacgctgtgtttctcgcccgccctgatcgccagcaaggatgacattgaccacatctgcgacgcggtggacggtgcgctgtcgcgcgttttcggctaa

SEQ ID NO: 67 - protein, 463 aa, Roseobacter denitrificans
MDGNFNENDISRVVEADRAHIWHHLSQHKPYETTDPRIIVEGKGMKVWDQKGKEHLDAVSGGVWTVNVGYGRERIANAVRDQLVKLNYFAGSAGSIPGAMFAERLIEKMPGLSRVYYCNSGSEANEKAFKMVRQIAHKRYGGKKHKVLYRERDYHGTTISALSAGGQDERNAQYGPFTPGFVRVPHCLEYRAFEQEGAPQENYGVWAADQIEKVILAEGPDTVGGLCLEPVTAGGGVITPPDGYWERVQEICHKYDILLHIDEVVCGVGRTGTWFGYQHYGIQPDMVTMAKGVASGYAAIACLVTNEKVFDMFKDDASDPLNYFRDISTFGGCTAGPAAALENLSIIEEEGLLDNTTEQGAYMLDCLGGLMDKHKIIGQVRGKGLFLGAELVEDRDTRKPVDERLAQAVVADCMQQGVIIGVTNRSLPGKNNTLCFSPALIASKDDIDHICDAVDGALSRVFG

SEQ ID NO: 68 - DNA, 3415 bp, Escherichia coli
  source 1..3415: mol_type = genomic DNA
atggcaatttcatcgcgtaacacacttcttgccgcactggcattcatcgcttttcaggcacaggcggtgaacgtcaccgtggcgtatcaaacctcagccgaaccggcgaaagtggctcaggccgacaacacctttgctaaagaaagcggagcaaccgtggactggcgtaagtttgacagcggagccagcatcgtgcgggcgctggcttcaggcgacgtgcaaatcggcaacctcggttccagcccgttagcggttgcagccagccaacaggtgccgattgaagtcttcttgctggcgtcaaaactgggtaactccgaagcgctggtggtaaagaaaactatcagcaaaccggaagatctgattggcaaacgcatcgccgtaccgtttatctccaccacccactacagcctgctggcggcactgaaacactggggcattaaacccgggcaagtggagattgtgaacctgcagccgcccgcgattatcgctgcctggcagcggggagatattgatggtgcttatgtctgggcaccggcggttaacgccctggaaaaagacggcaaggtgttgaccgattctgaacaggtcgggcagtggggcgcgccaacgctggacgtctgggtggtgcgcaaagattttgccgagaaacatcctgaggtcgtgaaagcgttcgctaaaagcgccatcgatgctcagcaaccgtacattgctaacccagacgtgtggctgaaacagccggaaaacatcagcaaactggcgcgtttaagcggcgtgcctgaaggtgacgttccggggctggtgaaggggaatacctatctgacgccgcagcaacaaacggcagaactgaccggaccggtgaacaaagcgatcatcgacaccgcgcagtttttgaaagagcagggcaaggtcccggctgtagcgaatgattacagccagtacgttacctcgcgcttcgtgcaataaaaggaggcgcagatgctgcaaatctctcatctttacgccgattatggcggcaaaccggcactggaagatatcaacctgacgctggaaagcggcgagctactggtggtgctggggccgtccggctgcggtaaaaccaccctgctgaatctgattgccggttttgtgccttatcagcatggcagcattcaactggcgggtaagcgtattgagggaccgggagcagagcgtggcgtagtttttcagaatgaagggctactaccgtggcgcaatgtacaggacaacgtggcgttcggcctgcaattggcaggtatagagaaaatgcagcgactggaaatcgcgcaccagatgctgaaaaaagtggggctggaaggcgcagaaaaacgctacatctggcagctttccggtggtcaacgtcagcgggtggggattgctcgtgcgctggcggcgaatccccagctgttattactcgacgaaccgtttggtgcgctggacgccttcacccgcgaccagatgcaaaccctgctgctgaaactctggcaggagacgggcaagcaggtgctgttgattacccacgatatagaagaagcggtgtttatggcgactgaactggttctgctttcatccggccctggccgtgtgctggagcggctgccgctcaactttgctcgccgctttgttgcgggagagtcgagccgcagcatcaagtccgatccacaattcatcgccatgcgcgaatatgttttaagccgcgtatttgagcaacgggaggcgttctcatgagtgtgctcattaatgaaaaactgcattcgcggcggctgaaatggcgctggccgctctcgcgtcaggtgaccttaagcattggcacgttagcggttttactcaccgtatggtggacggtggcgacgctgcaactgattagcccgctatttttgccgccgccgcaacaggtactggaaaaactactcaccattgccggaccgcaaggctttatggacgccacgctgtggcagcatctggcagccagtctgacgcgcattatgctggcgctatttgcagcggtgttgttcggtattccggtcgggatcgcgatgggacttagccctacggtacgcggcattctggatccgataatcgagctttatcgtccggtgccgccgctggcttatttgccgctgatggtgatctggtttggtattggtgaaacctcgaagatcttactgatctatttagcgatttttgcaccggtggcgatgtcggcgctggcgggggtgaaaagcgtgcagcaggttcgcattcgtgccgcccagtcgctgggtgccagccgtgcgcaggtgctgtggtttgtcattttgcccggtgcgctgccggaaatcctcaccggattacgtattggtctgggggtgggctggtctacgctggtggcggcggagctgattgccgcgacgcgcggtttaggatttatggttcagtcagcgggtgaatttctcgcaactgacgtggtgctggcggggatcgcggtgattgcgattatcgcctttcttttagaactgggtctgcgcgcgttacagcgccgcctgacgccctggcatggagaagtacaatgagtgaacgtctgagcattaccccgctggggccgtatatcggcgcacaaatttcgggtgccgacctgacgcgcccgttaagcgataatcagtttgaacagctttaccatgcggtgctgcgccatcaggtggtgtttctacgcgatcaagctattacgccgcagcagcaacgcgcgctggcccagcgttttggcgaattgcatattcaccctgtttacccgcatgccgaaggggttgacgagatcatcgtgctggatacccataacgataatccgccagataacgacaactggcataccgatgtgacatttattgaaacgccacccgcaggggcgattctggcagctaaagagttaccttcgaccggcggtgatacgctctggaccagcggtattgcggcctatgaggcgctctctgttcccttccgccagctgctgagtgggctgcgtgcggagcatgatttccgtaaatcgttcccggaatacaaataccgcaaaaccgaggaggaacatcaacgctggcgcgaggcggtcgcgaaaaacccgccgttgctacatccggtggtgcgaacgcatccggtgagcggtaaacaggcgctgtttgtgaatgaaggctttactacgcgaattgttgatgtgagcgagaaagagagcgaagccttgttaagttttttgtttgcccatatcaccaaaccggagtttcaggtgcgctggcgctggcaaccaaatgatattgcgatttgggataaccgcgtgacccagcactatgccaatgccgattacctgccacagcgacggataatgcatcgggcgacgatccttggggataaaccgttttatcgggcggggtaa

SEQ ID NO: 69 - DNA, 4236 bp, Escherichia coli
  source 1..4236: mol_type = genomic DNA
atgcgtgtcatcaccctggcgggtagtcctcgctttccttctcgctccagctccttgctggaatatgcgcgggaaaaactaaatggcctggatgtagaggtttatcactggaatctgcaaaacttcgccccggaagatctactttatgctcgtttcgatagtccggcactcaagaccttcaccgaacagctgcaacaggccgatgggctgattgtcgccacgcctgtgtataaagccgcctattccggtgcgttgaaaaccctgctcgacctgctgccagaacgcgctttgcaaggcaaagtggtgctaccgctggcgacgggcggtaccgtggcccatctgctggcggtcgattatgcccttaaaccagttttaagcgcactgaaagctcaggagatcctgcacggcgtgtttgccgatgactcacaagtaattgattaccatcacagaccccagttcacgccaaatctgcaaacccgtcttgataccgcgctagaaactttctggcaggcattgcaccgccgcgatgttcaggttcctgaccttctgtctctgcgaggtaatgcccatgcgtaacatcattaaactggcgctggcgggattgcttagcgtctctacttttgcggttgctgcagaatcctcgcctgaagcgttacgtataggctatcagaaaggcagtattggtatggtgctggcaaaaagccaccagttactggaaaaacgctatccggaatcaaaaatctcctgggtggagttccccgcgggtccgcaaatgttggaagcgttaaacgttggcagtattgatctcggcagtaccggggatattccgccaatctttgcccaggctgccggggctgatttggtgtacgtgggcgtcgagccaccgaagcccaaagccgaagtgattctggtggcagaaaacagcccgatcaaaaccgtagccgatcttaaaggtcacaaagttgcctttcagaaaggttccagttcacacaaccttttactgcgtgcactgcgtcaggccggacttaagtttaccgatatccaacccacttacctgacgcccgctgatgcccgcgccgcgttccagcaaggtaacgttgacgcctgggctatctgggatccctactactctgctgcattattacagggcggcgtgcgggtgctgaaagacggcaccgatctcaatcaaactggatcgttttatctggcagctcgtccctatgcagaaaaaaacggcgcttttattcagggcgtactggcaacctttagtgaggccgatgcgttaacccgcagccagcgcgagcaaagcatcgctttactggcaaaaacgatgggcttaccggcaccggtgattgcctcttacttagatcatcgccctcctaccaccatcaaaccggttaacgccgaggttgccgccttacagcagcaaacggcagatctgttttatgaaaatcgtctggtgccgaaaaaagtcgatattcgccagcgcatctggcagcccactcaactggaaggaaaacaattatgagtctgaatatgttctggtttttaccgacccacggtgacgggcattatctgggaacggaagaaggttcacgcccggttgatcacggttatctgcaacaaattgcgcaagcggcggatcgtcttggctataccggtgtgctaattccaacggggcgctcctgcgaagatgcgtggctggttgccgcatcgatgatcccggtgacgcagcggctgaagtttcttgtcgccctgcgtcccagcgtaacctcacctaccgttgccgcccgccaggccgccacgcttgaccgtctctcaaatggacgtgcgttgtttaacctggtcacaggcagcgatccacaagagctggcaggcgacggagtgttccttgatcatagcgagcgctacgaagcctcggcggaatttacccaggtctggcggcgtttattgcagagagaaaccgtcgatttcaacggtaaacatattcatgtgcgcggagcaaaactgctcttcccggcgattcaacagccgtatccgccactttactttggcggatcgtcagatgtcgcccaggagctggcggcagaacaggttgatctctacctcacctggggcgaaccgccggaactggttaaagagaaaatcgaacaagtgcgggcgaaagctgccgcgcatggacgcaaaattcgtttcggtattcgtctgcatgtgattgttcgtgaaactaacgacgaagcgtggcaggccgccgagcggttaatctcgcatcttgatgatgaaactatcgccaaagcacaggccgcattcgcccggacggattccgtagggcaacagcgaatggcggcgttacataacggcaagcgcgacaatctggagatcagccccaatttatgggcgggcgttggcttagtgcgcggcggtgccgggacggcgctggtgggcgatggtcctacggtcgctgcgcgaatcaacgaatatgccgcgcttggcatcgacagttttgtgctttcgggctatccgcatctggaagaagcgtatcgggttggcgagttgctgttcccgcttctggatgtcgccatcccggaaattccccagccgcagccgctgaatccgcaaggcgaagcggtggcgaatgattttatcccccgtaaagtcgcgcaaagctaaggaagaaataatggcaacgccagtgaagaagtggttattgcgcgttgccccctggtttttaccggtgggcatcgtggcggtgtggcaactggcctcctcggttggctggctgtcgacgcgtattttgccttcaccggaaggagtggtgacggcgttctggacgctctccgccagcggcgaactgtggcagcatctggcgatcagctcctggcgggcgctgattggcttttcaattggcggatcgctggggttgattttgggactgattagcgggctatctcgctggggagaacggctgctggatacctcaattcagatgttgcgcaacgtgccgcatctggcgctgatcccgctggtgattttgtggtttggcattgatgaatccgcaaaaatctttctggtggcgctcggtacgctattccccatttatatcaacacctggcatggcatccgtaatatcgatcgcgggttggtggagatggcgcgtagctatggattatccggcataccgctgtttatccatgtgatcctgcctggtgccctgccctcaattatggtcggcgtgcgttttgcgttgggcctgatgtggctgacgctgattgttgccgaaaccatttctgccaattcaggcattggttatctggcgatgaatgcgcgggagtttttgcaaacggacgtggtggtagtcgctattattctttacgccctgctcggcaaactggctgacgtcagcgcgcagctgttagagcgcctctggttgcgctggaacccggcttatcatttgaaggaggccacggtatgaatactgctcgtctgaaccagggcacgccattgttgctcaatgcagtaagcaaacattacgcggaaaatatcgtcctgaaccaactggatttacatattccggcaggtcagtttgtggcggtggtgggccgcagcggtggtggcaaaagtaccctgctgcgcctgctggcaggtctggaaacgccaaccgcaggcgatgtgttagcgggcaccacaccgctggctgaaattcaggaagacacgcgaatgatgtttcaggatgcgcgtctgctaccgtggaaatcggtgattgataacgttgggttaggccttaaaggccagtggcgcgatgccgcacgtcgagcgttggctgcggtaggactggagaatcgcgcaggggaatggcctgccgcactttctggcgggcagaaacagcgagtggcgctggcaagagcgttgattcatcgaccgggattattgttgcttgatgaaccgctcggggcgctggacgccttaacgcgactcgagatgcaggatttgattgtgtcactttggcaggagcacggctttaccgtactgttggtgacgcatgatgtgagcgaagcggtagcgatggctgaccgggtgctgttaattgaagagggaaaaattggtctggatttgacagtggatatcccccgtccgcgtcgattagggtcggtgcgattagcggagctggaagcggaggtgttgcaacgggtgatgcaacgtggtgaatctgaaactcgcctgagaaaacaggggtaa

SEQ ID NO: 70 - DNA, 1146 bp, Escherichia coli
  source 1..1146: mol_type = genomic DNA
atgagtctgaatatgttctggtttttaccgacccacggtgacgggcattatctgggaacggaagaaggttcacgcccggttgatcacggttatctgcaacaaattgcgcaagcggcggatcgtcttggctataccggtgtgctaattccaacggggcgctcctgcgaagatgcgtggctggttgccgcatcgatgatcccggtgacgcagcggctgaagtttcttgtcgccctgcgtcccagcgtaacctcacctaccgttgccgcccgccaggccgccacgcttgaccgtctctcaaatggacgtgcgttgtttaacctggtcacaggcagcgatccacaagagctggcaggcgacggagtgttccttgatcatagcgagcgctacgaagcctcggcggaatttacccaggtctggcggcgtttattgcagagagaaaccgtcgatttcaacggtaaacatattcatgtgcgcggagcaaaactgctcttcccggcgattcaacagccgtatccgccactttactttggcggatcgtcagatgtcgcccaggagctggcggcagaacaggttgatctctacctcacctggggcgaaccgccggaactggttaaagagaaaatcgaacaagtgcgggcgaaagctgccgcgcatggacgcaaaattcgtttcggtattcgtctgcatgtgattgttcgtgaaactaacgacgaagcgtggcaggccgccgagcggttaatctcgcatcttgatgatgaaactatcgccaaagcacaggccgcattcgcccggacggattccgtagggcaacagcgaatggcggcgttacataacggcaagcgcgacaatctggagatcagccccaatttatgggcgggcgttggcttagtgcgcggcggtgccgggacggcgctggtgggcgatggtcctacggtcgctgcgcgaatcaacgaatatgccgcgcttggcatcgacagttttgtgctttcgggctatccgcatctggaagaagcgtatcgggttggcgagttgctgttcccgcttctggatgtcgccatcccggaaattccccagccgcagccgctgaatccgcaaggcgaagcggtggcgaatgattttatcccccgtaaagtcgcgcaaagctaa

SEQ ID NO: 71 - protein, 381 aa, Escherichia coli
MSLNMFWFLPTHGDGHYLGTEEGSRPVDHGYLQQIAQAADRLGYTGVLIPTGRSCEDAWLVAASMIPVTQRLKFLVALRPSVTSPTVAARQAATLDRLSNGRALFNLVTGSDPQELAGDGVFLDHSERYEASAEFTQVWRRLLQRETVDFNGKHIHVRGAKLLFPAIQQPYPPLYFGGSSDVAQELAAEQVDLYLTWGEPPELVKEKIEQVRAKAAAHGRKIRFGIRLHVIVRETNDEAWQAAERLISHLDDETIAKAQAAFARTDSVGQQRMAALHNGKRDNLEISPNLWAGVGLVRGGAGTALVGDGPTVAARINEYAALGIDSFVLSGYPHLEEAYRVGELLFPLLDVAIPEIPQPQPLNPQGEAVANDFIPRKVAQS

SEQ ID NO: 72 - DNA, 1146 bp, Corynebacterium glutamicum
  source 1..1146: mol_type = genomic DNA
atgacattaactttccattggttcctatccacttcaggcgattcccgcggcatcatcggcggcggtcacggtgcagaaaaatccggcacctcccgcgaattgagccacagctacctcaagcagttggcgctagctgccgagaccaacggttttgaatctgtcctgacaccaacgggcacgtggtgcgaagatgcgtggattactgacgcttctttgattgaggcgacaaaacgcttgaagttcctcgttgcgcttcgccctgggcagattggacctacgctgtctgctcaaatggcttctactttccagcgtctgtctggcaaccgtttgctgatcaatgtggtcaccggtggggaagatgcggagcagcgtgcgtttggtgatttcttgaacaaggaggagcgctacgcccgtaccggagaattcttggatatcgtgagccgcttgtggcgaggcgaaaccgtcacgcaccacggtgaacacctgcaggtggagcaagctagccttgcgcatccgccagagattattccggagattctttttggtggatcgtcgccagctgcaggtgaggtggctgcacgttatgcggacacctatctcacgtggggtgaaactcccgatcaggtggcgcagaaaatcaactggatcaacgagctagcagcacagcgcggccgggaactgcgccatggaatccgcttccatgtgatcacccgcgatacgtctgaagaagcatgggtggtggcagagaagttgattagcggggtcactccagaacaggtcgctaaggctcaagccgggtttgcaacgtctaagtcggaggggcagcgccggatggctgagctgcacagcaagggtcgtgcctttactagtggctcaactgctcgtgatctggaggtgtatcccaatgtgtgggcaggcgtcggtttgcttcgcggaggtgcaggaacagcccttgtgggctcgcatgaagaggtcgccgatcgcatcgaagaatacgcagcactcggcttggatcagtttgtactgtcgggttatccaaacttggaggaggccttccacttcggtgagggtgtgattccggagctgctgcgccgcggtgtggatatcaaaaatcaagaatcacgagttttggaacctgttgggtaa

SEQ ID NO: 73 - protein, 381 aa, Corynebacterium glutamicum
MTLTFHWFLSTSGDSRGIIGGGHGAEKSGTSRELSHSYLKQLALAAETNGFESVLTPTGTWCEDAWITDASLIEATKRLKFLVALRPGQIGPTLSAQMASTFQRLSGNRLLINVVTGGEDAEQRAFGDFLNKEERYARTGEFLDIVSRLWRGETVTHHGEHLQVEQASLAHPPEIIPEILFGGSSPAAGEVAARYADTYLTWGETPDQVAQKINWINELAAQRGRELRHGIRFHVITRDTSEEAWVVAEKLISGVTPEQVAKAQAGFATSKSEGQRRMAELHSKGRAFTSGSTARDLEVYPNVWAGVGLLRGGAGTALVGSHEEVADRIEEYAALGLDQFVLSGYPNLEEAFHFGEGVIPELLRRGVDIKNQESRVLEPVG

SEQ ID NO: 74 - DNA, 576 bp, Escherichia coli
  source 1..576: mol_type = genomic DNA
atgcgtgtcatcaccctggcgggtagtcctcgctttccttctcgctccagctccttgctggaatatgcgcgggaaaaactaaatggcctggatgtagaggtttatcactggaatctgcaaaacttcgccccggaagatctactttatgctcgtttcgatagtccggcactcaagaccttcaccgaacagctgcaacaggccgatgggctgattgtcgccacgcctgtgtataaagccgcctattccggtgcgttgaaaaccctgctcgacctgctgccagaacgcgctttgcaaggcaaagtggtgctaccgctggcgacgggcggtaccgtggcccatctgctggcggtcgattatgcccttaaaccagttttaagcgcactgaaagctcaggagatcctgcacggcgtgtttgccgatgactcacaagtaattgattaccatcacagaccccagttcacgccaaatctgcaaacccgtcttgataccgcgctagaaactttctggcaggcattgcaccgccgcgatgttcaggttcctgaccttctgtctctgcgaggtaatgcccatgcgtaa

SEQ ID NO: 75 - protein, 191 aa, Escherichia coli
MRVITLAGSPRFPSRSSSLLEYAREKLNGLDVEVYHWNLQNFAPEDLLYARFDSPALKTFTEQLQQADGLIVATPVYKAAYSGALKTLLDLLPERALQGKVVLPLATGGTVAHLLAVDYALKPVLSALKAQEILHGVFADDSQVIDYHHRPQFTPNLQTRLDTALETFWQALHRRDVQVPDLLSLRGNAHA

SEQ ID NO: 76 - DNA, 924 bp, Corynebacterium glutamicum
  source 1..924: mol_type = genomic DNA
atgacgtccccgcataattttgtcagtggtgctattgatctgggtgaggtgaaagcgcgtgcggatgcgcgccagaaggcccatgagcaggggccggtaactcagggcattgctagttcccttgatgtgaccatggagaacctggagaatgaggtgctgcgtcgttccacgcaggttccggtgattgttctcgtgggtaccccgcgcagccctgattcggagcagttgaagtcggatctgaccacgcttgctgctgaaagtggcaggaagttcattttcggttatgtcaatgctgataccgatgctgatgtggcccaggtgtttggggtgcagggcttgccgtcggtgattgctgtggcagcgggacgccctctggctgatttccagggcggacagccagcggatgcactaaagcagtggactgatcaggtggttcaggctgtgggtggacagctggaaggactgccagaggaggccacagacggcgaacaagaagacgctcctgtggaagacccccgcttcgatgctgccactgatgctctaaaccgtggcgctttcgatgaggcgattgcggtttatgagtccattttggcgcaggagccaaacaacgctgatgcgaagcaggcacgcgataccgcaaagctgttgggccggcttgccacggtggatccttcggtggatgttgtcgctgctgcagatgctgatccaacaaacgttgatctggcctacacagcagctgacgcggctgttgttgcgggtgatcctgaggctgcctttgatcgtttaattgctctgctgaccatcagcgctggcgatcagaagaatcaggtgaaggaacgtttgctggagctgtttggcatgtttgagaccgccgatccccgtgtgctgcaggcgcgaggaaagatggccagcgcgctgttctaa

SEQ ID NO: 77 - protein, 307 aa, Corynebacterium glutamicum
MTSPHNFVSGAIDLGEVKARADARQKAHEQGPVTQGIASSLDVTMENLENEVLRRSTQVPVIVLVGTPRSPDSEQLKSDLTTLAAESGRKFIFGYVNADTDADVAQVFGVQGLPSVIAVAAGRPLADFQGGQPADALKQWTDQVVQAVGGQLEGLPEEATDGEQEDAPVEDPRFDAATDALNRGAFDEAIAVYESILAQEPNNADAKQARDTAKLLGRLATVDPSVDVVAAADADPTNVDLAYTAADAAVVAGDPEAAFDRLIALLTISAGDQKNQVKERLLELFGMFETADPRVLQARGKMASALF

SEQ ID NO: 78 - DNA, 2727 bp, unidentified
  source 1..2727: mol_type = other DNA
atgcggacatacacccgatcttccgagggtccccgcccattccacgagagcgcgccgtcgccagtgcgcgcctccccagtacgccgcgccgttcacatcgccgacgcgagcgccggtgctgtcacttccgatcactaccggcgcgctcgctcgaacggcgccgcgcgcgccgtgcgcggcgccgacgagacgtcgtcttcagggtttgattcaacagaggcgtcgctcgagtccgtcgacgacgccctcgagagcgatctccagcgacgccgacgacctggaggagctcctgaacttagattacccggtggacatgccggagttcgagaacgcgctgccgacgataagcactggcggtcgacgcacgcgcgaatcgcaaacggagcggtggtgccacagcagctcatcggtgggacgccgatgatcgatcttagtgagtttagtgcgaacccaaaggtgaagatctatgggaagtgcgagtacatgaatccgagtgggagcattaaagatcggattgcgcaggagattttgactcgggcgctggagacgggcgagttgaaacccgggatgacggtcgtggcggcaacgagtgggaacaccggggcggcgatcgcgatggcgtgcgcgattcgtgggtttgattacatcgtgatcacgaataagaagacgagtaaggagaagattgacgccatgaaggcgtacggggggcaagtcatcgtcgcggagagtggggtcccggcagatcatccggatcattatcagaacatcgagacgacgatgtgcgcgcagaacccgaactattacggcgtaaatcagtacgataatccgtacaacgcggatgcgtacgagaagactcttggtcccgagatttggtcgcaaaccaagggggcagtgacgcacttcatcgcgggcggttccacaggaggcaccatcactggtaccggtcgctacttgaagagcgtagatccaacgatcaaaatcatgttagctgatcccaagggtagcgttttgtgggactatttcgtcaatgacgtgcccgaagaggatcttgtggcgaagagttgggaagtcgagggcgtcggcaaggactccattccgggtgttttgcagacagaatacatcgacggtgccgtgaaaggttgcgacgcgagctcattccgaatttgccgaatggtggccgaatcttcgggcatcttgctcggcggtagctccggtctgaacctgcacgccgctcgagtgctctcgagccagatcaaggagggtgttatagtcacggttttgtgcgacagcggtgtcaagtatttgtcaaagatcttcaacgacgaatggctcgaatcgaagaatttgaatcagccattgtcggatgtcaagaacttccaagtcgcttggaaaaaggaccagtctgaggcgagtgacgacgaagacgcagaccacggtctgtggagtcgcgacgatgaggagaaggagcttcgctttctagacgaaatcgcgacgcacatggttgagtactaccgcaactccgcgcgcgccgccgacccggtcagtacgtacaactctccgctcgcccttcacgaaaagttcaaggaggtgggtattccgttggccatcggtacgggtgaggagccggtctcgatgagcttactcaccaccgcgatgaacacggtgatccagaacagcgctcgcacctcgcacccaatgttaggaggaacgcgcggaggaagagacggaggaaggatgaggcggatgggtgagttgtcgtgggagctcggggcggcgggtgaggaggaggaggaggaagagcaggggcaggagggcgaggaaatctctcgagaggaggtgctggcgctctcggtggagctcatcgaatccatcgcttccgggaaggaaccgctcgatgcggcgcgtcttgggagcttgctttggactttgagcaacgcgctgttggaggatttgacggacagggacgatttacgcgttccggggaactcgctcgagacgtttccggtggagctggtgcgaaacgtcatgggcgccgtcgaaaccctggtcgttgcgctgacgttcgagccggaagatgcggtgacgcaacgaagtgggcacgcaattccggccatcggcacacatcgcgtcgctgcggcggagatcatcgccgtgctcttgcaaatcgggtgccaagacattgacgagcgcatcgcaaagctcaagctgccgaacgacggtcagttcgtcctcgtgtcgctcgtacgcatgtttttcaagtattcttggagttcggcgctgcacgcgaccgtggtgagactgatcttggccgcgctggtgagcccacacgagccgctctgggcacccatgtttgagggcggggacgagagtcttcagggctctctggcagcgtccatgaagacggcgctcgcgacgaagcccatctccacgagagacggtaacgtcgggagcgtgattattctcgccaacgctctgcacgagctcgaaacttgcgatgacgtcgagcgccagagcgtgcggacgacgttgcaggaggatgcaacgtggcgagccgccattgacggtgaggatagcccgttggcgaacctcaacaacgaacaggctggcggactttgtggaccaaagccgcagaagtcgcctgttttcatggactccggcatgggcgccaacgtcatcagcagccaagagctgctcaggatgttgcagcacatctcgcttggtcaatga

SEQ ID NO: 79 - protein, 908 aa, unidentified
MRTYTRSSEGPRPFHESAPSPVRASPVRRAVHIADASAGAVTSDHYRRARSNGAARAVRGADETSSSGFDSTEASLESVDDALESDLQRRRRPGGAPELRLPGGHAGVRERAADDKHWRSTHARIANGAVVPQQLIGGTPMIDLSEFSANPKVKIYGKCEYMNPSGSIKDRIAQEILTRALETGELKPGMTVVAATSGNTGAAIAMACAIRGFDYIVITNKKTSKEKIDAMKAYGGQVIVAESGVPADHPDHYQNIETTMCAQNPNYYGVNQYDNPYNADAYEKTLGPEIWSQTKGAVTHFIAGGSTGGTITGTGRYLKSVDPTIKIMLADPKGSVLWDYFVNDVPEEDLVAKSWEVEGVGKDSIPGVLQTEYIDGAVKGCDASSFRICRMVAESSGILLGGSSGLNLHAARVLSSQIKEGVIVTVLCDSGVKYLSKIFNDEWLESKNLNQPLSDVKNFQVAWKKDQSEASDDEDADHGLWSRDDEEKELRFLDEIATHMVEYYRNSARAADPVSTYNSPLALHEKFKEVGIPLAIGTGEEPVSMSLLTTAMNTVIQNSARTSHPMLGGTRGGRDGGRMRRMGELSWELGAAGEEEEEEEQGQEGEEISREEVLALSVELIESIASGKEPLDAARLGSLLWTLSNALLEDLTDRDDLRVPGNSLETFPVELVRNVMGAVETLVVALTFEPEDAVTQRSGHAIPAIGTHRVAAAEIIAVLLQIGCQDIDERIAKLKLPNDGQFVLVSLVRMFFKYSWSSALHATVVRLILAALVSPHEPLWAPMFEGGDESLQGSLAASMKTALATKPISTRDGNVGSVIILANALHELETCDDVERQSVRTTLQEDATWRAAIDGEDSPLANLNNEQAGGLCGPKPQKSPVFMDSGMGANVISSQELLRMLQHISLGQ

SEQ ID NO: 80 - DNA, 387 bp, Escherichia coli
  source 1..387: mol_type = genomic DNA
atgagcaaaactatcgcgacggaaaatgcaccggcagctatcggtccttacgtacagggcgttgatctgggcaatatgatcatcacctccggtcagatcccggtaaatccgaaaacgggcgaagtaccggcagacgtcgctgcacaggcacgtcagtcgctggataacgtaaaagcgatcgtcgaagccgctggcctgaaagtgggcgacatcgttaaaactaccgtgtttgtaaaagatctgaacgacttcgcaaccgtaaacgccacttacgaagccttcttcaccgaacacaacgccaccttcccggcacgttcttgcgttgaagttgcccgtctgccgaaagacgtgaagattgagatcgaagcgatcgctgttcgtcgctaa

SEQ ID NO: 81 - protein, 128 aa, Escherichia coli
MSKTIATENAPAAIGPYVQGVDLGNMIITSGQIPVNPKTGEVPADVAAQARQSLDNVKAIVEAAGLKVGDIVKTTVFVKDLNDFATVNATYEAFFTEHNATFPARSCVEVARLPKDVKIEIEAIAVRR

SEQ ID NO: 82 - DNA, 390 bp, Escherichia coli
  source 1..390: mol_type = genomic DNA
atgaaaaagattatcgaaacgcaacgtgccccaggcgcaatcggcccttatgttcagggcgttgatttaggcagcatggtcttcacctccgggcaaattccggtttgcccacagaccggtgagatcccggctgatgtgcaagatcaggcgcgtttaagcctcgaaaacgtcaaagcgatcgtggttgctgccgggctgagtgtgggcgatatcatcaagatgaccgtgttcatcaccgatctgaatgattttgccaccatcaatgaagtctataagcagttcttcgatgaacatcaggcgacctacccgacccggagctgtgtgcaggtcgcgcgtttgccgaaagatgtgaagctggaaattgaagccgtcgcagtacgtagtgcgtaa

SEQ ID NO: 83 - protein, 129 aa, Escherichia coli
MKKIIETQRAPGAIGPYVQGVDLGSMVFTSGQIPVCPQTGEIPADVQDQARLSLENVKAIVVAAGLSVGDIIKMTVFITDLNDFATINEVYKQFFDEHQATYPTRSCVQVARLPKDVKLEIEAVAVRSA

SEQ ID NO: 84 - DNA, 387 bp, Escherichia coli
  source 1..387: mol_type = genomic DNA
atgccaaaatccgtaattattcccgctggcagcagcgcaccgctggcccccttcgttcccggcacgctggctgatggcgtggtgtatgtctccggtacgctggcttttgatcaacataataacgtgctgtttgccgatgacccaaaggcgcaaacccgccacgttctggaaactatccgcaaggtgatcgagacggcgggtggcacgatggcggatgtgaccttcaacagcatctttattaccgactggaaaaattacgccgcgattaacgaaatctacgccgagttttttccgggtgataaaccggcgcgattctgcattcagtgcggactggtaaaacctgacgcgctggtggaaatcgccacaattgcgcatatcgccaagtga

SEQ ID NO: 85 - protein, 128 aa, Escherichia coli
MPKSVIIPAGSSAPLAPFVPGTLADGVVYVSGTLAFDQHNNVLFADDPKAQTRHVLETIRKVIETAGGTMADVTFNSIFITDWKNYAAINEIYAEFFPGDKPARFCIQCGLVKPDALVEIATIAHIAK

SEQ ID NO: 86 - DNA, 1017 bp, unidentified
  source 1..1017: mol_type = genomic DNA
atgcatcttgcccgctatccccgtcgttttctcgcccatctgccgactccgctggaacggctggaccgtttgaccaaggaattgaacggtcctgaaatctggatcaagcgggatgattgcaccggccttagcaccggcggcaacaagacccgcaagctggaattcctgatggcagaggccgaattgcagggcgcggatatggtgatgacccaaggggccacgcaatccaaccatgcgcgtcaaacggcggcctttgccgccaaactgggaatggattgccacctgctgcttgaggatcgcacgggttcgaacaacgccaattacaacaatggcggcaacgttctgttggatcacatgcacggggcgaccacggaaaagcgccccggtggcggcgacatgaatgccgagatggaaaaggtcgcggacaagttccgtgccgatgggcgcaatgtctataccattcccggtggtggctcgaacccgacaggcgcgcttggctatgttaactgcgccttcgagatgctggcgcagttcaatgatcgggcgctcaaggtggatcacattgttcatgctaccggcagtgctggcacgcaggcggggctgatcaccggtctcaaggcgatgaacgcgcaaattccgctcttgggcattggcgtccgcgcgcccaaggccaagcaagaagagaacgtctataacctcgcctgcgcgaccgctgaaaagctgggctgcgccggagtggtcaagcgtgaggatgtggttgccaataccgattatgttggcgaaggctatggcattcccacggaatccggcatcgaagccattcacatgtttgccgaattggaggcaatcctgctcgatccggtgtattcggccaaaggggctgcgggctttatcgacctgatccgcaagggtcatttcaagaaaggtgagcgcgtcgtctttctgcataccggggggtctgtcgcgctgtttggctatgacagcgcctttgatttctcgggccgctggaaaaagtaa

SEQ ID NO: 87 - protein, 338 aa, unidentified
MHLARYPRRFLAHLPTPLERLDRLTKELNGPEIWIKRDDCTGLSTGGNKTRKLEFLMAEAELQGADMVMTQGATQSNHARQTAAFAAKLGMDCHLLLEDRTGSNNANYNNGGNVLLDHMHGATTEKRPGGGDMNAEMEKVADKFRADGRNVYTIPGGGSNPTGALGYVNCAFEMLAQFNDRALKVDHIVHATGSAGTQAGLITGLKAMNAQIPLLGIGVRAPKAKQEENVYNLACATAEKLGCAGVVKREDVVANTDYVGEGYGIPTESGIEAIHMFAELEAILLDPVYSAKGAAGFIDLIRKGHFKKGERVVFLHTGGSVALFGYDSAFDFSGRWKK

SEQ ID NO: 88 - DNA, 951 bp, Escherichia coli
  source 1..951: mol_type = genomic DNA
atgaatttccaacaactaaagataatccgcgaggctgcacgtcaggattacaacctgacagaggttgcgaatatgctttttacctcacagtcaggcgtcagccgtcatattcgggaactggaggatgaacttggcatcgaaatatttgttcgacgaggtaagcgactgctgggcatgactgaaccgggcaaagcattactggtcattgcagaacgtattctgaatgaagccagtaatgttcgtcggcttgcagacctgtttaccaacgatacgtctggcgttctcactattgcaacgacgcatactcaggcacgttatagcttgccagaggtcattaaagcttttcgcgaacttttcccggaggttcggctcgagctaatccaggggacgccacaggaaattgcgacattgttgcaaaatggcgaagctgatattggtatcgccagcgagcgtttgagtaatgacccgcagctcgtcgccttcccgtggtttcgttggcaccatagtttgcttgttccacacgatcatcccttgacgcaaatttcaccattgacgctggaatcaatagcgaagtggccgttaatcacttaccgacaggggattacggggcgctcacgtattgatgacgcatttgcccgcaaaggtttgctggcagatattgtattaagtgcgcaggattctgatgtcattaaaacctatgttgctcttgggcttgggatcggattagttgccgagcaatccagtggcgaacaagaggaagagaatttaatccgcctggatacgcggcatctttttgatgctaatactgtctggttgggactgaagcgaggacaacttcagcgtaactatgtctggcgctttctggaactttgtaatgcaggactgtcagttgaggatatcaagcggcaggtgatggaaagcagtgaagaggaaattgattatcagatatag

SEQ ID NO: 89 - protein, 316 aa, Escherichia coli
MNFQQLKIIREAARQDYNLTEVANMLFTSQSGVSRHIRELEDELGIEIFVRRGKRLLGMTEPGKALLVIAERILNEASNVRRLADLFTNDTSGVLTIATTHTQARYSLPEVIKAFRELFPEVRLELIQGTPQEIATLLQNGEADIGIASERLSNDPQLVAFPWFRWHHSLLVPHDHPLTQISPLTLESIAKWPLITYRQGITGRSRIDDAFARKGLLADIVLSAQDSDVIKTYVALGLGIGLVAEQSSGEQEEENLIRLDTRHLFDANTVWLGLKRGQLQRNYVWRFLELCNAGLSVEDIKRQVMESSEEEIDYQI

SEQ ID NO: 90 - DNA, 927 bp, Corynebacterium glutamicum
  source 1..927: mol_type = genomic DNA
atggacaacgacggcggagacatgcgaatcgacgacctacgcagcttcatttcagtcgcccaatcaggccacctcaccgaaaccgccgaaagattaggcatcccgcagcccacactttccagacgaatcagccgagtggaaaaacacgcaggcaccccacttttcgaccgcgccggccgcaaactcgtcctcaaccaacgaggccacgccttcctcaaccacgccagcgccatcgtcgcagaattcaactccgccgcaactgaaatcaaacgcctcatggacccagaaaaaggcacaatccgactggacttcatgcattccttgggcacttggatggtccccgaacttatccgaacattccgcgccgaacaccccaatgtagaattccaactccaccaagcggcagcaatgctcctggtagatcgtgttttggctgatgaaactgacctcgcattagttggccccaaacctgccgaggttggtacctctttagggtgggcgccactgcttcgtcaacgacttgccctagctgttcccgcagatcaccggcttgcctctttttctggccaaggagaattgccgttgattagtgcgacggaagaacctttcgtggcgatgcgagcaggtttcggcacccgactcctcatggatgcattagccgaagaagccggttttgttcccaatgtggttttcgaatccatggagctcaccaccgtcgcagggcttgtcagcgcaggtctcggcgttggtgtggttccgatggatgatccgtaccttcccacagtgggaatcgtgcaacgcccacttagtccacccgcatatagggaactcggtctggtatggaggcttaacgcgggacctgcaccggccgtggataacttccggaagttcgtggcgggatcgagatatgcattagaagagggctga

SEQ ID NO: 91 - protein, 308 aa, Corynebacterium glutamicum
MDNDGGDMRIDDLRSFISVAQSGHLTETAERLGIPQPTLSRRISRVEKHAGTPLFDRAGRKLVLNQRGHAFLNHASAIVAEFNSAATEIKRLMDPEKGTIRLDFMHSLGTWMVPELIRTFRAEHPNVEFQLHQAAAMLLVDRVLADETDLALVGPKPAEVGTSLGWAPLLRQRLALAVPADHRLASFSGQGELPLISATEEPFVAMRAGFGTRLLMDALAEEAGFVPNVVFESMELTTVAGLVSAGLGVGVVPMDDPYLPTVGIVQRPLSPPAYRELGLVWRLNAGPAPAVDNFRKFVAGSRYALEEG

SEQ ID NO: 92 - DNA, 1362 bp, Corynebacterium glutamicum
  source 1..1362: mol_type = genomic DNA
atgcttgccgaccttcccatcgccttaaacccacacgaaccaacatccatccccacgcagctcacagaacagatccgtcgtctcgtggcgaggggaattctcaccccaggagacccgcttcccagcagtcgctcactatccacccaattgggggtatcccgcggcagtgtggtgaccgcttatgaccaattggccggtgaaggctacctcagcaccgcccgcggttccggtacaacgatcaacccagatctgcatttgttgaagcctgtggaaattgagaagaaggagacgtcgagaagcgtcccgcccccgctgctcaacctgagccccggcgtgcccgataccgcgacgctcgccgattccgcatggcgcgctgcgtggcgcgaagcctgcgccaagccacccacgcactcccctgagcagggacttttgaggctgcggatcgagatcgccgaccacctgcgccagatgcgtggcctcatggtcgagccggagcagatcatcgtcaccgccggcgcgcgcgaggggctgagtctgctgctgcgcaccatggatgcgcctgcccgcatcggcgtcgaatcgcccggctaccccagcctgcgccgcatcccgcaggtgcttggccatgagacgatcgatgtgccgaccgacgaatccggcctcgtaccccgcgcgctgccccacgacctcaacgcgctactggtaacccctagccatcaatatccctacggcggctcgctgcccgccgatcgccgcaccgcgctagtcgcgtgggctgaggcaaacgatgcgttgcttattgaagacgacttcgattctgagctgcgctacgtcggtatgccgcttccgccgctgcgtgcgctggcgcccgatcgcacgattctgctcggcacgttttcctccgtgatcacaccacaagtcgcctgcggatacctcatcgcgccgacgccccaggcgcgcgtgctcgccacgcttcgcgggattctcggccagccagtcggcgccatcacccaacacgcgctcgcgtcctacctcgcctcaggcgctttacgacgccgcacccaacgtttgcggcgcctttaccgacaccgccgctccatcgtccaagacaccctcggtgacctcccgaatacgcagcttcgccccatcaacggtggcctccacgcagttctcctttgcgacaaaccccaagacctcgtcgtcaccacactcgcctcccgaggccttaacgtcaccgcgctttcccactactggggcggcaccggcgcagacaacggcatcgtcttcggcttcggctcccacgacgaagacaccctcagatgggtgcttgctgagatcagcgatgcggtgtctctaggctaa

SEQ ID NO: 93 - protein, 453 aa, Corynebacterium glutamicum
MLADLPIALNPHEPTSIPTQLTEQIRRLVARGILTPGDPLPSSRSLSTQLGVSRGSVVTAYDQLAGEGYLSTARGSGTTINPDLHLLKPVEIEKKETSRSVPPPLLNLSPGVPDTATLADSAWRAAWREACAKPPTHSPEQGLLRLRIEIADHLRQMRGLMVEPEQIIVTAGAREGLSLLLRTMDAPARIGVESPGYPSLRRIPQVLGHETIDVPTDESGLVPRALPHDLNALLVTPSHQYPYGGSLPADRRTALVAWAEANDALLIEDDFDSELRYVGMPLPPLRALAPDRTILLGTFSSVITPQVACGYLIAPTPQARVLATLRGILGQPVGAITQHALASYLASGALRRRTQRLRRLYRHRRSIVQDTLGDLPNTQLRPINGGLHAVLLCDKPQDLVVTTLASRGLNVTALSHYWGGTGADNGIVFGFGSHDEDTLRWVLAEISDAVSLG

SEQ ID NO: 94 - DNA, 1464 bp, unidentified
  source 1..1464: mol_type = genomic DNA
atggcgatttcccccgaaaccttctttctcgcctccgaagccgagggcacgctccagacccggatccgccagatggtggccgaggggatcctgaccggccgcttccgcccgggcgagaaactgccctcctcgcgcaagctcgccgcgcatctgggcgtcagccggatcaccgtgacactcgcctataccgaacttcaggccgacgattacatcacctcgcgcggccggtcgggctattacgtgtccgacaacgcgcccgaaccgccgtcctttcccgcgcgcgcacccgggcagtcgtcggtcgactggtcgcgcgccatcggccagcggtttcgcggcaccgaaccgcattcgaaacccggcaactgggccgatttccgctatccgttcatctatggccaggccgatccgaccctgttcgacgcggccaattggcggctctgtgccctccaggccctggggcgcaaggatttcgccgcgctgaccaccgattacaacgacagcgacgatcccgaattgctggatttcatcgcccgccagatcctgccccgccgcggcatcctggccgggccggacgaaatcctgctgacgctgggcgcccagaacgcgctgtggctgaccgcccaggtgctgctgacccagcgccggaccgcggcgatcgaggatccctgctacccggccctgcgcggcatcctgacccaggcgcgctgccacctgcacgcggtcccggtggatcgcgacgggttgccgcccgaggcgatccccgacggcaccaacgtggtgttctgcacccccagccaccaatgcccgaccaccgcgaccatgccgatgtcgcgccgccatgccctgctggaacgcgccgaggccgaggatttcctgatcgtcgaggatgattacgaattcgagatgtcgttcctcaaatccccctcgccggcgctgaaatcgctcgaccggcacgggcgggtgatctacgtcggctctttctccaaatcgctgtttccgggcctgcgcctgggctatctggtcggccccgagccgttcatccgggaggcccgcgccctgcgtgccagcgtgctgcgccacccgccgggccatatccagcgcaccgtcacctatttcctgtctcttggccattacgacgccctgatccgccgcatgggccgggcctatcacgaccggcgccggatcatggaccgcgccctgcacgatcacgggctgaccgtcgccggatcgggctccttcggcggctcgtccttctggatgcgcgcgcccgcgggcgtcgatacggccgagctggcccgccgcctgtccgccgacagcgtgctgatcgaaccgggccagccgttctttgccggcaccgcgccgcccgggcggttctaccgcctggcctatagttcgatctctggcccgcgcatccccgacgggatcgcgcggatcgccgccgcgctggagaactggtcctag

SEQ ID NO: 95 - protein, 487 aa, unidentified
MAISPETFFLASEAEGTLQTRIRQMVAEGILTGRFRPGEKLPSSRKLAAHLGVSRITVTLAYTELQADDYITSRGRSGYYVSDNAPEPPSFPARAPGQSSVDWSRAIGQRFRGTEPHSKPGNWADFRYPFIYGQADPTLFDAANWRLCALQALGRKDFAALTTDYNDSDDPELLDFIARQILPRRGILAGPDEILLTLGAQNALWLTAQVLLTQRRTAAIEDPCYPALRGILTQARCHLHAVPVDRDGLPPEAIPDGTNVVFCTPSHQCPTTATMPMSRRHALLERAEAEDFLIVEDDYEFEMSFLKSPSPALKSLDRHGRVIYVGSFSKSLFPGLRLGYLVGPEPFIREARALRASVLRHPPGHIQRTVTYFLSLGHYDALIRRMGRAYHDRRRIMDRALHDHGLTVAGSGSFGGSSFWMRAPAGVDTAELARRLSADSVLIEPGQPFFAGTAPPGRFYRLAYSSISGPRIPDGIARIAAALENWS

SEQ ID NO: 96 - DNA, 642 bp, Corynebacterium glutamicum
  source 1..642: mol_type = genomic DNA
atggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttag

SEQ ID NO: 97 - protein, 213 aa, Corynebacterium glutamicum
MAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 98 - DNA, 912 bp, Escherichia coli
  source 1..912: mol_type = genomic DNA
atgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcggggaaattaaaccgggtgatgtcttaatcgaagccaccagtggtaacaccggcattgcgctggcaatgattgccgcgctgaaaggctatcgcatgaaattgctgatgcccgacaacatgagccaggaacgccgtgcggcgatgcgtgcttatggtgcggaactgattcttgtcaccaaagagcagggcatggaaggtgcgcgcgatctggcgctggagatggcgaatcgtggcgaaggaaagctgctcgatcagttcaataatcccgataacccttatgcgcattacaccaccactgggccggaaatctggcagcaaaccggcgggcgcatcactcattttgtctccagcatggggacgaccggcactatcaccggcgtctcacgctttatgcgcgaacaatccaaaccggtgaccattgtcggcctgcaaccggaagagggcagcagcattcccggcattcgccgctggcctacggaatatctgccggggattttcaacgcttctctggtggatgaggtgctggatattcatcagcgcgatgcggaaaacaccatgcgcgaactggcggtgcgggaaggaatattctgtggcgtcagctccggcggcgcggttgccggagcactgcgggtggcaaaagctaaccctgacgcggtggtggtggcgatcatctgcgatcgtggcgatcgctacctttctaccggggtgtttggggaagagcattttagccagggggcggggatttaa

SEQ ID NO: 99 - protein, 303 aa, Escherichia coli
MSTLEQTIGNTPLVKLQRMGPDNGSEVWLKLEGNNPAGSVKDRAALSMIVEAEKRGEIKPGDVLIEATSGNTGIALAMIAALKGYRMKLLMPDNMSQERRAAMRAYGAELILVTKEQGMEGARDLALEMANRGEGKLLDQFNNPDNPYAHYTTTGPEIWQQTGGRITHFVSSMGTTGTITGVSRFMREQSKPVTIVGLQPEEGSSIPGIRRWPTEYLPGIFNASLVDEVLDIHQRDAENTMRELAVREGIFCGVSSGGAVAGALRVAKANPDAVVVAIICDRGDRYLSTGVFGEEHFSQGAGI

SEQ ID NO: 100 - DNA, 1365 bp, Escherichia coli
  source 1..1365: mol_type = genomic DNA
atgattagtctattcgacatgtttaaggtggggattggtccctcatcttcccataccgtagggcctatgaaggcaggtaaacagttcgtcgatgatctggtcgaaaaaggcttactggatagcgttactcgcgttgccgtggacgtttatggttcactgtcgctgacgggtaaaggccaccacaccgatatcgccattattatgggtcttgcaggtaacgaacctgccaccgtggatatcgacagtattcccggttttattcgcgacgtagaagagcgcgaacgtctgctgctggcacagggacggcatgaagtggatttcccgcgcgacaacgggatgcgttttcataacggcaacctgccgctgcatgaaaacggtatgcaaatccacgcctataacggcgatgaagtcgtctacagcaaaacttattattccatcggcggcggttttatcgtcgatgaagaacactttggtcaggatgctgccaacgaagtaagcgtgccgtatccgttcaaatctgccaccgaactgctcgcgtactgtaatgaaaccggctattcgctgtctggtctcgctatgcagaacgaactggcgctgcacagcaagaaagagatcgacgagtatttcgcgcatgtctggcaaaccatgcaggcatgtatcgatcgcgggatgaacaccgaaggtgtactgccaggcccgctgcgcgtgccacgtcgtgcgtctgccctgcgccggatgctggtttccagcgataaactgtctaacgatccgatgaatgtcattgactgggtaaacatgtttgcgctggcagttaacgaagaaaacgccgccggtggtcgtgtggtaactgcgccaaccaacggtgcctgcggtatcgttccggcagtgctggcttactatgaccactttattgaatcggtcagcccggacatctatacccgttactttatggcagcgggcgcgattggtgcattgtataaaatgaacgcctctatttccggtgcggaagttggttgccagggcgaagtgggtgttgcctgttcaatggctgctgcgggtcttgcagaactgctgggcggtagcccggaacaggtttgcgtggcggcggaaattggcatggaacacaaccttggtttaacctgcgacccggttgcagggcaggttcaggtgccgtgcattgagcgtaatgccattgcctctgtgaaggcgattaacgccgcgcggatggctctgcgccgcaccagtgcaccgcgcgtctcgctggataaggtcatcgaaacgatgtacgaaaccggtaaggacatgaacgccaaataccgcgaaacctcacgcggtggtctggcaatcaaagtccagtgtgactaa

SEQ ID NO: 101 - protein, 454 aa, Escherichia coli
MISLFDMFKVGIGPSSSHTVGPMKAGKQFVDDLVEKGLLDSVTRVAVDVYGSLSLTGKGHHTDIAIIMGLAGNEPATVDIDSIPGFIRDVEERERLLLAQGRHEVDFPRDNGMRFHNGNLPLHENGMQIHAYNGDEVVYSKTYYSIGGGFIVDEEHFGQDAANEVSVPYPFKSATELLAYCNETGYSLSGLAMQNELALHSKKEIDEYFAHVWQTMQACIDRGMNTEGVLPGPLRVPRRASALRRMLVSSDKLSNDPMNVIDWVNMFALAVNEENAAGGRVVTAPTNGACGIVPAVLAYYDHFIESVSPDIYTRYFMAAGAIGALYKMNASISGAEVGCQGEVGVACSMAAAGLAELLGGSPEQVCVAAEIGMEHNLGLTCDPVAGQVQVPCIERNAIASVKAINAARMALRRTSAPRVSLDKVIETMYETGKDMNAKYRETSRGGLAIKVQCD

SEQ ID NO: 102 - DNA, 1350 bp, Corynebacterium glutamicum
  source 1..1350: mol_type = genomic DNA
atggctatcagtgttgttgatctatttagcatcggtatcggaccatcatcctcacataccgtcggccccatgagagccgccctcacgtatatctctgaatttcccagctcacatgtcgatatcacgttgcacggatcccttgccgccaccggtaaaggccactgcactgaccgggcggtattactgggtctggtgggatgggaaccaacgatagttcccattgatgctgcaccctcacccggcgcgccgattcctgcgaaaggttctgtgaacgggccaaagggaacggtgtcgtattccctgacgtttgatcctcatcctcttccagaacaccccaatgccgttacctttaaaggatcaaccacaaggacttatttgtcggtgggtggtgggttcattatgacgttggaggatttccggaagctggacgatatcggatcaggtgtgtcaaccattcatccagaggcagaggtgccttgtccttttcagaagagttcccaattactcgcatatgggcgcgattttgcggaggtcatgaaggataatgagcgcttaatccacggggatcttggcacagtggatgcccatttggatcgagtgtggcagattatgcaggagtgcgtggcacaaggcatcgcaacgcctgggattttaccgggtgggttgaatgtgcaacgtcgggcgccgcaggtacacgcgctgattagcaacggggatacgtgtgagctgggtgctgatcttgatgctgtggagtgggtgaatctgtacgccttggcggtgaatgaagaaaacgccgctggtggtcgtgtggttactgctccgactaatggtgctgcggggattattccggcggtgatgcactatgcgcgggattttttgacaggttttggggcggagcaggcgcggacgtttttgtataccgcgggtgcggtgggcatcatcattaaggaaaatgcctcgatctctggcgcggaggtggggtgtcagggtgaggttggttcagcgtccgcgatggcggctgccgggttgtgtgcagtcttaggtggttctccgcaacaggtggaaaacgccgcggagattgcgttggagcacaatttgggattgacgtgcgatccggtgggcgggttagtgcagattccgtgtattgaacgcaacgctattgctgccatgaagtccatcaatgcggcaaggcttgcccggattggtgatggcaacaatcgcgtgagtttggatgatgtggtggtcacgatggctgccaccggtcgggacatgttgaccaaatacaaggaaacgtcccttggtggtttggcaaccaccttgggcttcccggtgtcgatgacggagtgttag

SEQ ID NO: 103 - protein, 449 aa, Corynebacterium glutamicum
MAISVVDLFSIGIGPSSSHTVGPMRAALTYISEFPSSHVDITLHGSLAATGKGHCTDRAVLLGLVGWEPTIVPIDAAPSPGAPIPAKGSVNGPKGTVSYSLTFDPHPLPEHPNAVTFKGSTTRTYLSVGGGFIMTLEDFRKLDDIGSGVSTIHPEAEVPCPFQKSSQLLAYGRDFAEVMKDNERLIHGDLGTVDAHLDRVWQIMQECVAQGIATPGILPGGLNVQRRAPQVHALISNGDTCELGADLDAVEWVNLYALAVNEENAAGGRVVTAPTNGAAGIIPAVMHYARDFLTGFGAEQARTFLYTAGAVGIIIKENASISGAEVGCQGEVGSASAMAAAGLCAVLGGSPQQVENAAEIALEHNLGLTCDPVGGLVQIPCIERNAIAAMKSINAARLARIGDGNNRVSLDDVVVTMAATGRDMLTKYKETSLGGLATTLGFPVSMTEC

SEQ ID NO: 104 - DNA, 1254 bp, Escherichia coli
  source 1..1254: mol_type = genomic DNA
atgttaaagcgtgaaatgaacattgccgattatgatgccgaactgtggcaggctatggagcaggaaaaagtacgtcaggaagagcacatcgaactgatcgcctccgaaaactacaccagcccgcgcgtaatgcaggcgcagggttctcagctgaccaacaaatatgctgaaggttatccgggcaaacgctactacggcggttgcgagtatgttgatatcgttgaacaactggcgatcgatcgtgcgaaagaactgttcggcgctgactacgctaacgtccagccgcactccggctcccaggctaactttgcggtctacaccgcgctgctggaaccaggtgataccgttctgggtatgaacctggcgcatggcggtcacctgactcacggttctccggttaacttctccggtaaactgtacaacatcgttccttacggtatcgatgctaccggtcatatcgactacgccgatctggaaaaacaagccaaagaacacaagccgaaaatgattatcggtggtttctctgcatattccggcgtggtggactgggcgaaaatgcgtgaaatcgctgacagcatcggtgcttacctgttcgttgatatggcgcacgttgcgggcctggttgctgctggcgtctacccgaacccggttcctcatgctcacgttgttactaccaccactcacaaaaccctggcgggtccgcgcggcggcctgatcctggcgaaaggtggtagcgaagagctgtacaaaaaactgaactctgccgttttccctggtggtcagggcggtccgttgatgcacgtaatcgccggtaaagcggttgctctgaaagaagcgatggagcctgagttcaaaacttaccagcagcaggtcgctaaaaacgctaaagcgatggtagaagtgttcctcgagcgcggctacaaagtggtttccggcggcactgataaccacctgttcctggttgatctggttgataaaaacctgaccggtaaagaagcagacgccgctctgggccgtgctaacatcaccgtcaacaaaaacagcgtaccgaacgatccgaagagcccgtttgtgacctccggtattcgtgtaggtactccggcgattacccgtcgcggctttaaagaagccgaagcgaaagaactggctggctggatgtgtgacgtgctggacagcatcaatgatgaagccgttatcgagcgcatcaaaggtaaagttctcgacatctgcgcacgttacccggtttacgcataa

SEQ ID NO: 105 - protein, 417 aa, Escherichia coli
MLKREMNIADYDAELWQAMEQEKVRQEEHIELIASENYTSPRVMQAQGSQLTNKYAEGYPGKRYYGGCEYVDIVEQLAIDRAKELFGADYANVQPHSGSQANFAVYTALLEPGDTVLGMNLAHGGHLTHGSPVNFSGKLYNIVPYGIDATGHIDYADLEKQAKEHKPKMIIGGFSAYSGVVDWAKMREIADSIGAYLFVDMAHVAGLVAAGVYPNPVPHAHVVTTTTHKTLAGPRGGLILAKGGSEELYKKLNSAVFPGGQGGPLMHVIAGKAVALKEAMEPEFKTYQQQVAKNAKAMVEVFLERGYKVVSGGTDNHLFLVDLVDKNLTGKEADAALGRANITVNKNSVPNDPKSPFVTSGIRVGTPAITRRGFKEAEAKELAGWMCDVLDSINDEAVIERIKGKVLDICARYPVYA

SEQ ID NO: 106 - DNA, 1305 bp, Corynebacterium glutamicum
  source 1..1305: mol_type = genomic DNA
atgaccgatgcccaccaagcggacgatgtccgttaccagccactgaacgagcttgatcctgaggtggctgctgccatcgctggggaacttgcccgtcaacgcgatacattagagatgatcgcgtctgagaacttcgttccccgttctgttttgcaggcgcagggttctgttcttaccaataagtatgccgagggttaccctggccgccgttactacggtggttgcgaacaagttgacatcattgaggatcttgcacgtgatcgtgcgaaggctctcttcggtgcagagttcgccaatgttcagcctcactctggcgcacaggctaatgctgctgtgctgatgactttggctgagccaggcgacaagatcatgggtctgtctttggctcatggtggtcacttgacccacggaatgaagttgaacttctccggaaagctgtacgaggttgttgcgtacggtgttgatcctgagaccatgcgtgttgatatggatcaggttcgtgagattgctctgaaggagcagccaaaggtaattatcgctggctggtctgcataccctcgccaccttgatttcgaggctttccagtctattgctgcggaagttggcgcgaagctgtgggtcgatatggctcacttcgctggtcttgttgctgctggtttgcacccaagcccagttccttactctgatgttgtttcttccactgtccacaagactttgggtggacctcgttccggcatcattctggctaagcaggagtacgcgaagaagctgaactcttccgtattcccaggtcagcagggtggtcctttgatgcacgcagttgctgcgaaggctacttctttgaagattgctggcactgagcagttccgtgaccgtcaggctcgcacgttggagggtgctcgcattcttgctgagcgtctgactgcttctgatgcgaaggccgctggcgtggatgtcttgaccggtggcactgatgtgcacttggttttggctgatctgcgtaactcccagatggatggccagcaggcggaagatctgctgcacgaggttggtatcactgtgaaccgtaacgcggttcctttcgatcctcgtccaccaatggttacttctggtctgcgtattggtactcctgcgctggctacccgtggtttcgatattcctgcattcactgaggttgcagacatcattggtactgctttggctaatggtaagtccgcagacattgagtctctgcgtggccgtgtagcaaagcttgctgcagattacccactgtatgagggcttggaagactggaccatcgtctaa

SEQ ID NO: 107 - protein, 420 aa, Corynebacterium glutamicum
MTDAHQADDVRYQPLNELDPEVAAAIAGELARQRDTLEMIASENFVPRSVLQAQGSVLTNKYAEGYPGRRYYGGCEQVDIIEDLARDRAKALFGAEFANVQPHSGAQANAAVLMTLAEPGDKIMGLSLAHGGHLTHGMKLNFSGKLYEVVAYGVDPETMRVDMDQVREIALKEQPKVIIAGWSAYPRHLDFEAFQSIAAEVGAKLWVDMAHFAGLVAAGLHPSPVPYSDVVSSTVHKTLGGPRSGIILAKQEYAKKLNSSVFPGQQGGPLMHAVAAKATSLKIAGTEQFRDRQARTLEGARILAERLTASDAKAAGVDVLTGGTDVHLVLADLRNSQMDGQQAEDLLHEVGITVNRNAVPFDPRPPMVTSGLRIGTPALATRGFDIPAFTEVADIIGTALANGKSADIESLRGRVAKLAA

SEQ ID NO: 108 - DNA, 1416 bp, Escherichia coli
  source 1..1416: mol_type = genomic DNA
atggaaaactttaaacatctccctgaaccgttccgcattcgtgttattgagccagtaaaacgtaccactcgcgcttatcgtgaagaggcaattattaaatccggtatgaacccgttcctgctggatagcgaagatgtttttatcgatttactgaccgacagcggcaccggggcggtgacgcagagcatgcaggctgcgatgatgcgcggcgacgaagcctacagcggcagtcgtagctactatgcgttagccgagtcagtgaaaaatatctttggttatcaatacaccattccgactcaccagggccgtggcgcagagcaaatctatattccggtactgattaaaaaacgcgagcaggaaaaaggcctggatcgcagcaaaatggtggcgttctctaactatttctttgataccacgcagggccatagccagatcaacggctgtaccgtgcgtaacgtctatatcaaagaagccttcgatacgggcgtgcgttacgactttaaaggcaactttgaccttgagggattagaacgcggtattgaagaagttggtccgaataacgtgccgtatatcgttgcaaccatcaccagtaactctgcaggtggtcagccggtttcactggcaaacttaaaagcgatgtacagcatcgcgaagaaatacgatattccggtggtaatggactccgcgcgctttgctgaaaacgcctatttcatcaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtacaccgagtgcagaaccctttgcgtggtgcaggaaggcttcccgacatatggcggcctggaaggcggcgcgatggagcgtctggcggtaggtctgtatgacggcatgaatctcgactggctggcttatcgtatcgcgcaggtacagtatctggtcgatggtctggaagagattggcgttgtctgccagcaggcgggcggtcacgcggcattcgttgatgccggtaaactgttgccgcatatcccggcagaccagttcccggcacaggcgctggcctgcgagctgtataaagtcgccggtatccgtgcggtagaaattggctctttcctgttaggccgcgatccgaaaaccggtaaacaactgccatgcccggctgaactgctgcgtttaaccattccgcgcgcaacatatactcaaacacatatggacttcattattgaagcctttaaacatgtgaaagagaacgcggcgaatattaaaggattaacctttacgtacgaaccgaaagtattgcgtcacttcaccgcaaaacttaaagaagtttaa

SEQ ID NO: 109 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 110 - DNA, 3811 bp, Escherichia coli
  source 1..3811: mol_type = genomic DNA
atggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcgcggcctgggcgatgtgctgattagcttcgaatcggaagtgaacaacatccgtaaacagtatgaagcgcagggctttgaagtggtgattccgaaaaccaacattctggcggaattcccggtggcgtgggttgataaaaacgtgcaggccaacggtacggaaaaagccgccaaagcctatctgaactggctctatagcccgcaggcgcaaaccatcatcaccgactattactaccgcgtgaataacccggaggtgatggacaaactgaaagacaaattcccgcagaccgagctgttccgcgtggaagacaaatttggctcctggccggaagtgatgaaaacccacttcaccagcggcggcgagttagacaagctgttagcggcggggcgtaactgatgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacggaagtgacgtcgctgatgatttttgtgcgcttacaggagtttgattacccggcagcgagcgcgattgcttcggtgatcctcgcggcatctctgctgctgctgttctcaattaacactctgcaaagtcgctttggtcggcgtgtggtaggtcattaatggcggaagttacccaattgaagcgttatgacgcgcgcccgattaactggggcaaatggtttctgattggcatcgggatgctggtttcggcgttcatcctgctggtgccgatgatttacatcttcgtgcaggcattcagcaaggggctgatgccggttttacagaatctggccgatccggacatgctgcacgccatctggctgacggtgatgatcgcgctgattgccgtaccggtaaacctggtgttcggcattctgctggcctggctggtgacgcgctttaacttccctggacgccagttactgctgacgctactggacattccgtttgccgtatcgccggtggttgccggtctggtgtatttgctgttctacggctctaacggcccgctcggcggttggctcgacgagcataacctgcaaattatgttctcctggccgggaatggtgctggtcaccatcttcgtgacgtgtccgtttgtggtgcgcgaactggtgccggtgatgttaagccagggcagccaggaagacgaagcggcgattttgcttggcgcgtccggctggcagatgttccgtcgcgtcacattaccgaacatccgctgggcgctgctttatggcgtggtgttgaccaacgcccgcgcaattggcgagtttggcgcggtgtcggtggtttccggctcgattcgcggcgaaaccctgtcgctgccgttacagattgaattgctggagcaggactacaacaccgtcggctcctttaccgctgcggcgctgttaacgctgatggcgattatcaccctgtttttaaaaagtatgttgcagtggcgcctggagaatcaggaaaaacgcgcacagcaggaggaacatcatgagcattgagattgccaatattaagaagtcgtttggtcgcacccaggtgctgaacgatatctcactggatattccttcaggtcagatggtcgcgttgctggggccgtccggttccgggaaaaccacgctgctgcgcattatcgccgggctggagcatcaaaccagcgggcatattcgcttccacggcaccgacgtgagccgcctgcacgcacgtgatcgtaaagtcggtttcgtgttccagcattacgcgctgttccgccatatgacggtgttcgacaatatcgcttttggcctgacggtgctgccgcgtcgcgagcgcccgaatgccgcagccatcaaagcgaaagtgacaaaattgctggaaatggtccagcttgcccatctggcggatcgttatccggcgcagctttccggcggccagaaacagcgcgtggcgctggcgcgcgcgctggctgtggaaccgcaaattctgctgcttgatgaaccgtttggcgcgctggatgcgcaggtgcgtaaagagctgcgtcgctggctgcgtcaactccatgaagaactaaaattcaccagcgtttttgtgacccacgatcaggaagaagcgaccgaagtagctgatcgtgtagttgtgatgagccagggcaatattgaacaggctgacgcgccggatcaggtatggcgcgaaccggcgacccgttttgtgctcgaatttatgggcgaagtgaaccgcctgcagggaaccattcgcggcgggcagttccatgttggcgcgcatcgctggccgctgggctacacacctgcgtatcaggggccggtggatctcttcctgcgcccttgggaagtggatatcagccgccgtaccagcctcgattcgccgctgccggtacaggtactggaagccagcccgaaaggtcactacacccaattagtggtgcagccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctga

SEQ ID NO: 111 - protein, 338 aa, Escherichia coli
MAVNLLKKNSLALVASLLLAGHVQATELLNSSYDVSRELFAALNPPFEQQWAKDNGGDKLTIKQSHAGSSKQALAILQGLKADVVTYNQVTDVQILHDKGKLIPADWQSRLPNNSSPFYSTMGFLVRKGNPKNIHDWNDLVRSDVKLIFPNPKTSGNARYTYLAAWGAADKADGGDKGKTEQFMTQFLKNVEVFDTGGRGATTTFAERGLGDVLISFESEVNNIRKQYEAQGFEVVIPKTNILAEFPVAWVDKNVQANGTEKAAKAYLNWLYSPQAQTIITDYYYRVNNPEVMDKLKDKFPQTELFRVEDKFGSWPEVMKTHFTSGGELDKLLAAGRN

SEQ ID NO: 112 - protein, 277 aa, Escherichia coli
MFAVSSRRVLPGFTLSLGTSLLFVCLILLLPLSALVMQLAQMSWAQYWEVITNPQVVAAYKVTLLSAFVASIFNGVFGLLMAWILTRYRFPGRTLLDALMDLPFALPTAVAGLTLASLFSVNGFYGEWLAKFDIKVTYTWLGIAVAMAFTSIPFVVRTVQPVLEELGPEYEEAAETLGATRWQSFCKVVLPELSPALVAGVALSFTRSLGEFGAVIFIAGNIAWKTEVTSLMIFVRLQEFDYPAASAIASVILAASLLLLFSINTLQSRFGRRVVGH

SEQ ID NO: 113 - protein, 291 aa, Escherichia coli
MAEVTQLKRYDARPINWGKWFLIGIGMLVSAFILLVPMIYIFVQAFSKGLMPVLQNLADPDMLHAIWLTVMIALIAVPVNLVFGILLAWLVTRFNFPGRQLLLTLLDIPFAVSPVVAGLVYLLFYGSNGPLGGWLDEHNLQIMFSWPGMVLVTIFVTCPFVVRELVPVMLSQGSQEDEAAILLGASGWQMFRRVTLPNIRWALLYGVVLTNARAIGEFGAVSVVSGSIRGETLSLPLQIELLEQDYNTVGSFTAAALLTLMAIITLFLKSMLQWRLENQEKRAQQEEHHEH

SEQ ID NO: 114 - protein, 365 aa, Escherichia coli
MSIEIANIKKSFGRTQVLNDISLDIPSGQMVALLGPSGSGKTTLLRIIAGLEHQTSGHIRFHGTDVSRLHARDRKVGFVFQHYALFRHMTVFDNIAFGLTVLPRRERPNAAAIKAKVTKLLEMVQLAHLADRYPAQLSGGQKQRVALARALAVEPQILLLDEPFGALDAQVRKELRRWLRQLHEELKFTSVFVTHDQEEATEVADRVVVMSQGNIEQADAPDQVWREPATRFVLEFMGEVNRLQGTIRGGQFHVGAHRWPLGYTPAYQGPVDLFLRPWEVDISRRTSLDSPLPVQVLEASPKGHYTQLVVQPLGWYNEPLTVVMHGDDAPQRGERLFVGLQHARLYNGDERIETRDEELALAQSA

SEQ ID NO: 115 - DNA, 1444 bp, synthetic construct
  misc_feature 1..1444: note = Synthetic Construct
  source 1..1444: mol_type = other DNA
tttgttacggtagtatcacaatcttttaattcaattggcacatcaatttgcacgccagaaacctgaccattggtttgcaacttcgtggcactaacatctggcaaagtcaccacggaatctttcgtcgcagctaccagggtacaagctggagcaataactttaccattaaatgttactgagccatccgtgatagcattatcatcagcagccataacgctggacgttgcacataccacagttataaacgccgtcaatacactttttttcatcgttatcatcctgatctcttcgtaaatatgataatgcctttaaatgacattagacttaataataacaataattgtccgccatttaaatcaagcatactaaatccaataagtgacaaatctttaatttatagatttcatgaatatttaaatattaactgaacgagtaattcattgattcaacatctaatgactatccaataaataaatatgcgtgatatataaagccctttctttagtttattttcagaaaaagatacacaaaatttatatttggaattttcttgtctctccgacagactgaccgaattgttatcaatgttaacaaaaaaagaacaattggttataaggagagagtattccccgaatggcggcgctattgccgccattcggttatttatcaagccagtgctttagttatcttctcgtacagatcgccagagagattttccagccctttcaactgttccagcgccgcgcgcattttctcctgacgtttggcatcgtaacgtttcaggcgaatcagcggttcaatcagacgtgaagccacctgcgggttacggctgttgaggtcggtaagcatttccaccaggaacaggtaaccgctgccatcttcggcatggaacgctgccggattgctgcccgcaaacgcgccaatcaacgaacgaatacggttcgggttgctcatggtaaatgagcgatgctgcaacaggccgcgcaccgtctccagcacattcgccgccgggctggtggcttgcaggataaaccatttatccatcaccagaccgttctgatgccacttgtcgtcgtactcctgcatcagcgcgtcacggcaaggcagctgtgcggcaaccgccgcagaaagcgccgccagcgcatcagtcatattgtttgcttcgtggaactgcttgctcaccagcacatcagccagatgcgtttcaccaaaagcgaggaaacgcaggcaggcattacgcagagtgcgttttgcaatatcttcatgctcaacacggtactcgctctggtaattcgcgttgtaaatagccagtagctcatccgccagttcagtcgccagagtacgagtgagtgcttcgcgtacttcggcaatagcaatcgggtcgatgatatcgaacaattcagccatttcattgacagaaggcagcgtcaggatttctgccgcc

SEQ ID NO: 116 - DNA, 854 bp, synthetic construct
  misc_feature 1..854: note = Synthetic Construct
  source 1..854: mol_type = other DNA
aagtgtttgtatgtttggtatgtatagttattttgttttatacattgtatttagatgtgatttagtttgtcaattaattgcatttaaaaaatatgttctgtgaacaagcattgtttatatacattatgtgaatgtaatatgcgagtgattgagaatgatacagtgatatatacaatgcgaatataatagttttattatatgtattgatattgatagaaataatgaagtaataaatctcgtaatgtggttgtttatgcatcactaaaatgaagtgtagtaattttcccaattgttagaacggagtaattgcatatttaatctttccttagccgtttttttgctaagaataaaatcatctgtgcgataacgactaattcttttaatgaatgtttttattcctgaatactgctcccataacaagacaggggagcagacaatcatggcaatttcatcgcgtaacaccggtcccctcgcccccttggggagagggttagggtgagggggcgttcaccgtactttcaacaggttaactccccctttctgagaggaaacaaaattaacgcagaatcttcttctcagccaaatccagcgcaaagtagctgaaaatcagatccgcacccgcacgcttaatcgaacctaagctttcgagcacgactttctcttcatctatagcacccgccagcgcggcgaacttaatcatcgcatactcaccgctcacctgatacgcgccaatcggcaattcagtacgttcacgcagctcacgcacgatgtcgaggtacgctccagcaggtttaaccatcaggcagtctgcgccctgggcttcatccagcagtgattcacgaatcgcctcacga

SEQ ID NO: 117 - DNA, 1636 bp, synthetic construct
  misc_feature 1..1636: note = Synthetic Construct
  source 1..1636: mol_type = other DNA
ctcagcgcctaatttcccttcctcttcgccgctggtcgccacaaatcgaataccatactcggtaggcgtatttttcaggcgttctgccaattccagcatgacacctaaacctgcggcgttatcatccattccttgtaacgtcagcccgccgagattggcatcggcgtcagcatcgctcagcggggcgtaagtatccagatgcgccataatgatgatctgctgcggcgctttgccttcatgagcggcaatcaccgtacttcccgtcacgttatgccagctcttacgattatcgcgggcggtataaatataccgactattaaatgtccgaatatcactgcgataacccatttgctgaaactgttggcgaatataatcggcagataacatttctgcaggagttccggtcatgcgtcccggaaagaaagtagcaatatgtcgtgcctgagtattagcaaaatcgccaggtttaggtgacgaggcgtgtacggggagaataaagcatacgccgagcgccagggcagcggtacggtggcgcaatgcggaaaacatagtgagtccttaaataccatgcaaatttttttaccgccatagtatgaaactgccgctgcgctaaaacaatttcaaatcttcctaaacgcccgaaatccggtgccttgacaattaatcatccggctcgtataatgtgtggaattgtgagcggataacaatttcacacaggaaacagaccatggatcaaatacgacttactcacctgcggcaactggaggcggaaagcatccacattattcgcgaggtggcggcagaattctcaaatccggtgatgctctactctatcggtaaagattccagcgtcatgctgcatctggcgcgcaaggcgttttatccaggtacgctgcctttcccgttgctgcatgtcgataccggctggaaattccgcgagatgtatgagttccgcgatcgtactgctaaagcctacggctgcgaactgctggtgcataaaaacccggaaggcgtggcgatggggattaatccattcgtgcacggcagcgcgaaacataccgatattatgaaaactgaaggcctgaaacaggcgctgaacaaatacggttttgatgccgccttcggtggtgcgcgccgtgacgaagagaaatcccgcgctaaagagcgaatttactctttccgtgaccgcttccatcgctgggatccgaaaaatcagcgcccggagctgtggcacaactacaacgggcaaattaacaaaggcgaaagcatccgcgtcttcccgctctctaactggaccgagcaggatatctggcaatacatctggctggaaaatatcgacattgttccgctatatctcgctgcggaacgtccggttctggaacgcgacggtatgttgatgatgattgatgacaaccgtatcgacctgcaaccgggcgaagtgattaaaaaacggatggtgcgtttccgtacgctgggctgctggccgctgaccggtgcggtggagtcaaatgcacaaacactgccggaaatcatcgaagagatgctggtttccaccaccagtgaacgtcagggccgcgtgattgaccgcgaccaggcggggtctatggagctgaaaaaacgtcaggggtatttttaa

SEQ ID NO: 118 - DNA, 1325 bp, synthetic construct
  misc_feature 1..1325: note = Synthetic Construct
  source 1..1325: mol_type = other DNA
attagacctcagcgacccggcgtcggtagccgcagctatcaaacgcgcgaaggaaaaagaagggcgcattgatatcctggtgaataacgcaggcgtttgtcgtctgggcagtttcctcgatatgagcgatgacgatcgcgatttccatattgacatcaatattaaaggcgtatggaacgtcacgaaggcggtgctgccggagatgattgcccgcaaagatggtcgcattgtgatgatgtcttcagtcactggtgatatggtggccgatcctggcgaaacggcgtacgccttaacgaaagcggcgattgttggcctgacaaaatcgctggcggtggagtacgcgcagtctggtattcgcgttaacgccatttgcccgggatacgtgcgcacaccaatggcggaaagcattgcccgccagtcgaacccggaagatccagagtcggtgctgactgaaatggcgaaagcaatcccgatgcgtcgcctcgccgatccgctggaagtcggcgaactggcggccttcctcgcatcggatgaatccagctatttaaccggtacacagaatgtgattgatggcggcagcacactgccggagacggttagcgtcggtatctgattgacaattaatcatccggctcgtataatgtgtggaattgtgagcggataacaatttcacacaggaaacagaccatggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcggcgatcgcatattat

SEQ ID NO: 119 - DNA, 2000 bp, synthetic construct
  misc_feature 1..2000: note = Synthetic Construct
  source 1..2000: mol_type = other DNA
tggtgacctgaaatatggccgcaccgttcactccctgactcaggcgttagcgaagttcgacggcaaccgtttttacttcatcgcgccggacgcgctggcaatgccgcaatacattctggatatgctcgatgaaaaagggatcgcatggagtctgcacagctctattgaagaagtgatggcggaagtagacatcctgtacatgacccgcgtgcaaaaagagcgtctggacccgtccgagtacgccaacgtgaaagcgcagtttgttcttcgcgccagcgatctccacaacgccaaagccaatatgaaagtgctgcatccgctgccgcgtgttgatgagattgcgacggatgttgataaaacgccacacgcctggtacttccagcaggcaggcaacgggattttcgctcgccaggcgttactggcactggttctgaatcgcgatctggtactgtaaggggaaatagagatgacacacgataataaattgcaggttgaagctattaaacgcggcacggtaattgaccatatccccgcccagatcggttttaagctgttgagtctgttcaagctgaccgaaacggatcagcgcatcaccattggtctgaacctgccttctggcgagatgggccgcaaagatctgatcaaaatcgaaaatacctttttgagtgaagatcaagtagatcaactggcattgtatgcgccgcaagccacggttaaccgtatcgacaactatgaagtggtgggtaaatcgcgcccaagtctgccggagcgcatcgacaatgtgctggtctgcccgaacagcaactgtatcagccatgccgaaccggtttcatccagctttgccgtgcgaaaacgcgccaatgatatcgcgctcaaatgcaaatactgtgaaaaagagttttcccataatgtggtgctggccaattaattgcggttggtaataaaagtctggctccctataatgagccagactttttaccgctgtaataaaggagaaatctcttgatggaaatccgggctatcatgcccggattaagtctgatgacaaacgcaaaatcgcctgatgcgctacgcttatcaggcctacgtgattcctgcaatttattgaatttgttggccggataaggcatttacgccgcatccggcatgaacaaaactcactttgtctacaatctgaatcggggctatcgtgcccagtttattctttattgccagccgtaacgacggctatagaaccctttcaccaactgggttaatgtcatataccctgccagaatcgcaaccagccacgggaaatagcttaacggcagcgcctgtaattgcagataactggccagcggtgaaaacggcaatgcgatcccgacaatcatcacgatcacggtcatgatcattaacggccacgatgcacagctctgaataaacggcacacggcgggtgcggatcatatgcacaatcagcgtttgcgacagtaagcccaccacaaaccatcccgactggaacagcgtttgcgtttccggcgtgttggcatggaatacccaccacatcaggcaaaacgtcaaaatatcgaagatcgagctgatcggtccgaagaagatcataaagcgccccagatccgccggattccaacgctgcggcttttgaatttgctcgtcgtcgacgttatcaaacgggatcgccacctgtgacacatcgtacagcaggttctgaataagcaagtgtaacggcaacatcggcaggaagggcaagaaagcactcgctaccagcacgctgaacacattaccgaagttagagctcgccgtcattttgatgtatttcagcatgttggcgaaagtgcgacgtccctcaataaccccctcttccagcaccatcaggcttttttccagcaggatgatatcagccgcttcacgggcaatatctaccgcgccgtccacagaaatgccgatatccgcagcgcgtaaggcgggcgcatcattaataccatcgcccataaagccaaccac

SEQ ID NO: 120 - DNA, 2000 bp, synthetic construct
  misc_feature 1..2000: note = Synthetic Construct
  source 1..2000: mol_type = other DNA
tttggtgcacgcgctaacctggcgaaaacgctgctctacgcaattaacggcggggtggacgagaagctgaagattcaggtcgggccgaaaacagcaccgctgatggacgacgtgctggattacgacaaagtgatggacagcctcgatcacttcatggactggctggcggtgcagtacatcagcgcgctgaatatcattcactacatgcacgacaagtacagctacgaagcttcgctgatggcgctgcacgatcgtgatgtctatcgcactatggcatgcggcatcgcgggcctgtcggtggcgacggactccctgtctgccatcaaatatgcccgcgtgaaaccaatccgtgacgaaaacggcctggcggtggactttgaaatcgacggtgaatatccgcagtacggcaacaacgacgagcgcgtagacagcattgcctgcgacctggttgaacgctttatgaagaaaattaaagcgctgccaacctatcgcaacgccgtccctacccagtcgattctgactatcacttctaacgtggtgtacggccagaaaaccggtaatacgccggacggtcgtcgcgccggaacaccgttcgcgccgggcgctaacccgatgcatggtcgtgaccgcaaaggtgccgtggcctcattgacgtcggtggcgaaactgccgttcacctacgccaaagatgggatctcgtacaccttctcaatcgttcctgcggcgctgggcaaagaagatccagtacgtaaaaccaaccttgtcggcctgctggatgggtatttccaccacgaagcggatgtcgaaggcggtcaacacctcaacgtcaacgtaatgaatcgggaaatgctgctggatgccatcgagcacccggaaaaatatcctaacctgacaatccgtgtctctggctacgccgtgcgcttcaacgcactgacccgtgaacagcaacaggatgttatttcacgtacctttacccaggcgctctgacgccggaggatgtagcctcgtgagcgggacggtcgtaaggtcgttccgctccacttcactgaacggcaatccgagggtgtggatatgattagtgcattcgatattttcaaaattgggattggtccctccagttcgcataccgtggggccaatgaatgccggaaaaagttttattgatcggctggaaagtagcggcttattaaccgcgacgagccatattgtggtcgatctgtacgggtcgttgtcactgacgggcaaaggccatgccacggatgtcgccatcatcatgggactggcaggaaacagtccgcaggatgttgtcattgatgagatccctgcatttatagagttagtaacgcgcagcgggcggctgccagtggcatctggtgcgcatattgttgattttcctgtagcaaagaacattatcttccatcccgaaatgttgcctcgccatgagaacggaatgcggatcactgcctggaagggacaggaagagctattaagtaaaacctattactctgtcggcggcgggtttattgtcgaagaagaacacttcggcctgtcgcacgatgtcgaaacgtccgtaccttacgatttccactcagcaggtgaactgctgaaaatgtgtgattacaacggcctgtctatatctggtctgatgatgcacaacgagctagcgctgcgcagcaaagcggaaattgacgccggttttgcccgtatctggcaagtgatgcatgacggtattgaacgtgggatgaacactgaaggcgtgctgcctggtccgctcaatgtgccgcgccgtgccgtagcgctgcgtcgtcagctggtttccagcgataacatctctaacgatccgatgaatgtcatcgactggatcaacatgtacgcgctggcggttagtgaagaaaacgcagctggcgggcgcgtggtaacggcaccgactaacggtgcgtgcggcattattccggcagtactggcttattacgataagttccgtcgtccggtaaacgagcg

SEQ ID NO: 121 - DNA, 1000 bp, synthetic construct
  misc_feature 1..1000: note = Synthetic Construct
  source 1..1000: mol_type = other DNA
tggtttcaaaatggctgggatgaacagtatgttgaggctggcggccccggctcaccgaattatcataaatcgaacgccctgaaaaccatgcgtaatcagccgctgctgcaggggaaattgctgacgaaaggctcctgggattatcaactggtggatgaactgatgccgcagcctggcgatattgtgctgccgaagccgcgctacagcggtttcttcaatacgccgctggacagcattttgcgcagccgtggaatacgccatctggttttcaccggtatcgctaccaacgtctgcgtcgaatcgacgctacgcgacggcttttttctggagtatttcggcgtggtgctggaagacgcaacacaccaggcggggccggaatttgcacagaaagccgcgttgttcaatatcgaaaccttttttggctgggtcagcgacgtcgaaacgttctgcgacgcgctttcttccacgtcctttgctcgtatcgcttaaggagtttaacgggccgcgatgaaactttcactctcacctcccccttatgctgatgcgcccgtagtggtgttgatttcgggtcttggcggtagcggcagttactggttaccgcaactggcggtgctggatcaggagtatcaggtagtctgttacgaccagcgcggcaccggcaataatcccgacacgctggcagaagattacagtatcgcccagatggcagcggaactgcatcaggcgctggtagccgcagggattgagcgttacgcggtggtcggccatgcgctcggtgcgctggtgggaatgcagctggcgctggattatcccgcgtcggtaactgtgctggtcagcgttaacggctggctacgaataaacgcccatacgcgccgctgttttcaggttcgcgaacagttactacatagcggcggcgcgcaggcatgggtggaagcgctgccgttgttcctctatcccgccgactggatggcggcccgcgcacctcgccttgaggcagaag

SEQ ID NO: 122 - DNA, 29 bp, synthetic construct
  misc_feature 1..29: note = Synthetic Construct
  source 1..29: mol_type = other DNA
cgcggatccgcataattttgtcagtggtg

SEQ ID NO: 123 - DNA, 40 bp, synthetic construct
  misc_feature 1..40: note = Synthetic Construct
  source 1..40: mol_type = other DNA
acctgattcttctgatcgccctggcgcgcatccgcacgcg

SEQ ID NO: 124 - DNA, 41 bp, synthetic construct
  misc_feature 1..41: note = Synthetic Construct
  source 1..41: mol_type = other DNA
cgcgtgcggatgcgcgccagggcgatcagaagaatcaggtg

SEQ ID NO: 125 - DNA, 30 bp, synthetic construct
  misc_feature 1..30: note = Synthetic Construct
  source 1..30: mol_type = other DNA
cccaagctttagaacagcgcgctggccatc

SEQ ID NO: 126 - DNA, 29 bp, synthetic construct
  misc_feature 1..29: note = Synthetic Construct
  source 1..29: mol_type = other DNA
cgcggatcccaacgacggcggagacatgc

SEQ ID NO: 127 - DNA, 43 bp, synthetic construct
  misc_feature 1..43: note = Synthetic Construct
  source 1..43: mol_type = other DNA
ccatcggaaccacaccaacgccggccggcgcggtcgaaaagtg

SEQ ID NO: 128 - DNA, 43 bp, synthetic construct
  misc_feature 1..43: note = Synthetic Construct
  source 1..43: mol_type = other DNA
cacttttcgaccgcgccggccggcgttggtgtggttccgatgg

SEQ ID NO: 129 - DNA, 30 bp, synthetic construct
  misc_feature 1..30: note = Synthetic Construct
  source 1..30: mol_type = other DNA
cccaagcttggccggcgcggtcgaaaagtg

SEQ ID NO: 130 - DNA, 30 bp, synthetic construct
  misc_feature 1..30: note = Synthetic Construct
  source 1..30: mol_type = other DNA
cgcggatccccttcccatcgccttaaaccc

SEQ ID NO: 131 - DNA, 41 bp, synthetic construct
  misc_feature 1..41: note = Synthetic Construct
  source 1..41: mol_type = other DNA
cgaggtcttggggtttgtcgcctgggaagcgggtctcctgg

SEQ ID NO: 132 - DNA, 41 bp, synthetic construct
  misc_feature 1..41: note = Synthetic Construct
  source 1..41: mol_type = other DNA
ccaggagacccgcttcccaggcgacaaaccccaagacctcg

SEQ ID NO: 133 - DNA, 31 bp, synthetic construct
  misc_feature 1..31: note = Synthetic Construct
  source 1..31: mol_type = other DNA
cccaagcttgcctagagacaccgcatcgctg

SEQ ID NO: 134 - DNA, 1365 bp, synthetic construct
  misc_feature 1..1365: note = Synthetic Construct
  source 1..1365: mol_type = other DNA
attagacctcagcgacccggcgtcggtagccgcagctatcaaacgcgcgaaggaaaaagaagggcgcattgatatcctggtgaataacgcaggcgtttgtcgtctgggcagtttcctcgatatgagcgatgacgatcgcgatttccatattgacatcaatattaaaggcgtatggaacgtcacgaaggcggtgctgccggagatgattgcccgcaaagatggtcgcattgtgatgatgtcttcagtcactggtgatatggtggccgatcctggcgaaacggcgtacgccttaacgaaagcggcgattgttggcctgacaaaatcgctggcggtggagtacgcgcagtctggtattcgcgttaacgccatttgcccgggatacgtgcgcacaccaatggcggaaagcattgcccgccagtcgaacccggaagatccagagtcggtgctgactgaaatggcgaaagcaatcccgatgcgtcgcctcgccgatccgctggaagtcggcgaactggcggccttcctcgcatcggatgaatccagctatttaaccggtacacagaatgtgattgatggcggcagcacactgccggagacggttagcgtcggtatctgattgacaattaatcatccggctcgtataatgtgtggaattgtgagcggataacaatttcacacaggaaacagaccatgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacgg

SEQ ID NO: 135 - DNA, 1100 bp, synthetic construct
  misc_feature 1..1100: note = Synthetic construct
  source 1..1100: mol_type = other DNA
tcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaataggaaaaatgcctgatagcgcttcgcttatcaggcctacccgcgcgacaacgtcatttgtggttcggcaaaatcttccagaatgcctcaattagcggctcatgtagccgctttttctgcgcacacacgcccagctcaaacggcgttttctcatcgctgcgctctaaaatcatcacgcggttacgcaccggttcggggctgttttccagcaccacttccggcaacaatgccacgccacagccgagtgccaccatcgataccatcgcttcatgcccgccaaccgtggcgtaaatcatcgggttactgattttattgcgtcgaaaccacagttcaatgcggcggcgtaccggcccctgatcggccataataaacggcaccgttgaccagtccggcttctctaccgacacctgattacgcaccgggcagggcagcgcgggggcaatcagcactactgccagattctccagcatcgaaaacgccactgcgccgggcaaggtttccggtttacccgcaatcgccagatccgcttcaccagtgaccaccttttccatc

SEQ ID NO: 136 - DNA, 1545 bp, Escherichia coli
  source 1..1545: mol_type = genomic DNA
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 137 - protein, 514 aa, Escherichia coli [sequence omitted]

SEQ ID NO: 138 - DNA, 1000 bp, synthetic construct
  misc_feature 1..1000: note = Synthetic Construct
  source 1..1000: mol_type = other DNA
aaggtttcggtggctgcagtaagttgctgtcgggccgctacctggaggtgaatcagacgggacagcggaaggtagacttctgccacttcagcgaggtcaatgttttctccgatgcctcgaagttcaatgacttctttttgggtcagcacctgaggcattgagtttctcagctcgcgccattgtgcgcggtcgaaatcaaggtaggggctgaaatctggtgtgcgtggggaaggtttcacaccagttgtgcttgcagcgttttgctctgccatgaatccattgtgcaccttagctactccactagtgtgatcggggttattttttcacttcaatgggtggctaaaagacgtgggcacgtgagtaaactcatgcgcgcgaaacgatgggagtgaacccatacttttatatatgggtatcggcggtctatgcttgtgggcgtacctgtcccgcgagtgaggtcttacgcgcgggattcgtcttgtgaaaggttagctgacctggtttttctttgagttttcatatgtagaaggcatcgtcggcttcggcctggcggtgcttttctcgttgttttgtggttttgtcagaggatgtcatgcgcgttttaattattgataattatgattctttcacgtttaatctcgccacctatgtggaagaggttacgggtcaggcacctgtggtggtgcctaatgatcaagaaatagatgagatgcttttcgacgccgtcatcctctcacctggcccgggccacgccggcgttgcggctgattttggtatctgtgcaggcgtcattgagcgtgcacgcgttccgattttgggtgtgtgtttaggccaccagggcattgcgttggcctatggcggtgatgttgatttggcgcccaggccggtccacggtgaggtttcgcagatcacccatgatggttcaggtttatttgcaggcatccctgaaacgtttgaggcggtgcgttatcactcgatggtggcaacccgcttgccggag

SEQ ID NO: 139 - DNA, 1000 bp, synthetic construct
  misc_feature 1..1000: note = Synthetic Construct
  source 1..1000: mol_type = other DNA
cgctgatttcatcgtcatcatcgctggtgtctttgccggaggagctcaagcttggggccgcaggaatgtcgccattgctgagcattgagctgccttcagagctgcctggccaggtttcgtttccatcgactggatttccatcatcatcaaggatctgtgatgaggtgatgttgtctgagagctgtgtcagtgcgtcagaggactgagcctgggcaactggagtgaacacggacaatgccacagcgcttgctgtaacaagggtcaaagtacttcgacgcaaagacaaaacttttctcctggcaataaatatgcggatttactatggaaacaagatagaagattggatagcgaaagctatcctcaactcgtggaaagtgtagtgccacaaccacagtattggctagaaaacaatctatagcattgttctacaaagagcttgttggaaataaaacctatgccaaagtaggtgcaattctaggagaagattacactagtcaaccacatagctgaaggccacctcaatcgaggtggcctttttctagtttcgggtcaggatcgcaaagccccacggctgaagggttgtggaggtgtcggtgacggtgggggaagtgaagctgtaaatcagctcgccgccaagcgggacggtgatggtgtcgtcggagaaattcgccagaattcggccgcgaccattggccatcgatagccagttctcgccgtgctcaacctcgagtgtgagcaagtttggttgggagaagcccaaggtgtgccgcaggtgcaacagctgcttgtaagcgtcgttgatgcggcgctgctccgcagtgaactcccaatcgagtttggaggaggtgaaggtggattccagctcgggggaggggatgtcgtcggcgttccagccaaggcgtgcgaattcccgtttgcggccctcggaggttaggcggttgagctcggggtcggtgtgggagcaaaagaaggcgaatggggtggtggctccgaattcttc

SEQ ID NO: 140 - DNA, 1311 bp, Corynebacterium glutamicum
  source 1..1311: mol_type = genomic DNA
atgagtgaaacatacgtgtctgagaaaagtccaggagtgatggctagcggagcggagctgattcgtgccgccgacattcaaacggcgcaggcacgaatttcctccgtcattgcaccaactccattgcagtattgccctcgtctttctgaggaaaccggagcggaaatctaccttaagcgtgaggatctgcaggatgttcgttcctacaagatccgcggtgcgctgaactctggagcgcagctcacccaagagcagcgcgatgcaggtatcgttgccgcatctgcaggtaaccatgcccagggcgtggcctatgtgtgcaagtccttgggcgttcagggacgcatctatgttcctgtgcagactccaaagcaaaagcgtgaccgcatcatggttcacggcggagagtttgtctccttggtggtcactggcaataacttcgacgaagcatcggctgcagcgcatgaagatgcagagcgcaccggcgcaacgctgatcgagcctttcgatgctcgcaacaccgtcatcggtcagggcaccgtggctgctgagatcttgtcgcagctgacttccatgggcaagagtgcagatcacgtgatggttccagtcggcggtggcggacttcttgcaggtgtggtcagctacatggctgatatggcacctcgcactgcgatcgttggtatcgaaccagcgggagcagcatccatgcaggctgcattgcacaatggtggaccaatcactttggagactgttgatccctttgtggacggcgcagcagtcaaacgtgtcggagatctcaactacaccatcgtggagaagaaccagggtcgcgtgcacatgatgagcgcgaccgagggcgctgtgtgtactgagatgctcgatctttaccaaaacgaaggcatcatcgcggagcctgctggcgcgctgtctatcgctgggttgaaggaaatgtcctttgcacctggttctgtcgtggtgtgcatcatctctggtggcaacaacgatgtgctgcgttatgcggaaatcgctgagcgctccttggtgcaccgcggtttgaagcactacttcttggtgaacttcccgcaaaagcctggtcagttgcgtcacttcctggaagatatcctgggaccggatgatgacatcacgctgtttgagtacctcaagcgcaacaaccgtgagaccggtactgcgttggtgggtattcacttgagtgaagcatcaggattggattctttgctggaacgtatggaggaatcggcaattgattcccgtcgcctcgagccgggcacccctgagtacgaatacttgacctaa

SEQ ID NO: 141 - protein, 436 aa, Corynebacterium glutamicum
MSETYVSEKSPGVMASGAELIRAADIQTAQARISSVIAPTPLQYCPRLSEETGAEIYLKREDLQDVRSYKIRGALNSGAQLTQEQRDAGIVAASAGNHAQGVAYVCKSLGVQGRIYVPVQTPKQKRDRIMVHGGEFVSLVVTGNNFDEASAAAHEDAERTGATLIEPFDARNTVIGQGTVAAEILSQLTSMGKSADHVMVPVGGGGLLAGVVSYMADMAPRTAIVGIEPAGAASMQAALHNGGPITLETVDPFVDGAAVKRVGDLNYTIVEKNQGRVHMMSATEGAVCTEMLDLYQNEGIIAEPAGALSIAGLKEMSFAPGSVVVCIISGGNNDVLRYAEIAERSLVHRGLKHYFLVNFPQKPGQLRHFLEDILGPDDDITLFEYLKRNNRETGTALVGIHLSEASGLDSLLERMEESAIDSRRLEPGTPEYEYLT

SEQ ID NO: 142 - DNA, 1000 bp, synthetic construct
  misc_feature 1..1000: note = Synthetic Construct
  source 1..1000: mol_type = other DNA
tggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaactttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacccaatagagtgcgattgctgtggggaatggtcctgtgaggccaagggacagtgggaagatcggcgcgagg

SEQ ID NO: 143 - DNA, 246 bp, Escherichia coli
  source 1..246: mol_type = genomic DNA
atgcgcattactatttacactcgtaacgattgcgttcagtgccacgccaccaaacgggcgatggaaaaccggggctttgattttgaaatgattaatgtcgatcgcgttcctgaagcggcagaagcgttgcgtgctcagggctttcgtcagttgccggtagtgattgctggcgatcttagctggtctggtttccgtccggacatgattaaccgtctgcatccagcgccacacgcggccagtgcatga

SEQ ID NO: 144 - protein, 81 aa, Escherichia coli
MRITIYTRNDCVQCHATKRAMENRGFDFEMINVDRVPEAAEALRAQGFRQLPVVIAGDLSWSGFRPDMINRLHPAPHAASA

SEQ ID NO: 145 - DNA, 1000 bp, synthetic construct
  misc_feature 1..1000: note = Synthetic Construct
  source 1..1000: mol_type = other DNA
ctccagtgtgcaaggccttgggggcgcaaccgcgaaccaagacgctgacctttccttccgtcgcaccactggcgtgaccccgggagagaacttcagcagctttgccgatcttctcaaaggacacgacggaaacggctaaattcgcggatctccgtttaaggcattgaagcatttggaggccccaagacatgacccagaccctgtaaagcgcttaaacggcgttttagagggtcatagttttgggacaagtgggacaagtgtgaatcctgaaagcttccagggcaaggatccaccacaaaccggccatcgccctttggaatcggtccgaaaattgcaggtacagagccttttaccgagaaaatccaccacagattgctgaaatttcgtgatctgtggtggattcgtgcaacttcagactcttacggaggcgatggaccaaaaacaactacaatcaagcagatcaccttgtacaccaccatagaaaaggcccaccctcagcccggtacggctttaacacggcttggattttgtcttgcttggcgaggtaggactggcactgggcgtcgataagctcagctgaccacccggtgacctgcgccattgcttcggccgtcgcacgcacggcagccggttgcacataacccagcgtgccgagcacgatgcgacgatccagcacgtccgccaggtcgacggccgcctcctcggcgacagcaaaaacggcctgcgccgcgatatcaaggttgtctgggtcaagtcggcgccccaggtcgggttgctttgcgacgagatccagcactttttcatgctcagttccatacagtctggccagatgcacgcggatttcatcatccacatccagctcggggtggctgcgaagcgctgactcaaaggaatcagccacggactcatacgcgccaaaagaagtactcaacggcatctgcctggagtcaaagtggcgcagcggaggtaacaccgcagcttggtgctccaccacatgctc

SEQ ID NO: 146 - DNA, 1200 bp, synthetic construct
  misc_feature 1..1200: note = Synthetic Construct
  source 1..1200: mol_type = other DNA
ctgcgtaaacacgctggacaagtggcattccctggaggtgcagtcgatgacacggacgcatcagctatcgccgccgcgctgcgcgaagctgaagaagaggtcgctataccgccttccgccgttgaagttatcggcgtgctgccgcccgtcgatagcgtcactggctaccaggtaaccccagtggtcggcattatcccgcccgatctgccgtatcgcgccagtgaagatgaagtctcggcggtgtttgaaatgccgctcgcccaggcattacatctgggtcgttatcaccctttagatatctaccgccgtggtgattcacatcgggtatggctgtcctggtacgaacagtattttgtatggggaatgaccgcaggcataattcgtgagctggcgctgcaaattggtgtgaaaccctgactatacttatctttacatctacaaaacactacttgagacaatcatcgcaatattagttaaatcgcggtttttgattagtttaattcatgtgaatagttaagccagtcgccgcgttccctcttacactatgcgctgttattagttcgttactggaagtccagtcaccttgtcaggagtattatctacttcttactcgcccatctgcaacggatgggcgaatttatacccgctttctcgtctgctgtaatattccccactacacttccactgttgcgtcaggcgtttgtcgccatacgcttacagggtggcccgcatgcaaaaagcacaacggatcattaaaacctatcgtcgtaatcgaatgattgtttgtacgatttgcgcactcgttacgctcgcttcgaccctgagcgtgcgatttatttcacagcgtaacttaaatcaacaacgggtagtacaattcgccaatcacgctgtagaggaattagataaagtactgcttcccctacaggcaggtagcgaagtcttgcttccgctgattggtctgccctgctctgtcgcccatttgccattacgtaaacaggcggcaaaactccaaactgtgcgatccattggcctggtgcaagacggcacactttattgctccagcatttttggttatcgcaatgtgcccgtcgtggacattctggctgaacttcctgcaccgcaaccacttttacgcctgacgatcgaccgtgccctgattaaaggcagtccggttttgattcaatggacgcctgcagcggg

SEQ ID NO: 147 - DNA, 936 bp, Corynebacterium glutamicum
  source 1..936: mol_type = genomic DNA
atgggcaatgtgtacaacaacatcaccgaaaccatcggccacaccccactggtaaagctgaacaagctcaccgaaggcctcgacgcaactgtcctggtcaagcttgagtcattcaacccagcaaactccgtcaaggaccgtatcggtctggccatcgttgaagatgcagagaagtccggtgcactgaagccaggcggcaccatcgttgaagcaacctccggcaacaccggtatcgcactggcaatggtcggcgctgcacgcggatacaacgttgttctcaccatgccggagaccatgtccaacgagcgtcgcgttctcctccgcgcttacggtgcagagatcgttcttaccccaggtgcagcaggcatgcagggtgcaaaggacaaggcagacgaaatcgtcgctgaacgcgaaaacgcagtccttgctcgccagttcgagaacgaggcaaacccacgcgtccaccgcgacaccaccgcgaaggaaatcctcgaagacaccgacggcaacgttgatatcttcgttgcaagcttcggcaccggcggaaccgtcaccggcgttggccaggtcctgaaggaaaacaacgcagacgtacaggtctacaccgtcgagccagaagcgtccccacttctgaccgctggcaaggctggtccacacaagatccagggcatcggcgcaaacttcatccccgaggtcctggaccgcaaggttctcgacgacgtgctgaccgtctccaacgaagacgcaatcgcattctcccgcaagctcgctaccgaagagggcatcctcggcggtatctccaccggcgcaaacatcaaggcagctcttgaccttgcagcaaagccagagaacgctggcaaaaccatcgtcaccgttgtcaccgacttcggcgagcgctacgtctccaccgttctttacgaagacatccgcgactaa

SEQ ID NO: 148 - protein, 311 aa, Corynebacterium glutamicum
MGNVYNNITETIGHTPLVKLNKLTEGLDATVLVKLESFNPANSVKDRIGLAIVEDAEKSGALKPGGTIVEATSGNTGIALAMVGAARGYNVVLTMPETMSNERRVLLRAYGAEIVLTPGAAGMQGAKDKADEIVAERENAVLARQFENEANPRVHRDTTAKEILEDTDGNVDIFVASFGTGGTVTGVGQVLKENNADVQVYTVEPEASPLLTAGKAGPHKIQGIGANFIPEVLDRKVLDDVLTVSNEDAIAFSRKLATEEGILGGISTGANIKAALDLAAKPENAGKTIVTVVTDFGERYVSTVLYEDIRD

SEQ ID NO: 149 - DNA, 387 bp, Corynebacterium glutamicum
  source 1..387: mol_type = genomic DNA
atgcctgtgcagctccctccaggacctgctgaacgcactgtttattcctacggtgttcgcgcagggggtcagttgcacatttcaggaatggtggcttttgattccgaggcgaatattgtcggcgaaggggacatcgaagcgcagactgaacaagtctttaagaacctgcaagcagtagtcgaggaggcaggtggaacgatcaacaacattgtttccaccactacttatcttgccgatgtcaccgatgctcctgtggttaatgccgctcgcgcccgctatttcaccggagatgtattgcctacccacaccgtaattggggttgctgctctggctcgcccacagtttctagtcgagatctcggcggtggcctatttgggggacctttag

SEQ ID NO: 150 - protein, 128 aa, Corynebacterium glutamicum
MPVQLPPGPAERTVYSYGVRAGGQLHISGMVAFDSEANIVGEGDIEAQTEQVFKNLQAVVEEAGGTINNIVSTTTYLADVTDAPVVNAARARYFTGDVLPTHTVIGVAALARPQFLVEISAVAYLGDL

SEQ ID NO: 151 - DNA, 396 bp, Corynebacterium glutamicum
  source 1..396: mol_type = genomic DNA
atgcctgttccggcgcttcccttcggggaaacagctatggttggccacatgtcgaatgatcacccatactcccccgcaaaacgcgtcggaaatttcatcttcgtctccggcgctctttcagtagacaaggactaccaaccagtcgtcggtcgtaaagaagcagttgatgcagcacttgaacgcatgcgtgaacgcgtcgccaccgctggtggtgaactcaaagacgttgtgaaactcacttactttgtcaccgacatcagcctgcgcgaagaatgcaacgagcaattccgagagcatttcctcgaaggccgcccggcacgctctttcgtgggtgcatcatcgcttccttatggtgcaactgtggaaattgatgcgattgcgatgatcgaggactaa

SEQ ID NO: 152 - protein, 131 aa, Corynebacterium glutamicum
MPVPALPFGETAMVGHMSNDHPYSPAKRVGNFIFVSGALSVDKDYQPVVGRKEAVDAALERMRERVATAGGELKDVVKLTYFVTDISLREECNEQFREHFLEGRPARSFVGASSLPYGATVEIDAIAMIED

SEQ ID NO: 153 - DNA, 70 bp, Escherichia coli
  source 1..70: mol_type = genomic DNA
ctgcgcgcgttacagcgccgcctgacgccctggcatggagaagtacaatgattccggggatccgtcgacc

SEQ ID NO: 154 - DNA, 70 bp, Escherichia coli
  source 1..70: mol_type = genomic DNA
agggggcgaggggaccgtccactctcgtattaccccgcccgataaaacggtgtaggctggagctgcttcg

SEQ ID NO: 155 - DNA, 70 bp, Escherichia coli
  source 1..70: mol_type = genomic DNA
attcgccagcgcatctggcagcccactcaactggaaggaaaacaattatgattccggggatccgtcgacc

SEQ ID NO: 156 - DNA, 70 bp, Escherichia coli
  source 1..70: mol_type = genomic DNA
tcttcactggcgttgccattatttcttccttagctttgcgcgactttacgtgtaggctggagctgcttcg

SEQ ID NO: 157 - DNA, 70 bp, Escherichia coli
  source 1..70: mol_type = genomic DNA
gttatcaatgttaacaaaaaaagaacaattggttataaggagagagtatgattccggggatccgtcgacc

SEQ ID NO: 158 - DNA, 70 bp, Escherichia coli
  source 1..70: mol_type = genomic DNA
gcaatcccgccagcgccagtttaatgatgttacgcatgggcattacctcgtgtaggctggagctgcttcg

SEQ ID NO: 159 - DNA, 1200 bp, synthetic construct
  misc_feature 1..1200: note = Synthtic Construct
  source 1..1200: mol_type = other DNA
tttgatctggaagctggtgtgcttctgcgcgattttttctaccgctggcagcagcgcaggcaggttttcaatattactggcgtaggcggcaatagcgttaaacagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcgggcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatccacggttctccactttgccaggcagcgattaactgcgctgtagtcagtttttcaggatcaagttgagtactaaccggttgtgattttagccatacccgcgcctgtttttttaatgcgctt

SEQ ID NO: 160 - DNA, 990 bp, Escherichia coli
  source 1..990: mol_type = genomic DNA
atgaacaagtggggcgtagggttaacatttttgctggcggcaaccagcgttatggcaaaggatattcagcttcttaacgtttcatatgatccaacgcgcgaattgtacgaacagtacaacaaggcattcagcgcccactggaaacagcaaactggtgataacgtggtgattcgtcagtcacacggtggctcaggtaaacaagcgacgtcggtaatcaacggtattgaagctgatgttgtcacgctggctctggcctatgacgtggacgcaattgcggaacgcgggcggattgataaagagtggatcaaacgtctgccggataactccgcaccgtacacttccaccattgttttcctggtacgtaagggaaatccgaagcagatccatgactggaacgatctgattaaaccgggtgtttcggtgatcacgcctaatccgaaaagctctggtggcgcgcgctggaactacctggcagcctggggctacgcgctgcatcacaacaacaacgatcaggcaaaagcacaggattttgttcgggcactgtataaaaacgtcgaagttctggattctggcgcgcgtggctccactaacacttttgtcgagcgcggaattggcgatgtactgattgcctgggaaaacgaagctctgctggcagcgaatgaactggggaaagataaattcgaaatcgtcacgccgagtgagtctatcctcgcagagccaaccgtgtcggtggtcgataaagtggtcgagaaaaaaggtactaaagaggtggcggaagcctacctgaaatatctctactcgccagaaggtcaggaaattgccgcgaaaaactactaccgtccgcgcgacgctgaggtggcgaaaaagtacgaaaatgcgtttccaaagctgaagttattcaccattgatgaagagttcggcggctggacgaaagcgcaaaaagagcattttgctaacggcggtacgttcgatcagatcagcaaacgctga

SEQ ID NO: 161 - protein, 329 aa, Escherichia coli
MNKWGVGLTFLLAATSVMAKDIQLLNVSYDPTRELYEQYNKAFSAHWKQQTGDNVVIRQSHGGSGKQATSVINGIEADVVTLALAYDVDAIAERGRIDKEWIKRLPDNSAPYTSTIVFLVRKGNPKQIHDWNDLIKPGVSVITPNPKSSGGARWNYLAAWGYALHHNNNDQAKAQDFVRALYKNVEVLDSGARGSTNTFVERGIGDVLIAWENEALLAANELGKDKFEIVTPSESILAEPTVSVVDKVVEKKGTKEVAEAYLKYLYSPEGQEIAAKNYYRPRDAEVAKKYENAFPKLKLFTIDEEFGGWTKAQKEHFANGGTFDQISKR

SEQ ID NO: 162 - DNA, 771 bp, Corynebacterium glutamicum
  source 1..771: mol_type = genomic DNA
atgactacaaccttgactcgccccaaaatcgcgctgcccgcgcgcatctattcaccgcttgcggtgcttgttttctggcagctcggctcgagcctgggcgccatcccggagcggattctgccggcaccaaccacgatcttggccgccagctgggaggtcgccacaaatggcacgcttctcgacgccctcctcgtctcaagccaacgcgtccttctaggcttcgccctcggtgctgtcctaggcatttccctaggtgtattgacaggcatgtccagatttgcagacaccgccgttgatccgctcattcaagctgcccgcgcgctgcctcacctgggtcttgtgccgctgtttatcatctggttcggtatcggtgagctgccgaaagtactgattattagcctcggcgtgctgtatccgctgtacctcaacaccgccagcgggttcaggcaaattgatccaaagcttctggaagccggccacgtgatgggcttcggatttttccagaggttgcggaccatcatcattccttctgccgcgccgcaactttttgtcggcctgcgccaagcaagtgcggccgcctggctctcactgatcgtggcggaacaggtcaacgcccgcgaaggactcggcttcctcatcaacaatgcgcgcgatttttaccgcaccgacctcgttattttcggcctcattgtctacgccagcctcggtctgctgtctgaagcgctgatcagagcttgggaacgtcacaccttccgctaccgaaacgcataa

SEQ ID NO: 163 - protein, 256 aa, Corynebacterium glutamicum
MTTTLTRPKIALPARIYSPLAVLVFWQLGSSLGAIPERILPAPTTILAASWEVATNGTLLDALLVSSQRVLLGFALGAVLGISLGVLTGMSRFADTAVDPLIQAARALPHLGLVPLFIIWFGIGELPKVLIISLGVLYPLYLNTASGFRQIDPKLLEAGHVMGFGFFQRLRTIIIPSAAPQLFVGLRQASAAAWLSLIVAEQVNAREGLGFLINNARDFYRTDLVIFGLIVYASLGLLSEALIRAWERHTFRYRNA

SEQ ID NO: 164 - DNA, 732 bp, Corynebacterium glutamicum
  source 1..732: mol_type = genomic DNA
atgactgccacattgtcactcaaacccgcagccactgtccgtggattgcgcaaatcatacggaactaaagaagtcctccaaggaatcgacctcaccatcaactgcggcgaagtaaccgcgctgatcggacgctcaggttcaggaaaatccaccatcctgcgcgtgttggcgggcctatctaaagagcattccggctctgtagaaatttccggaaacccggccgttgccttccaagagcctcgcctgttgccgtggaaaacggtgctcgataatgtgacctttggcctcaaccgcactgatatttcctggtcagaagcacaagaacgcgcctcggcactgcttgcagaagtcaaacttcccgactccgacgccgcctggcccctcacgctctccggcggccaagcccagcgcgtctcccttgcgcgagcgctcatctccgagccagagcttttgcttctcgacgaacccttcggcgccctcgatgctctgacaagactgacagcccaagacctgctgctcaaaaccgtgaacacccgaaacttgggagttctgctggtcacccatgatgtttccgaggccatcgccctggccgaccacgtccttcttcttgacgacggcgccatcacacacagtttgactgtagatatccccggcgatcgccgcacccacccctcctttgcctcctacaccgctcaactccttgagtggctcgaaatcaccacacctgcctag

SEQ ID NO: 165 - protein, 243 aa, Corynebacterium glutamicum
MTATLSLKPAATVRGLRKSYGTKEVLQGIDLTINCGEVTALIGRSGSGKSTILRVLAGLSKEHSGSVEISGNPAVAFQEPRLLPWKTVLDNVTFGLNRTDISWSEAQERASALLAEVKLPDSDAAWPLTLSGGQAQRVSLARALISEPELLLLDEPFGALDALTRLTAQDLLLKTVNTRNLGVLLVTHDVSEAIALADHVLLLDDGAITHSLTVDIPGDRRTHPSFASYTAQLLEWLEITTPA

SEQ ID NO: 166 - DNA, 960 bp, Corynebacterium glutamicum
  source 1..960: mol_type = genomic DNA
atgaaatttaagaaaatcgccctcgttctcgccttcggtctaggccttgcatcctgctcatcagcttctggcgatcccgccaccaacgccgatggatccatcgatctgagcaaagtaacccttaacatcggtgatcaaatcgccggaacagaacaagtgctccaagcttcaggggagctagatgatgtcccttataaaatcgaatggtcatcatttacctctggaccaccccaaatcgaagcattaaacgcaggtcaaattgatttcgcgatcaccggaaacaccccaccgatcatcggcggccccaccaacaccaaagtggtctccgcctacaacaacgatgctttaggtgatgtcatcttggtcgccccggattcttcaataacctcggtggctgaccttgctggaaagaaagtggctgtcgcccgcggatccagcgcccacggacacctcatccaacaactagaaaaagcaggcgtgagcgttgacgacgtagaaatcaacctcctccaaccctccgacgccaaagccgctttccaaaacggccaggtagatgcgtgggcagtgtgggatccctacagctcacaggcggaactggaaggagctcaagttttggtcaggggagcgggactggtcagtgggcatggatttggtgtcgcaagtgatgaagcgctcgatgaccccgcaaaggaagccgccttggcagatttcctcgatcgcgtggccgactcttatgaatgggctgaagacaacaccgatgaatgggcgacgattttcagccaagaatccggctttgatccggaggcctctcaactgaacacccgcagcctgcgccatcaggtgccgctcgacgagtccgtcaacacctatcagaacgcgcttatcgacgctttcgtctccgcgggtctcgttgaggactttaatttcgaggacaccgtagacacccgatttgagggctaa

SEQ ID NO: 167 - protein, 319 aa, Corynebacterium glutamicum
MKFKKIALVLAFGLGLASCSSASGDPATNADGSIDLSKVTLNIGDQIAGTEQVLQASGELDDVPYKIEWSSFTSGPPQIEALNAGQIDFAITGNTPPIIGGPTNTKVVSAYNNDALGDVILVAPDSSITSVADLAGKKVAVARGSSAHGHLIQQLEKAGVSVDDVEINLLQPSDAKAAFQNGQVDAWAVWDPYSSQAELEGAQVLVRGAGLVSGHGFGVASDEALDDPAKEAALADFLDRVADSYEWAEDNTDEWATIFSQESGFDPEASQLNTRSLRHQVPLDESVNTYQNALIDAFVSAGLVEDFNFEDTVDTRFEG

SEQ ID NO: 168 - DNA, 3679 bp, Corynebacterium glutamicum
  source 1..3679: mol_type = genomic DNA
atgacattaactttccattggttcctatccacttcaggcgattcccgcggcatcatcggcggcggtcacggtgcagaaaaatccggcacctcccgcgaattgagccacagctacctcaagcagttggcgctagctgccgagaccaacggttttgaatctgtcctgacaccaacgggcacgtggtgcgaagatgcgtggattactgacgcttctttgattgaggcgacaaaacgcttgaagttcctcgttgcgcttcgccctgggcagattggacctacgctgtctgctcaaatggcttctactttccagcgtctgtctggcaaccgtttgctgatcaatgtggtcaccggtggggaagatgcggagcagcgtgcgtttggtgatttcttgaacaaggaggagcgctacgcccgtaccggagaattcttggatatcgtgagccgcttgtggcgaggcgaaaccgtcacgcaccacggtgaacacctgcaggtggagcaagctagccttgcgcatccgccagagattattccggagattctttttggtggatcgtcgccagctgcaggtgaggtggctgcacgttatgcggacacctatctcacgtggggtgaaactcccgatcaggtggcgcagaaaatcaactggatcaacgagctagcagcacagcgcggccgggaactgcgccatggaatccgcttccatgtgatcacccgcgatacgtctgaagaagcatgggtggtggcagagaagttgattagcggggtcactccagaacaggtcgctaaggctcaagccgggtttgcaacgtctaagtcggaggggcagcgccggatggctgagctgcacagcaagggtcgtgcctttactagtggctcaactgctcgtgatctggaggtgtatcccaatgtgtgggcaggcgtcggtttgcttcgcggaggtgcaggaacagcccttgtgggctcgcatgaagaggtcgccgatcgcatcgaagaatacgcagcactcggcttggatcagtttgtactgtcgggttatccaaacttggaggaggccttccacttcggtgagggtgtgattccggagctgctgcgccgcggtgtggatatcaaaaatcaagaatcacgagttttggaacctgttgggtaaacgggaagaacgagacgtcgataagcaaatttcttaaggaacctgacatgactacaaccttgactcgccccaaaatcgcgctgcccgcgcgcatctattcaccgcttgcggtgcttgttttctggcagctcggctcgagcctgggcgccatcccggagcggattctgccggcaccaaccacgatcttggccgccagctgggaggtcgccacaaatggcacgcttctcgacgccctcctcgtctcaagccaacgcgtccttctaggcttcgccctcggtgctgtcctaggcatttccctaggtgtattgacaggcatgtccagatttgcagacaccgccgttgatccgctcattcaagctgcccgcgcgctgcctcacctgggtcttgtgccgctgtttatcatctggttcggtatcggtgagctgccgaaagtactgattattagcctcggcgtgctgtatccgctgtacctcaacaccgccagcgggttcaggcaaattgatccaaagcttctggaagccggccacgtgatgggcttcggatttttccagaggttgcggaccatcatcattccttctgccgcgccgcaactttttgtcggcctgcgccaagcaagtgcggccgcctggctctcactgatcgtggcggaacaggtcaacgcccgcgaaggactcggcttcctcatcaacaatgcgcgcgatttttaccgcaccgacctcgttattttcggcctcattgtctacgccagcctcggtctgctgtctgaagcgctgatcagagcttgggaacgtcacaccttccgctaccgaaacgcataagaaagttgctcgccatgactgccacattgtcactcaaacccgcagccactgtccgtggattgcgcaaatcatacggaactaaagaagtcctccaaggaatcgacctcaccatcaactgcggcgaagtaaccgcgctgatcggacgctcaggttcaggaaaatccaccatcctgcgcgtgttggcgggcctatctaaagagcattccggctctgtagaaatttccggaaacccggccgttgccttccaagagcctcgcctgttgccgtggaaaacggtgctcgataatgtgacctttggcctcaaccgcactgatatttcctggtcagaagcacaagaacgcgcctcggcactgcttgcagaagtcaaacttcccgactccgacgccgcctggcccctcacgctctccggcggccaagcccagcgcgtctcccttgcgcgagcgctcatctccgagccagagcttttgcttctcgacgaacccttcggcgccctcgatgctctgacaagactgacagcccaagacctgctgctcaaaaccgtgaacacccgaaacttgggagttctgctggtcacccatgatgtttccgaggccatcgccctggccgaccacgtccttcttcttgacgacggcgccatcacacacagtttgactgtagatatccccggcgatcgccgcacccacccctcctttgcctcctacaccgctcaactccttgagtggctcgaaatcaccacacctgcctagaaagaaatcatgaaatttaagaaaatcgccctcgttctcgccttcggtctaggccttgcatcctgctcatcagcttctggcgatcccgccaccaacgccgatggatccatcgatctgagcaaagtaacccttaacatcggtgatcaaatcgccggaacagaacaagtgctccaagcttcaggggagctagatgatgtcccttataaaatcgaatggtcatcatttacctctggaccaccccaaatcgaagcattaaacgcaggtcaaattgatttcgcgatcaccggaaacaccccaccgatcatcggcggccccaccaacaccaaagtggtctccgcctacaacaacgatgctttaggtgatgtcatcttggtcgccccggattcttcaataacctcggtggctgaccttgctggaaagaaagtggctgtcgcccgcggatccagcgcccacggacacctcatccaacaactagaaaaagcaggcgtgagcgttgacgacgtagaaatcaacctcctccaaccctccgacgccaaagccgctttccaaaacggccaggtagatgcgtgggcagtgtgggatccctacagctcacaggcggaactggaaggagctcaagttttggtcaggggagcgggactggtcagtgggcatggatttggtgtcgcaagtgatgaagcgctcgatgaccccgcaaaggaagccgccttggcagatttcctcgatcgcgtggccgactcttatgaatgggctgaagacaacaccgatgaatgggcgacgattttcagccaagaatccggctttgatccggaggcctctcaactgaacacccgcagcctgcgccatcaggtgccgctcgacgagtccgtcaacacctatcagaacgcgcttatcgacgctttcgtctccgcgggtctcgttgaggactttaatttcgaggacaccgtagacacccgatttgagggctaa

SEQ ID NO: 169 - DNA, 492 bp, Corynebacterium glutamicum
  source 1..492: mol_type = genomic DNA
atgacttcagcaatcaccaccgcaactgatcttcgctccgtactgcgaaacgtaccaaccccaattagcttcattgcaacccacaccgatcagcctctgggcatgatcgttggttcattcgtcagcattagcgccgaaccaccattggtaggcatcttcttgcagaagagctcttcttcatggccagctatcgagcaggcattagttaccggccaagagctaggcatttctatcctcggcggggcacacgcagaccatgtgcgtaagctttctggcccatccgaccagcgctttgaaaaccttgggtgggcatccaccgaaaacggtgcgattcaccttgaaggcgctgatgcacaactaaccacgaaacttcatgatctccaggaaatcggcgatcacttctttgcagttctagaagttattgacgcttccgctgaccaagacttcagctcagcgctggtgtaccaccgctcacaggtgtcctcgctgtag

SEQ ID NO: 170 - protein, 163 aa, Corynebacterium glutamicum
MTSAITTATDLRSVLRNVPTPISFIATHTDQPLGMIVGSFVSISAEPPLVGIFLQKSSSSWPAIEQALVTGQELGISILGGAHADHVRKLSGPSDQRFENLGWASTENGAIHLEGADAQLTTKLHDLQEIGDHFFAVLEVIDASADQDFSSALVYHRSQVSSL

SEQ ID NO: 171 - DNA, 599 bp, synthetic construct
  misc_feature 1..599: note = Synthetic Construct
  source 1..599: mol_type = other DNA
tgagaattaaatacggctgtcacaagcttactcattgtgttgtcgcgaaacactggagtccacccctagagtgaggtgggtaatagaaagtgtgagaaataaggcacaatccactcctaaatgtgggggtgcaggcttgtttaacgtccaaaattcggcggtaggtgtacctaaaaacaggggtgggaaatacttggggattgctctcatcggttcgatatagactgaattgtctaggttaatatccaatatggaagaaaaactagacagttaagtagactgaatggcctactaggtgcgacactaaattttaagagggcgcgtcgaatcttcgacgtgccctcttttaaaattcgtggaacgctttgtggtgagtgtggagactttcctcttgttcggtgttaagtggttcgcgtcctcttttctcgcgaatgattctcacgtgattgataaaggcagcggagtcagggttttcgattgcggttttaagctccattccgtacagtttttgctcttctgttgtgaaataatctgttctgttgcggaagtccagtcccataagttcaaccagtgcgtcatcggataggaaaccgttaaaa

SEQ ID NO: 172 - DNA, 1464 bp, Corynebacterium glutamicum
  source 1..1464: mol_type = genomic DNA
atggccacggccgagaacacaacacaggagaatcggaaaatcctgttcaacgcatttgatatgaactgcgttgcgcatcagtccccaggactgtggacacacccgaaggataaggcgcgagactacaacactcttgattactgggtgcaccttgccaagactttggagaagggccttttcgacggccttttcatcgcagatgtgcttggaacttacgatgtttatggttctagtaatgaagcggcgttgagcagtggtgcgcaggtgcctgtcaatgatccgatccttcttgtttctgcgatggcctatgccacaaagaacctcgggtttggcattactgcaggtactgcctatgagcacccgtatccttttgcgcggcgtctggccacacttgatcacctgactaatgggcgtgtggggtggaatgtggttactggctatcttccctctgctgctcaaaacatgggtgacaccgatcagctgccacatgatgagcgctatgacaaagcagatgaatacctggaagtgatctacaagcttctcgagggctcctgggaagacgatgctgttcaaaacaatacggagacgagtgtctttacggactcctccaaagtgcacgccattaatcatcatggcaagtactttgatgtgccgggcattgccatcactgagccgagtgtgcagcgtacgccggtgatctaccaggcgggtgcatcgccgcgcggattgaaattcgctggtgagaatgcagaagcagtgtttatcaattccagcaccgtggaggcaatcaccaagactgtcgcaaaaattcgcgctgctgcggtcgctgcgggacgtgatccacatgcggtgaagatctttgcgatgcaaaccatcatcactggtgaaacagaagcagatgcgcaggcaaagctggaggaatacagtcgctatatcgatcctgtcggtggtctgaccttgatgtctggatggaccggcgcggatctgtcgcagtatgacctggatgaaccgatcaccaatattgagtcaaacgctattcagtccactgcagccaccattagcaacggcaccggtgaaggtgcgtggacggtacgcaaactgggtgaggcaaccggcatcggcggcttcggaccagtgcttgtgggatctggcgctaacgttgccgcggaacttgcacgcatccaggatctcagcgatgttgatggtttcaaccttgcttatgccatcaccccaggaacttttgaagatgtcgtggactttgtggtgcctgagctgcaaaaacttagccgctacaagacggaatacgcgccgggttccttgcgcaacaaattgctcggtaaaggtgatcgcctggacgatacccaccgcggcgcaagctaccgcctaggcgctcggaactccaccgccactattgatctcagttccatatccgcccaactagtttcccagggagcccactcatga

SEQ ID NO: 173 - protein, 487 aa, Corynebacterium glutamicum
MATAENTTQENRKILFNAFDMNCVAHQSPGLWTHPKDKARDYNTLDYWVHLAKTLEKGLFDGLFIADVLGTYDVYGSSNEAALSSGAQVPVNDPILLVSAMAYATKNLGFGITAGTAYEHPYPFARRLATLDHLTNGRVGWNVVTGYLPSAAQNMGDTDQLPHDERYDKADEYLEVIYKLLEGSWEDDAVQNNTETSVFTDSSKVHAINHHGKYFDVPGIAITEPSVQRTPVIYQAGASPRGLKFAGENAEAVFINSSTVEAITKTVAKIRAAAVAAGRDPHAVKIFAMQTIITGETEADAQAKLEEYSRYIDPVGGLTLMSGWTGADLSQYDLDEPITNIESNAIQSTAATISNGTGEGAWTVRKLGEATGIGGFGPVLVGSGANVAAELARIQDLSDVDGFNLAYAITPGTFEDVVDFVVPELQKLSRYKTEYAPGSLRNKLLGKGDRLDDTHRGASYRLGARNSTATIDLSSISAQLVSQGAHS

SEQ ID NO: 174 - DNA, 1062 bp, Corynebacterium glutamicum
  source 1..1062: mol_type = genomic DNA
atgatctcaccgcaaacaatcatcgacaatcttgctccagtcctcgcggagatcgcagcaacggctgcgcagcgcgaacaggatcgagaattcagccgtgatttggcaaagcagcttagtgccggtgggttcactaaactccgcatgggttttagccttccagaagcatttgaggtgctggtggcagcggccgctgcagattcaaatatcgcgcagggattgcggcctcacttcttggcagtggagagcttattgatcgctccttattcagagcaccgcaccaagtggctgcgaaaaatcgctgagaaaggcgtggtcattggcaatgcgttgactgaagtgggaaacaagccaggtgagctgaaaaccaagatccgaaaggaaggcgagtcttacgttctcagcgacaccaagttctattccacgggcagtctttacgccgactggattcaagtacatgcgaaagatgaggaagatcaagatgttttcgcctttgttgatcgcgacgcatcaggcgttgttttggtggattactgggacgaatttgggcaacaactatttgcctccggaaccagcttctttgaaaaagtcgtggtagatccactggacattgtcacccgtgattacaccgcacccagcgctttccaggcgctggcgcagtcacatcatttgtctacgttgactggtatttcgcaggcaatcacccgtgacattgttacctatgtacaaaaccgcacccgtattttcagccacggaagtggtgaccttccacgctttgatccgcaggtgcaacaggtggtgggtgaggtgaaagccaagtcgtatgcagtcgagaaaatctttcagggtttcgcacaagaactggatcttgtcgtcgataaggcaaaagccggcactgctacggaggtcgatctggccgctgtcgacctgagcgcctaccaagcccagttagcggtggcacctttggtgctgagccaggccacccaggcctttgaggttggcgcagatgccttaaacggcggccacgtggctgcccaatacacgatcggatccttgtcatga

SEQ ID NO: 175 - protein, 360 aa, Corynebacterium glutamicum
MISPQTIIDNLAPVLAEIAATAAQREQDREFSRDLAKQLSAGGFTKLRIPVEFGGLGFSLPEAFEVLVAAAAADSNIAQGLRPHFLAVESLLIAPYSEHRTKWLRKIAEKGVVIGNALTEVGNKPGELKTKIRKEGESYVLSDTKFYSTGSLYADWIQVHAKDEEDQDVFAFVDRDASGVVLVDYWDEFGQQLFASGTSFFEKVVVDPLDIVTRDYTAPSAFQALAQSHHLSTLTGISQAITRDIVTYVQNRTRIFSHGSGDLPRFDPQVQQVVGEVKAKSYAVEKIFQGFAQELDLVVDKAKAGTATEVDLAAVDLSAYQAQLAVAPLVLSQATQAFEVGADALNGGHVAAQYTIGSLS

SEQ ID NO: 176 - DNA, 1200 bp, Corynebacterium glutamicum
  source 1..1200: mol_type = genomic DNA
atgagccaagaaattttgagccattttgcacccgcattagagcgcattcgaagcggcgccgtcgagcgcgaacagcagcgcgccttgccagtggaagagattaaagagctggtagagctaggttttactgggcttcgagtgcccgaagaactaggcggtgcgggcgcttccctggaaagcgtagttgagttactgatcgagatcgcgggcgccgattccaatatcgcccaagccctgcgcggacattttgccttcgtggaactactcctggaagcgccggagagcgaattccgcacccattggctgcgcgaagtcgccaccggaagacttgtgggcaacgccgaaagtgagaaacgcggcgtttacggcgatccgcagaccttcatcgatgaggtggagactgaaaacggaccgattttcgtgctcaacggcactaagttttataccaccggcacctattttgcggactacacctggaccaccgcgctgctgcgcaacctcaacggccaagaaactttggtcagtttgccggtcgatctgcacgcgccgggcgtggatgttgttgatgattggagcgggtttgggcaaaagctcaccgcctctggaacgaccacgtttaaagacctggaggtggatccgcggtggatcattccacgcactgatgcgcccacgctggtgtggacgtatctgcagctgagcctgctgaccgtgctggttggcagtgccgcagcagctgtcgatgaggtggttgcccgcgcccaatcctccaccagaaatgcgtggaaccctggcgtcgagcgccgcagcgatccggccgcaaccatagcgatcggcgacgcacgcagccgagtcaccgttattcgtggagcgcttctcgacgccacccgccacgtttccaacgccgccacgatcgtaacccccgaagccttcaacgaggcggacgctattgttgcagcgctctggcccatcgtctccggacaagctttggtggtgacatccaacgttttcgatgcggtgggtgcatctgcagtgcttggtgagcattccattgatcgccactggcgcaatgtgcgtaccgtgtcctcaaacaacccggtgttcctggccaagaatgcagtgggggagtatgccctcaacggcactcctgtgggtaccaacattggtaaagcactgagccgtccggtgagcctaagcagctag

SEQ ID NO: 177 - protein, 399 aa, Corynebacterium glutamicum
MSQEILSHFAPALERIRSGAVEREQQRALPVEEIKELVELGFTGLRVPEELGGAGASLESVVELLIEIAGADSNIAQALRGHFAFVELLLEAPESEFRTHWLREVATGRLVGNAESEKRGVYGDPQTFIDEVETENGPIFVLNGTKFYTTGTYFADYTWTTALLRNLNGQETLVSLPVDLHAPGVDVVDDWSGFGQKLTASGTTTFKDLEVDPRWIIPRTDAPTLVWTYLQLSLLTVLVGSAAAAVDEVVARAQSSTRNAWNPGVERRSDPAATIAIGDARSRVTVIRGALLDATRHVSNAATIVTPEAFNEADAIVAALWPIVSGQALVVTSNVFDAVGASAVLGEHSIDRHWRNVRTVSSNNPVFLAKNAVGEYALNGTPVGTNIGKALSRPVSLSS

SEQ ID NO: 178 - DNA, 1179 bp, Corynebacterium glutamicum
  source 1..1179: mol_type = genomic DNA
atgagtgaaaacaatcccactaccttgcactggttcctacccacctatggcgattctcgcggaatcacagccggcgggcatggcttcggcttccactccggaagccggacagcagacctcgattacctctcccaaattgccctggccgctgaacgaaacggttttgaatccgtcctgactcccactggattgtggtgcgaagatgcgtggatcaccaccgcagcgctgctgtctaggacatcaaaactgaaattcctcgttgctattcgaccaggccaagttagccccaccatcatcgcgcagcagggtgctgccttccagaaattctcaaataaccgcctgctcatcaacgtcgtggtgggtggcgaagaccatgaacagcgcgctttcgctgattattcttccaaagaggagcgctaccacaaggctgatgaaaccttagagatcatcgatcacctatggaacagcgcagaacctctaaatttccagggtgaattcctcagtgtggaaaacgcggtattgaaggaacagcccgaggtttccccaccgatttactttggcggatcctcacaactcggcatcgaaatcgcagcccaacattccgatgtttatctcacctggggtgaacctgcggaaaaggtagaggagaagcttgcccgggtgcgcgccgaagcagataagcgaaaccgcgaactagactatggcatccgcctgcatgtcattgctcgaccaactgaggatgaagcctggtcagtggctcaaaatcttcttgaccaacttgatcaggaagaggttgcccgcattcaggaagggcttgcgcgttctcaatcggaaggtcagcgtcgcatgacggaacttcatggacaaggggcagcattcacagcaggagcagatgctcgctcccttgaaattgcaccgaatctctgggcaggtgttgggctagtccgcggtggcgccggcacagcgttggtgggttcctatgagcaagtcgcgcaagcaattttgcgataccgcgatattggtctgagccacttcattttctccggctatccacatttggaggaaacctatcacgtgggcgaaggagtggtacctgagctcctcaaattgggtgttccggtgaacaaccatgaagaacaacgcaacgacgtggtagcgactccgtttatttccagatag

SEQ ID NO: 179 - protein, 392 aa, Corynebacterium glutamicum
MSENNPTTLHWFLPTYGDSRGITAGGHGFGFHSGSRTADLDYLSQIALAAERNGFESVLTPTGLWCEDAWITTAALLSRTSKLKFLVAIRPGQVSPTIIAQQGAAFQKFSNNRLLINVVVGGEDHEQRAFADYSSKEERYHKADETLEIIDHLWNSAEPLNFQGEFLSVENAVLKEQPEVSPPIYFGGSSQLGIEIAAQHSDVYLTWGEPAEKVEEKLARVRAEADKRNRELDYGIRLHVIARPTEDEAWSVAQNLLDQLDQEEVARIQEGLARSQSEGQRRMTELHGQGAAFTAGADARSLEIAPNLWAGVGLVRGGAGTALVGSYEQVAQAILRYRDIGLSHFIFSGYPHLEETYHVGEGVVPELLKLGVPVNNHEEQRNDVVATPFISR

SEQ ID NO: 180 - DNA, 5028 bp, Corynebacterium glutamicum
  source 1..5028: mol_type = genomic DNA
atggccacggccgagaacacaacacaggagaatcggaaaatcctgttcaacgcatttgatatgaactgcgttgcgcatcagtccccaggactgtggacacacccgaaggataaggcgcgagactacaacactcttgattactgggtgcaccttgccaagactttggagaagggccttttcgacggccttttcatcgcagatgtgcttggaacttacgatgtttatggttctagtaatgaagcggcgttgagcagtggtgcgcaggtgcctgtcaatgatccgatccttcttgtttctgcgatggcctatgccacaaagaacctcgggtttggcattactgcaggtactgcctatgagcacccgtatccttttgcgcggcgtctggccacacttgatcacctgactaatgggcgtgtggggtggaatgtggttactggctatcttccctctgctgctcaaaacatgggtgacaccgatcagctgccacatgatgagcgctatgacaaagcagatgaatacctggaagtgatctacaagcttctcgagggctcctgggaagacgatgctgttcaaaacaatacggagacgagtgtctttacggactcctccaaagtgcacgccattaatcatcatggcaagtactttgatgtgccgggcattgccatcactgagccgagtgtgcagcgtacgccggtgatctaccaggcgggtgcatcgccgcgcggattgaaattcgctggtgagaatgcagaagcagtgtttatcaattccagcaccgtggaggcaatcaccaagactgtcgcaaaaattcgcgctgctgcggtcgctgcgggacgtgatccacatgcggtgaagatctttgcgatgcaaaccatcatcactggtgaaacagaagcagatgcgcaggcaaagctggaggaatacagtcgctatatcgatcctgtcggtggtctgaccttgatgtctggatggaccggcgcggatctgtcgcagtatgacctggatgaaccgatcaccaatattgagtcaaacgctattcagtccactgcagccaccattagcaacggcaccggtgaaggtgcgtggacggtacgcaaactgggtgaggcaaccggcatcggcggcttcggaccagtgcttgtgggatctggcgctaacgttgccgcggaacttgcacgcatccaggatctcagcgatgttgatggtttcaaccttgcttatgccatcaccccaggaacttttgaagatgtcgtggactttgtggtgcctgagctgcaaaaacttagccgctacaagacggaatacgcgccgggttccttgcgcaacaaattgctcggtaaaggtgatcgcctggacgatacccaccgcggcgcaagctaccgcctaggcgctcggaactccaccgccactattgatctcagttccatatccgcccaactagtttcccagggagcccactcatgatctcaccgcaaacaatcatcgacaatcttgctccagtcctcgcggagatcgcagcaacggctgcgcagcgcgaacaggatcgagaattcagccgtgatttggcaaagcagcttagtgccggtgggttcactaaactccgcattccagttgaatttggcggtttgggttttagccttccagaagcatttgaggtgctggtggcagcggccgctgcagattcaaatatcgcgcagggattgcggcctcacttcttggcagtggagagcttattgatcgctccttattcagagcaccgcaccaagtggctgcgaaaaatcgctgagaaaggcgtggtcattggcaatgcgttgactgaagtgggaaacaagccaggtgagctgaaaaccaagatccgaaaggaaggcgagtcttacgttctcagcgacaccaagttctattccacgggcagtctttacgccgactggattcaagtacatgcgaaagatgaggaagatcaagatgttttcgcctttgttgatcgcgacgcatcaggcgttgttttggtggattactgggacgaatttgggcaacaactatttgcctccggaaccagcttctttgaaaaagtcgtggtagatccactggacattgtcacccgtgattacaccgcacccagcgctttccaggcgctggcgcagtcacatcatttgtctacgttgactggtatttcgcaggcaatcacccgtgacattgttacctatgtacaaaaccgcacccgtattttcagccacggaagtggtgaccttccacgctttgatccgcaggtgcaacaggtggtgggtgaggtgaaagccaagtcgtatgcagtcgagaaaatctttcagggtttcgcacaagaactggatcttgtcgtcgataaggcaaaagccggcactgctacggaggtcgatctggccgctgtcgacctgagcgcctaccaagcccagttagcggtggcacctttggtgctgagccaggccacccaggcctttgaggttggcgcagatgccttaaacggcggccacgtggctgcccaatacacgatcggatccttgtcatgagccaagaaattttgagccattttgcacccgcattagagcgcattcgaagcggcgccgtcgagcgcgaacagcagcgcgccttgccagtggaagagattaaagagctggtagagctaggttttactgggcttcgagtgcccgaagaactaggcggtgcgggcgcttccctggaaagcgtagttgagttactgatcgagatcgcgggcgccgattccaatatcgcccaagccctgcgcggacattttgccttcgtggaactactcctggaagcgccggagagcgaattccgcacccattggctgcgcgaagtcgccaccggaagacttgtgggcaacgccgaaagtgagaaacgcggcgtttacggcgatccgcagaccttcatcgatgaggtggagactgaaaacggaccgattttcgtgctcaacggcactaagttttataccaccggcacctattttgcggactacacctggaccaccgcgctgctgcgcaacctcaacggccaagaaactttggtcagtttgccggtcgatctgcacgcgccgggcgtggatgttgttgatgattggagcgggtttgggcaaaagctcaccgcctctggaacgaccacgtttaaagacctggaggtggatccgcggtggatcattccacgcactgatgcgcccacgctggtgtggacgtatctgcagctgagcctgctgaccgtgctggttggcagtgccgcagcagctgtcgatgaggtggttgcccgcgcccaatcctccaccagaaatgcgtggaaccctggcgtcgagcgccgcagcgatccggccgcaaccatagcgatcggcgacgcacgcagccgagtcaccgttattcgtggagcgcttctcgacgccacccgccacgtttccaacgccgccacgatcgtaacccccgaagccttcaacgaggcggacgctattgttgcagcgctctggcccatcgtctccggacaagctttggtggtgacatccaacgttttcgatgcggtgggtgcatctgcagtgcttggtgagcattccattgatcgccactggcgcaatgtgcgtaccgtgtcctcaaacaacccggtgttcctggccaagaatgcagtgggggagtatgccctcaacggcactcctgtgggtaccaacattggtaaagcactgagccgtccggtgagcctaagcagctagacgtgtgatttcgctggtttttagatggttttcaagccagcgagaccacattagtttcacgctggttgaaacctttgagatcaatatagaccgtgtggtctactcgaggaatgagtgaaaacaatcccactaccttgcactggttcctacccacctatggcgattctcgcggaatcacagccggcgggcatggcttcggcttccactccggaagccggacagcagacctcgattacctctcccaaattgccctggccgctgaacgaaacggttttgaatccgtcctgactcccactggattgtggtgcgaagatgcgtggatcaccaccgcagcgctgctgtctaggacatcaaaactgaaattcctcgttgctattcgaccaggccaagttagccccaccatcatcgcgcagcagggtgctgccttccagaaattctcaaataaccgcctgctcatcaacgtcgtggtgggtggcgaagaccatgaacagcgcgctttcgctgattattcttccaaagaggagcgctaccacaaggctgatgaaaccttagagatcatcgatcacctatggaacagcgcagaacctctaaatttccagggtgaattcctcagtgtggaaaacgcggtattgaaggaacagcccgaggtttccccaccgatttactttggcggatcctcacaactcggcatcgaaatcgcagcccaacattccgatgtttatctcacctggggtgaacctgcggaaaaggtagaggagaagcttgcccgggtgcgcgccgaagcagataagcgaaaccgcgaactagactatggcatccgcctgcatgtcattgctcgaccaactgaggatgaagcctggtcagtggctcaaaatcttcttgaccaacttgatcaggaagaggttgcccgcattcaggaagggcttgcgcgttctcaatcggaaggtcagcgtcgcatgacggaacttcatggacaaggggcagcattcacagcaggagcagatgctcgctcccttgaaattgcaccgaatctctgggcaggtgttgggctagtccgcggtggcgccggcacagcgttggtgggttcctatgagcaagtcgcgcaagcaattttgcgataccgcgatattggtctgagccacttcattttctccggctatccacatttggaggaaacctatcacgtgggcgaaggagtggtacctgagctcctcaaattgggtgttccggtgaacaaccatgaagaacaacgcaacgacgtggtagcgactccgtttatttccagatag

SEQ ID NO: 181 - DNA, 1181 bp, synthetic construct
  misc_feature 1..1181: note = Synthetic Construct
  source 1..1181: mol_type = other DNA
ttgctgtggcagcgggacgccctctggctgatttccagggcggacagccagcggatgcactaaagcagtggactgatcaggtggttcaggctgtgggtggacagctggaaggactgccagaggaggccacagacggcgaacaagaagacgctcctgtggaagacccccgcttcgatgctgccactgatgctctaaaccgtggcgctttcgatgaggcgattgcggtttatgagtccattttggcgcaggagccaaacaacgctgatgcgaagcaggcacgcgataccgcaaagctgttgggccggcttgccacggtggatccttcggtggatgttgtcgctgctgcagatgctgatccaacaaacgttgatctggcctacacagcagctgacgcggctgttgttgcgggtgatcctgaggctgcctttgatcgtttaattgctctgctgaccatcagcgctggcgatcagaagaatcaggtgaaggaacgtttgctggagctgtttggcatgtttgagaccgccgatccccgtgtgctgcaggcgcgaggaaagatggccagcgcgctgttctaaaaccactctctatccagaaaaatatagaccgcttagtcttttccaggactaagtggtctacatttttacccaaaatgcagctcacgcaatagacatctcggtctatatacttgccaccttcgcgccctgcaaatccccacactacttatatccagcccgaaaataatacttctctctagacgaagcggtctgtttaagtatgtgccgtatgtctgagtatggcaaaggggcgttgaggtgctcagacggccatacaaagcatcgtagtttgtgcatatgagggctgctttttgccgctgtgagttggctgttcaggaatccgtcctcgggttggggagaggggctgaaaccgaacgaatattcgcataaatctttcgaattggcgcgaatattcgatcccgctggcctccgaactagccattttggtagaaaatttgcatccttgagccggtattggcccaaggatgcattttcctgtgtcctacttctgcagtgagtagaactgagcactcatcgctgggacatgcagtgagagtgagtaggcgaaattatcgcgtggtgtcgctgctgcttgaacggaagtagcgatatcgttttctgcaccga

SEQ ID NO: 182 - DNA, 851 bp, synthetic construct
  misc_feature 1..851: note = Synthetic Construct
  source 1..851: mol_type = other DNA
gctgcccatgccttggtgtgctcgtttgcatactggccgtagccggctggcattgcttcgtcgaggacggggaagttgaaatcggcgatatcaacgagctcgtactgtgcatcgttgcgctcttgtgcgcgctcgagaacccagttggctacgtcaatgttgacgcggcctggacgggtggaaccggtgatgatggcgatcttgctcatggtgtactcctctaaaagttaattaagttgatgtatcaacaacttgacgtacctagcctatgttaactttccgcgtcatgcaagcgctttgggattccagtctatttctcaaggtcaacagggtgggggagtcgtcgaggttttcattcagggtgaatgctctccttgtttcagatgttcaacgctccataaagtagaccgcaatctagacaaagatgtctattttaattaaggagcagaacatcacggatcggctgctttagcagaggcaaaagccgggattcagttggtgcggcttccagaggttgtcacaaaccgtcttatatgaccaacggccaggagatgtgaggttggtcggtcacctagtttggtcggtgcttggacgccagaaaaactggccgggccccaccaaaagtgacctctcagattcgcctctaagcgcccaaagccctgaaccatacaaatactcaatcttgatcttcgacctcttaaatcgcatttgcactggcatccctcgtagggatccccaagcaaaaaaccgccgaccacaatggccggcggtttttcgaactaagagctcttaggtcgcggtggtgtagtcaaccacggagtattcctgcagcttggacagctggtggacag

SEQ ID NO: 183 - DNA, 599 bp, synthetic construct; identical to SEQ ID NO: 171
  misc_feature 1..599: note = Synthetic Construct
  source 1..599: mol_type = other DNA

SEQ ID NO: 184 - DNA, 1536 bp, Escherichia coli
  source 1..1536: mol_type = genomic DNA
atggctacatcagtacagacaggtaaagctaagcagctcacattacttggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctggggccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttatctgcactccggtgcccccgttgctatcgaaatggattcgaagaccttcttccctgacttctctaaagtgggcaccctggtagtatttgttgccttcattttgagttatatgggcgtagaagcatccgcaacccacgtcaatgaaatgagcaacccagggcgcgactatccgttggctatgttactgctgatggtggcggcaatctgcttaagctctgttggtggtttgtctattgcgatggtcattccgggtaatgaaatcaacctctccgcaggggtaatgcaaacctttaccgttctgatgtcccatgtggcaccagaaattgagtggacggttcgcgtgatctccgcactgctgttgctgggtgttctggcggaaatcgcctcctggattgttggtccttctcgcgggatgtatgtaacagcgcagaaaaacctgctgccagcggcattcgctaaaatgaacaaaaatggcgtaccggtaacgctggtcatttcgcagctggtgattacgtctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcaaataccggcgtcactctggagccaatcaacagtcagaacgcaccaaaaggtcacttcttcctgcacccgcgtgcacgttcaccacactatattgtgatgaatgacaagaaacactaa

SEQ ID NO: 185 - protein, 511 aa, Escherichia coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRKGKANTGVTLEPINSQNAPKGHFFLHPRARSPHYIVMNDKKH

SEQ ID NO: 186 - DNA, 1413 bp, Escherichia coli
  source 1..1413: mol_type = genomic DNA
atggctacatcagtacagacaggtaaagctaagcagctcacattacttggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctggggccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttatctgcactccggtgcccccgttgctatcgaaatggattcgaagaccttcttccctgacttctctaaagtgggcaccctggtagtatttgttgccttcattttgagttatatgggcgtagaagcatccgcaacccacgtcaatgaaatgagcaacccagggcgcgactatccgttggctatgttactgctgatggtggcggcaatctgcttaagctctgttggtggtttgtctattgcgatggtcattccgggtaatgaaatcaacctctccgcaggggtaatgcaaacctttaccgttctgatgtcccatgtggcaccagaaattgagtggacggttcgcgtgatctccgcactgctgttgctgggtgttctggcggaaatcgcctcctggattgttggtccttctcgcgggatgtatgtaacagcgcagaaaaacctgctgccagcggcattcgctaaaatgaacaaaaatggcgtaccggtaacgctggtcatttcgcagctggtgattacgtctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaataa

SEQ ID NO: 187 - protein, 470 aa, Escherichia coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRK

SEQ ID NO: 188 - DNA, 1507 bp, Lactobacillus brevis
  source 1..1507: mol_type = genomic DNA
agtggatgaaaataagtctgaacagcaaattgatacgcaaaaggcaaatcgaataacgggctttcagttattttccatgacgacttccatggtcatgacggtttacggttttgcggcatttgctaagcaaggaccaaccgctttcttttacctgtttttagccgggatcttatggtttttaccggtgactcggacctcaggagagatggcgtccatcgatggctggagtaaaggcggtatctttacttggtcacggaatatgttaggtgaacgcgcaggttggtcggcattattctaccaatggattcacattacggttgggatgaatacaatgatgtatttcattatcggctgtttttcagtaacgtttggcttacccattatcacggataacccgttagttaagtttattttgatgatgatcattttatggggccttatctttctacagcaaaaaggaacgagtgttaccggtaagattgcccaatggtgtttcacgttaggggtgattatcccagtcttcttcttactctttctatttattttgtacctcgtacaaggaaacccagcgatgattcacgtcaacatgcacaccatttttccatcgagttggaatgggagtgtcttagtgggatttgttccgtttatcttggcttttgccggtgccgaagggtcggcaccacacgttaaggacttggataaacctagcatctatcctaaggttatgatggcactagcagttgctgcgatctgttccgatattatcggcagtatggcgattgctatgacgattccgaataatcagattcagttaagtaatgggattgtttacgcctatggggccttagttgcccgctacggtgtgggtgtggtctttgtcgaaaagctaacagggttcttattagccgttggtgtcttgggtgaaatcagcagttgggtcgttggtcctaacgcggggatgtttgaagcagctaaagctggctacttaccaccgcgcttctctaaggccaataaatatgggattgaaaccaatgtcatggtccttcaaggggtcatcgtgtcgattgttggggcgctgttgacgtttggggctggcgggaacaaggcgagcctttccttccagactgcaatgagtttgacggtagccttatacaccttgatgtatatgctgatgtttattagttatttggtcttacagttcaagtatgtcgatttgcatcgggactttgtggccgctaagtcgcgttggttgcggatcacttacggtatcctagggtttattttatcggcctttggatttgtcgtgaccttcttcccgccagcagatttatccgttgcctcaaaacacacgtacctaatgttgttagtgagtgcctttgtggtcatgctcgttttgccatttatcttgtaccgtttccacgacaaatgggccttgcagctaggtgtcaatgtggatgaagtggcagcaccgaccggtcatgaggcgttggaaaaagaaaccaagtag

SEQ ID NO: 189 - protein, 501 aa, Lactobacillus brevis
MDENKSEQQIDTQKANRITGFQLFSMTTSMVMTVYGFAAFAKQGPTAFFYLFLAGILWFLPVTRTSGEMASIDGWSKGGIFTWSRNMLGERAGWSALFYQWIHITVGMNTMMYFIIGCFSVTFGLPIITDNPLVKFILMMIILWGLIFLQQKGTSVTGKIAQWCFTLGVIIPVFFLLFLFILYLVQGNPAMIHVNMHTIFPSSWNGSVLVGFVPFILAFAGAEGSAPHVKDLDKPSIYPKVMMALAVAAICSDIIGSMAIAMTIPNNQIQLSNGIVYAYGALVARYGVGVVFVEKLTGFLLAVGVLGEISSWVVGPNAGMFEAAKAGYLPPRFSKANKYGIETNVMVLQGVIVSIVGALLTFGAGGNKASLSFQTAMSLTVALYTLMYMLMFISYLVLQFKYVDLHRDFVAAKSRWLRITYGILGFILSAFGFVVTFFPPADLSVASKHTYLMLLVSAFVVMLVLPFILYRFHDKWALQLGVNVDEVAAPTGHEALEKETK

SEQ ID NO: 190 - DNA, 1054 bp, Escherichia coli
  source 1..1054: mol_type = genomic DNA
agtgaacatatatatcgggtggcttttcaaattaatccctttgattatgggcttaatttgtatcgcgttaggtggctttgtgctggaaagttcagggcaaagcgagtatttcgtcgcgggtcatgtgctgatttctctggcggccatatgcctggcattattcactaccgcatttattatcatttcgcagctcacgcgcggcgttaatacgttttacaatacattgttccccattattggctatgcggggtcaattatcaccatgatatggggttgggcactgttagcaggcaatgatgtgatggcagacgagtttgtcgccggccatgttattttcggcgttggtatgattgccgcctgtgtatcgacggtggcagcgtcatccggtcactttctgctcattcccaaaaatgcagcggggagcaagagcgacggaacaccggtacaggcttattcttcattaatcggtaactgcctcattgccgttcccgttttactcaccctgctcggtttcatttggtctattacgctgttacgtagtgctgacataactccgcattatgtcgcgggtcacgtattgcttgggttaaccgcaatctgtgcctgtctaattggccttgttgccacaattgtccatcaaacacgtaatacgttttcaactaaagaacactggctgtggtgttattgggttatttttctcggctcaatcacggtactgcaggggatatacgtcttagtcagttccgatgcaagcgcccgactggctcccggcattattcttatttgcctcggaatgatctgttacagcatattctcaaaagtctggctactggcactggtatggagacgtacctgttcgttagccaacagaataccgatgattcccgtcttcacctgcctgttttgccttttcctggcatcgtttcttgcggaaatggcgcagaccgacatgggatattttattccttcgcgagttctggtcggtttgggagcggtatgctttacgttgttctcaatcgtttcaatattagaagcgggttctgctaaaaaataa

SEQ ID NO: 191 - protein, 350 aa, Escherichia coli
MNIYIGWLFKLIPLIMGLICIALGGFVLESSGQSEYFVAGHVLISLAAICLALFTTAFIIISQLTRGVNTFYNTLFPIIGYAGSIITMIWGWALLAGNDVMADEFVAGHVIFGVGMIAACVSTVAASSGHFLLIPKNAAGSKSDGTPVQAYSSLIGNCLIAVPVLLTLLGFIWSITLLRSADITPHYVAGHVLLGLTAICACLIGLVATIVHQTRNTFSTKEHWLWCYWVIFLGSITVLQGIYVLVSSDASARLAPGIILICLGMICYSIFSKVWLLALVWRRTCSLANRIPMIPVFTCLFCLFLASFLAEMAQTDMGYFIPSRVLVGLGAVCFTLFSIVSILEAGSAKK

SEQ ID NO: 192 - DNA, 1422 bp, unidentified
  source 1..1422: mol_type = genomic DNA
atgatcagtgatcaagttgcagcaccaagagtgcttggcctgtttggccttgcaatgatcaacatcgttgccatcgccagtcttcgcgatcttccacagatggcaagctatggcgttggggccatttttttctatgtgttagccgcgctcgtctttttcgtgccggtctctttggtagcagcggagttggcaacagcgtggcccgaacggggaggcgtctatatctgggtaagagaagcttttggaaaacgctggggctttgttgccgtttttctgcaatggtttcagaacctttgctggtttccagttgtactcacctttggagccgcatcactggcctatgccatcgcgcctggagattccgaagcactggccaataacaaaacctttattgtcagcatcgttctcactacttattgggctgcagttctttttaacttccgaggcctctccggttcagcaaaattaagcgccctcggtgctttctctggtgtatttattcctggcataacactgatcatcttggcaggagttgggctggcgacaggctatccatcacgattgctagagaatggcaaccaggtcattccagatatcagccagttttccaatatcacttttgcagtcagtgtctggcttgcttttgccgggatggaaatgaccgccgcccacgcgcgcgaagtcaaatcaccaggcagaaactttccgctaggaatcatgcttgcggcgatgatcatattgactatttttattcttggtgctctttcaattgcaacagctctggctccaggcgaatacgaactgcaaagtggagtgatcaaagcgctcaagcatatgctgagtcgctacaaacttgacgggtgggctcggctgtttgcacttgggatggcattgggtgttttcggaagcgtcaatgcctggatcgttggacccagcaaaggcatgctggcttcaactgaagacggcgctttgccgccaactctgctgaaagtgaaccagaacaacgtgccaactcatatcctgctgattcaaagtgcagttgtctcaatcatctgcttggcatttctattacaaccaacagtcgcttcagcctatttcatgattagcgttctgaccattcagatgtatctttgcatgtacgtgatgatgttccttgccgccttgagactaaggcaaaaacatccaaatcattcaggcagatttcgcatccccggaggtaaatttggcctgtggagcacttgcatcataggaggccttggttcactttttgcaatcatccttggcttctttccccctgaccaacttgagagttccggaatgaattctcaatccttcattttgtttttagccacaggattatcagtggcaatagcatctccgcacctgataggcttcaggaggcagggtcgacatgcacattaa

SEQ ID NO: 193 - protein, 473 aa, unidentified
MISDQVAAPRVLGLFGLAMINIVAIASLRDLPQMASYGVGAIFFYVLAALVFFVPVSLVAAELATAWPERGGVYIWVREAFGKRWGFVAVFLQWFQNLCWFPVVLTFGAASLAYAIAPGDSEALANNKTFIVSIVLTTYWAAVLFNFRGLSGSAKLSALGAFSGVFIPGITLIILAGVGLATGYPSRLLENGNQVIPDISQFSNITFAVSVWLAFAGMEMTAAHAREVKSPGRNFPLGIMLAAMIILTIFILGALSIATALAPGEYELQSGVIKALKHMLSRYKLDGWARLFALGMALGVFGSVNAWIVGPSKGMLASTEDGALPPTLLKVNQNNVPTHILLIQSAVVSIICLAFLLQPTVASAYFMISVLTIQMYLCMYVMMFLAALRLRQKHPNHSGRFRIPGGKFGLWSTCIIGGLGSLFAIILGFFPPDQLESSGMNSQSFILFLATGLSVAIASPHLIGFRRQGRHAH

SEQ ID NO: 194 - DNA, 1494 bp, unidentified
  source 1..1494: mol_type = genomic DNA
atgctaaacgtagaatctccacgtgcttcaaacgcccctttgcatccgttacagaaggccaaaccattaactctactccctttagttgctcttattttctacgacgtctctggaggtccctttggaattgaagatgcagtcagcaagggagcaccttttcttgccataactgggtttttaatattaccatttgcatggagtatccctgaagcattagtcacagctgaattagctactacattccctgaaaatagtggatttgtcgcttgggtgacggcagcgtttggaccttggtggggttttcaagaaggattttggaagtggttgagtggagtaacagataatgcagtttatccagtgatttttttgacttatctacagcaattagtgccagtattagagactggattgcctcgaacgctgtttttagtaggacttaatttggcactgacgtacatgaattacagagggttgcatgttgtcggtaaagtagctgtagctatgacagcatttactctactgccctttattctcatgtgtctgttaggtgctcctcatgtggatccttcaaattggctggttgttgactggaaagcagtgcaatgggttcctttcttaaatgttatgttttggtcattgaattactgggactctgtatctacgcttgctggtgaagttagtgatcccgcaaggacctttccacgtgcgttgagtattgctgttgttcttgtcattagtgcttaccttctcccattattgataggagtaggagtggagactggatcgacgagtttagattggacattgggctactttgccaccgttgcggaacacgtcggtgggaaatggctcgcttggtggattgttgtagcagcagcaatcagtcagttgggtcagtttgaagctgaaatgagcactgactcttatcagctattaggaatggccgaacgcggttttatcccttcgatttttgcacagcgatcttctcacggcaccccaacctatgctatattagctagtagtctcggtattgcagcaatggcaagctttaatttcttagagattgtagaactgctgaatgttgtctattgcatggctgaattgcttgaatttacagcgttcctgtatctacgcttgaggtacccaaatcttcacagaccatttcgagtgcctttgtcaactttaggatgtgccatcatgctcgcccctgcgtttatcctgctttccatcatgttaatgttgcctattttcagaggcgattggcgcgtcgtcggatttacttttttggctttcgttttgggtgctgtcctgtaccctttaatgcagtacgctcgtcaggaaggctggtgtgcctttgaaggtgtgacccctgatgagtttaaggagcatttgtatactacatatacccctgtacgacccgttggtagcagcagagaaagcggtgatatgatgactgttatggaagatcagcaacagggagaacaaattaattga

SEQ ID NO: 195 - protein, 497 aa, unidentified
MLNVESPRASNAPLHPLQKAKPLTLLPLVALIFYDVSGGPFGIEDAVSKGAPFLAITGFLILPFAWSIPEALVTAELATTFPENSGFVAWVTAAFGPWWGFQEGFWKWLSGVTDNAVYPVIFLTYLQQLVPVLETGLPRTLFLVGLNLALTYMNYRGLHVVGKVAVAMTAFTLLPFILMCLLGAPHVDPSNWLVVDWKAVQWVPFLNVMFWSLNYWDSVSTLAGEVSDPARTFPRALSIAVVLVISAYLLPLLIGVGVETGSTSLDWTLGYFATVAEHVGGKWLAWWIVVAAAISQLGQFEAEMSTDSYQLLGMAERGFIPSIFAQRSSHGTPTYAILASSLGIAAMASFNFLEIVELLNVVYCMAELLEFTAFLYLRLRYPNLHRPFRVPLSTLGCAIMLAPAFILLSIMLMLPIFRGDWRVVGFTFLAFVLGAVLYPLMQYARQEGWCAFEGVTPDEFKEHLYTTYTPVRPVGSSRESGDMMTVMEDQQQGEQIN

SEQ ID NO: 196 - DNA, 1473 bp, unidentified
  source 1..1473: mol_type = genomic DNA
atgaccgttcctttttcagagcaggccatggtaagcacaagttccaaactcacgaatacagcgtttgatggtctggcaattgaggaaggtgccgagacggatgctactgcagttgctgtggtggcaccggacaaggtcagtccgccccgctcgctcggcctgctccagctcgttggcatgttattccttttaacctctggtggcggttatggcttagaacctctggtagggtccgcaggtccacgatggtcgattcttgctttggtggttgcgccttggctctgggcgttaccgcaggcgttgatggcatcggagttggcgacgttgataccagaggacggtggctatgtgatctgggtggaggcagcgctcggttcgttctggggattccaacaaggttggtggagttttgtggactcacttgtggacaatgctcttttcccgcgtcttttcagtgattatacgctgcgtgttgccccagcattcggtatgcttggcagctggatggtcggggtattggtattggtgatctgcaccttggtcaatattctcggactgggtcttgtaggctgggcagcggtgctctttacagtgcttgtcgtgtccccctttatgttcatttgcatcgtcggctttcgaaggacggtgttgagttccttgtggcaggcgcgacctcttcacgagatcaactggcgtttgtttttggcctcgttgctatggaactggtgcggattcgattcctgctcgacggttgctggtgaaattcgcgatgtacatcgatcgtttcctcgagcaatggtcatcgtcctatcactgaccacaatggttttcacgttgccgatagctgcctcggtgacggtcaatcacgtctggagtgagtggcgtgatgcgttctggccgactgctgcgaatcgtctcgctggcggccactggctcggtattctcgtctgtgttggtggtatgtgcagtgctgcaggcatgttgagcggcttggttgcgacctcatcgcgggcgttgtatgggatgacccgcaagaatatgttgccgccagagctcggtgcgttgcatcaacgatttcgtaccccttggatttgtatcctcttgattgggctgggatcagcctgcttcactgcgctaccctttaacgtactaatccagatagactcgacgttgtactgtctgaaagtcgcattggagttcatcgcgcttgcggtactgcgacggaaatggcccgatcgacagcgaccattccggattggcggaggtatctgggggctctcctatgttgtcggcagtgggctagtctgttgcctggcgatggcggtgctcagtggtctctggtcagcgatttcagcagcagtgaccatcgtgaccggcctcgtgctttattcggtgatcgggcgagttctcaacggtcgatggatgcccagcaccatcgctccgacacctacctcaactccagtggagatataa

SEQ ID NO: 197 - protein, 490 aa, unidentified
MTVPFSEQAMVSTSSKLTNTAFDGLAIEEGAETDATAVAVVAPDKVSPPRSLGLLQLVGMLFLLTSGGGYGLEPLVGSAGPRWSILALVVAPWLWALPQALMASELATLIPEDGGYVIWVEAALGSFWGFQQGWWSFVDSLVDNALFPRLFSDYTLRVAPAFGMLGSWMVGVLVLVICTLVNILGLGLVGWAAVLFTVLVVSPFMFICIVGFRRTVLSSLWQARPLHEINWRLFLASLLWNWCGFDSCSTVAGEIRDVHRSFPRAMVIVLSLTTMVFTLPIAASVTVNHVWSEWRDAFWPTAANRLAGGHWLGILVCVGGMCSAAGMLSGLVATSSRALYGMTRKNMLPPELGALHQRFRTPWICILLIGLGSACFTALPFNVLIQIDSTLYCLKVALEFIALAVLRRKWPDRQRPFRIGGGIWGLSYVVGSGLVCCLAMAVLSGLWSAISAAVTIVTGLVLYSVIGRVLNGRWMPSTIAPTPTSTPVEI

SEQ ID NO: 198 - DNA, 3291 bp, unidentified
  source 1..3291: mol_type = genomic DNA
atgctgtgcataaacatcaccccgaagttcgtgcggatacgccgaggcgttgcgttatcaactcattcgtcgtctttgcgaaattgtcaaattttgtcgtcaaacgcagagagaggaacgaaaacgcagcgccttcttcttagtggtaatcaaaaactcgtaattgattcttcgacgagcgatgatgcttcgatgggaagccatttcagtcgtcgtgcacgtcgtcacttttcctctccattcgcgagggtttccgacaagcggagacacgcgctcgtcacttccgcatcctcgaacaacaacaacaaaatcacgagtgcaaaaagtggtttgaacagcaccagtggtggtgaatctcgtctccgcgcgatcaacaatccctcgcagcagcgagaacaacaagggttagcggacgcactcgctgcggggaaatctgcaaaatcgccgccggggtcgtcacgaggacaagaagaaaaagaaatgatcgaccagacgtacgagttgaacggtaacaacttcgcggaacaaacgatcgcgacgccgtcggatggaccgatcgatgtgttgcacgcctcttcgaagcagacgaagagttggcaagcggtgaggagagcggtgacgactgggccggtgcaatcgaacgcgttgattggagggacgccgttgattgacgtgacgagcgtgttgagtttgaatccttcaaaggtgaagatttacgcgaagtgcgagtatatgaatccgagtggaagcattaaggatcgaattgcgagttatattttacaagcggcgattgatgctggggatttgaaggaggggatgacggtggtggcggcgacgagcgggaacaccgggagcgccatcgcggcggcgtgcgccattcgcgggtttgattacatcgtcattacgaataagaaatgttcgatagagaagatcgatagcatgagagcgtatggtgggacggttattgtcgcgaagagtggcgtagctgcggacgatccagagcattatcaaaatatcgagaacacgatggtggcggaaaatccggggaagtatttcggcgtgaaccagtacgataacttgaacaacgcgttggcttacgaggcgacgttagggccggaaattatcgcgcaaacgaaaggactcatcacgcactttgtcgctgggtcgagtacgggtgggacgctgacgggtaccagtcggtatttgaaggcggtgaacccggagatcaagtccgtgttggcggatccgcgagggtcggtattgtgggataatttcgttaacgacgttccggaaaacgagttaattgttggtaagtgggagattgaaggcgtcgggaaagattctattccgggtgttttgtcctgggacgtcgtcgatggcgcgcaaagaggagacgacgcgtcgagtttttacacgtgtcgcaaagtcgcgagagatttgggcgttttagttggaggctccgccgggttgaatttacacgcgtgcgcggtcttgagcggtaaaatcgacaacggcgtcatcgtgaccgttttgccagactctggggtgaaatacctgtccaagattttcaacgacgattggatgaacgagaaaggattcacgggcaaggaaaagagtcccgaggatggagaaatctattggcgaccgggcgcgaacgatccgtgcgagaaccaaaagagtagcagaatgtttccgataaacgcgtgctctttacaacacgacacgagaaagcaatgcaacgacgacgagttgaacccgagagaacaaacggaagcggagttgagatttttggaagagacggcggcgagaatggtcgaataccatcgtcaatccgtaaaaattggcgaagaaccggtagtattgatgaatactccggaaagcattcgagcgatgttttccgaagctggcgttgggatgacctttgagcaccaagaaccggcgtggagcgagcaacagttgagagacgccgtgacgaccatcttgcaaacgtccgtgcgttcatcgtcgccgttatttttgaaccaattatacgccggtgtcgacccggtcgcgttagccggcgaatgggtctccgcggcgttgaactcaaacgtgcacacgttcgaagtcgcgccatcgttgacggagattgagaaatcatgcttggagaaagtcgcgcggtgttggttgaagaccaacgacggcgtcgagactcccgaacacgacggtttgttcgtcccaggtggatcgttatcgattttatattccatcttgttagccagagacgtcgcggactcttccattcgcaaggctggcatggacagaaataacaagttggtagctttttgcagcgaaaacgcgcattactcgtacaaaaaatccgccatcgttaccggtttaggcgaagaaaacttggtcgcggtgaaatgtctgccgaacggtgccatggatccgggcgctttgcgtgcggcgattgcatcggccatcgcggcaggcaaaacgccgttctacgtcggcacgaccgcgggtacaactgttttaggcgcttttgatccgttcagcgaaatctttgacgtcgtcgacgagtttcaaaatgccaacgggaaatcgcaacgcatttggacccacatcgacggcgcgtggggcggtggtgccatgctttccaaagagcacaatcatctcatggacggcgcggaacgctcggattctttctcttggaacccgcacaaaatgctcggcatgcctttgcaatgctcggtgtttgtctgcaagcacgccggctctttatctaaagcgaacggcgccaaggcggaatatctcttccaaccggacaagaataactctggcgcggacttgggcgaccgaaccattcagtgtggacgcaaagcagacgccgtgaagttatggttggcgtggaaattgcgaggcgacgaaggtttcgccaagtgcatcgatcggtcctttcacttggcgaaattcgtccaactcgaagttgaaaactcggacggcaagttcgttctcgtccaaccggcccaatgctccaacgtcgggttctggtacgttccgccgcgcttgcgcccattcaaccggacaactgcgactgaagaagattgggccgaactcggttacgtcgctccaaagttgaaaaacgccatgcaaaaagccggcgacgccatgattggcttccaacccatcgccagcatgggctacgtcaactttttccgcctggtgttaccgaacccgagacacatcacggaaatggatctgagagcgatgttggatcgcatggacacctacggccaagaattttaa

SEQ ID NO: 199 - protein, 1096 aa, unidentified
MLCINITPKFVRIRRGVALSTHSSSLRNCQILSSNAERGTKTQRLLLSGNQKLVIDSSTSDDASMGSHFSRRARRHFSSPFARVSDKRRHALVTSASSNNNNKITSAKSGLNSTSGGESRLRAINNPSQQREQQGLADALAAGKSAKSPPGSSRGQEEKEMIDQTYELNGNNFAEQTIATPSDGPIDVLHASSKQTKSWQAVRRAVTTGPVQSNALIGGTPLIDVTSVLSLNPSKVKIYAKCEYMNPSGSIKDRIASYILQAAIDAGDLKEGMTVVAATSGNTGSAIAAACAIRGFDYIVITNKKCSIEKIDSMRAYGGTVIVAKSGVAADDPEHYQNIENTMVAENPGKYFGVNQYDNLNNALAYEATLGPEIIAQTKGLITHFVAGSSTGGTLTGTSRYLKAVNPEIKSVLADPRGSVLWDNFVNDVPENELIVGKWEIEGVGKDSIPGVLSWDVVDGAQRGDDASSFYTCRKVARDLGVLVGGSAGLNLHACAVLSGKIDNGVIVTVLPDSGVKYLSKIFNDDWMNEKGFTGKEKSPEDGEIYWRPGANDPCENQKSSRMFPINACSLQHDTRKQCNDDELNPREQTEAELRFLEETAARMVEYHRQSVKIGEEPVVLMNTPESIRAMFSEAGVGMTFEHQEPAWSEQQLRDAVTTILQTSVRSSSPLFLNQLYAGVDPVALAGEWVSAALNSNVHTFEVAPSLTEIEKSCLEKVARCWLKTNDGVETPEHDGLFVPGGSLSILYSILLARDVADSSIRKAGMDRNNKLVAFCSENAHYSYKKSAIVTGLGEENLVAVKCLPNGAMDPGALRAAIASAIAAGKTPFYVGTTAGTTVLGAFDPFSEIFDVVDEFQNANGKSQRIWTHIDGAWGGGAMLSKEHNHLMDGAERSDSFSWNPHKMLGMPLQCSVFVCKHAGSLSKANGAKAEYLFQPDKNNSGADLGDRTIQCGRKADAVKLWLAWKLRGDEGFAKCIDRSFHLAKFVQLEVENSDGKFVLVQPAQCSNVGFWYVPPRLRPFNRTTATEEDWAELGYVAPKLKNAMQKAGDAMIGFQPIASMGYVNFFRLVLPNPRHITEMDLRAMLDRMDTYGQEF

SEQ ID NO: 200 - DNA, 2988 bp, unidentified
  source 1..2988: mol_type = genomic DNA
atgcagaccatccccgcttcgacccgggttctgtccaaccaaccccgttcaatccgcatgttcggaagcgggtcccaggtcgcgtccggccccgcgggcgtcgcccttcgccgggcggcggtcatcgtcgactccaggagccgacgcggcatccgcggccgcgtggtgacccgcgccgcgcagagacccgagcccccgcgcgcgttctccacggatctctccgacaaccagaacctcgagagcctgatggagcgcgaggcgcagcccgccgtgcagaccgaggccgacctcaagcagaagcaggcggaggcgcgggcgaaccagcccgataacaactaccccgtcaccaccggccagctcatcggcagcaccccgctcctggacctctcgcagtactcgctcaacgagaaggttaagatcttcgccaagtgcgagtacctcaacccttccggttccatcaaggacaggatcgcccagcacatcatcggtcaggccgaggcgagcggcgagttaaagcccgggatgaccgtcgtcgccgccacgagcggcaacaccggcgcggcgatcgcgatggcgtgcgcacttcgcgggtacgattacatcgtcatcaccaacgagaagacgtcgaaggagaaggtggatgccatgagggcgtacggcggcgaggtcatcgtctcgccctcgggcgtcgctcccgatcacccggaccactaccagaacattgagaaccgcctgtgcgccgagaaccccggcacgtactacggcgtggatcagtacaacaacccgtacaacgctgaagcgtacgaggctacgctgggtccggaaatttggcgacagacttcgggcgacgtgacgcactttgtcgccggcggaagtaccggcggcacggtcagcggcaccgggcgctacctcaagagcgagaacgcggacgtccgcgtggtcatggcggaccccaagggttccgtcttttgggaccacgtcgtcaagggggtctccgccgacgacgtgtgcgtttccaagagttgggagatggagggtgtcggaaaggactccatcccgggttgcttcgacatcaacgtcatcgacggcatggtgcgcggtaacgacgaggacgcgttcgccatgtgccgcgaggtggccgccaacgacggccttctcctcggcggatccgcgggccttaacctccacgccgctcgcgtcctctccggcgaggttgacgacggctccgtcatcgtaaccgtcttccccgacaacggcgtcaagtacctctccaagatttacaacgacagctggctcgagtccaagggtctggagggcgccgccgaagggaacggcgcggtgtcccccgacgtcgacgagaaggagcacgactcgaggatacactggaagcccgagattgtcaccaggcaggagcaagccgccgccgccgccgccgccgccgccgccgccgtcgaaggccaccacctcctcaccgaggaacagacggaagccgagctgaagttcctggaagacatcgcccctaagctcgtgcggtaccaccgcgaatccatcggcggcgccgagcgcgtgcactcgcgacttcagagcccggatgatctcgccgagacgttcgccgccgcgggggtgcccctggacctggcggatggcgccccggtggacgaactcgccctcgacgccgcggttcaggccgtcatggacaacagcgtccgatcctcgcacccgctcttcctcaaccagctctactctggcgtcgaccccgtcgccctcgccggagattggctcgcgtctgcgctcaacgccaacgtgcacaccttcgaggttgcgccggtgctcaccgagattgaaaaggcggtgctcgcaaagacggcgaggatgtggctccaacccacgctcggctccgcggcccaaaccccgtcgcacgacgggctcatcgtccccggcggatcgctcgcgaacatgtactcgctgctcctcgcgcgggatcgcgtcgacccggacgcgcgcaaccggggaacgaccatggagctcgtcgccttttgcagcgaacagtcgcactactcgttccgaaagtccgcgatggtcatgggcatcggtatggacaacatggtcaaggtgaagtgcgacgccgacggcgccatgatcgccgaggagctcgagaaggaggtgctcgccgcgaaggcacgcggcgccgtgcccttctacgtgggcacaaccgcgggtagcaccgtgctcggcgcctttgacgactatcacggctgcgcggatgtgtgcaagcggcacggactgtggatgcacgtcgacggcgcgtggggcggcgccgccgccctgagccccgcgcgaaggtcggtcctggacggcttcgagaaggcggactcgttctgctggaacccgcacaagatgctgggtctgcccctgcagtgcagcatcttcgtcacgaagcagcccgggtccctggccaaggccaacgcggccaaggctgactacctgttccagcccgataagaacaacgcgggcgcggatctcggcgaccgcaccatccagtgcggccgcaaggctgacgcgctcaagatctggctcagctggaagcaccgcggcgataagggctggtccgacatggtggacaggtccttcggactcgccgaggaggttgagcgaatgattctcgagcgggaaaatcgcgacgggtcctttgtcctcgccgctcacgctcagtgcgccaacgtcggtttctggtacgttccccccaggcttcgacccttcaagcacgcgggggcgtcggcgaaccagctcacggagattggcttcgtcgcgcccaagctgaaggatcgcatgcagcgggagggcgacgccatgattggctttcagccaatcgattccatgaacttgcccaactttttccgcctcgtgctgcccaacagccgccacctctctaagcagatgctgagcgacatgctggaccgcatggacgagatgggcaagaacctttga

SEQ ID NO: 201 - protein, 995 aa, unidentified
MQTIPASTRVLSNQPRSIRMFGSGSQVASGPAGVALRRAAVIVDSRSRRGIRGRVVTRAAQRPEPPRAFSTDLSDNQNLESLMEREAQPAVQTEADLKQKQAEARANQPDNNYPVTTGQLIGSTPLLDLSQYSLNEKVKIFAKCEYLNPSGSIKDRIAQHIIGQAEASGELKPGMTVVAATSGNTGAAIAMACALRGYDYIVITNEKTSKEKVDAMRAYGGEVIVSPSGVAPDHPDHYQNIENRLCAENPGTYYGVDQYNNPYNAEAYEATLGPEIWRQTSGDVTHFVAGGSTGGTVSGTGRYLKSENADVRVVMADPKGSVFWDHVVKGVSADDVCVSKSWEMEGVGKDSIPGCFDINVIDGMVRGNDEDAFAMCREVAANDGLLLGGSAGLNLHAARVLSGEVDDGSVIVTVFPDNGVKYLSKIYNDSWLESKGLEGAAEGNGAVSPDVDEKEHDSRIHWKPEIVTRQEQAAAAAAAAAAAVEGHHLLTEEQTEAELKFLEDIAPKLVRYHRESIGGAERVHSRLQSPDDLAETFAAAGVPLDLADGAPVDELALDAAVQAVMDNSVRSSHPLFLNQLYSGVDPVALAGDWLASALNANVHTFEVAPVLTEIEKAVLAKTARMWLQPTLGSAAQTPSHDGLIVPGGSLANMYSLLLARDRVDPDARNRGTTMELVAFCSEQSHYSFRKSAMVMGIGMDNMVKVKCDADGAMIAEELEKEVLAAKARGAVPFYVGTTAGSTVLGAFDDYHGCADVCKRHGLWMHVDGAWGGAAALSPARRSVLDGFEKADSFCWNPHKMLGLPLQCSIFVTKQPGSLAKANAAKADYLFQPDKNNAGADLGDRTIQCGRKADALKIWLSWKHRGDKGWSDMVDRSFGLAEEVERMILERENRDGSFVLAAHAQCANVGFWYVPPRLRPFKHAGASANQLTEIGFVAPKLKDRMQREGDAMIGFQPIDSMNLPNFFRLVLPNSRHLSKQMLSDMLDRMDEMGKNL

SEQ ID NO: 202 - DNA, 3495 bp, unidentified
  source 1..3495: mol_type = genomic DNA
atgtctgcggtgcgcgcagcagtccctcctgggtcctccaaggggggtgctctcaggggccctcatgggtctcgctctcgtggtggtggtctctctggctttcacaggttacatgcttcgaatcctgacaggagtcgtggtgcacccggtcgttccgtggacctgggagaagaatcgttgttgtctcagaattctgagagagagacctctgcagcagcttctcagggcccgggggcttcttcatcgttggagagcgacgagaatgtggatgctgagagatcaaattccaaaatcaacacaaacgacaccacggcggaaaatacgcagacgacgctcgagtctgaatcagttccgcagacaagtcctccgccgttcgcggcgggaaacacgggtgccgttgtcggcaacacgcctttgctggacctcagccggctgtccaaggcggagaacgtcaaaattctcgcgaaaatggagttcctcaaccccacgggatccatcaaagaccgcatcgccctgcacatgatctcacgcgccgagtcggaggggaaaatcaaaccaggcgtcaccaccatcgtcgcatccacgtcaggaaacaccgggtgcagtgtcgcgatggtgtgcgccctgaaaggatacgactatatcgtcatcacctccaagaagtgctcggaagaaaaaaggggaagcatgagagcgttcgggggacaggtcatcatcgccgacgacggactggctgccgaccacccggagcactacgtgaacctcgaaagcacgctgtgtgagcagaacgaaaactactttggaatcgaccagtacgagaaccagcacaatccggaggcgtactacacgtcactcggaccggagatctggcagcagacgcgcggacgtgtcacgcacttcgttgccggtgggtcgaccggaggcacggtcacgggcacgggcgcctacctgaagcacgtctccgacaaccaagtgcaggtgctcatgcccgatccggtgggatccgtcatcttcgagcactgggcaaaacacgtcccggagaacgagctgcaggcgtcggggtacaaggtggaaggcgttggaaaggactccatacccggaacgctcgacctgtcgttcgtcgatgccatgatctccgtcacagacaagcagtcgatccaaatgtgccagacgctctcctccacagctggcgcgctcgtcggaggtagctccggcctaaacgtgtacgccgcggtggaactcagcaaggaactgggcaggcagaatccctcgcgggagttcaccatcgtcaccgtgctgccggacagtgggctgaagtacttgagcaagatctacaaccagacgtggtgcgaggagaatggctttatggacgacgaatgtgacccacaggatgaggtgaaccgctcaagatgccgtgacaccgccgtgcctcgtgcaagcgccacggaggttgatgacgatctcgcggctgaggaaaacaagtctcccctgttttctcgcgaggttgcgaatgccttcctgaaggatgccgcggacattcttacggaatatgccgccgcggaggatgtgcatgtcgtagacgataccagcggtgggaacagcaatagcggcaacggcagtggcggtggcggcggcggaaacagcaacagcaacaacagcaacagcaacaacgagcaggaagacaacgatgagacatcgcccttgttgtcttcgtcatcgtacaacgcgccaattgtgtcaaccacggcactgggcggaggaacaaatcccgcattccctcgcgcaaaactcgcggatccggcaacgccaattccacccgtcatcacgcttgctagcccggaagaaattgacgcggcgtatcgcgagcgcgctagcatgagcctcagcttgcttgcaaatggcggtggcgggtcgcacacgacggataagctcctcgaggcactcaaggtgaccttggacatgtcggcgagaacgcatcacccgctcttcctgaatcagctgtactctggtgtcgatcccatcggcctcgcgggcgagtggctgaccgcggcgacgaatacaaacgttcacacgtacgaggtggctcccgtcttcacgctcattgagcgcgaggtgctcgctaagatggcatccatctggctcgcggaccctgacgaggtgacgagcgtggccaagcccacgacaaatcgcaagtcgagcttccagaaattcctttcgagctcagaggctgctgaccctatgccaaccccctcggcggagaacagcctcgacgatgccgtggcttgcgcacccccgcatgacgggctgtttgttcctggtggatccatcagcaacatgtacgggatgctgctggcgcgacaccgcgcggacccagagtacgtgaatcgtggcgcgtacggtgcgcctcgcctagtcgctttcgtgagcgaccaggctcactactcgttctcgaagactgcaggcgttatgggacttgggcgtgacaacgtctggtccgtcgagagtgatgcgagcggcgcaatgatccccgacgcgctcgagaggagcatcgagaaggctgtggcagagggagcaacgccgttcttcgttggcgcaactgccgccacgacggtgatgggtgcctttgatccattcgtttcgatccgtgagatttgcgatcggcactcgttcacgcctgccggtgcctcggaagacgagcgcgtgccaatctggatgcacgtcgacgcggcgtggggaggcatgtacttcatgagcccgaaactgcgaaagcagacgctgcgcggcgctgagcgtgcggacagcctaacgtggaacccgcacaagatgatgaacatgccgctaacgtgcagcgtgattctcacgcgtgagcccggcagccttcaggcgatgaatgcgagcaatgccgcgtatctcttccagccagacaaaaactttgtcgagtacgatcttggcgaccgctcgattcagtgtgggcgcaaggcggatgcgttcaagctttggctcgcttggaaggcgaagggcgatcttggttggtgtctcgaggtggagcacgtcaacgcgctgggcgaaggcctcgagaggatgattatggaccagcaggctacgagctccgtggctggctgctcgtttgaggggcgcttcctgatggcgacgccgcgatcgtgcacgaatgtttgcttctggtacatccccccacgcctcgttggtgagctcgacatgcacattgtcacggtggacagcatgggtgagcccgtcagccccgtcagcctcgtgggtgtgcctgacgagcttcgcagcgaaattgacggcgtcgcgccgaagattaaggatcgcatgcagcgcatggctggcgatggcagcgcgctgattggattccagcgcaacaagggtcttccgaacttcttccgtctcgtgttgcctggtggcaaggggttgagcgacgaggttctcctgcgcatgctcaagcgcatggacgagcttggaaaggatctctaa

SEQ ID NO: 203 - protein, 1164 aa, unidentified
MSAVRAAVPPGSSKGGALRGPHGSRSRGGGLSGFHRLHASNPDRSRGAPGRSVDLGEESLLSQNSERETSAAASQGPGASSSLESDENVDAERSNSKINTNDTTAENTQTTLESESVPQTSPPPFAAGNTGAVVGNTPLLDLSRLSKAENVKILAKMEFLNPTGSIKDRIALHMISRAESEGKIKPGVTTIVASTSGNTGCSVAMVCALKGYDYIVITSKKCSEEKRGSMRAFGGQVIIADDGLAADHPEHYVNLESTLCEQNENYFGIDQYENQHNPEAYYTSLGPEIWQQTRGRVTHFVAGGSTGGTVTGTGAYLKHVSDNQVQVLMPDPVGSVIFEHWAKHVPENELQASGYKVEGVGKDSIPGTLDLSFVDAMISVTDKQSIQMCQTLSSTAGALVGGSSGLNVYAAVELSKELGRQNPSREFTIVTVLPDSGLKYLSKIYNQTWCEENGFMDDECDPQDEVNRSRCRDTAVPRASATEVDDDLAAEENKSPLFSREVANAFLKDAADILTEYAAAEDVHVVDDTSGGNSNSGNGSGGGGGGNSNSNNSNSNNEQEDNDETSPLLSSSSYNAPIVSTTALGGGTNPAFPRAKLADPATPIPPVITLASPEEIDAAYRERASMSLSLLANGGGGSHTTDKLLEALKVTLDMSARTHHPLFLNQLYSGVDPIGLAGEWLTAATNTNVHTYEVAPVFTLIEREVLAKMASIWLADPDEVTSVAKPTTNRKSSFQKFLSSSEAADPMPTPSAENSLDDAVACAPPHDGLFVPGGSISNMYGMLLARHRADPEYVNRGAYGAPRLVAFVSDQAHYSFSKTAGVMGLGRDNVWSVESDASGAMIPDALERSIEKAVAEGATPFFVGATAATTVMGAFDPFVSIREICDRHSFTPAGASEDERVPIWMHVDAAWGGMYFMSPKLRKQTLRGAERADSLTWNPHKMMNMPLTCSVILTREPGSLQAMNASNAAYLFQPDKNFVEYDLGDRSIQCGRKADAFKLWLAWKAKGDLGWCLEVEHVNALGEGLERMIMDQQATSSVAGCSFEGRFLMATPRSCTNVCFWYIPPRLVGELDMHIVTVDSMGEPVSPVSLVGVPDELRSEIDGVAPKIKDRMQRMAGDGSALIGFQRNKGLPNFFRLVLPGGKGLSDEVLLRMLKRMDELGKDL

SEQ ID NO: 204 - DNA, 450 bp, Escherichia coli
  source 1..450: mol_type = genomic DNA
atgttctcaccgcagtcacgcttgcgtcatgcagttgcagatacgttcgcgatggttgtttactgttctgtcgtgaacatgtgtattgaagttttcctctccggaatgagcttcgaacagtctttttattccagattggtagcgattccggtgaacatcttaattgcatggccatacggtatgtaccgtgatctgtttatgcgcgcggcacgcaaagttagcccgtcgggctggataaaaaatctggcggatatcctggcttatgtgacgttccagtcaccggtgtatgtggcgatcttgttagtggtgggcgcagactggcatcagattatggcggcggtcagttcaaacatcgttgtttcgatgttgatgggggcggtttatggctacttcctcgattattgccgccgactgtttaaagtcagccgttaccagcaggtaaaagcctga

SEQ ID NO: 205 - protein, 149 aa, Escherichia coli
MFSPQSRLRHAVADTFAMVVYCSVVNMCIEVFLSGMSFEQSFYSRLVAIPVNILIAWPYGMYRDLFMRAARKVSPSGWIKNLADILAYVTFQSPVYVAILLVVGADWHQIMAAVSSNIVVSMLMGAVYGYFLDYCRRLFKVSRYQQVKA

SEQ ID NO: 206 - DNA, 588 bp, Pantoea ananatis
  source 1..588: mol_type = genomic DNA
atgctcgatccttccttctttagttatgtcaccgtgatgtcgattacgccggggccaaataatctgttgctcgccacttccggtgttaactttggtatgcgccggacgttaccgatggtgtttggcattttgattggctgtgcgctacaaaccgccattgcggggctggcgctggaagtcctgctgcactggatggcatctgtgcgtctgccgctgacgctggtgggatgtggttatttgttatggctgtcatggaaaatatttcgggcgaatgcccccgaagcgcgcgcaaagccgcagcccatgaccctggtgggtggggcctgttttcaggctatcaatccaaaagcctggttaatggccagcaacgtggcactgctctacagcgccagcagtggcgtgctgaccgtgatgggggcttttatgttgctcaatctgccgtgcattttattgtgggctgcactgggtgaccgcctgcgcggtcacctgcaaatcaactggaaacgccagctgtttaacagcctgatggcgctgtcgctggtggcgaccacgatctggatgctggtcgatgccttcaggcttctctag

SEQ ID NO: 207 - protein, 195 aa, Pantoea ananatis
MLDPSFFSYVTVMSITPGPNNLLLATSGVNFGMRRTLPMVFGILIGCALQTAIAGLALEVLLHWMASVRLPLTLVGCGYLLWLSWKIFRANAPEARAKPQPMTLVGGACFQAINPKAWLMASNVALLYSASSGVLTVMGAFMLLNLPCILLWAALGDRLRGHLQINWKRQLFNSLMALSLVATTIWMLVDAFRLL

SEQ ID NO: 208 - DNA, 906 bp, Pantoea ananatis
  source 1..906: mol_type = genomic DNA
atgaacgcattactctatgctgtggtcgtggttatctggggtacaacctggattgccatctttttgcaacaagggcccgttgatgtcacggtgtccgttttctggcgcttcgccatagccagtattttattgctgacgctcctgcagataatggggcgcctgcgcagattatcgccgcgcgatcatctgttttgcgctttgcagggctgctgtgtattttgctttaatttttggtgcttttacaccgccgccggttatatcaataccggtttagagtccgtgattttttccatggcggtgctgtataacgcagtaaacagttatctgttttttgggcagcgcccccagccgcgtttctggtttgcggtcacgctgggattaacgggcatgatcctgttgttttgggacgatctgcacatcagcggcgcaagcggtggactgtggcgaggcatcggcctgtcggcgttgggcacgtttggcttttctctgggcaacatgatcagcctgcggcatcagcgcagacagctggaaaccatgaccactaacgcctgggctatgttatacggcgcaatggtcatgggcattatcgctctacttcgtggcgtgagtttctcaccgctctggacactgagctacctcagtgccttgctttatttgtccatattcggttcggtcattgcgttttgggcgtatttcaccctggtcagacgtacgggcgccgctaaagccgcatacacaacactgatgtttccgttggttgccctcaccttatcgacggtttatgaaggctatcactggcaaagccacagcgtggtgggattgctgatgatcctgggcggtaacctggtgatgttcggacgtcccttgcgctggccaaaaccgaaacccatgccgggcacgctataa

SEQ ID NO: 209 - protein, 301 aa, Pantoea ananatis
MNALLYAVVVVIWGTTWIAIFLQQGPVDVTVSVFWRFAIASILLLTLLQIMGRLRRLSPRDHLFCALQGCCVFCFNFWCFYTAAGYINTGLESVIFSMAVLYNAVNSYLFFGQRPQPRFWFAVTLGLTGMILLFWDDLHISGASGGLWRGIGLSALGTFGFSLGNMISLRHQRRQLETMTTNAWAMLYGAMVMGIIALLRGVSFSPLWTLSYLSALLYLSIFGSVIAFWAYFTLVRRTGAAKAAYTTLMFPLVALTLSTVYEGYHWQSHSVVGLLMILGGNLVMFGRPLRWPKPKPMPGTL

SEQ ID NO: 210 - DNA, 900 bp, Escherichia coli
  source 1..900: mol_type = genomic DNA
atgtcgcgaaaagatggggtgttggcgctactggtagtggtcgtatgggggctaaattttgtggtcatcaaagtggggcttcataacatgccaccgctgatgctggccggtttgcgctttatgctggtcgcttttccggctatcttttttgtcgcacgaccgaaagtaccactgaatttgctgctggggtatggattaaccatcagttttgcgcagtttgcttttcttttttgtgccattaacttcggtatgcctgctggactggcttcgctggtgttacaggcacaggcgttttttactatcatgcttggcgcgtttactttcggggagcgactgcatggcaaacaattggcggggatcgccttagcgatttttggcgtactggtgttaatcgaagatagtctgaacggtcagcatgtggcgatgctcggctttatgttgaccctggcggcagcatttagttgggcgtgtggcaacatcttcaataaaaagatcatgtcgcactcaacgcgtccggcggtgatgtcgctggtaatctggagcgctttaatcccaatcattcccttctttgttgcctcgctgattctcgatggttccgcaaccatgattcacagtctggttactatcgatatgaccaccatcttgtctctgatgtatctggcgtttgtggcgacaattgttggttatgggatctgggggacgttactgggacgctatgaaacctggcgggttgcaccgttatcgttactggtgcccgtagtaggactggcaagtgcggcactattgttggatgaacgcttaacgggtctgcaatttttaggtgcggtgctcattatgaccgggctgtatatcaatgtatttggcttgcggtggcgtaaagcggtaaaggtgggaagttaa

SEQ ID NO: 211 - protein, 299 aa, Escherichia coli
MSRKDGVLALLVVVVWGLNFVVIKVGLHNMPPLMLAGLRFMLVAFPAIFFVARPKVPLNLLLGYGLTISFAQFAFLFCAINFGMPAGLASLVLQAQAFFTIMLGAFTFGERLHGKQLAGIALAIFGVLVLIEDSLNGQHVAMLGFMLTLAAAFSWACGNIFNKKIMSHSTRPAVMSLVIWSALIPIIPFFVASLILDGSATMIHSLVTIDMTTILSLMYLAFVATIVGYGIWGTLLGRYETWRVAPLSLLVPVVGLASAALLLDERLTGLQFLGAVLIMTGLYINVFGLRWRKAVKVGS

SEQ ID NO: 212 - DNA, 1401 bp, Pantoea ananatis
  source 1..1401: mol_type = genomic DNA
atggggcaatcatcgcaaccacatgagttaggcggcgggctgaagtcacgccacgtcaccatgttgtctattgccggtgttatcggcgcaagtctgtttgtcggttccagcgtcgccatcgccgaagcgggcccggcggtattactggcctatctgttcgccgggctactggtggttatgattatgcggatgttggcggaaatggcggttgccacgcccgataccggttcgttttccacctatgccgataaagccattggtcgctgggcgggctataccatcggctggttgtactggtggttttgggtactggttatcccgctggaagccaacatcgccgccatgatcctgcactcatgggttccaggcattcccatctggttattttccctcgtcattaccctcgccttaactggcagtaacttattaagcgttaaaaactacggcgaatttgagttctggctggcgctgtgcaaagtcatcgctatcctggcctttattttccttggtgcagtcgcaattagcggtttttacccgtatgccgaagtgagcgggatctcaagattgtgggatagcggcggctttatgcccaacggtttcggtgcggtattaagcgcgatgttgatcaccatgttctcgtttatgggcgcagaaattgtcaccattgccgccgcggaatccgacacgccggaaaaacatattgtccgcgccaccaactcggttatctggcgtatttctatcttctatttatgttctatttttgtcgtcgtggcattaattccgtggaatatgcccggactaaaagccgtcggttcttatcgctcggtactggaattgctcaatattccccatgcgaaattaatcatggactgcgtgatattactttccgtaaccagttgcctgaactcggcgctgtataccgcgtcaaggatgctctactccttaagtcgtcgcggtgatgctcccgcggtaatgggtaaaatcaaccgcagtaaaaccccgtacgtggcggtgttactctccaccggcgcggcattcttaacggtggtggtgaactattacgcgcctgcgaaggtatttaaatttctgatcgacagctccggcgctatcgccctgctggtttatttagtcatcgccgtttcacagttgcggatgcgcaaaattctgcgagcagaaggaagcgaaattcgcttgcgaatgtggctttatccgtggctcacctggctagtcatcggctttattacctttgtgttggtagtgatgctattccgtccggcgcaacagttagaagtgatctccaccggcttattagcgatagggattatctgtaccgtgccaattatggctcgctggaaaaagctggtattgtggcaaaaaacacccgttcataatacgcgctga

SEQ ID NO: 213 - protein, 466 aa, Pantoea ananatis
MGQSSQPHELGGGLKSRHVTMLSIAGVIGASLFVGSSVAIAEAGPAVLLAYLFAGLLVVMIMRMLAEMAVATPDTGSFSTYADKAIGRWAGYTIGWLYWWFWVLVIPLEANIAAMILHSWVPGIPIWLFSLVITLALTGSNLLSVKNYGEFEFWLALCKVIAILAFIFLGAVAISGFYPYAEVSGISRLWDSGGFMPNGFGAVLSAMLITMFSFMGAEIVTIAAAESDTPEKHIVRATNSVIWRISIFYLCSIFVVVALIPWNMPGLKAVGSYRSVLELLNIPHAKLIMDCVILLSVTSCLNSALYTASRMLYSLSRRGDAPAVMGKINRSKTPYVAVLLSTGAAFLTVVVNYYAPAKVFKFLIDSSGAIALLVYLVIAVSQLRMRKILRAEGSEIRLRMWLYPWLTWLVIGFITFVLVVMLFRPAQQLEVISTGLLAIGIICTVPIMARWKKLVLWQKTPVHNTR

SEQ ID NO: 214 - DNA, 888 bp, Escherichia coli
  source 1..888: mol_type = genomic DNA
atgcctggttcattacgtaaaatgccggtctggttaccaatagtcatattgctcgttgccatggcgtctattcagggtggagcctcgttagctaagtcactttttcctctggtgggcgcaccgggtgtcactgcgctgcgtctggcattaggaacgctgatcctcatcgcgttctttaagccatggcgactgcgctttgccaaagagcaacggttaccgctgttgttttacggcgtttcgctgggtgggatgaattatcttttttatctttctattcagacagtaccgctgggtattgcggtggcgctggagttcaccggaccactggcggtggcgctgttctcttctcgtcgcccggtagatttcgtctgggttgtgctggcggttcttggtctgtggttcctgctaccgctggggcaagacgtttcccatgtcgatttaaccggctgtgcgctggcactgggggccggggcttgttgggctatttacattttaagtgggcaacgcgcaggagcggaacatggccctgcgacggtggcaattggttcgttgattgcagcgttaattttcgtgccaattggagcgcttcaggctggtgaagcactctggcactggtcggttattccattgggtctggctgtcgctattctctcgaccgctctgccttattcgctggaaatgattgccctcacccgtttgccaacacggacatttggtacgctgatgagcatggaaccggcgctggctgccgtttccgggatgattttcctcggagaaacactgacacccatacagctactggcgctcggcgctatcatcgccgcttcaatggggtctacgctgacagtacgcaaagagagcaaaataaaagaattagacattaattaa

SEQ ID NO: 215 - protein, 295 aa, Escherichia coli
MPGSLRKMPVWLPIVILLVAMASIQGGASLAKSLFPLVGAPGVTALRLALGTLILIAFFKPWRLRFAKEQRLPLLFYGVSLGGMNYLFYLSIQTVPLGIAVALEFTGPLAVALFSSRRPVDFVWVVLAVLGLWFLLPLGQDVSHVDLTGCALALGAGACWAIYILSGQRAGAEHGPATVAIGSLIAALIFVPIGALQAGEALWHWSVIPLGLAVAILSTALPYSLEMIALTRLPTRTFGTLMSMEPALAAVSGMIFLGETLTPIQLLALGAIIAASMGSTLTVRKESKIKELDIN

SEQ ID NO: 216 - DNA, 621 bp, Escherichia coli
  source 1..621: mol_type = genomic DNA
atgaccttagaatggtggtttgcctacctgctgacatcgatcattttaagcctgtcgccaggctctggtgcaatcaacactatgaccacctcgctcaaccacggttatcgcggcgcggtggcgtctattgctgggcttcagaccggactggcgattcatattgtgctggttggcgtggggttggggacgctattttcccgctcagtgattgcgtttgaagtgttgaagtgggcaggcgcggcttacttgatttggctgggaatccagcagtggcgcgccgctggtgcaattgaccttaaatcgctggcctctactcaatcgcgtcgacatttgttccagcgcgcagtttttgtgaatctcaccaatcccaaaagtattgtgtttctggcggcgctatttccgcaattcatcatgccgcaacagccgcaactgatgcagtatatcgtgctcggcgtcaccactattgtggtcgatattattgtgatgatcggttacgccacccttgctcaacggattgctctatggattaaaggaccaaagcagatgaaggcgctgaataagattttcggctcgttgtttatgctggtgggagcgctgttagcatcggcgaggcatgcgtga

SEQ ID NO: 217 - protein, 206 aa, Escherichia coli
MTLEWWFAYLLTSIILSLSPGSGAINTMTTSLNHGYRGAVASIAGLQTGLAIHIVLVGVGLGTLFSRSVIAFEVLKWAGAAYLIWLGIQQWRAAGAIDLKSLASTQSRRHLFQRAVFVNLTNPKSIVFLAALFPQFIMPQQPQLMQYIVLGVTTIVVDIIVMIGYATLAQRIALWIKGPKQMKALNKIFGSLFMLVGALLASARHA

SEQ ID NO: 218 - DNA, 621 bp, Escherichia coli
  source 1..621: mol_type = genomic DNA
atgttgatgttatttctcaccgtcgccatggtgcacattgtggcgcttatgagccccggtcccgatttcttttttgtctctcagaccgctgtcagtcgttcccgtaaagaagcgatgatgggcgtgctgggcattacctgcggcgtaatggtttgggctgggattgcgctgcttggcctgcatttgattatcgaaaaaatggcctggctgcatacgctgattatggtgggcggtggcctgtatctctgctggatgggttaccagatgctacgtggtgcactgaaaaaagaggcggtttctgcacctgcgccacaggtcgagctggcgaaaagtgggcgcagtttcctgaaaggtttactgaccaatctcgctaatccgaaagcgattatctactttggctcggtgttctcattgtttgtcggtgataacgttggcactaccgcgcgctggggcatttttgcgctgatcattgtcgaaacgctggcgtggtttaccgtcgttgccagcctgtttgccctgccgcaaatgcgccgtggttatcaacgtctggcgaagtggattgatggttttgccggggcgttatttgccggatttggcattcatttgattatttcgcggtga

SEQ ID NO: 219 - protein, 206 aa, Escherichia coli
MLMLFLTVAMVHIVALMSPGPDFFFVSQTAVSRSRKEAMMGVLGITCGVMVWAGIALLGLHLIIEKMAWLHTLIMVGGGLYLCWMGYQMLRGALKKEAVSAPAPQVELAKSGRSFLKGLLTNLANPKAIIYFGSVFSLFVGDNVGTTARWGIFALIIVETLAWFTVVASLFALPQMRRGYQRLAKWIDGFAGALFAGFGIHLIISR

SEQ ID NO: 220 - DNA, 588 bp, Escherichia coli
  source 1..588: mol_type = genomic DNA
atgacaccgacccttttaagtgctttttggacttacaccctgattaccgctatgacgccaggaccgaacaatattctcgcccttagctctgctacgtcgcatggatttcgtcaaagtacccgcgtgctggcagggatgagtctgggatttttgattgtgatgttactgtgtgcgggcatttcattttcactggcagtgattgacccggcagcggtacaccttttgagttgggcgggggcggcatatattgtctggctggcgtggaaaatcgccaccagcccaacaaaggaagacggacttcaggcaaaaccaatcagcttttgggccagctttgctttgcagtttgtgaacgtcaaaatcattttgtacggtgttacggcactgtcgacgtttgttctgccgcaaacacaggcgttaagctgggtagttggcgtcagcgttttgctggcgatgattgggacgtttggcaatgtgtgctgggcgctggcggggcatctgtttcagcgattgtttcgccagtatggtcgccagttaaatatcgtgcttgccctgttgctggtctattgcgcggtacgcattttctattaa

SEQ ID NO: 221 - protein, 195 aa, Escherichia coli
MTPTLLSAFWTYTLITAMTPGPNNILALSSATSHGFRQSTRVLAGMSLGFLIVMLLCAGISFSLAVIDPAAVHLLSWAGAAYIVWLAWKIATSPTKEDGLQAKPISFWASFALQFVNVKIILYGVTALSTFVLPQTQALSWVVGVSVLLAMIGTFGNVCWALAGHLFQRLFRQYGRQLNIVLALLLVYCAVRIFY

SEQ ID NO: 222 - DNA, 1440 bp, Acinetobacter ursingii
  source 1..1440: mol_type = genomic DNA
atgtatgattcagattcaaatcaaagtttaaaaagtgctttaaaacctcgtcatgtccaaatgatggcactcggtggtgttattggaatggggttatttgtgggtagttctgtagtgattcaatctgcaggtccagccgccattttgtccttcctgattacgggtgtattaattattttagtgatgcggatgctgggtgagctagcaaccgcctctcctgtatcaggctccttctatgcatatgcacgccatgctttccaagattcgccttatttatctcgactcgcaggcttcatgacgggatggatgtattggtacttctgggtcatcgtgattgcattagaagcgattgtcggtgcaaaactctttgcttattggttacctgatacaccgcaatggatcgtcagtctggttttactcgccctattaacagcaaccaatttattctccgtaaaatcctttggtgaattcgaattttggttctcatctattaaagttgctgcgattggtcttttcattttcctttgttctttatatatgttcggtctttggccaaaccatcaagccgctggatttggtgaattgttaaatcatggtggctttatgccaaatggttggggaccagtattatcgggcgctgttgctgcaactggattttactttggcgctgagatcgtcacaattgcagccgctgaaactaaagaccctgaaaaatctgttgccaaagcgactcaatccgtgatttctcgcgtcctgattttctatgtttgctctattttattagtggtctgtattgtcccttgggacagtgcagaaattgcgacaccttacgtaagtgcgctaaaagtattgaatattccgtttgttgcggacattatgaatgttgtgattctggtggctgtactcagttgcttaaattctggagtttatgctgcaagccgtatgttgtttgctttaacccaaaatggtgatgcacctcgtagcttgaccaaactttcaaaaaatggtgttccaattcgtgccattctattcagtacattatttggttatttctcaattttagcttcatacttctcaccagatgctgtatttcctttcttggtcgaatcttatggaactgtagccttatttgtctatatcgtgattgcattctctcaaatccgtttgcgtcgccacttagaacgtgttgcacctgaacgtttaaaagttcgtatgtggtgttttccatacctgagttattttgcaatcatcggcatgctttcaatctttgctgcaatggcttttatacctagccttcaaaaagcattctggttcggcatcgcaagtgcaggaattctattctgtatctttattattcgagattatgtagagctagttaaattacgtcatcaaagtactcaacttgatgtgctcatccctgccaaacaaaccaaaatttaa

SEQ ID NO: 223 - protein, 479 aa, Acinetobacter ursingii
MYDSDSNQSLKSALKPRHVQMMALGGVIGMGLFVGSSVVIQSAGPAAILSFLITGVLIILVMRMLGELATASPVSGSFYAYARHAFQDSPYLSRLAGFMTGWMYWYFWVIVIALEAIVGAKLFAYWLPDTPQWIVSLVLLALLTATNLFSVKSFGEFEFWFSSIKVAAIGLFIFLCSLYMFGLWPNHQAAGFGELLNHGGFMPNGWGPVLSGAVAATGFYFGAEIVTIAAAETKDPEKSVAKATQSVISRVLIFYVCSILLVVCIVPWDSAEIATPYVSALKVLNIPFVADIMNVVILVAVLSCLNSGVYAASRMLFALTQNGDAPRSLTKLSKNGVPIRAILFSTLFGYFSILASYFSPDAVFPFLVESYGTVALFVYIVIAFSQIRLRRHLERVAPERLKVRMWCFPYLSYFAIIGMLSIFAAMAFIPSLQKAFWFGIASAGILFCIFIIRDYVELVKLRHQSTQLDVLIPAKQTKI

SEQ ID NO: 224 - DNA, 1413 bp, Escherichia coli
  source 1..1413: mol_type = genomic DNA
atggtagatcaggtaaaagtcgttgccgatgatcaggctccggctgaacagtcgctacggcgcaatctcacaaaccgacatattcagcttattgccattggcggtgccattggtacggggttgtttatggggtctggcaaaacgattagccttgccgggccgtcgatcattttcgtttatatgatcattggttttatgctctttttcgtgatgcgggcaatgggggaattgctgctttcgaatctggaatacaaatcttttagtgacttcgcttccgatttactcgggccgtgggcaggatatttcaccggctggacttactggttctgctgggttgtaaccggtatggcagacgtggtggcgatcacggcttatgctcagttctggttccccgatctctccgactgggtcgcctcgctggcggtgatagtgctgctgctgacgctcaatctcgccaccgtgaaaatgttcggtgagatggagttctggtttgcgatgatcaaaatcgtcgccattgtgtcgctgattgtcgtcggcctggtcatggtggcgatgcactttcagtcaccgactggtgtggaagcgtcattcgcgcatttgtggaatgacggcggctggttcccgaaaggtttaagtggcttctttgccggattccagatagcggttttcgctttcgtggggattgagctggtaggtacaacagctgcggaaaccaaagatccagagaaatcactgccacgcgcgattaactccattccgatccgtatcattatgttctacgtcttcgcgctgattgtgattatgtccgtgacgccgtggagttcggtagtcccggagaaaagcccgtttgttgaactgttcgtgttggtagggctgcctgctgccgcaagcgtgatcaactttgtggtgctgacctctgcggcgtcttccgctaacagcggcgtcttctctaccagccgtatgctgtttggtctggcgcaggaaggtgtggcaccgaaagcgttcgctaaactttctaagcgcgcagtacccgcgaaagggctgacgttctcgtgtatctgtctgctcggcggcgtggtgatgttgtatgtgaatcctagtgtgattggcgcgttcacgatgattacaaccgtttccgcgattctgtttatgttcgtctggacgattatcctttgctcgtaccttgtgtatcgcaaacagcgtcctcatctacatgagaagtcgatctacaagatgccgctcggcaagctgatgtgctgggtatgtatggcgttctttgtgttcgtggtcgtgttgctgacactggaagatgacactcgccaggcgctgctggttaccccgctgtggtttatcgcgctggggttgggctggctgtttattggtaagaagcgggctgctgaactgcggaaataa

SEQ ID NO: 225 - protein, 470 aa, Escherichia coli
MVDQVKVVADDQAPAEQSLRRNLTNRHIQLIAIGGAIGTGLFMGSGKTISLAGPSIIFVYMIIGFMLFFVMRAMGELLLSNLEYKSFSDFASDLLGPWAGYFTGWTYWFCWVVTGMADVVAITAYAQFWFPDLSDWVASLAVIVLLLTLNLATVKMFGEMEFWFAMIKIVAIVSLIVVGLVMVAMHFQSPTGVEASFAHLWNDGGWFPKGLSGFFAGFQIAVFAFVGIELVGTTAAETKDPEKSLPRAINSIPIRIIMFYVFALIVIMSVTPWSSVVPEKSPFVELFVLVGLPAAASVINFVVLTSAASSANSGVFSTSRMLFGLAQEGVAPKAFAKLSKRAVPAKGLTFSCICLLGGVVMLYVNPSVIGAFTMITTVSAILFMFVWTIILCSYLVYRKQRPHLHEKSIYKMPLGKLMCWVCMAFFVFVVVLLTLEDDTRQALLVTPLWFIALGLGWLFIGKKRAAELRK

SEQ ID NO: 226 - DNA, 1269 bp, Escherichia coli
  source 1..1269: mol_type = genomic DNA
atgagtgagagcgagtggcgagaagcgatcaaattcgacagtactgacaccggctgggtgattatgagtatcgggatggcgattggcgcggggattgtttttctcccggtgcaggtcggtttgatgggattgtgggtatttttgctctcatcggtgattggttacccggcaatgtatctgtttcagcgattgtttattaatacgctggcagaatcaccagaatgtaaagattacccgagcgtcattagcggttatttaggtaaaaactggggcatcctgttaggtgcgctctatttcgtaatgctggtgatttggatgttcgtctattccaccgccatcaccaacgatagtgcttcctacctgcataccttcggcgtgacggaagggttgctgtcagacagtcccttttatggtctggtactgatttgcattctggtggcgatctcctcacgcggcgagaaattgttattcaaaatttcgaccggcatggtgctgaccaagctgctggtggtcgcggcgctgggcgtgtcgatggtgggaatgtggcatctgtataacgtcggttcgctaccgccgctggggctgctggtgaaaaacgccattattacgctgccgtttaccctgacgtcgattctgtttatccagacgttaagtccgatggtgatctcttatcgctcgcgggaaaaatcaattgaagtggcgcggcataaagcattgcgggcaatgaatatcgcgtttggcattttgtttgtcaccgtctttttctacgccgtgtcgttcacgctggcgatgggacatgacgaagcggtaaaagcctatgagcagaatatttccgcgctggcgattgccgcacagtttattagcggtgacggcgcagcgtgggtgaaagtggtcagcgtcattctcaatatctttgctgtcatgactgcattctttggcgtctatttaggctttcgcgaagcaacgcaagggatcgtaatgaacatcctgcgtcgcaagatgcctgccgagaagattaacgaaaatctcgttcagcgcggcatcatgattttcgccattttgctggcctggagcgccatcgtactgaacgcaccggtgttgagcttcacctctatctgtagcccgattttcggcatggtagggtgcctgatcccggcgtggctggtttacaaagtaccagcattgcacaaatacaaagggatgtctctgtacctgattatcgtcactggtttgttgctgtgtgtttctccgttcctggcattttcttga

SEQ ID NO: 227 - protein, 422 aa, Escherichia coli
MSESEWREAIKFDSTDTGWVIMSIGMAIGAGIVFLPVQVGLMGLWVFLLSSVIGYPAMYLFQRLFINTLAESPECKDYPSVISGYLGKNWGILLGALYFVMLVIWMFVYSTAITNDSASYLHTFGVTEGLLSDSPFYGLVLICILVAISSRGEKLLFKISTGMVLTKLLVVAALGVSMVGMWHLYNVGSLPPLGLLVKNAIITLPFTLTSILFIQTLSPMVISYRSREKSIEVARHKALRAMNIAFGILFVTVFFYAVSFTLAMGHDEAVKAYEQNISALAIAAQFISGDGAAWVKVVSVILNIFAVMTAFFGVYLGFREATQGIVMNILRRKMPAEKINENLVQRGIMIFAILLAWSAIVLNAPVLSFTSICSPIFGMVGCLIPAWLVYKVPALHKYKGMSLYLIIVTGLLLCVSPFLAFS

SEQ ID NO: 228 - DNA, 1290 bp, Escherichia coli
  source 1..1290: mol_type = genomic DNA
atggaaacgactcaaaccagcacgattgcgtcgaaagactctcgtagtgcctggcgcaagacagacaccatgtggatgctgggcctttacggcacggcaatcggcgcgggcgtgctgttcctgccaatcaacgccggtgttggcggtatgatcccgctgatcatcatggctatccttgcgttcccgatgacgttttttgctcaccgcggcctgactcgcttcgtactgtctggtaaaaacccgggcgaagacatcaccgaggttgtagaagaacactttggtattggcgcaggtaaactgattaccctgctctacttcttcgctatctacccgatcctgctggtttatagcgtggcaatcaccaataccgttgaaagcttcatgtctcaccagctgggtatgacgccaccgccgcgtgcgattctgtcgctgatcctgatcgtgggtatgatgaccatcgttcgcttcggtgagcagatgatcgttaaagcgatgagtattctggtattcccgtttgttggcgtactgatgctgctggctctgtacctgatcccgcagtggaacggcgctgcactggaaacgctgtctctggacactgcatctgcaaccggaaacggtctgtggatgaccctgtggctggcaattccggtaatggtgttctcgttcaaccactctccgatcatctcttctttcgccgttgcgaagcgtgaagagtacggcgatatggcagaacagaaatgctcgaagatcctggcattcgcacacatcatgatggtgctgaccgtaatgttcttcgtcttcagctgtgtactgagcctgactccggcagacctggctgcggctaaagagcagaacatctcgattctgtcttacctggctaaccactttaacgcaccggttatcgcgtggatggctccgattatcgcgattatcgctatcaccaaatccttcctcggtcactacctgggcgcacgtgaaggcttcaacggtatggtgattaaatctctgcgtggtaaaggtaagtctatcgaaatcaacaagctgaaccgtatcactgcgctgttcatgctggtaacgacctggattgttgccaccctgaacccgagcatcctgggtatgattgaaaccctgggcggtccaatcatcgcgatgatcctgttcctgatgccgatgtacgcaattcagaaagtaccggcaatgcgtaagtacagcggtcacatcagcaacgtattcgttgtcgtgatgggtctgattgcaatctccgcaatcttctactctctgttcagctaa

SEQ ID NO: 229 - protein, 429 aa, Escherichia coli
METTQTSTIASKDSRSAWRKTDTMWMLGLYGTAIGAGVLFLPINAGVGGMIPLIIMAILAFPMTFFAHRGLTRFVLSGKNPGEDITEVVEEHFGIGAGKLITLLYFFAIYPILLVYSVAITNTVESFMSHQLGMTPPPRAILSLILIVGMMTIVRFGEQMIVKAMSILVFPFVGVLMLLALYLIPQWNGAALETLSLDTASATGNGLWMTLWLAIPVMVFSFNHSPIISSFAVAKREEYGDMAEQKCSKILAFAHIMMVLTVMFFVFSCVLSLTPADLAAAKEQNISILSYLANHFNAPVIAWMAPIIAIIAITKSFLGHYLGAREGFNGMVIKSLRGKGKSIEINKLNRITALFMLVTTWIVATLNPSILGMIETLGGPIIAMILFLMPMYAIQKVPAMRKYSGHISNVFVVVMGLIAISAIFYSLFS

SEQ ID NO: 230 - DNA, 1245 bp, Escherichia coli
  source 1..1245: mol_type = genomic DNA
atgactacgcaacgttcaccggggctattccggcgtctggctcatggcagcctggtaaaacaaatcctggtcggccttgttctggggattcttctggcatggatctcaaaacccgcggcggaagctgttggtctgttaggtactttgttcgtcggcgcactgaaagccgttgcccccatcctggtgttgatgctggtgatggcatctattgctaaccaccagcacgggcagaaaaccaatatccgccctattttgttcctctatctactgggcaccttctctgctgctctggccgcagtagtcttcagctttgccttcccttctaccctgcatttatccagtagcgcgggtgatatttcgccgccgtcaggcattgtcgaagtgatgcgcgggctggtaatgagcatggtttccaaccccatcgacgcgctgctgaaaggtaactacatcgggattctggtgtgggcgatcggcctcggcttcgcactgcgtcacggtaacgagaccaccaaaaacctggttaacgatatgtcgaatgccgttacctttatggtgaaactggtcattcgcttcgcaccgattggtatttttgggctggtttcttctaccctggcaaccaccggtttctccacactgtggggctacgcgcaactgctggtcgtgctggttggctgtatgttactggtggcgctggtggttaacccattgctggtgtggtggaaaattcgtcgtaacccgttcccgctggtgctgctgtgcctgcgcgaaagcggtgtgtatgccttcttcacccgcagctctgcagcgaacattccggtgaatatggcgctgtgtgaaaagctgaatctggatcgcgatacctattccgtttctattccgctgggagccaccatcaatatggcgggcgcagcaatcactattaccgtgttgacgctggctgcggttaatacgctgggtattccggtcgatctgcccacggcgctgctgttgagcgtggtggcttctctgtgtgcctgtggcgcatccggcgtggcgggggggtctctgctgctgatcccactggcctgtaatatgttcggtatttcgaacgatatcgccatgcaggtggttgccgtcggctttatcatcggcgtattgcaggactcttgcgaaaccgcgctgaactcttcaactgacgtgctgttcactgcggcagcttgccaggcagaagacgatcgtctggcaaatagcgccctgcgtaattaa

SEQ ID NO: 231 - protein, 414 aa, Escherichia coli
MTTQRSPGLFRRLAHGSLVKQILVGLVLGILLAWISKPAAEAVGLLGTLFVGALKAVAPILVLMLVMASIANHQHGQKTNIRPILFLYLLGTFSAALAAVVFSFAFPSTLHLSSSAGDISPPSGIVEVMRGLVMSMVSNPIDALLKGNYIGILVWAIGLGFALRHGNETTKNLVNDMSNAVTFMVKLVIRFAPIGIFGLVSSTLATTGFSTLWGYAQLLVVLVGCMLLVALVVNPLLVWWKIRRNPFPLVLLCLRESGVYAFFTRSSAANIPVNMALCEKLNLDRDTYSVSIPLGATINMAGAAITITVLTLAAVNTLGIPVDLPTALLLSVVASLCACGASGVAGGSLLLIPLACNMFGISNDIAMQVVAVGFIIGVLQDSCETALNSSTDVLFTAAACQAEDDRLANSALRN

SEQ ID NO: 232 - DNA, 1332 bp, Escherichia coli
  source 1..1332: mol_type = genomic DNA
atgagtacttcagatagcattgtatccagccagacaaaacaatcgtcctggcgtaaatcagataccacatggacgttaggcttgtttggtacggcaatcggcgccggggtgctgttcttccctatccgcgcaggttttggcggactgatcccgattcttctgatgttggtattggcataccccatcgcgttttattgccaccgggcgctggcgcgtctgtgtctttctggctctaacccttccggcaacattacggaaacggtggaagagcattttggtaaaactggcggcgtggttatcacgttcctgtacttcttcgcgatttgcccactgctgtggatttatggcgttactattaccaatacctttatgacgttctgggaaaaccagctcggctttgcaccgctgaatcgcggctttgtggcgctgttcctgttgctgctgatggctttcgtcatctggtttggtaaggatctgatggttaaagtgatgagctacctggtatggccgtttatcgccagcctggtgctgatttctttgtcgctgatcccttactggaactctgcagttatcgaccaggttgacctcggttcgctgtcgttaaccggtcatgacggtatcctgatcactgtctggctggggatttccatcatggttttctcctttaacttctcgccaatcgtctcttccttcgtggtttctaagcgtgaagagtatgagaaagacttcggtcgcgacttcaccgaacgtaaatgttcccaaatcatttctcgtgccagcatgctgatggttgcagtggtgatgttctttgcctttagctgcctgtttactctgtctccggccaacatggcggaagccaaagcgcagaatattccagtgctttcttatctggctaaccactttgcgtccatgaccggtaccaaaacaacgttcgcgattacactggaatatgcggcttccatcatcgcactcgtggctatcttcaaatctttcttcggtcactatctgggaacgctggaaggtctgaatggcctggtcctgaagtttggttataaaggcgacaaaactaaagtgtcgctgggtaaactgaacactatcagcatgatcttcatcatgggctccacctgggttgttgcctacgccaacccgaacatccttgacctgattgaagccatgggcgcaccgattatcgcatccctgctgtgcctgttgccgatgtatgccatccgtaaagcgccgtctctggcgaaataccgtggtcgtctggataacgtgtttgttaccgtgattggtctgctgaccatcctgaacatcgtatacaaactgttttaa

SEQ ID NO: 233 - protein, 443 aa, Escherichia coli
MSTSDSIVSSQTKQSSWRKSDTTWTLGLFGTAIGAGVLFFPIRAGFGGLIPILLMLVLAYPIAFYCHRALARLCLSGSNPSGNITETVEEHFGKTGGVVITFLYFFAICPLLWIYGVTITNTFMTFWENQLGFAPLNRGFVALFLLLLMAFVIWFGKDLMVKVMSYLVWPFIASLVLISLSLIPYWNSAVIDQVDLGSLSLTGHDGILITVWLGISIMVFSFNFSPIVSSFVVSKREEYEKDFGRDFTERKCSQIISRASMLMVAVVMFFAFSCLFTLSPANMAEAKAQNIPVLSYLANHFASMTGTKTTFAITLEYAASIIALVAIFKSFFGHYLGTLEGLNGLVLKFGYKGDKTKVSLGKLNTISMIFIMGSTWVVAYANPNILDLIEAMGAPIIASLLCLLPMYAIRKAPSLAKYRGRLDNVFVTVIGLLTILNIVYKLF

SEQ ID NO: 234 - DNA, 1191 bp, Escherichia coli
  source 1..1191: mol_type = genomic DNA
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 235 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 236 - DNA, 1104 bp, Escherichia coli
  source 1..1104: mol_type = genomic DNA
atgaaaaatgttggttttatcggctggcgcggtatggtcggctccgttctcatgcaacgcatggttgaagagcgcgacttcgacgccattcgccctgtcttcttttctacttctcagcttggccaggctgcgccgtcttttggcggaaccactggcacacttcaggatgcctttgatctggaggcgctaaaggccctcgatatcattgtgacctgtcagggcggcgattataccaacgaaatctatccaaagcttcgtgaaagcggatggcaaggttactggattgacgcagcatcgtctctgcgcatgaaagatgacgccatcatcattcttgaccccgtcaatcaggacgtcattaccgacggattaaataatggcatcaggacttttgttggcggtaactgtaccgtaagcctgatgttgatgtcgttgggtggtttattcgccaatgatcttgttgattgggtgtccgttgcaacctaccaggccgcttccggcggtggtgcgcgacatatgcgtgagttattaacccagatgggccatctgtatggccatgtggcagatgaactcgcgaccccgtcctctgctattctcgatatcgaacgcaaagtcacaaccttaacccgtagcggtgagctgccggtggataactttggcgtgccgctggcgggtagcctgattccgtggatcgacaaacagctcgataacggtcagagccgcgaagagtggaaagggcaggcggaaaccaacaagatcctcaacacatcttccgtaattccggtagatggtttatgtgtgcgtgtcggggcattgcgctgccacagccaggcattcactattaaattgaaaaaagatgtgtctattccgaccgtggaagaactgctggctgcgcacaatccgtgggcgaaagtcgttccgaacgatcgggaaatcactatgcgtgagctaaccccagctgccgttaccggcacgctgaccacgccggtaggccgcctgcgtaagctgaatatgggaccagagttcctgtcagcctttaccgtgggcgaccagctgctgtggggggccgcggagccgctgcgtcggatgcttcgtcaactggcgtaa

SEQ ID NO: 237 - protein, 367 aa, Escherichia coli
MKNVGFIGWRGMVGSVLMQRMVEERDFDAIRPVFFSTSQLGQAAPSFGGTTGTLQDAFDLEALKALDIIVTCQGGDYTNEIYPKLRESGWQGYWIDAASSLRMKDDAIIILDPVNQDVITDGLNNGIRTFVGGNCTVSLMLMSLGGLFANDLVDWVSVATYQAASGGGARHMRELLTQMGHLYGHVADELATPSSAILDIERKVTTLTRSGELPVDNFGVPLAGSLIPWIDKQLDNGQSREEWKGQAETNKILNTSSVIPVDGLCVRVGALRCHSQAFTIKLKKDVSIPTVEELLAAHNPWAKVVPNDREITMRELTPAAVTGTLTTPVGRLRKLNMGPEFLSAFTVGDQLLWGAAEPLRRMLRQLA

SEQ ID NO: 238 - DNA, 2463 bp, Escherichia coli
  source 1..2463: mol_type = genomic DNA
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccagaaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctga

SEQ ID NO: 239 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQKAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 240 - DNA, 933 bp, Escherichia coli
  source 1..933: mol_type = genomic DNA
atggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaa

SEQ ID NO: 241 - protein, 310 aa, Escherichia coli
MVKVYAPASSANMSVGFDVLGAAVTPVDGALLGDVVTVEAAETFSLNNLGRFADKLPSEPRENIVYQCWERFCQELGKQIPVAMTLEKNMPIGSGLGSSACSVVAALMAMNEHCGKPLNDTRLLALMGELEGRISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 242 - DNA, 954 bp, Corynebacterium glutamicum
  source 1..954: mol_type = genomic DNA
atgactgaaagtggatctaccgctagtccactttgtggcgttggatcatctgtcatgaccgaaactcaagaaacttaccaagcaaccactcgtgtgaagcgcggccttgccgacatgctcaagggtggtgtgatcatggatgtggtcacccctgaacaagcgcgcatcgccgaagatgcaggtgccagcgcagttatggcactcgagcgcgttcccgccgatatccgttctcagggcggcgttgctcgcatgagtgatcctgacctgatcgaaggaatcgtcaatgcggtctccatcccggtcatggcgaaagctcgcatcggtcacttcgtggaagctcaggttctggaagctctcggtgttgatttcatcgacgagtccgaagttctcagccctgccgactacacgcaccacatcaacaagtggaagttcgacgttcctttcgtctgtggcgcgaccaacctcggcgaagctttgcgacgcatcaccgaaggcgctgcaatgatccgttccaagggcgaagccggcaccggcgatgtctctgaagctgtccgtcacctgcgcaccatccgcggcgacatcaatcgcctgcgctccctggatgaggatgaactcttcgtcgccgccaaggaattccaggcaccatacgacctggtccgcgaagtcgcctccaccggcaagctccctgtggtcaccttcgttgcaggtggcgtcgcaaccccagccgacgctgcactcgtgcgccaaatgggcgccgaaggcgtctttgtcggctccggcatcttcaaatccggcaatccagccgcccgcgccgcagcgatcgtcaaggctgcaacgcttttcgacgacccctccgtcattgccgacgtatcccgcggcctgggtgaagccatggtgggcatcaacgtatccgacgtcccagcaccacaccgactcgccgagcgcggctggtga

SEQ ID NO: 243 - protein, 317 aa, Corynebacterium glutamicum
MTESGSTASPLCGVGSSVMTETQETYQATTRVKRGLADMLKGGVIMDVVTPEQARIAEDAGASAVMALERVPADIRSQGGVARMSDPDLIEGIVNAVSIPVMAKARIGHFVEAQVLEALGVDFIDESEVLSPADYTHHINKWKFDVPFVCGATNLGEALRRITEGAAMIRSKGEAGTGDVSEAVRHLRTIRGDINRLRSLDEDELFVAAKEFQAPYDLVREVASTGKLPVVTFVAGGVATPADAALVRQMGAEGVFVGSGIFKSGNPAARAAAIVKAATLFDDPSVIADVSRGLGEAMVGINVSDVPAPHRLAERGW

SEQ ID NO: 244 - DNA, 885 bp, Bacillus subtilis
  source 1..885: mol_type = genomic DNA
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggtaa

SEQ ID NO: 245 - protein, 294 aa, Bacillus subtilis
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 246 - DNA, 603 bp, Corynebacterium glutamicum
  source 1..603: mol_type = genomic DNA
atgatcgttggagttttagctctccagggcggggtggaagaacacctcaccgccttggaagctctcggagcgacgacccgaaaagtacgtgtgccaaaggaccttgatggtctcgaaggcatcgtcatccccggcggggaatccaccgtgttggacaaactggctcggacattcgacgtggtagaacctctagcgaatctcattcgcgacggcctacccgttttcgctacctgcgctggcctgatctatctggcgaaacacctcgacaacccagcaaggggacaacaaaccttggcggtagtggacgtggtggtgcgtcgaaacgcatttggcgcccaacgcgaatccttcgacaccaccgtggatgtttccttcgacggtgcaacattccccggagtgcaggcctcgtttatccgagctcccatcgtcactgcttttggtcctacggtagaagcgatcgctgctctcaacggtggggaggtggttggtgtacgccaaggcaacatcatcgcgctgtctttccatcccgaagaaaccggcgattaccgcatccaccaagcctggctggacctggtgagaaaacacgctgaactggcgatttga

SEQ ID NO: 247 - protein, 200 aa, Corynebacterium glutamicum
MIVGVLALQGGVEEHLTALEALGATTRKVRVPKDLDGLEGIVIPGGESTVLDKLARTFDVVEPLANLIRDGLPVFATCAGLIYLAKHLDNPARGQQTLAVVDVVVRRNAFGAQRESFDTTVDVSFDGATFPGVQASFIRAPIVTAFGPTVEAIAALNGGEVVGVRQGNIIALSFHPEETGDYRIHQAWLDLVRKHAELAI

SEQ ID NO: 248 - DNA, 591 bp, Bacillus subtilis
  source 1..591: mol_type = genomic DNA
atgttaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgtataa

SEQ ID NO: 249 - protein, 196 aa, Bacillus subtilis
MLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 250 - DNA, 1176 bp, Escherichia coli
  source 1..1176: mol_type = genomic DNA
atggatacgtcactggctgaggaagtacagcagaccatggcaacacttgcgcccaatcgctttttctttatgtcgccgtaccgcagttttacgacgtcaggatgtttcgcccgcttcgatgaaccggctgtgaacggggattcgcccgacagtcccttccagcaaaaactcgccgcgctgtttgccgatgccaaagcgcagggcatcaaaaatccggtgatggtcggggcgattcccttcgatccacgtcagccttcgtcgctgtatattcctgaatcctggcagtcgttctcccgtcaggaaaaacaagcttccgcacgccgtttcacccgcagccagtcgctgaatgtggtggaacgccaggcaattccggagcaaaccacgtttgaacagatggttgcccgcgccgccgcacttaccgccacgccgcaggtcgacaaagtggtgttgtcacggttgattgatatcaccactgacgccgccattgatagtggcgtattgctggaacggttgattgcgcaaaacccggttagttacaacttccatgttccgctggctgatggtggcgtcctgctgggggccagcccggaactgctgctacgtaaagacggcgagcgttttagctccattccgttagccggttccgcgcgtcgtcagccggatgaagtgctcgatcgcgaagcaggtaatcgtctgctggcgtcagaaaaagatcgccatgaacatgaactggtgactcaggcgatgaaagaggtactgcgcgaacgcagtagtgagttacacgttccttcttctccacagctgatcaccacgccgacgctgtggcatctcgcaactccctttgaaggtaaagcgaattcgcaagaaaacgcactgactctggcctgtctgctgcatccgacccccgcgctgagcggtttcccgcatcaggccgcgacccaggttattgctgaactggaaccgttcgaccgcgaactgtttggcggcattgtgggttggtgtgacagcgaaggtaacggcgaatgggtggtgaccatccgctgcgcgaagctgcgggaaaatcaggtgcgtctgtttgccggagcggggattgtgcctgcgtcgtcaccgttgggtgagtggcgcgaaacaggcgtcaaactttctaccatgttgaacgtttttggattgcattaa

SEQ ID NO: 251 - protein, 391 aa, Escherichia coli
MDTSLAEEVQQTMATLAPNRFFFMSPYRSFTTSGCFARFDEPAVNGDSPDSPFQQKLAALFADAKAQGIKNPVMVGAIPFDPRQPSSLYIPESWQSFSRQEKQASARRFTRSQSLNVVERQAIPEQTTFEQMVARAAALTATPQVDKVVLSRLIDITTDAAIDSGVLLERLIAQNPVSYNFHVPLADGGVLLGASPELLLRKDGERFSSIPLAGSARRQPDEVLDREAGNRLLASEKDRHEHELVTQAMKEVLRERSSELHVPSSPQLITTPTLWHLATPFEGKANSQENALTLACLLHPTPALSGFPHQAATQVIAELEPFDRELFGGIVGWCDSEGNGEWVVTIRCAKLRENQVRLFAGAGIVPASSPLGEWRETGVKLSTMLNVFGLH

SEQ ID NO: 252 - DNA, 945 bp, synthetic construct
  misc_feature 1..945: note = Synthetic construct
  source 1..945: mol_type = other DNA
aactgcggctattcctgaaagcaaaagtcctgttaatagaagggcgttgcggtagagcggggcgagtctcacaaatcagcttcctgttattaataaggttaagggcgtaatgacaaattcgacaaagcgcacaatccgtcccctcgcccctttggggagagggttagggtgaggggaacagccagcactggtgcgaacattaaccctcaccccagccctcaccctggaagggagagggggcagaacggcgcaggacatcacattgcgcttatgcgaatccatcaataatgcttctcattttcattgtaaccacaaccagatgcaaccccgagttgcagattgcgttacctcaagagttgacatagtgcgcgtttgcttttaggttagcgaccgaaaatataaatgataatcattattaaagcctttatcattttgtggaggatgatggagcgaggatgagcattccattcacccgctggccggaagagtttgcccgtcgctatcgggaaaaaggctactggcaggatttgccgctgaccgacattctgacgcgacatgctgcgagtgacagcatcgcggttatcgacggcgagcgacagttgagttatcgggagctgaatcaggcggcggataacctcgcgtgtagtttacgccgtcagggcattaaacctggtgaaaccgcgctggtacaactgggtaacgtcgctgaattgtatattacctttttcgcgctgctgaaactgggcgttgcgccggtgctggcgttgttcagccatcagcgtagtgaactgaacgcctatgccagccagattgaacccgcattgctgattgccgatcgccaacatgcgctgtttagcggggatgatttcctcaatactttcgtcacagaacattcctccattcgcgtggtgcaactgctcaacgacagcggtgagcataacttgc

SEQ ID NO: 253 - DNA, 1269 bp, Methanospirillum hungatei
  source 1..1269: mol_type = genomic DNA
atgaccggatatacgctcacctgtccggtatgtaacaaggagttttctgattcctacacccttacctgtccgggtgggtgccagggacttattcgggcgaaatatgctgcacgtcagataacgctccatgatgcaccgggggttttcaaatatatggactggcttccggtcacaggagtccttcgcacccgggctgaaccggtctgtttcaaaagcgaaggtcttgcacgggcattgggtctttcagatctctggattgtcttctccgggtactggcctgaagtgggggcctttgccgtgagcggttcatttaaggagtttgaagcattcccgacgatgcagcggctttccgagcggacgaagggaatcatccaggtttcctctgccgggaatacgggccgggcctttgccgaggtatcggcagagacctgtcagccggttatcattgtcgttcctgaatcagcacgcgaccggctattcacgacctcaccagcacatgataccctcctgatcaccatctccggagattatacggatgccatcaatctgggaagcaggatctgcaccctccctggcatattcccggaaggaggggcaaaaaatgttgccaggcgggatggcatgggcaccgtcatgctggcaggaacccttgcgatgggaacacttccagactggtatctgcaagcagtcgggagcggtaccgggggcatcgctgcatatgaggcctcgctcaggcttattgcagacggccggtttgggacaagaatgccccgccttcttctcttccagaacgaaccgttcattccgatggtcagagcctggcaggagaagagacgggagataaaagatgaggatatgcctgatgctgaacaagccatctcccaggtatattcagatgtcctgacaaaccggacacctccctatggcattgtcggaggggtatttgatacgctcatcgcaacaaacggacttatggccggtgtttcatcagcagatgcacaggaggcaggaaagctcttctcttcatctgaaggaatagatcctgatcctgctgctgcggtgtgtgtggcaggtctcatgagggccgttcgttcaggtgtaatcaagccggatgaaaagatcctccttaatatcaccgggggaggatacgccagagggagaaaagatctcccccggtttgtaaaagcgcctgatatcatggtatctaaagagacaccgtttgagaagatatctgcaaaggtgcaggaatggatgagatattatgcatga

SEQ ID NO: 254 - protein, 422 aa, Methanospirillum hungatei
MTGYTLTCPVCNKEFSDSYTLTCPGGCQGLIRAKYAARQITLHDAPGVFKYMDWLPVTGVLRTRAEPVCFKSEGLARALGLSDLWIVFSGYWPEVGAFAVSGSFKEFEAFPTMQRLSERTKGIIQVSSAGNTGRAFAEVSAETCQPVIIVVPESARDRLFTTSPAHDTLLITISGDYTDAINLGSRICTLPGIFPEGGAKNVARRDGMGTVMLAGTLAMGTLPDWYLQAVGSGTGGIAAYEASLRLIADGRFGTRMPRLLLFQNEPFIPMVRAWQEKRREIKDEDMPDAEQAISQVYSDVLTNRTPPYGIVGGVFDTLIATNGLMAGVSSADAQEAGKLFSSSEGIDPDPAAAVCVAGLMRAVRSGVIKPDEKILLNITGGGYARGRKDLPRFVKAPDIMVSKETPFEKISAKVQEWMRYYA

SEQ ID NO: 255 - DNA, 1281 bp, synthetic construct
  misc_feature 1..1281: note = Synthetic construct
  source 1..1281: mol_type = other DNA
atgcctcgccactatcagctgaaatgtcaggtttgtggtagccgttttgatgatgatggttttaccctggaatgtccgcatgaacatccgcctggtctgctggcagccgaatattttgcacgtgaatttaaaccggatctggccagcaaaggtatttttcgttatcagagctggctgcctgcactgcatgaaattggtagcgttggtagcaccgttacctttcagagcaaacaaattagccgtgttctgggtctgccgaatctgtggattacctttagcggttattggctggaaaaaggtgcaaccctggaaaccgcaacctttaaagaactggaagcatatagcgttctgagccgtctgtcaccggaacatcgtgaagttctggttgttgcaagcgcaggtaataccgcagcagcatttgcacatgtttgtagccagaatcaggttccgtgtctgattatcattccggcacgtggtctggcacgtatggaatttctggaagcactgcatccgtgtgttcgtgttgttagcctgaccggtctgaccgattatagtgatgcaattgcccaggcagatgaagttagccgtctggatggtttctttgccgaaggtggtgttaaaaacattggtcgtcgtgatggtattggcaccaccatgctgaatgcagttgaaacaatgggtcagctgccggattattactttcaggcaattggtagtggtgccggtggtattgcagtgcatgaagcagcaaaacgtctggtggatgatggtcgttttggtcagaaaatgccgcatctgatgctgagtcagaatctgccgtttgttccgatttatcaggcatggaaaagccagcgtcgtgaactgattaaagttaatcgtgatgtgggtaagaaacagacccagcagattgttgcagaagttctgagcaatcagcgtccgccttatagcattaaaggtggcgtttttgatgttctgatggaaagccagggtaatatgttagcagcagataatagcaaagcactgtatgccaccgatttctttcaggccagcgaaggtgttgatattgatccagcagccggtgttgcactggcaacactgaaagaagcagccagcaccggtcagctggacccggaagcagttattctgctgcatgttaccggtggtggtcgtaatcgtcgtcgtctgaatcataaactggttccggttaaaccgacactggaactggatgccggtgaaattggcacaaagaaagcactggaaaagattgttggtctgttttaa

SEQ ID NO: 256 - protein, 426 aa, unidentified
MPRHYQLKCQVCGSRFDDDGFTLECPHEHPPGLLAAEYFAREFKPDLASKGIFRYQSWLPALHEIGSVGSTVTFQSKQISRVLGLPNLWITFSGYWLEKGATLETATFKELEAYSVLSRLSPEHREVLVVASAGNTAAAFAHVCSQNQVPCLIIIPARGLARMEFLEALHPCVRVVSLTGLTDYSDAIAQADEVSRLDGFFAEGGVKNIGRRDGIGTTMLNAVETMGQLPDYYFQAIGSGAGGIAVHEAAKRLVDDGRFGQKMPHLMLSQNLPFVPIYQAWKSQRRELIKVNRDVGKKQTQQIVAEVLSNQRPPYSIKGGVFDVLMESQGNMLAADNSKALYATDFFQASEGVDIDPAAGVALATLKEAASTGQLDPEAVILLHVTGGGRNRRRLNHKLVPVKPTLELDAGEIGTKKALEKIVGLF

SEQ ID NO: 257 - DNA, 969 bp, synthetic construct
  misc_feature 1..969: note = Synthetic Construct
  source 1..969: mol_type = other DNA
atgcctaacattacctggtgcgacctgcctgaagatgtctctttatggccgggtctgcctctttcattaagtggtgatgaagtgatgccactggattaccacgcaggtcgtagcggctggctgctgtatggtcgtgggctggataaacaacgtctgacccaataccagagcaaactgggtgcggcgatggtgattgttgccgcctggtgcgtggaagattatcaggtgattcgtctggcaggttcactcaccgcacgggctacacgcctggcccacgaagcgcagctggatgtcgccccgctggggaaaatcccgcacctgcgcacgccgggtttgctggtgatggatatggactccaccgccatccagattgaatgtattaacgaaattgccaaactggccggaacgggcgagatggtggcggaagtaaccgaacgggcgatgcgcggcgaactcgattttaccgccagcctgcgcagccgtgtggcgacgctgaaaggcgctgacgccaatattctgcaacaggtgcgtgaaaatctgccgctgatgccaggcttaacgcaactggtgctcaagctggaaacgctgggctggaaagtggcgattgcctccggcggctttactttctttgctgaatacctgcgcgacaagctgcgcctgaccgccgtggtagccaatgaactggagatcatggacggtaaatttaccggcaatgtgatcggcgacatcgtagacgcgcagtacaaagcgaaaactctgactcgcctcgcgcaggagtatgaaatcccgctggcgcagaccgtggcgattggcgatggagccaatgacctgccgatgatcaaagcggcagggctggggattgcctaccatgccaagccaaaagtgaatgaaaaggcggaagtcaccatccgtcacgctgacctgatgggggtattctgcatcctctcaggcagcctgaatcagaagtaa

SEQ ID NO: 258 - protein, 322 aa, synthetic construct
  REGION 1..322: note = Synthetic Construct
MPNITWCDLPEDVSLWPGLPLSLSGDEVMPLDYHAGRSGWLLYGRGLDKQRLTQYQSKLGAAMVIVAAWCVEDYQVIRLAGSLTARATRLAHEAQLDVAPLGKIPHLRTPGLLVMDMDSTAIQIECINEIAKLAGTGEMVAEVTERAMRGELDFTASLRSRVATLKGADANILQQVRENLPLMPGLTQLVLKLETLGWKVAIASGGFTFFAEYLRDKLRLTAVVANELEIMDGKFTGNVIGDIVDAQYKAKTLTRLAQEYEIPLAQTVAIGDGANDLPMIKAAGLGIAYHAKPKVNEKAEVTIRHADLMGVFCILSGSLNQK

SEQ ID NO: 259 - DNA, 969 bp, synthetic construct
  misc_feature 1..969: note = Synthetic Construct
  source 1..969: mol_type = other DNA
atgcctaacattacctggtgcgacctgcctgaagatgtctctttatggccgggtctgcctctttcattaagtggtgatgaagtgatgccactggattaccacgcaggtcgtagcggctggctgctgtatggtcgtgggctggataaacaacgtctgacccaataccagagcaaactgggtgcggcgatggtgattgttgccgcctggtgcgtggaagattatcaggtgattcgtctggcaggttcactcaccgcacgggctacacgcctggcccacgaagcgcagctggatgtcgccccgctggggaaaatcccgcacctgcgcacgccgggtttgctggtgatggatatggactccaccgccatccagattgaatgtattgatgaaattgccaaactggccggaacgggcgagatggtggcggaagtaaccgaacgggcgacccgcggcgaactcgattttaccgccagcctgcgcagccgtgtggcgacgctgaaaggcgctgacgccaatattctgcaacaggtgcgtgaaaatctgccgctgatgccaggcttaacgcaactggtgctcaagctggaaacgctgggctggaaagtggcgattgcctccggcggctttactttctttgctgaatacctgcgcgacaagctgcgcctgaccgccgtggtagccaatgaactggagatcatggacggtaaatttaccggcaatgtgatcggcgacatcgtagacgcgcagtacaaagcgaaaactctgactcgcctcgcgcaggagtatgaaatcccgctggcgcagaccgtggcgattggcgatggagccaatgacctgccgatgatcaaagcggcagggctggggattgcctaccatgccaagccaaaagtgaatgaaaaggcggaagtcaccatccgtcacgctgacctgatgggggtattctgcatcctctcaggcagcctgaatcagaagtaa

SEQ ID NO: 260 - protein, 322 aa, synthetic construct
  REGION 1..322: note = Synthetic Construct
MPNITWCDLPEDVSLWPGLPLSLSGDEVMPLDYHAGRSGWLLYGRGLDKQRLTQYQSKLGAAMVIVAAWCVEDYQVIRLAGSLTARATRLAHEAQLDVAPLGKIPHLRTPGLLVMDMDSTAIQIECIDEIAKLAGTGEMVAEVTERATRGELDFTASLRSRVATLKGADANILQQVRENLPLMPGLTQLVLKLETLGWKVAIASGGFTFFAEYLRDKLRLTAVVANELEIMDGKFTGNVIGDIVDAQYKAKTLTRLAQEYEIPLAQTVAIGDGANDLPMIKAAGLGIAYHAKPKVNEKAEVTIRHADLMGVFCILSGSLNQK

SEQ ID NO: 261 - DNA, 969 bp, synthetic construct
  misc_feature 1..969: note = Synthetic Construct
  source 1..969: mol_type = other DNA
atgcctaacattacctggtgcgacctgcctgaagatgtctctttatggccgggtctgcctctttcattaagtggtgatgaagtgatgccactggattaccacgcaggtcgtagcggctggctgctgtatggtcgtgggctggataaacaacgtctgacccaataccagagcaaactgggtgcggcgatggtgattgttgccgcctggtgcgtggaagattatcaggtgattcgtctggcaggttcactcaccgcacgggctacacgcctggcccacgaagcgcagctggatgtcgccccgctggggaaaatcccgcacctgcgcacgccgggtttgctggtgatggatatggactccaccgccatccagattgaatgtattaacgaaattgccaaactggccggaacgggcgagatggtggcggaagtaaccgaacgggcgacccgcggcgaactcgattttaccgccagcctgcgcagccgtgtggcgacgctgaaaggcgctgacgccaatattctgcaacaggtgcgtgaaaatctgccgctgatgccaggcttaacgcaactggtgctcaagctggaaacgctgggctggaaagtggcgattgcctccggcggctttactttctttgctgaatacctgcgcgacaagctgcgcctgaccgccgtggtagccaatgaactggagatcatggacggtaaatttaccggcaatgtgatcggcgacatcgtagacgcgcagtacaaagcgaaaactctgactcgcctcgcgcaggagtatgaaatcccgctggcgcagaccgtggcgattggcgatggagccaatgacctgccgatgatcaaagcggcagggctggggattgcctaccatgccaagccaaaagtgaatgaaaaggcggaagtcaccatccgtcacgctgacctgatgggggtattctgcatcctctcaggcagcctgaatcagaagtaa

SEQ ID NO: 262 - protein, 322 aa, synthetic construct
  REGION 1..322: note = Synthetic Construct
MPNITWCDLPEDVSLWPGLPLSLSGDEVMPLDYHAGRSGWLLYGRGLDKQRLTQYQSKLGAAMVIVAAWCVEDYQVIRLAGSLTARATRLAHEAQLDVAPLGKIPHLRTPGLLVMDMDSTAIQIECINEIAKLAGTGEMVAEVTERATRGELDFTASLRSRVATLKGADANILQQVRENLPLMPGLTQLVLKLETLGWKVAIASGGFTFFAEYLRDKLRLTAVVANELEIMDGKFTGNVIGDIVDAQYKAKTLTRLAQEYEIPLAQTVAIGDGANDLPMIKAAGLGIAYHAKPKVNEKAEVTIRHADLMGVFCILSGSLNQK